SEQUENCE LISTING (114 sequences)

SEQ ID NO: 1 - DNA, 717 bp, Artificial Sequence
  Description of Artificial Sequence Synthetic polynucleotide
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaagccctgtacctgctccggagggaccagaggctgccccccgatgcccacaagccccctgggggaggcagtttccggacccccatccaagaggagcaggccgacgcccactccaccctggccaagatc

SEQ ID NO: 2 - DNA, 450 bp, Artificial Sequence
  Description of Artificial Sequence Synthetic polynucleotide
atggcccgcggaagtcttcgccgtttgctccgtcttcttgttctcggcctgtggctcgctctgctccggagtgtagcgggcgaacaagcacctgggactgcaccgtgttcacggggctcctcatggtccgccgatcttgataaatgtatggattgtgccagctgtagagccaggccccattctgatttctgtcttgggtgtgccgctgccccaccggcaccttttagacttctgtggcctattctgggcggagccctctcattgacatttgtccttggacttctctccgggttccttgtatggcggcggtgtcggcgccgcgaagccctgtacctgctccggagggaccagaggctgccccccgatgcccacaagccccctgggggaggcagtttccggacccccatccaagaggagcaggccgacgcccactccaccctggccaagatc

SEQ ID NO: 3 - DNA, 891 bp, Artificial Sequence
  Description of Artificial Sequence Synthetic polynucleotide
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcgccctgtacctgctccggagggaccagaggctgccccccgatgcccacaagccccctgggggaggcagtttccggacccccatccaagaggagcaggccgacgcccactccaccctggccaagatc

SEQ ID NO: 4 - DNA, 657 bp, Artificial Sequence
  Description of Artificial Sequence Synthetic polynucleotide
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgccccca

SEQ ID NO: 5 - DNA, 324 bp, Artificial Sequence
  Description of Artificial Sequence Synthetic polynucleotide
atggcccgcggaagtcttcgccgtttgctccgtcttcttgttctcggcctgtggctcgctctgctccggagtgtagcgggcgaacaagcacctgggactgcaccgtgttcacggggctcctcatggtccgccgatcttgataaatgtatggattgtgccagctgtagagccaggccccattctgatttctgtcttgggtgtgccgctgccccaccggcaccttttagacttctgtggcctattctgggcggagccctctcattgacatttgtccttggacttctctccgggttccttgtatggcggcggtgtcggcgccgcgaa

SEQ ID NO: 6 - DNA, 801 bp, Artificial Sequence
  Description of Artificial Sequence Synthetic polynucleotide
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccaccta

SEQ ID NO: 7 - DNA, 1521 bp, Artificial Sequence
  Description of Artificial Sequence Synthetic polynucleotide
atggcgggtgcgggcccgaagcggcgcgcgctagcggcgccggcggccgaggagaaggaagaggcgcgggagaagatgctggccgccaagagcgcggacggctcggcgccggcaggcgagggcgagggcgtgaccctgcagcggaacatcacgctgctcaacggcgtggccatcatcgtggggaccattatcggctcgggcatcttcgtgacgcccacgggcgtgctcaaggaggcaggctcgccggggctggcgctggtggtgtgggccgcgtgcggcgtcttctccatcgtgggcgcgctctgctacgcggagctcggcaccaccatctccaaatcgggcggcgactacgcctacatgctggaggtctacggctcgctgcccgccttcctcaagctctggatcgagctgctcatcatccggccttcatcgcagtacatcgtggccctggtcttcgccacctacctgctcaagccgctcttccccacctgcccggtgcccgaggaggcagccaagctcgtggcctgcctctgcgtgctgctgctcacggccgtgaactgctacagcgtgaaggccgccacccgggtccaggatgcctttgccgccgccaagctcctggccctggccctgatcatcctgctgggcttcgtccagatcgggaagggtgatgtgtccaatctagatcccaacttctcatttgaaggcaccaaactggatgtggggaacattgtgctggcattatacagcggcctctttgcctatggaggatggaattacttgaatttcgtcacagaggaaatgatcaacccctacagaaacctgcccctggccatcatcatctccctgcccatcgtgacgctggtgtacgtgctgaccaacctggcctacttcaccaccctgtccaccgagcagatgctgtcgtccgaggccgtggccgtggacttcgggaactatcacctgggcgtcatgtcctggatcatccccgtcttcgtgggcctgtcctgcttcggctccgtcaatgggtccctgttcacatcctccaggctcttcttcgtggggtcccgggaaggccacctgccctccatcctctccatgatccacccacagctcctcacccccgtgccgtccctcgtgttcacgtgtgtgatgacgctgctctacgccttctccaaggacatcttctccgtcatcaacttcttcagcttcttcaactggctctgcgtggccctggccatcatcggcatgatctggctgcgccacagaaagcctgagcttgagcggcccatcaaggtgaacctggccctgcctgtgttcttcatcctggcctgcctcttcctgatcgccgtctccttctggaagacacccgtggagtgtggcatcggcttcaccatcatcctcagcgggctgcccgtctacttcttcggggtctggtggaaaaacaagcccaagtggctcctccagggcatcttctccacgaccgtcctgtgtcagaagctcatgcaggtggtcccccaggagaca

SEQ ID NO: 8 - DNA, 1566 bp, Artificial Sequence
  Description of Artificial Sequence Synthetic polynucleotide
atgtgggaagcccaatttctcggcctgcttttcttgcaacccctgtgggttgcgcctgtcaaacccctgcaacctggtgccgaagtgcccgtcgtttgggcacaagaaggagcaccagctcaacttccatgttcccctaccattcctcttcaagaccttagtctgttgcgccgggccggagttacgtggcaacaccaacccgattccggaccaccagcggctgcacctggacacccattggcgcctgggccacatccagccgccccgtctagctggggacctagacctagacggtatacagtattgtccgtcggccctggtggactccggtctggtcgactcccgcttcaaccaagagtgcaactcgacgaacgcgggagacaacgtggagattttagcctgtggttgcgtcctgcgagacgtgccgatgctggggaatatcgtgcagccgtccatttgcgagatagagctctttcatgtcggctgcgcctcaggctcggtcaagcaagcatgaccgcgtccccgccgggttcactgcgcgcttcagattgggtgatcctcaattgtagcttttccagaccagatagacccgcttcagtacactggtttaggaatcgtggtcaagggcgcgttccggtacgcgaatctcctcaccatcatctggctgagtcctttctgtttcttccacaggtgtcccctatggattccggaccttggggttgtattcttacatatcgggacggttttaatgtgtcaattatgtacaatctgaccgtcttggggctcgaaccacccacgccgctgactgtatatgccggcgccggatcacgagttggccttccatgtagattgcccgccggagtcggcacgaggtcatttctgaccgctaaatggaccccacccggtggtggaccagatttgttggtaaccggcgataacggagatttcacactcagacttgaagacgtatctcaagctcaagccggaacatatacttgtcacatacacttgcaagagcaacaattgaacgcgaccgttacgctggctattattactgtaacgcctaagtcattcggttctcccgggtcattgggcaaacttctctgcgaagttacgcctgtcagcggccaggagcggttcgtttggtccagcttggatactcctagccaacgaagcttttctggtccctggcttgaagcccaagaagcacaactgctgtcacaaccctggcagtgtcaactttatcaaggcgaacgcctgctgggtgccgcggtatattttacggaactttcctcacccggcgcacagcgttcaggacgggcaccagcgcccgctcgcgaacccggccattctcctcaaattatatcattcttcctcgcacttaccagtacggcccttctctttcttttgttctttctgactcttcgcttttcagtggtgaagcgaggtcgcaagaagctgctctacatctttaagcagcctttcatgcggcccgtgcagacgacccaggaagaggacggttgctcatgtagattccctgaggaagaagagggcggctgcgagttg

SEQ ID NO: 9 - DNA, 2421 bp, Artificial Sequence
  Description of Artificial Sequence Synthetic polynucleotide
atggaacaacggggacagaacgccccggccgcttcgggggcccggaaaaggcacggcccaggacccagggaggcgcggggagccaggcctgggccccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagtcccagctgtggaggagacggtgacctccagcccagggactcctgcctctccctgttctctctcaggcatcatcataggagtcacagttgcagccgtagtcttgattgtggctgtgtttgtttgcaagtctttactgtggaagaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctct

SEQ ID NO: 10 - DNA, 2514 bp, Artificial Sequence
  Description of Artificial Sequence Synthetic polynucleotide
atggctccacccccggctagagttcacctcggcgcttttcttgctgtcacacctaacccaggttcagccgcaagcggaactgaagctgcggcagctactccttctaaggtttggggaagcagcgctggtcgcatcgaaccccggggtggtggtaggggtgctcttccgacatctatgggtcaacatggtccttcagctcgagcaagggccggaagagcaccggggccacggcctgcccgtgaggctagtccccgcctgcgagtacataaaacatttaaattcgtggtagtgggagttcttcttcaagttgtgccaagtagtgccgctactattaagctccacgaccagagcattgggacccaacagtgggagcacagtccacttggcgaactgtgcccacccggcagtcaccgctctgagcaccccggggcgtgcaatcgatgtactgaaggcgtaggctatacgaacgcatcaaataacctgttcgcctgtcttccctgcaccgcctgcaagtccgacgaggaagaaaggtcaccatgtactacaacacgcaataccgcctgccaatgtaagcccgggacatttcgcaacgataactcagccgaaatgtgtcgtaaatgttctaggggatgtccaaggggcatggtaaaagtgaaagactgcacaccttggagcgatatagaatgcgttcacaaggagtccggaaacggtcacaacatttgggtcatccttgtcgtcaccctcgtggtacctctgcttctggtcgcagtcctcatcgtttgctgctgtattggatccggatgcggcaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctct

SEQ ID NO: 11 - DNA, 2424 bp, Artificial Sequence
  Description of Artificial Sequence Synthetic polynucleotide
atgggcctctccaccgtgcctgacctgctgctgccactggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctct

SEQ ID NO: 12 - DNA, 2472 bp, Artificial Sequence
  Description of Artificial Sequence Synthetic polynucleotide
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaagagccacccttctctctgcaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctct

SEQ ID NO: 13 - DNA, 876 bp, Artificial Sequence
  Description of Artificial Sequence Synthetic polynucleotide
atggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgacccta

SEQ ID NO: 14 - DNA, 1536 bp, Artificial Sequence
  Description of Artificial Sequence Synthetic polynucleotide
atgtgggaagcacaatttctcggactcctcttccttcaacctctgtgggtcgcacccgttaaacccctgcaacccggcgccgaagtacctgtcgtatgggctcaagaaggagcaccggcgcaacttccgtgttcaccaactattcctctgcaagacttgtctctcttgaggcgggcaggagtgacctggcaacaccaacccgattccggaccccctgcagcagctccaggacacccactcgcgcctgggccccatcctgctgccccgtcttcttggggacctcgccctaggagatataccgtccttagtgtaggcccaggcggattgagatctggtcgacttccgctccaacctcgagttcaattggacgaacggggacgccaaaggggtgacttttcactctggctcagacctgcacgccgggctgatgctggagaatatcgtgctgccgttcatcttcgggatcgtgcgttgtcatgtcgtctgcggctccgtctcggacaagcttctatgacagcttctccgcccggcagcctgcgggcttctgattgggtgatcctcaattgttcttttagtcgacccgatagacccgcttcagttcactggtttcgcaataggggacaaggacgtgtgcccgtgagggaaagtcctcaccatcatcttgctgagtcttttctgtttctgccgcaggtgtctccaatggatagtggcccatggggttgtattttgacgtatagggacgggtttaatgtaagtataatgtacaatttgacagtcttggggcttgaaccaccgacccctctgaccgtttatgcaggcgcggggtctcgcgtcggactcccttgtcgacttccagcaggcgtcggcacaagatcctttctgacagcaaaatggacgccaccaggtggcggtccagatttgctcgtcacaggcgataacggagatttcacactcagactcgaagacgtaagtcaagcacaagcaggcacatatacgtgtcacattcacttgcaagagcaacaactgaacgctaccgtaaccctggccattattactgttacccctaagagtttcggtagcccaggcagccttggcaaactcctctgcgaagtcacgcccgtgtcaggccaggagcggttcgtttggtccagtcttgatacaccgtctcaaagatcttttagtggtccatggctcgaagcccaagaagctcaacttttgtcacaaccatggcagtgtcaactttatcaaggagaacgcctgttgggcgccgctgtctattttaccgaacttagttctcccggggcacagcgaagcggaagggctcacatctacgagtcccagctctgctgtcaactcaaattttggctgccaattggctgcgcggctttcgtcgtcgtgtgtatcctgggctgtatcctgatctgctggctgacgaagaagaaatactcctcaagcgtccatgatccaaatggagagtatatgtttatgcgagctgtcaatacggcgaagaagtcacgactgaccgacgttacattg

SEQ ID NO: 15 - DNA, 375 bp, Artificial Sequence
  Description of Artificial Sequence Synthetic polynucleotide
atgcctcacagctccgacagcagtgactccagcttcagccgctctcctccccctggcaaacaggactcatctgatgatgtgagaagagttcagaggagggagaaaaatcgtattgccgcccagaagagccgacagaggcagacacagaaggccgacaccctgcacctggagagcgaagacctggagaaacagaacgcggctctacgcaaggagatcaagcagctcacagaggaactgaagtacttcacgtcggtgctgaacagccacgagcccctgtgctcggtgctggccgccagcacgccctcgccccccgaggtggtgtacagcgcccacgcattccaccaacctcatgtcagctccccgcgcttccagccc

SEQ ID NO: 16 - DNA, 381 bp, Artificial Sequence
  Description of Artificial Sequence Synthetic polynucleotide
atgtcgcaagggctcccggccgccggcagcgtcctgcagaggagcgtcgcggcgcccgggaaccagccgcagccgcagccgcagcagcagagccctgaggatgatgacaggaaggtccgaaggagagaaaaaaaccgagttgctgctcagagaagtcggaagaagcagacccagaaggctgacaagctccatgaggaatatgagagcctggagcaagaaaacaccatgctgcggagagagatcgggaagctgacagaggagctgaagcacctgacagaggcactgaaggagcacgagaagatgtgcccgctgctgctctgccctatgaactttgtgccagtgcctccccggccggaccctgtggccggctgcttgccccga

SEQ ID NO: 17 - DNA, 381 bp, Artificial Sequence; identical to SEQ ID NO: 16
  Description of Artificial Sequence Synthetic polynucleotide

SEQ ID NO: 18 - DNA, 402 bp, Artificial Sequence
  Description of Artificial Sequence Synthetic polynucleotide
atgaaagccttcagtcccgtgaggtccgttaggaaaaacagcctgtcggaccacagcctgggcatctcccggagcaaaacccctgtggacgacccgatgagcctgctatacaacatgaacgactgctactccaagctcaaggagctggtgcccagcatcccccagaacaagaaggtgagcaagatggaaatcctgcagcacgtcatcgactacatcttggacctgcagatcgccctggactcgcatcccactattgtcagcctgcatcaccagagacccgggcagaaccaggcgtccaggacgccgctgaccaccctcaacacggatatcagcatcctgtccttgcaggcttctgaattcccttctgagttaatgtcaaatgacagcaaagcactgtgtggc

SEQ ID NO: 19 - DNA, 357 bp, Artificial Sequence
  Description of Artificial Sequence Synthetic polynucleotide
atgaaggcgctgagcccggtgcgcggctgctacgaggcggtgtgctgcctgtcggaacgcagtctggccatcgcccggggccgagggaagggcccggcagctgaggagccgctgagcttgctggacgacatgaaccactgctactcccgcctgcgggaactggtacccggagtcccgagaggcactcagcttagccaggtggaaatcctacagcgcgtcatcgactacattctcgacctgcaggtagtcctggccgagccagcccctggaccccctgatggcccccaccttcccatccagacagccgagctcactccggaacttgtcatctccaacgacaaaaggagcttttgccac

SEQ ID NO: 20 - DNA, 1278 bp, Artificial Sequence
  Description of Artificial Sequence Synthetic polynucleotide
atgtgtgaccggaatggtggtcggcggcttcgacagtggctgatcgagcagattgacagtagcatgtatccaggactgatttgggagaatgaggagaagagcatgttccggatcccttggaaacacgctggcaagcaagattataatcaggaagtggatgcctccatttttaaggcctgggcagtttttaaagggaagtttaaagaaggggacaaagctgaaccagccacttggaagacgaggttacgctgtgctttgaataagagcccagattttgaggaagtgacggaccggtcccaactggacatttccgagccatacaaagtttaccgaattgttcctgaggaagagcaaaaatgcaaactaggcgtggcaactgctggctgcgtgaatgaagttacagagatggagtgcggtcgctctgaaatcgacgagctgatcaaggagccttctgtggacgattacatggggatgatcaaaaggagcccttccccgccggaggcctgtcggagtcagctccttccagactggtgggcgcagcagcccagcacaggcgtgccgctggtgacggggtacaccacctacgacgcgcaccattcagcattctcccagatggtgatcagcttctactatgggggcaagctggtgggccaggccaccaccacctgccccgagggctgccgcctgtccctgagccagcctgggctgcccggcaccaagctgtatgggcccgagggcctggagctggtgcgcttcccgccggccgacgccatccccagcgagcgacagaggcaggtgacgcggaagctgttcgggcacctggagcgcggggtgctgctgcacagcagccggcagggcgtgttcgtcaagcggctgtgccagggccgcgtgttctgcagcggcaacgccgtggtgtgcaaaggcaggcccaacaagctggagcgtgatgaggtggtccaggtcttcgacaccagccagttcttccgagagctgcagcagttctataacagccagggccggcttcctgacggcagggtggtgctgtgctttggggaagagtttccggatatggcccccttgcgctccaaactcattctcgtgcagattgagcagctgtatgtccggcaactggcagaagaggctgggaagagctgtggagccggctctgtgatgcaggcccccgaggagccgccgccagaccaggtcttccggatgtttccagatatttgtgcctcacaccagagatcatttttcagagaaaaccaacagatcaccgtc

SEQ ID NO: 21 - DNA, 1317 bp, Artificial Sequence
  Description of Artificial Sequence Synthetic polynucleotide
atgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcg

SEQ ID NO: 22 - DNA, 2298 bp, Artificial Sequence
  Description of Artificial Sequence Synthetic polynucleotide
atggcggacggaggagcagcgagtcaagatgagagttcagccgcggcggcagcagcagcagactcaagaatgaacaatccgtcagaaaccagtaaaccatctatggagagtggagatggcaacacaggcacacaaaccaatggtctggactttcagaagcagcctgtgcctgtaggaggagcaatctcaacagcccaggcgcaggctttccttggacatctccatcaggtccaactcgctggaacaagtttacaggctgctgctcagtctttaaatgtacagtctaaatctaatgaagaatcgggggattcgcagcagccaagccagccttcccagcagccttcagtgcaggcagccattccccagacccagcttatgctagctggaggacagataactgggcttactttgacgcctgcccagcaacagttactactccagcaggcacaggcacaggcacagctgctggctgctgcagtgcagcagcactccgccagccagcagcacagtgctgctggagccaccatctccgcctctgctgccacgcccatgacgcagatccccctgtctcagcccatacagatcgcacaggatcttcaacaactgcaacagcttcaacagcagaatctcaacctgcaacagtttgtgttggtgcatccaaccaccaatttgcagccagcgcagtttatcatctcacagacgccccagggccagcagggtctcctgcaagcgcaaaatcttctaacgcaactacctcagcaaagccaagccaacctcctacagtcgcagccaagcatcaccctcacctcccagccagcaaccccaacacgcacaatagcagcaaccccaattcagacacttccacagagccagtcaacaccaaagcgaattgatactcccagcttggaggagcccagtgaccttgaggagcttgagcagtttgccaagaccttcaaacaaagacgaatcaaacttggattcactcagggtgatgttgggctcgctatggggaaactatatggaaatgacttcagccaaactaccatctctcgatttgaagccttgaacctcagctttaagaacatgtgcaagttgaagccacttttagagaagtggctaaatgatgcagagaacctctcatctgattcgtccctctccagcccaagtgccctgaattctccaggaattgagggcttgagccgtaggaggaagaaacgcaccagcatagagaccaacatccgtgtggccttagagaagagtttcttggagaatcaaaagcctacctcggaagagatcactatgattgctgatcagctcaatatggaaaaagaggtgattcgtgtttggttctgtaaccgccgccagaaagaaaaaagaatcaacccaccaagcagtggtgggaccagcagctcacctattaaagcaattttccccagcccaacttcactggtggcgaccacaccaagccttgtgactagcagtgcagcaactaccctcacagtcagccctgtcctccctctgaccagtgctgctgtgacgaatctttcagttacaggcacttcagacaccacctccaacaacacagcaaccgtgatttccacagcgcctccagcttcctcagcagtcacgtccccctctctgagtccctccccttctgcctcagcctccacctccgaggcatccagtgccagtgagaccagcacaacacagaccacctccactcctttgtcctcccctcttgggaccagccaggtgatggtgacagcatcaggtttgcaaacagcagcagctgctgcccttcaaggagctgcacagttgccagcaaatgccagtcttgctgccatggcagctgctgcaggactaaacccaagcctgatggcaccctcacagtttgcggctggaggtgccttactcagtctgaatccagggaccctgagcggtgctctcagcccagctctaatgagcaacagtacactggcaactattcaagctcttgcttctggtggctctcttccaataacatcacttgatgcaactgggaacctggtatttgccaatgcgggaggagcccccaacatcgtgactgcccctctgttcctgaaccctcagaacctctctctgctcaccagcaaccctgttagcttggtctctgccgccgcagcatctgcagggaactctgcacctgtagccagccttcacgccacctccacctctgctgagtccatccagaactctctcttcacagtggcctctgccagcggggctgcgtccaccaccaccaccgcctccaaggcacag

SEQ ID NO: 23 - DNA, 1014 bp, Artificial Sequence
  Description of Artificial Sequence Synthetic polynucleotide
atggagtatttcatggtgcccactcagaaggtgccctctttgcaacatttcaggaaaacagagaaagaagtgataggagggctctgtagccttgccaacattccactaacccccgagactcagcgggaccaggagcggcggattcggcgggagatcgccaacagcaacgagcggagacgcatgcagagcatcaacgcgggattccagtccctcaagaccctcatcccccacacagacggagagaagctcagcaaggcagccattctccagcagacagccgagtacatcttctccctggagcaggagaagaccaggctcttgcagcagaacacacagctcaagcgcttcatccaggagctgagcggctcgtcccccaagcgacggcgggcagaggacaaggacgaaggcataggctccccggacatctgggaggacgagaaggcggaggacctgcggcgggagatgattgagctgcggcagcagctggacaaggagcgctcggtgcgcatgatgctggaggagcaggtgcgctcgctggaggcccacatgtacccggaaaagctcaaggtgattgcgcagcaggtgcagctgcagcagcagcaggaacaggtgaggctgctgcaccaggagaagctggagcgggaacagcagcagctgcggacccagcttctgccccctccggcccccacccaccaccccacggtgatcgtgccagcaccgcctcctcctccctcccaccacatcaatgtcgtcaccatgggcccctcctcggtcatcaactctgtttccacatcccggcaaaatctggacaccatcgtgcaggcaatccagcacatcgagggcacccaggaaaagcaggagctggaggaggagcagcggcgagctgtcatcgtgaagcctgtccgcagctgcccggaggcccccacctctgacaccgcctccgactccgaggcctcagacagtgacgccatggaccagagccgggaggagccgtcgggggacggggagcttccc

SEQ ID NO: 24 - DNA, 1656 bp, Artificial Sequence
  Description of Artificial Sequence Synthetic polynucleotide
atggacaatatgtctattacgaatacaccaacaagtaatgatgcctgtctgagcattgtgcatagtttgatgtgccatagacaaggtggagagagtgaaacatttgcaaaaagagcaattgaaagtttggtaaagaagctgaaggagaaaaaagatgaattggattctttaataacagctataactacaaatggagctcatcctagtaaatgtgttaccatacagagaacattggatgggaggcttcaggtggctggtcggaaaggatttcctcatgtgatctatgcccgtctctggaggtggcctgatcttcacaaaaatgaactaaaacatgttaaatattgtcagtatgcgtttgacttaaaatgtgatagtgtctgtgtgaatccatatcactacgaacgagttgtatcacctggaattgatctctcaggattaacactgcagagtaatgctccatcaagtatgatggtgaaggatgaatatgtgcatgactttgagggacagccatcgttgtccactgaaggacattcaattcaaaccatccagcatccaccaagtaatcgtgcatcgacagagacatacagcaccccagctctgttagccccatctgagtctaatgctaccagcactgccaactttcccaacattcctgtggcttccacaagtcagcctgccagtatactggggggcagccatagtgaaggactgttgcagatagcatcagggcctcagccaggacagcagcagaatggatttactggtcagccagctacttaccatcataacagcactaccacctggactggaagtaggactgcaccatacacacctaatttgcctcaccaccaaaacggccatcttcagcaccacccgcctatgccgccccatcccggacattactggcctgttcacaatgagcttgcattccagcctcccatttccaatcatcctgctcctgagtattggtgttccattgcttactttgaaatggatgttcaggtaggagagacatttaaggttccttcaagctgccctattgttactgttgatggatacgtggacccttctggaggagatcgcttttgtttgggtcaactctccaatgtccacaggacagaagccattgagagagcaaggttgcacataggcaaaggtgtgcagttggaatgtaaaggtgaaggtgatgtttgggtcaggtgccttagtgaccacgcggtctttgtacagagttactacttagacagagaagctgggcgtgcacctggagatgctgttcataagatctacccaagtgcatatataaaggtctttgatttgcgtcagtgtcatcgacagatgcagcagcaggcggctactgcacaagctgcagcagctgcccaggcagcagccgtggcaggaaacatccctggcccaggatcagtaggtggaatagctccagctatcagtctgtcagctgctgctggaattggtgttgatgaccttcgtcgcttatgcatactcaggatgagttttgtgaaaggctggggaccggattacccaagacagagcatcaaagaaacaccttgctggattgaaattcacttacaccgggccctccagctcctagacgaagtacttcataccatgccgattgcagacccacaacctttagac

SEQ ID NO: 25 - DNA, 2829 bp, Artificial Sequence
  Description of Artificial Sequence Synthetic polynucleotide
atgcccagcacttcattccccgtgccctctaaattccccctgggtcccgcagccgccgtatttggtcgcggtgagaccctgggcccagcaccaagagcaggtggtactatgaaaagtgcagaagaggagcattacggatacgccagtagcaatgtgtcaccagctctcccactgcctactgcccatagcacgctccctgcgccttgtcataatctgcaaacatctacgcctggaattatacccccagccgaccatccatctggctatggcgccgcactggatggtggcccagccgggtattttctgtcatcagggcatactcgtccggacggagcaccagcactcgaatccccgcggattgaaatcactagctgtctgggactctatcataataacaatcaattctttcatgacgtagaagtcgaggatgtactgccctctagcaagaggtcaccaagcaccgctactctttctctcccatccttggaagcatatagggatccaagttgtctctctcccgcttcctcacttagcagtagaagttgtaatagcgaagcaagcagctatgaatcaaattatagctatccctatgcatcaccacaaacaagtccctggcaatccccatgtgtttcccctaaaacgactgatccagaagaaggattcccaaggggacttggagcttgtacgctccttggatcaccccgccatagtcctagtacttcaccacgagcatccgtaacagaagaatcctggctcggcgcgagaagcagtcggccggcctcaccatgtaataaacggaaatattctcttaatggtaggcaaccaccatatagtcctcatcattcccctacccctagcccccatggatctcccagagtgtcagtcactgatgattcttggctcgggaatacaacgcaatatacatcctcagcaattgtcgcggctattaatgctctcacgacagattccagtctcgatctcggggacggagtgcccgtgaaaagccggaaaacaacactcgaacaacccccatctgtcgcacttaaagtcgaacctgtaggagaagatctcggaagtccaccaccgcctgctgattttgcccctgaggattattctagttttcaacatattcgcaaaggtgggttttgtgatcaatatttggccgtccctcaacatccttatcaatgggccaaacctaaaccgctcagccccaccagctatatgtctcccacgttgccagcacttgattggcaactcccaagccattccgggccatacgaactccgaatcgaagtccaaccgaaatcacatcatcgcgcacattatgaaactgaagggtcacgtggcgctgtaaaagcgtccgctggcgggcatccaattgtccaactccacgggtatctggaaaacgaacctttgatgcttcaactctttatcggaaccgcagatgatcgacttctccggccacatgcattttatcaagttcatcggattaccggaaagacagtaagtacgacttctcatgaagcaatactgagtaatactaaggtgctcgaaattccccttctcccagaaaatagtatgagagctgtgatcgattgcgcaggtattctcaagttgaggaattctgatatcgagctcaggaagggcgaaacagatattggacgtaagaatacgcgcgtgcgactcgtctttcgggtgcatgtacctcagcctagtgggcggactctcagccttcaagttgcaagtaatccgattgagtgtagccaaagaagtgcccaagaattgccgttggtcgaaaagcaatctactgattcctaccctgtagttggtggcaagaagatggtactctcaggacataattttctccaagattctaaagtgatctttgtcgaaaaggcgcccgacggtcatcacgtatgggaaatggaagctaagaccgatagggatctctgtaaaccaaacagccttgtcgtcgaaattccgcccttcagaaaccaacgtatcacttctccggtgcatgtgtcattttatgtgtgtaatggcaaacgcaaacgttcccaatatcaacgctttacatatttgcctgcgaatgtacctatcattaagaccgagccaaccgacgactacgaaccagcccccacgtgcggccctgtttcccaaggcctctcacccctgccccgcccctattatagtcaacaactggcaatgccccctgatccttcttcttgtctggtcgcgggatttccaccatgcccccaacgttctactctcatgcccgccgctccaggtgttagtccgaaactgcatgatctgagccctgccgcatatactaaaggtgtggcatcacctggtcattgccatctggggctgccccaacccgcaggcgaagctcctgctgtgcaagatgtccctcgccctgttgctacacatccaggaagtccaggccaaccaccacctgcgctcttgccgcaacaagtctcagccccaccgtcctcttcatgtccgcccggcctggagcatagtctttgtccttcttcaccatcacccccgctgccaccagcgactcaggaaccaacatgtctccaaccgtgttctcccgcctgtccaccagcaaccggtaggccacaacatctccctagcaccgttaggcgcgatgaatcccctacagcgggccctaggttgctcccggaagttcacgaagatgggtctcccaaccttgctcccataccagtgaccgtgaaaagagaaccagaggaactggatcaactgtatcttgacgatgttaacgagatcatcaggaacgatctgagctctacatcaacacattct

SEQ ID NO: 26 - DNA, 2253 bp, Artificial Sequence
  Description of Artificial Sequence Synthetic polynucleotide
atgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattcttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatccct

SEQ ID NO: 27 - DNA, 2058 bp, Artificial Sequence
  Description of Artificial Sequence Synthetic polynucleotide
atgcaactcggagaacaactgctcgttagttctgtcaatcttcccggggcacatttctatcccctcgaatcagcaaggggcgggtcaggtggatccgccggtcatctgccttctgctgctccttcccctcaaaagctggatctcgataaggctagcaagaaattcagcggatccctgtcatgtgaagcagtatctggtgaaccagctgcggcgtctgctggtgctccagccgcaatgttgagcgatactgatgcaggagatgccttcgcaagtgcagcagctgtcgctaaaccaggaccacccgatgggagaaaagggagcccgtgtggcgaagaagaattgccgtctgctgccgccgcagcggctgctgctgctgcagccgccgccgctaccgcccgttattctatggattccttgagtagcgaaaggtattatcttcaaagtcctggcccgcaaggttctgaattggccgccccatgtagcctgtttccttatcaagccgctgccggcgctcctcatggtcccgtatatcccgccccaaatggcgccagatatccatatgggtcaatgcttccccctggtggatttcctgctgctgtatgtcccccaggacgggcccaatttgggcctggggcaggggctggttcaggggcaggtggctcttctggtggcggcggtgggccaggtacataccaatattcacaaggcgccccactgtatggtccatatccgggcgctgctgccgctgggagctgtggcggcctcggcgggcttggcgtgcctggaagcggttttagggcacatgtgtatttgtgtaatcgaccactttggctgaagtttcataggcatcagacggaaatgataatcactaagcaagggcgaaggatgttcccatttctgtcctttaatattaatggtctgaacccaaccgcacattataacgtctttgtggaagtcgtccttgcagatcctaatcattggcggtttcaaggcggaaagtgggttacgtgcggaaaggcggataacaatatgcaagggaataagatgtacgtccatcctgaatcaccgaacacagggagtcattggatgaggcaagaaataagctttggaaagctgaagctgacgaacaataagggagccaacaataataatactcaaatgatcgtgcttcagtcacttcataagtatcagccaaggcttcacatagtagaggtcacggaagacggggtcgaagatctgaacgaaccatccaaaacacaaaccttcacattttccgagacccagtttatcgccgtcacagcgtatcagaatacagacataacccagctcaaaatagaccacaatcctttcgccaagggatttcgcgataattacgactcctcacaccaaatagtgcccggcggcaggtatggtgtgcagagtttctttccagaaccgttcgtgaatacattgccccaggcacggtactacaacggggaacgaacagtcccccaaactaatggtttgctcagcccacagcaatccgaggaagttgcaaatccgccacaaagatggctcgtaactcccgtgcaacagcccggcacgaataagctggatatatctagctacgagtccgagtacacaagttccacccttcttccgtacgggatcaagagcctgccactgcaaacctcacacgcattgggctactatcccgatcccacattccccgccatggccggctggggcggcagaggctcatatcaacgcaaaatggccgcgggtttgccctggacaagccgcaccagtccgacagtgttttcagaggaccaactgagtaaagaaaaggtaaaggaagagatcggttcaagttggatcgaaaccccaccatcaattaagagcctcgacagtaacgacagcggcgtgtatacttccgcctgcaaaaggagacgtctcagcccctctaattcttccaacgagaactccccgagtattaaatgcgaagatatcaacgcagaggaatacagcaaggatacatctaaggggatgggtggctactacgccttctatactacacct

SEQ ID NO: 28 - DNA, 2289 bp, Artificial Sequence
  Description of Artificial Sequence Synthetic polynucleotide
atgcttactgaccctgatttacctcaggagtttgaaaggatgtcttccaagcgaccagcctctccgtatggggaagcagatggagaggtagccatggtgacaagcagacagaaagtggaagaagaggagagtgacgggctcccagcctttcaccttcccttgcatgtgagttttcccaacaagcctcactctgaggaatttcagccagtttctctgctgacgcaagagacttgtggccataggactcccacttctcagcacaatacaatggaagttgatggcaataaagttatgtcttcatttgccccacacaactcatctacctcacctcagaaggcagaagaaggtgggcgacagagtggcgagtccttgtctagtacagccctgggaactcctgaacggcgcaagggcagtttagctgatgttgttgacaccttgaagcagaggaaaatggaagagctcatcaaaaacgagccggaagaaacccccagtattgaaaaactactctcaaaggactggaaagacaagcttcttgcaatgggatcggggaactttggcgaaataaaagggactcccgagagcttagctgagaaagaaaggcaactcatgggtatgatcaaccagctgaccagcctccgagagcagctgttggctgcccacgatgagcagaagaaactagctgcctctcagattgagaaacagcgtcagcaaatggagctggccaagcagcaacaagaacaaattgcaagacagcagcagcagcttctacagcaacaacacaaaatcaatttgctccagcaacagatccaggttcaaggtcagctgccgccattaatgattcccgtattccctcctgatcaacggacactggctgcagctgcccagcaaggattcctcctccctccaggcttcagctataaggctggatgtagtgacccttaccctgttcagctgatcccaactaccatggcagctgctgccgcagcaacaccaggcttaggcccactccaactgcagcagttatatgctgcccagctagctgcaatgcaggtatctccaggagggaagctgccaggcataccccaaggcaaccttggtgctgctgtatctcctaccagcattcacacagacaagagcacaaacagcccaccacccaaaagcaaggatgaagtggcacagccactgaacctatcagctaaacccaagacctctgatggcaaatcacccacatcacccacctctccccatatgccagctctgagaataaacagtggggcaggccccctcaaagcctctgtcccagcagcgttagctagtccttcagccagagttagcacaataggttacttaaatgaccatgatgctgtcaccaaggcaatccaagaagctcggcaaatgaaggagcaactccgacgggaacaacaggtgcttgatgggaaggtggctgttgtgaatagtctgggtctcaataactgccgaacagaaaaggaaaaaacaacactggagagtctgactcagcaactggcagttaaacagaatgaagaaggaaaatttagccatgcaatgatggatttcaatctgagtggagattctgatggaagtgctggagtctcagagtcaagaatttatagggaatcccgagggcgtggtagcaatgaaccccacataaagcgtccaatgaatgccttcatggtgtgggctaaagatgaacggagaaagatccttcaagcctttcctgacatgcacaactccaacatcagcaagatattgggatctcgctggaaagctatgacaaacctagagaaacagccatattatgaggagcaagcccgtctcagcaagcagcacctggagaagtaccctgactataagtacaagcccaggccaaagcgcacctgcctggtggatggcaaaaagctgcgcattggtgaatacaaggcaatcatgcgcaacaggcggcaggaaatgcggcagtacttcaatgttgggcaacaagcacagatccccattgccactgctggtgttgtgtaccctggagccatcgccatggctgggatgccctcccctcacctgccctcggagcactcaagcgtgtctagcagcccagagcctgggatgcctgttatccagagcacttacggtgtgaaaggagaggagccacatatcaaagaagagatacaggccgaggacatcaatggagaaatttatgatgagtacgacgaggaagaggatgatccagatgtagattatgggagtgacagtgaaaaccatattgcaggacaagccaac

SEQ ID NO: 29 - DNA, 1761 bp, Artificial Sequence
  Description of Artificial Sequence Synthetic polynucleotide
atggctgctgctgtagccgtcgctgctgctagtcgccgccaatcctgttatttgtgcgatcttccgaggatgccttgggcaatgatttgggattttactgagcctgtgtgtcggggttgtgtgaattatgaaggggcagatagggtggaatttgtgattgaaactgctaggcaattgaaaagagcccatgggtgttttccagaaggcaggagcccgccaggtgcggctgcaagcgctgcagcaaaacctcctccattgtcagcgaaagatattctgctgcaacaacaacaacaactcggacatggtggaccagaagccgcacctcgggcaccccaagcactggaaaggtatcctctggcagcagctgcagaacggccgccaaggcttggttcagattttgggtcttcccgacctgccgccagtcttgctcaaccgcctacccctcaacctcctcctgtcaatggtattctcgtacctaatgggttttcaaaactcgaagaacccccagaactcaacaggcaatccccaaatcctagaaggggacatgctgtaccccctactttggttcctttgatgaatggatcagctacacctttgcctacggcccttggactgggcggtcgggcggctgctagcctcgctgctgttagcggcactgcagcagcatctctcggtagtgctcaaccaactgacctcggtgcacataaacgccccgcctctgtcagcagttcagccgctgttgaacatgaacaaagggaagcagccgcgaaagaaaagcagccacccccaccagctcataggggaccagcagattccctttcaactgccgctggtgcagcagaactttccgccgagggcgccggtaaatccagaggcagcggggaacaagattgggttaatcgccccaaaacagttagagatacattgcttgcgctccatcaacatggacattccggcccatttgaatctaaattcaagaaagaacctgcactcaccgctggtagactcctgggctttgaagcaaatggcgcaaatggatccaaggctgtggcccgcaccgctcggaagagaaaaccgtcccccgagcccgagggagaggttggtccacccaaaattaatggcgaagcgcaaccttggttgagtacgtctaccgaaggtcttaaaatacctatgacacccacctctagtttcgtcagcccgcccccaccaacagcgagcccccacagcaatcgcacgactccacccgaggccgctcaaaacggtcaatcacctatggccgcactcatacttgtggctgataacgcgggtggaagccacgctagtaaggacgcaaatcaagtgcattcaacaacacgtcggaactccaattccccaccatcccccagctcaatgaatcagcgccgacttggtccaagggaagtcggcggtcaaggggccggtaataccggcggcttggaacccgttcatccggcgtcccttcccgatagtagcctcgctacttctgcaccactctgttgtacgctttgtcatgaaagattggaagatactcacttcgttcaatgtcctagtgtgccatcccataaattttgttttccctgtagtaggcagagtataaagcaacaaggcgcatccggggaagtgtactgcccgtctggcgagaagtgtccgctggtcggatctaacgttccttgggctttcatgcagggtgagatcgctacaattctggccggggacgttaaggttaagaaggaaagggatagc

SEQ ID NO: 30 - DNA, 1338 bp, Artificial Sequence
  Description of Artificial Sequence Synthetic polynucleotide
atgagacccgtcagggaaaatagctctggggctcgctcacctcgcgtgcccgcggatcttgcccgaagtatcctgatctccctgccatttccacccgatagcctcgcgcatcggccaccatctagcgcacctactgaatctcaagggctctttacagtcgctgcccccgctcccggggccccctcaccccctgctacattggcccatctgctccctgcaccagctatgtatagtctgctcgaaacagagcttaagaatcctgttggcactccgactcaggccgctggaacaggtggaccagccgctcccggcggggccggtaaatcctcagcaaatgcagctggcggggcaaatagcggaggaggatcctcaggcggagcctcaggtggcggtggtggaaccgatcaagatagagtcaagcgccctatgaatgcatttatggtctggagtcggggtcaaagacggaagatggctctcgaaaatccaaagatgcataactcagaaatttctaaaagactgggtgcggattggaagcttttgacggatgcagagaaaaggccctttattgatgaagctaaaagactgagggctgtccatatgaaagaataccccgattataaatatcgccctagacggaaaaccaaaaccctcttgaagaaggacaaatatagccttccttccgggctgctcccgccaggagcagctgcggctgcggctgcagcggccgctgctgctgccgctgcgtcttcccccgttggtgttgggcaacggttggatacatatacacatgtaaatgggtgggcaaatggagcatatagtctcgttcaagaacaactcgggtatgctcaacccccttctatgagttccccaccccctcctcctgcacttccaccaatgcatcgttatgatatggctgggcttcaatatagtcccatgatgccaccaggtgcgcaatcttatatgaatgtagccgcagccgctgcagcagcatccggatatggcggaatggcaccgtctgctaccgccgcagcagctgctgcatatggccaacaaccagcaacggcggcagcagccgccgccgctgcggctgcaatgagtcttgggccaatgggaagcgtggttaaaagtgaaccatcatcaccgccccctgctattgcatcccatagtcaacgtgcctgtctgggagatctccgggatatgatatctatgtatctgcccccgggtggcgatgccgctgatgctgcttcccccttgccgggcggacggttgcatggtgtccatcaacattatcaaggggcaggtacagccgttaatgggacagttcccctcacacatatt

SEQ ID NO: 31 - DNA, 2475 bp, Artificial Sequence
  Description of Artificial Sequence Synthetic polynucleotide
atgttggatatttgcttggaaaaacgtgtgggtacgaccttggctgcccccaagtgtaactccagcactgtgaggtttcagggattggcagaggggaccaaggggaccatgaaaatggacatggaggatgcggatatgactctgtggacagaggctgagtttgaagagaagtgtacatacattgtgaacgaccacccctgggattctggtgctgatggcggtacttcggttcaggcggaggcatccttaccaaggaatctgcttttcaagtatgccaccaacagtgaagaggttattggagtgatgagtaaagaatacataccaaagggcacacgttttggacccctaataggtgaaatctacaccaatgacacagttcctaagaacgccaacaggaaatatttttggaggatctattccagaggggagcttcaccacttcattgacggctttaatgaagagaaaagcaactggatgcgctatgtgaatccagcacactctccccgggagcaaaacctggctgcgtgtcagaacgggatgaacatctacttctacaccattaagcccatccctgccaaccaggaacttcttgtgtggtattgtcgggactttgcagaaaggcttcactacccttatcccggagagctgacaatgatgaatctcacacaaacacagagcagtctaaagcaaccgagcactgagaaaaatgaactctgcccaaagaatgtcccaaagagagagtacagcgtgaaagaaatcctaaaattggactccaacccctccaaaggaaaggacctctaccgttctaacatttcacccctcacatcagaaaaggacctcgatgactttagaagacgtgggagccccgaaatgcccttctaccctcgggtcgtttaccccatccgggcccctctgccagaagactttttgaaagcttccctggcctacgggatcgagagacccacgtacatcactcgctcccccattccatcctccaccactccaagcccctctgcaagaagcagccccgaccaaagcctcaagagctccagccctcacagcagccctgggaatacggtgtcccctgtgggccccggctctcaagagcaccgggactcctacgcttacttgaacgcgtcctacggcacggaaggtttgggctcctaccctggctacgcacccctgccccacctcccgccagctttcatcccctcgtacaacgctcactaccccaagttcctcttgcccccctacggcatgaattgtaatggcctgagcgctgtgagcagcatgaatggcatcaacaactttggcctcttcccgaggctgtgccctgtctacagcaatctcctcggtgggggcagcctgccccaccccatgctcaaccccacttctctcccgagctcgctgccctcagatggagcccggaggttgctccagccggagcatcccagggaggtgcttgtcccggcgccccacagtgccttctcctttaccggggccgccgccagcatgaaggacaaggcctgtagccccacaagcgggtctcccacggcgggaacagccgccacggcagaacatgtggtgcagcccaaagctacctcagcagcgatggcagcccccagcagcgacgaagccatgaatctcattaaaaacaaaagaaacatgaccggctacaagacccttccctacccgctgaagaagcagaacggcaagatcaagtacgaatgcaacgtttgcgccaagactttcggccagctctccaatctgaaggtccacctgagagtgcacagtggagaacggcctttcaaatgtcagacttgcaacaagggctttactcagctcgcccacctgcagaaacactacctggtacacacgggagaaaagccacatgaatgccaggtctgccacaagagatttagcagcaccagcaatctcaagacccacctgcgactccattctggagagaaaccataccaatgcaaggtgtgccctgccaagttcacccagtttgtgcacctgaaactgcacaagcgtctgcacacccgggagcggccccacaagtgctcccagtgccacaagaactacatccatctctgtagcctcaaggttcacctgaaagggaactgcgctgcggccccggcgcctgggctgcccttggaagatctgacccgaatcaatgaagaaatcgagaagtttgacatcagtgacaatgctgaccggctcgaggacgtggaggatgacatcagtgtgatctctgtagtggagaaggaaattctggccgtggtcagaaaagagaaagaagaaactggcctgaaagtgtctttgcaaagaaacatggggaatggactcctctcctcagggtgcagcctttatgagtcatcagatctacccctcatgaagttgcctcccagcaacccactacctctggtacctgtaaaggtcaaacaagaaacagttgaaccaatggatcct

SEQ ID NO: 32 - DNA, 1737 bp, Artificial Sequence
  Description of Artificial Sequence Synthetic polynucleotide
atgctcaggtcaggtcccgcgtcaggtccaagcgttccaacagggcgagcgatgccaagccgacgggtggctcgcccacccgccgcacccgaactcggcgctctgggatctcctgatctgtcaagtctgtcattggctgtcagtcgtagtactgacgagcttgaaattattgatgaatatattaaagaaaatgggtttgggttggatggcggccaacctggtccaggagaaggactccctaggttggtctcccggggagccgccagcttgagtacagtgacactcgggccagttgccccaccggctactcctcctccgtggggatgtccacttggaagactggttagcccggctcccggaccaggaccccaaccccatcttgttataacagaacaaccaaaacaaaggggaatgcggtttaggtatgaatgtgaagggcggtctgcagggtccattctgggtgaatcatcaacggaagcgtcaaagacactcccagcaattgaattgagggactgcggcggcctcagagaagtcgaagtaaccgcttgtttggtctggaaagattggccccatagggttcatccgcattctctggtcggaaaggattgtacagatggtatatgtcgggtcagactgagaccccatgtgtcccctcgacattcattcaataatttgggtattcaatgcgtccgtaagaaagaaatcgaagcagcgatcgaaagaaagatacagttggggatagatccttataatgcaggtagccttaagaatcaccaagaggtcgatatgaacgtcgtccgcatatgttttcaagcaagctaccgagatcaacaagggcaaatgcggcgaatggacccggttctctcagaacctgtgtacgataagaagagcactaatactagcgaacttcgtatctgtcgcatcaataaagagtcaggcccatgtacaggcggggaagaattgtatcttctgtgtgataaagtacaaaaggaagatatctccgttgttttctccagagcttcttgggaaggccgagccgattttagtcaagctgatgtccataggcaaatcgctatcgtctttaaaacgcccccttatgaagatcttgaaatcgtggaaccggtcacggtaaatgttttccttcaaagactgacagacggcgtttgtagtgaaccccttccctttacatatcttccccgggatcacgattcctatggggttgataagaaaagaaagagaggtatgcctgatgtgctgggcgaactcaattcatccgatcctcacggtattgaatccaagaggagaaagaagaaaccagcgattttggatcattttctcccaaatcatggatccgggccctttctgcccccaagtgcactcttgccggatcccgatttctttagcggtacagtctcactccctgggttggaaccacccggtggacccgatcttctcgatgacggtttcgcatatgatcccactgcaccgaccctgtttactatgcttgatctcttgccacccgctccacctcatgcgagtgccgtggtttgttcaggtggcgcgggcgctgttgtgggtgaaacaccggggcccgagcctctcaccttggattcatatcaagcacccggacctggtgacggcggtacggcttccctggtcgggtctaatatgtttcctaaccactatagagaagctgcattcggtggtggtctgctgagtcctggtcccgaggctacc

SEQ ID NO: 33 - protein, 239 aa, Artificial Sequence
  Description of Artificial Sequence Synthetic polypeptide
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKALYLLRRDQRLPPDAHKPPGGGSFRTPIQEEQADAHSTLAKI

SEQ ID NO: 34 - protein, 150 aa, Artificial Sequence
  Description of Artificial Sequence Synthetic polypeptide
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREALYLLRRDQRLPPDAHKPPGGGSFRTPIQEEQADAHSTLAKI

SEQ ID NO: 35 - protein, 297 aa, Artificial Sequence
  Description of Artificial Sequence Synthetic polypeptide
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCALYLLRRDQRLPPDAHKPPGGGSFRTPIQEEQADAHSTLAKI

SEQ ID NO: 36 - protein, 297 aa, Artificial Sequence; identical to SEQ ID NO: 35
  Description of Artificial Sequence Synthetic polypeptide

SEQ ID NO: 37 - protein, 108 aa, Artificial Sequence
  Description of Artificial Sequence Synthetic polypeptide
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRRE

SEQ ID NO: 38 - protein, 538 aa, Artificial Sequence
  Description of Artificial Sequence Synthetic polypeptide
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 39 - protein, 507 aa, Artificial Sequence
  Description of Artificial Sequence Synthetic polypeptide
MAGAGPKRRALAAPAAEEKEEAREKMLAAKSADGSAPAGEGEGVTLQRNITLLNGVAIIVGTIIGSGIFVTPTGVLKEAGSPGLALVVWAACGVFSIVGALCYAELGTTISKSGGDYAYMLEVYGSLPAFLKLWIELLIIRPSSQYIVALVFATYLLKPLFPTCPVPEEAAKLVACLCVLLLTAVNCYSVKAATRVQDAFAAAKLLALALIILLGFVQIGKGDVSNLDPNFSFEGTKLDVGNIVLALYSGLFAYGGWNYLNFVTEEMINPYRNLPLAIIISLPIVTLVYVLTNLAYFTTLSTEQMLSSEAVAVDFGNYHLGVMSWIIPVFVGLSCFGSVNGSLFTSSRLFFVGSREGHLPSILSMIHPQLLTPVPSLVFTCVMTLLYAFSKDIFSVINFFSFFNWLCVALAIIGMIWLRHRKPELERPIKVNLALPVFFILACLFLIAVSFWKTPVECGIGFTIILSGLPVYFFGVWWKNKPKWLLQGIFSTTVLCQKLMQVVPQET

SEQ ID NO: 40 - protein, 522 aa, Artificial Sequence
  Description of Artificial Sequence Synthetic polypeptide
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPAPAREPGHSPQIISFFLALTSTALLFLLFFLTLRFSVVKRGRKKLLYIFKQPFMRPVQTTQEEDGCSCRFPEEEEGGCEL

SEQ ID NO: 41 - protein, 807 aa, Artificial Sequence
  Description of Artificial Sequence Synthetic polypeptide
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 42 - protein, 838 aa, Artificial Sequence
  Description of Artificial Sequence Synthetic polypeptide
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNIWVILVVTLVVPLLLVAVLIVCCCIGSGCGKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 43 - protein, 808 aa, Artificial Sequence
  Description of Artificial Sequence Synthetic polypeptide
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 44 - protein, 824 aa, Artificial Sequence
  Description of Artificial Sequence Synthetic polypeptide
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 45 - protein, 292 aa, Artificial Sequence
  Description of Artificial Sequence Synthetic polypeptide
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLFWLPIGCAAFVVVCILGCILICWLTKKKYSSSVHDPNGEYMFMRAVNTAKKSRLTDVTL

SEQ ID NO: 46 - protein, 512 aa, Artificial Sequence
  Description of Artificial Sequence Synthetic polypeptide
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAHIYESQLCCQLKFWLPIGCAAFVVVCILGCILICWLTKKKYSSSVHDPNGEYMFMRAVNTAKKSRLTDVTL

SEQ ID NO: 47 - protein, 125 aa, Artificial Sequence
  Description of Artificial Sequence Synthetic polypeptide
MPHSSDSSDSSFSRSPPPGKQDSSDDVRRVQRREKNRIAAQKSRQRQTQKADTLHLESEDLEKQNAALRKEIKQLTEELKYFTSVLNSHEPLCSVLAASTPSPPEVVYSAHAFHQPHVSSPRFQP

SEQ ID NO: 48 - protein, 127 aa, Artificial Sequence
  Description of Artificial Sequence Synthetic polypeptide
MSQGLPAAGSVLQRSVAAPGNQPQPQPQQQSPEDDDRKVRRREKNRVAAQRSRKKQTQKADKLHEEYESLEQENTMLRREIGKLTEELKHLTEALKEHEKMCPLLLCPMNFVPVPPRPDPVAGCLPR

SEQ ID NO: 49 - protein, 127 aa, Artificial Sequence; identical to SEQ ID NO: 48
  Description of Artificial Sequence Synthetic polypeptide

SEQ ID NO: 50 - protein, 134 aa, Artificial Sequence
  Description of Artificial Sequence Synthetic polypeptide
MKAFSPVRSVRKNSLSDHSLGISRSKTPVDDPMSLLYNMNDCYSKLKELVPSIPQNKKVSKMEILQHVIDYILDLQIALDSHPTIVSLHHQRPGQNQASRTPLTTLNTDISILSLQASEFPSELMSNDSKALCG

SEQ ID NO: 51 - protein, 119 aa, Artificial Sequence
  Description of Artificial Sequence Synthetic polypeptide
MKALSPVRGCYEAVCCLSERSLAIARGRGKGPAAEEPLSLLDDMNHCYSRLRELVPGVPRGTQLSQVEILQRVIDYILDLQVVLAEPAPGPPDGPHLPIQTAELTPELVISNDKRSFCH

SEQ ID NO: 52 - protein, 426 aa, Artificial Sequence
  Description of Artificial Sequence Synthetic polypeptide
MCDRNGGRRLRQWLIEQIDSSMYPGLIWENEEKSMFRIPWKHAGKQDYNQEVDASIFKAWAVFKGKFKEGDKAEPATWKTRLRCALNKSPDFEEVTDRSQLDISEPYKVYRIVPEEEQKCKLGVATAGCVNEVTEMECGRSEIDELIKEPSVDDYMGMIKRSPSPPEACRSQLLPDWWAQQPSTGVPLVTGYTTYDAHHSAFSQMVISFYYGGKLVGQATTTCPEGCRLSLSQPGLPGTKLYGPEGLELVRFPPADAIPSERQRQVTRKLFGHLERGVLLHSSRQGVFVKRLCQGRVFCSGNAVVCKGRPNKLERDEVVQVFDTSQFFRELQQFYNSQGRLPDGRVVLCFGEEFPDMAPLRSKLILVQIEQLYVRQLAEEAGKSCGAGSVMQAPEEPPPDQVFRMFPDICASHQRSFFRENQQITV

SEQ ID NO: 53 - protein, 439 aa, Artificial Sequence
  Description of Artificial Sequence Synthetic polypeptide
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 54 - protein, 766 aa, Artificial Sequence
  Description of Artificial Sequence Synthetic polypeptide
MADGGAASQDESSAAAAAAADSRMNNPSETSKPSMESGDGNTGTQTNGLDFQKQPVPVGGAISTAQAQAFLGHLHQVQLAGTSLQAAAQSLNVQSKSNEESGDSQQPSQPSQQPSVQAAIPQTQLMLAGGQITGLTLTPAQQQLLLQQAQAQAQLLAAAVQQHSASQQHSAAGATISASAATPMTQIPLSQPIQIAQDLQQLQQLQQQNLNLQQFVLVHPTTNLQPAQFIISQTPQGQQGLLQAQNLLTQLPQQSQANLLQSQPSITLTSQPATPTRTIAATPIQTLPQSQSTPKRIDTPSLEEPSDLEELEQFAKTFKQRRIKLGFTQGDVGLAMGKLYGNDFSQTTISRFEALNLSFKNMCKLKPLLEKWLNDAENLSSDSSLSSPSALNSPGIEGLSRRRKKRTSIETNIRVALEKSFLENQKPTSEEITMIADQLNMEKEVIRVWFCNRRQKEKRINPPSSGGTSSSPIKAIFPSPTSLVATTPSLVTSSAATTLTVSPVLPLTSAAVTNLSVTGTSDTTSNNTATVISTAPPASSAVTSPSLSPSPSASASTSEASSASETSTTQTTSTPLSSPLGTSQVMVTASGLQTAAAAALQGAAQLPANASLAAMAAAAGLNPSLMAPSQFAAGGALLSLNPGTLSGALSPALMSNSTLATIQALASGGSLPITSLDATGNLVFANAGGAPNIVTAPLFLNPQNLSLLTSNPVSLVSAAAASAGNSAPVASLHATSTSAESIQNSLFTVASASGAASTTTTASKAQ

SEQ ID NO: 55 - protein, 338 aa, Artificial Sequence
  Description of Artificial Sequence Synthetic polypeptide
MEYFMVPTQKVPSLQHFRKTEKEVIGGLCSLANIPLTPETQRDQERRIRREIANSNERRRMQSINAGFQSLKTLIPHTDGEKLSKAAILQQTAEYIFSLEQEKTRLLQQNTQLKRFIQELSGSSPKRRRAEDKDEGIGSPDIWEDEKAEDLRREMIELRQQLDKERSVRMMLEEQVRSLEAHMYPEKLKVIAQQVQLQQQQEQVRLLHQEKLEREQQQLRTQLLPPPAPTHHPTVIVPAPPPPPSHHINVVTMGPSSVINSVSTSRQNLDTIVQAIQHIEGTQEKQELEEEQRRAVIVKPVRSCPEAPTSDTASDSEASDSDAMDQSREEPSGDGELP

SEQ ID NO: 56 - protein, 552 aa, Artificial Sequence
  Description of Artificial Sequence Synthetic polypeptide
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 57 - protein, 943 aa, Artificial Sequence
  Description of Artificial Sequence Synthetic polypeptide
MPSTSFPVPSKFPLGPAAAVFGRGETLGPAPRAGGTMKSAEEEHYGYASSNVSPALPLPTAHSTLPAPCHNLQTSTPGIIPPADHPSGYGAALDGGPAGYFLSSGHTRPDGAPALESPRIEITSCLGLYHNNNQFFHDVEVEDVLPSSKRSPSTATLSLPSLEAYRDPSCLSPASSLSSRSCNSEASSYESNYSYPYASPQTSPWQSPCVSPKTTDPEEGFPRGLGACTLLGSPRHSPSTSPRASVTEESWLGARSSRPASPCNKRKYSLNGRQPPYSPHHSPTPSPHGSPRVSVTDDSWLGNTTQYTSSAIVAAINALTTDSSLDLGDGVPVKSRKTTLEQPPSVALKVEPVGEDLGSPPPPADFAPEDYSSFQHIRKGGFCDQYLAVPQHPYQWAKPKPLSPTSYMSPTLPALDWQLPSHSGPYELRIEVQPKSHHRAHYETEGSRGAVKASAGGHPIVQLHGYLENEPLMLQLFIGTADDRLLRPHAFYQVHRITGKTVSTTSHEAILSNTKVLEIPLLPENSMRAVIDCAGILKLRNSDIELRKGETDIGRKNTRVRLVFRVHVPQPSGRTLSLQVASNPIECSQRSAQELPLVEKQSTDSYPVVGGKKMVLSGHNFLQDSKVIFVEKAPDGHHVWEMEAKTDRDLCKPNSLVVEIPPFRNQRITSPVHVSFYVCNGKRKRSQYQRFTYLPANVPIIKTEPTDDYEPAPTCGPVSQGLSPLPRPYYSQQLAMPPDPSSCLVAGFPPCPQRSTLMPAAPGVSPKLHDLSPAAYTKGVASPGHCHLGLPQPAGEAPAVQDVPRPVATHPGSPGQPPPALLPQQVSAPPSSSCPPGLEHSLCPSSPSPPLPPATQEPTCLQPCSPACPPATGRPQHLPSTVRRDESPTAGPRLLPEVHEDGSPNLAPIPVTVKREPEELDQLYLDDVNEIIRNDLSSTSTHS

SEQ ID NO: 58 - protein, 751 aa, Artificial Sequence
  Description of Artificial Sequence Synthetic polypeptide
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHRKCNYSFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 59 - protein, 686 aa, Artificial Sequence
  Description of Artificial Sequence Synthetic polypeptide
MQLGEQLLVSSVNLPGAHFYPLESARGGSGGSAGHLPSAAPSPQKLDLDKASKKFSGSLSCEAVSGEPAAASAGAPAAMLSDTDAGDAFASAAAVAKPGPPDGRKGSPCGEEELPSAAAAAAAAAAAAAATARYSMDSLSSERYYLQSPGPQGSELAAPCSLFPYQAAAGAPHGPVYPAPNGARYPYGSMLPPGGFPAAVCPPGRAQFGPGAGAGSGAGGSSGGGGGPGTYQYSQGAPLYGPYPGAAAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTTP

SEQ ID NO: 60 - protein, 763 aa, Artificial Sequence [sequence omitted]

SEQ ID NO: 61 - protein, 587 aa, Artificial Sequence
  Description of Artificial Sequence Synthetic polypeptide
MAAAVAVAAASRRQSCYLCDLPRMPWAMIWDFTEPVCRGCVNYEGADRVEFVIETARQLKRAHGCFPEGRSPPGAAASAAAKPPPLSAKDILLQQQQQLGHGGPEAAPRAPQALERYPLAAAAERPPRLGSDFGSSRPAASLAQPPTPQPPPVNGILVPNGFSKLEEPPELNRQSPNPRRGHAVPPTLVPLMNGSATPLPTALGLGGRAAASLAAVSGTAAASLGSAQPTDLGAHKRPASVSSSAAVEHEQREAAAKEKQPPPPAHRGPADSLSTAAGAAELSAEGAGKSRGSGEQDWVNRPKTVRDTLLALHQHGHSGPFESKFKKEPALTAGRLLGFEANGANGSKAVARTARKRKPSPEPEGEVGPPKINGEAQPWLSTSTEGLKIPMTPTSSFVSPPPPTASPHSNRTTPPEAAQNGQSPMAALILVADNAGGSHASKDANQVHSTTRRNSNSPPSPSSMNQRRLGPREVGGQGAGNTGGLEPVHPASLPDSSLATSAPLCCTLCHERLEDTHFVQCPSVPSHKFCFPCSRQSIKQQGASGEVYCPSGEKCPLVGSNVPWAFMQGEIATILAGDVKVKKERDS

SEQ ID NO: 62 - protein, 446 aa, Artificial Sequence
  Description of Artificial Sequence Synthetic polypeptide
MRPVRENSSGARSPRVPADLARSILISLPFPPDSLAHRPPSSAPTESQGLFTVAAPAPGAPSPPATLAHLLPAPAMYSLLETELKNPVGTPTQAAGTGGPAAPGGAGKSSANAAGGANSGGGSSGGASGGGGGTDQDRVKRPMNAFMVWSRGQRRKMALENPKMHNSEISKRLGADWKLLTDAEKRPFIDEAKRLRAVHMKEYPDYKYRPRRKTKTLLKKDKYSLPSGLLPPGAAAAAAAAAAAAAAASSPVGVGQRLDTYTHVNGWANGAYSLVQEQLGYAQPPSMSSPPPPPALPPMHRYDMAGLQYSPMMPPGAQSYMNVAAAAAAASGYGGMAPSATAAAAAAYGQQPATAAAAAAAAAAMSLGPMGSVVKSEPSSPPPAIASHSQRACLGDLRDMISMYLPPGGDAADAASPLPGGRLHGVHQHYQGAGTAVNGTVPLTHI

SEQ ID NO: 63 - protein, 825 aa, Artificial Sequence
  Description of Artificial Sequence Synthetic polypeptide
MLDICLEKRVGTTLAAPKCNSSTVRFQGLAEGTKGTMKMDMEDADMTLWTEAEFEEKCTYIVNDHPWDSGADGGTSVQAEASLPRNLLFKYATNSEEVIGVMSKEYIPKGTRFGPLIGEIYTNDTVPKNANRKYFWRIYSRGELHHFIDGFNEEKSNWMRYVNPAHSPREQNLAACQNGMNIYFYTIKPIPANQELLVWYCRDFAERLHYPYPGELTMMNLTQTQSSLKQPSTEKNELCPKNVPKREYSVKEILKLDSNPSKGKDLYRSNISPLTSEKDLDDFRRRGSPEMPFYPRVVYPIRAPLPEDFLKASLAYGIERPTYITRSPIPSSTTPSPSARSSPDQSLKSSSPHSSPGNTVSPVGPGSQEHRDSYAYLNASYGTEGLGSYPGYAPLPHLPPAFIPSYNAHYPKFLLPPYGMNCNGLSAVSSMNGINNFGLFPRLCPVYSNLLGGGSLPHPMLNPTSLPSSLPSDGARRLLQPEHPREVLVPAPHSAFSFTGAAASMKDKACSPTSGSPTAGTAATAEHVVQPKATSAAMAAPSSDEAMNLIKNKRNMTGYKTLPYPLKKQNGKIKYECNVCAKTFGQLSNLKVHLRVHSGERPFKCQTCNKGFTQLAHLQKHYLVHTGEKPHECQVCHKRFSSTSNLKTHLRLHSGEKPYQCKVCPAKFTQFVHLKLHKRLHTRERPHKCSQCHKNYIHLCSLKVHLKGNCAAAPAPGLPLEDLTRINEEIEKFDISDNADRLEDVEDDISVISVVEKEILAVVRKEKEETGLKVSLQRNMGNGLLSSGCSLYESSDLPLMKLPPSNPLPLVPVKVKQETVEPMDP

SEQ ID NO: 64 - protein, 579 aa, Artificial Sequence
  Description of Artificial Sequence Synthetic polypeptide
MLRSGPASGPSVPTGRAMPSRRVARPPAAPELGALGSPDLSSLSLAVSRSTDELEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 65 - protein, 173 aa, Artificial Sequence
  Description of Artificial Sequence Synthetic polypeptide
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSN

SEQ ID NO: 66 - protein, 17 aa, Artificial Sequence
  Description of Artificial Sequence Synthetic peptide
LGWLCLLLLPIPLIVWV

SEQ ID NO: 67 - protein, 145 aa, Artificial Sequence
  Description of Artificial Sequence Synthetic polypeptide
KRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 68 - protein, 214 aa, Artificial Sequence
  Description of Artificial Sequence Synthetic polypeptide
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRA

SEQ ID NO: 69 - protein, 21 aa, Artificial Sequence
  Description of Artificial Sequence Synthetic peptide
VAAILGLGLVLGLLGPLAILL

SEQ ID NO: 70 - protein, 42 aa, Artificial Sequence
  Description of Artificial Sequence Synthetic polypeptide
ALYLLRRDQRLPPDAHKPPGGGSFRTPIQEEQADAHSTLAKI

SEQ ID NO: 71 - protein, 186 aa, Artificial Sequence
  Description of Artificial Sequence Synthetic polypeptide
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 72 - protein, 27 aa, Artificial Sequence
  Description of Artificial Sequence Synthetic peptide
IISFFLALTSTALLFLLFFLTLRFSVV

SEQ ID NO: 73 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 74 - protein, 140 aa, Artificial Sequence
  Description of Artificial Sequence Synthetic polypeptide
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLK

SEQ ID NO: 75 - protein, 21 aa, Artificial Sequence
  Description of Artificial Sequence Synthetic peptide
FWLPIGCAAFVVVCILGCILI

SEQ ID NO: 76 - protein, 38 aa, Artificial Sequence
  Description of Artificial Sequence Synthetic polypeptide
CWLTKKKYSSSVHDPNGEYMFMRAVNTAKKSRLTDVTL

SEQ ID NO: 77 - protein, 80 aa, Artificial Sequence
  Description of Artificial Sequence Synthetic polypeptide
MARGSLRRLLRLLVLGLWLALLRSVAGEQAPGTAPCSRGSSWSADLDKCMDCASCRARPHSDFCLGCAAAPPAPFRLLWP

SEQ ID NO: 78 - protein, 21 aa, Artificial Sequence
  Description of Artificial Sequence Synthetic peptide
ILGGALSLTFVLGLLSGFLVW

SEQ ID NO: 79 - protein, 28 aa, Artificial Sequence
  Description of Artificial Sequence Synthetic peptide
RRCRRREKFTTPIEETGGEGCPAVALIQ

SEQ ID NO: 80 - protein, 227 aa, Artificial Sequence
  Description of Artificial Sequence Synthetic polypeptide
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLM

SEQ ID NO: 81 - protein, 21 aa, Artificial Sequence
  Description of Artificial Sequence Synthetic peptide
LAVLLPLAFFLLLATVFSCIW

SEQ ID NO: 82 - protein, 187 aa, Artificial Sequence
  Description of Artificial Sequence Synthetic polypeptide
KSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 83 - protein, 450 aa, Artificial Sequence
  Description of Artificial Sequence Synthetic polypeptide
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 84 - protein, 21 aa, Artificial Sequence
  Description of Artificial Sequence Synthetic peptide
LLFLILGVLSLLLLVTGAFGF

SEQ ID NO: 85 - protein, 54 aa, Artificial Sequence
  Description of Artificial Sequence Synthetic polypeptide
HLWRRQWRPRRFSALEQGIHPPQAQSKIEELEQEPEPEPEPEPEPEPEPEPEQL

SEQ ID NO: 86 - protein, 210 aa, Artificial Sequence
  Description of Artificial Sequence Synthetic polypeptide
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCS

SEQ ID NO: 87 - protein, 21 aa, Artificial Sequence
  Description of Artificial Sequence Synthetic peptide
LSGIIIGVTVAAVVLIVAVFV

SEQ ID NO: 88 - protein, 209 aa, Artificial Sequence
  Description of Artificial Sequence Synthetic polypeptide
CKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 89 - protein, 232 aa, Artificial Sequence
  Description of Artificial Sequence Synthetic polypeptide
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 90 - protein, 24 aa, Artificial Sequence
  Description of Artificial Sequence Synthetic peptide
LLLGVSVSCIVILAVCLLCYVSIT

SEQ ID NO: 91 - protein, 569 aa, Artificial Sequence
  Description of Artificial Sequence Synthetic polypeptide
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 92 - protein, 239 aa, Artificial Sequence
  Description of Artificial Sequence Synthetic polypeptide
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHN

SEQ ID NO: 93 - protein, 23 aa, Artificial Sequence
  Description of Artificial Sequence Synthetic peptide
IWVILVVTLVVPLLLVAVLIVCC

SEQ ID NO: 94 - protein, 206 aa, Artificial Sequence
  Description of Artificial Sequence Synthetic polypeptide
CIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAREKIQDLLVDSGKFIYLEDGTGSAVSLE

SEQ ID NO: 95 - protein, 232 aa, Artificial Sequence; identical to SEQ ID NO: 89
  Description of Artificial Sequence Synthetic polypeptide

SEQ ID NO: 96 - protein, 24 aa, Artificial Sequence
  Description of Artificial Sequence Synthetic peptide
LLLGVSVSCIVILAVCLLCYVSIT

SEQ ID NO: 97 - protein, 569 aa, Artificial Sequence; identical to SEQ ID NO: 91
  Description of Artificial Sequence Synthetic polypeptide

SEQ ID NO: 98 - DNA, 690 bp, Artificial Sequence
  Description of Artificial Sequence Synthetic polynucleotide
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 99 - protein, 230 aa, Artificial Sequence
  Description of Artificial Sequence Synthetic polypeptide
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMRSKRSRLLHSDYMNMTPRRPGPTRKHYQPYAPPRDFAAYRS

SEQ ID NO: 100 - DNA, 915 bp, Artificial Sequence
  Description of Artificial Sequence Synthetic polynucleotide
atgctctgcccttggagaactgctaacctagggctactgttgattttgactatcttcttagtggccgcttcaagcagtttatgtatggatgaaaaacagattacacagaactactcgaaagtactcgcagaagttaacacttcatggcctgtaaagatggctacaaatgctgtgctttgttgccctcctatcgcattaagaaatttgatcataataacatgggaaataatcctgagaggccagccttcctgcacaaaagcctacaagaaagaaacaaatgagaccaaggaaaccaactgtactgatgagagaataacctgggtctccagacctgatcagaattcggaccttcagattcgtaccgtggccatcactcatgacgggtattacagatgcataatggtaacacctgatgggaatttccatcgtggatatcacctccaagtgttagttacacctgaagtgaccctgtttcaaaacaggaatagaactgcagtatgcaaggcagttgcagggaagccagctgcgcatatctcctggatcccagagggcgattgtgccactaagcaagaatactggagcaatggcacagtgactgttaagagtacatgccactgggaggtccacaatgtgtctaccgtgacctgccacgtctcccatttgactggcaacaagagtctgtacatagagctacttcctgttcatatttatgaatcacaactttgttgccagctgaagttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgacccta

SEQ ID NO: 101 - protein, 305 aa, Artificial Sequence
  Description of Artificial Sequence Synthetic polypeptide
MLCPWRTANLGLLLILTIFLVAASSSLCMDEKQITQNYSKVLAEVNTSWPVKMATNAVLCCPPIALRNLIIITWEIILRGQPSCTKAYKKETNETKETNCTDERITWVSRPDQNSDLQIRTVAITHDGYYRCIMVTPDGNFHRGYHLQVLVTPEVTLFQNRNRTAVCKAVAGKPAAHISWIPEGDCATKQEYWSNGTVTVKSTCHWEVHNVSTVTCHVSHLTGNKSLYIELLPVHIYESQLCCQLKFWLPIGCAAFVVVCILGCILICWLTKKKYSSSVHDPNGEYMFMRAVNTAKKSRLTDVTL

SEQ ID NO: 102 - DNA, 837 bp, Artificial Sequence
  Description of Artificial Sequence Synthetic polynucleotide
atggaacaacggggacagaacgccccggccgcttcgggggcccggaaaaggcacggcccaggacccagggaggcgcggggagccaggcctgggccccgggtccccaagacccttgtgctcgttgtcgccgcggtcctgctgttggtctcagctgagtctgctctgatcacccaacaagacctagctccccagcagagagcggccccacaacaaaagaggtccagcccctcagagggattgtgtccacctggacaccatatctcagaagacggtagagattgcatctcctgcaaatatggacaggactatagcactcactggaatgacctccttttctgcttgcgctgcaccaggtgtgattcaggtgaagtggagctaagtccctgcaccacgaccagaaacacagtgtgtcagtgcgaagaaggcaccttccgggaagaagattctcctgagatgtgccggaagtgccgcacagggtgtcccagagggatggtcaaggtcggtgattgtacaccctggagtgacatcgaatgtgtccacaaagaatcaggtacaaagcacagtggggaagtcccagctgtggaggagacggtgacctccagcccagggactcctgcctctccctgttctctctcaggcatcatcataggagtcacagttgcagccgtagtcttgattgtggctgtgtttgtttgcaagtctttactgtggaagaggagtaagaggagcaggctcctgcacagtgactacatgaacatgactccccgccgccccgggcccacccgcaagcattaccagccctatgccccaccacgcgacttcgcagcctatcgctcc

SEQ ID NO: 103 - protein, 279 aa, Artificial Sequence
  Description of Artificial Sequence Synthetic polypeptide
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKRSKRSRLLHSDYMNMTPRRPGPTRKHYQPYAPPRDFAAYRS

SEQ ID NO: 104 - DNA, 816 bp, Artificial Sequence
  Description of Artificial Sequence Synthetic polynucleotide
atggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatc

SEQ ID NO: 105 - protein, 272 aa, Artificial Sequence
  Description of Artificial Sequence Synthetic polypeptide
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 106 - protein, 161 aa, Artificial Sequence
  Description of Artificial Sequence Synthetic polypeptide
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 107 - protein, 21 aa, Artificial Sequence
  Description of Artificial Sequence Synthetic peptide
FLLWILAAVSSGLFFYSFLLT

SEQ ID NO: 108 - protein, 41 aa, Artificial Sequence
  Description of Artificial Sequence Synthetic polypeptide
AVSLSKMLKKRSPLTTGVYVKMPPTEPECEKQFQPYFIPIN

SEQ ID NO: 109 - protein, 152 aa, Artificial Sequence
  Description of Artificial Sequence Synthetic polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 110 - protein, 27 aa, Artificial Sequence
  Description of Artificial Sequence Synthetic peptide
FWVLVVVGGVLACYSLLVTVAFIIFWV

SEQ ID NO: 111 - protein, 41 aa, Artificial Sequence
  Description of Artificial Sequence Synthetic polypeptide
RSKRSRLLHSDYMNMTPRRPGPTRKHYQPYAPPRDFAAYRS

SEQ ID NO: 112 - protein, 243 aa, Artificial Sequence
  Description of Artificial Sequence Synthetic polypeptide
MLCPWRTANLGLLLILTIFLVAASSSLCMDEKQITQNYSKVLAEVNTSWPVKMATNAVLCCPPIALRNLIIITWEIILRGQPSCTKAYRKETNETKETNCTDERITWVSRPDQNSDLQIRPVAITHDGYYRCIMVTPDGNFHRGYHLQVLVTPEVTLFQNRNRTAVCKAVAGKPAAQISWIPEGDCATKQEYWSNGTVTVKSTCHWEVHNVSTVTCHVSHLTGNKSLYIELLPVPGAKKSAKL

SEQ ID NO: 113 - protein, 21 aa, Artificial Sequence
  Description of Artificial Sequence Synthetic peptide
YIPYIILTIIILTIVGFIWLL

SEQ ID NO: 114 - protein, 61 aa, Artificial Sequence
  Description of Artificial Sequence Synthetic polypeptide
KVNGCRKYKLNKTESTPVVEEDEMQPYASYTEKNNPLYDTTNKVKASEALQSEVDTDLHTL

(19) United States
(12) Patent Application Publication
(10) Pub. No.: US 2024/0392243 A1
(43) Pub. Date: Nov. 28, 2024

(54) DNA CONSTRUCTS FOR IMPROVED T CELL IMMUNOTHERAPY OF CANCER

(71) Applicant: The Regents of the University of California, Oakland, CA (US)

(72) Inventors: Theodore Lee ROTH, San Francisco, CA (US); Franziska BLAESCHKE, San Francisco, CA (US); Ryan APATHY, San Francisco, CA (US); Alexander MARSON, San Francisco, CA (US); Yan Yi Chen, San Francisco, CA (US)

(73) Assignee: The Regents of the University of California, Oakland, CA (US)

(21) Appl. No.: 18/246,155

(22) PCT Filed: Oct. 4, 2021
(86) PCT No.: PCT/US2021/053386

§ 371 Date: Mar. 21, 2023

Related U.S. Application Data
(60) Provisional application No. 63/087,078, filed on Oct. 2, 2020.

(51) Int. Cl. C12N 5/0783; A61P 37/04; C12N 15/85; C12N 15/86; C12N 15/90

(52) U.S. Cl. CPC C12N 5/0636; A61P 37/04; C12N 15/85; C12N 15/86; C12N 15/907; C12N 2510/00; C12N 2750/14143

(57) ABSTRACT

Provided herein are methods and compositions for modifying the genome of human T cells.

### PRIOR RELATED APPLICATIONS

This application claims the benefit of U.S. Provisional Application No. 63/087,078, filed on Oct. 2, 2020, which is hereby incorporated by reference in its entirety.

### BACKGROUND OF THE INVENTION

Current techniques for modification of ex vivo or intravitally gene edited cells for therapeutic use have focused on correction of an existing mutation, limiting therapeutic applicability to conditions caused by a single mutation resulting in a misfunctioning gene, or on integrating an entirely new synthetic gene, requiring extensive research and development into creating a new therapeutically useful synthetic DNA sequence. Therefore, there are limited options for genomic modifications. Given the importance of T cells in adoptive cellular therapeutics, the ability to obtain human T cells and modify them to produce edited T cells with desirable function(s) could be beneficial in the development and application of adoptive T cell therapies.

### BRIEF SUMMARY OF THE INVENTION

The present disclosure is directed f compositions and methods for modifying the genome of a T cell. The inventors have discovered that human T cells can be modified to alter T cell specificity and function. By inserting a nucleic acid encoding a polypeptide and a heterologous T cell receptor (TCR) or a synthetic antigen receptor (e.g., a chimeric antigen receptor (CAR)) into a specific endogenous site in the genome of the T cell, (e.g., a TCR locus), human T cells having the desired antigen specificity of the TCR or CAR and the function of the polypeptide can be made. Further, the compositions and methods described herein can be used to generate human T cells with altered specificity and functionality, while limiting the side effects associated with T cell therapies.

Provided herein is a human T cell that heterologously expresses one or more polypeptides, wherein the one or more polypeptides are encoded by a nucleic acid construct inserted into the TCR locus of the cell.

In some embodiments, the polypeptide comprises a human Fas extracellular domain or portion thereof linked to a human OX40 intracellular domain (and optionally, 1-10 (e.g., 7) amino acids of the Fas intracellular domain) via a transmembrane domain: (Fas-OX40).

In some embodiments, the polypeptide comprises a human TNFRSF12 extracellular domain linked to a human OX40 intracellular domain (and optionally 1-10 (e.g., 7) amino acids of the TNFRSF12 intracellular domain) via a transmembrane domain.

In some embodiments, the polypeptide comprises a human LTBR extracellular domain linked to a human OX40 intracellular domain (and optionally 1-10 (e.g. 7) amino acids of the LTBR intracellular domain) via a transmembrane domain.

In some embodiments, the polypeptide is a truncated human LTBR protein comprising the human LTBR extracellular domain, transmembrane domain and about 1-10 (e.g. 7) amino acids of the intracellular domain.

In some embodiments, the polypeptide is a truncated human TNFRSF12 protein comprising the human TNFRSF12 extracellular domain, transmembrane domain and about 1-10 (e.g. 7) amino acids of the intracellular domain.

In some embodiments, the polypeptide comprises a human LAG-3 extracellular domain linked to a human 4-1BB intracellular domain (and optionally 1-10 (e.g. 7) amino acids of the LAG3 intracellular domain) via a transmembrane domain.

In some embodiments, the polypeptide comprises a human DR5 extracellular domain linked to a human IL-4R intracellular domain (and optionally 1-10 (e.g. 7) amino acids of the DR5 intracellular domain) via a transmembrane domain.

In some embodiments, the polypeptide comprises a human DR4 extracellular domain linked to a human IL-4R intracellular domain (and optionally 1-10 (e.g. 7) amino acids of the DR4 intracellular domain) via a transmembrane domain.

In some embodiments, the polypeptide comprises a human TNFRSFIA extracellular domain linked to a human IL-4R intracellular domain (and optionally 1-10 (e.g. 7) amino acids of the TNFRSFIA intracellular domain) via a transmembrane domain.

In some embodiments, the polypeptide comprises a human LTBR extracellular domain linked to a human IL-4R intracellular domain (and optionally 1-10 (e.g. 7) amino acids of the LTBR intracellular domain) via a transmembrane domain.

In some embodiments, the polypeptide comprises a human IL-4RA extracellular domain linked to a human ICOS intracellular domain via a transmembrane domain.

In some embodiments, the polypeptide comprises a human LAG3 extracellular domain or a portion thereof (and optionally 1-20 amino acids of the ICOS extracellular domain) linked to a human ICOS intracellular domain via a transmembrane domain.

In some embodiments, the polypeptide comprises a human CTLA4 extracellular domain or a portion thereof (and optionally 1-10 (e.g. 7) amino acids of the CTLA4 intracellular domain) linked to a human CD28 intracellular domain via a transmembrane domain.

In some embodiments, the polypeptide comprises a human CD200R extracellular domain or a portion thereof (and optionally, the ICOS extracellular domain or a portion thereof) linked to a human ICOS intracellular domain via a transmembrane domain.

In some embodiments, the polypeptide comprises a human DR5 extracellular domain or a portion thereof (and optionally 1-10 (e.g. 7) amino acids of the DR5 intracellular domain) linked to a human CD28 intracellular domain via a transmembrane domain.

In some embodiments, the polypeptide comprises a full-length IL21R protein, LAT1 protein, BATF protein. BATF3 protein, BATF2 protein, ID2 protein, ID3 protein, IRF8 protein, MYC protein, POU2F1 protein, TFAP4 protein, SMAD4 protein. NFATCI protein. EZH2 protein, EOMES protein, SOX5 protein. IRF2BP2 protein, SOX3 protein, PRDMI protein. IL2RA, or RELB protein.

In some embodiments, the T cell heterologously expresses a polypeptide comprising an amino acid sequence that is at least 95% identical to an amino acid sequence selected from the group consisting of SEQ ID NO: 33-SEQ ID NO: 64, SEQ ID NO: 99, SEQ ID NO: 101. SEQ ID NO: 103 and SEQ ID NO: 105.

In some embodiments, the T cell comprises a heterologous nucleic acid sequence that is at least 95% identical to a nucleic acid sequence selected from the consisting of SEQ ID NO: 1-32, SEQ ID NO: 98, SEQ ID NO: 100, SEQ ID NO: 102 and SEQ ID NO: 104.

In some embodiments, the T cell expresses an antigen-specific T-cell receptor (TCR) or synthetic antigen receptor that recognizes a target antigen. In some embodiments, the T cell is a regulatory T cell, effector T cell, a memory T cell or naïve T cell. In some embodiments, the effector T cell is a CD8+ T cells or a CD4+ T cell. In some embodiments, the effector T cell is a CD8+CD4+ T cell. In some embodiments, the T cell is a primary cell.

In some embodiments, the target insertion site is in exon 1 of a TCR-alpha subunit constant gene (TRAC). In some embodiments, the target insertion site is in exon 1 of a TCR-beta subunit constant gene (TRBC).

In some embodiments, the heterologous nucleic acid inserted into the human T cell encodes, in the following order, (i) a first self-cleaving peptide sequence; (ii) a first heterologous TCR subunit chain, wherein the TCR subunit chain comprises a variable region and a constant region of the TCR subunit: (iii) a second self-cleaving peptide sequence: (iv) a heterologous polypeptide as described herein: (v) a third self-cleaving peptide sequence: (vi) a variable region of a second heterologous TCR subunit chain; and (vii) a portion of the N-terminus of the endogenous TCR subunit, wherein, if the endogenous TCR subunit of the cell is a TCR-alpha (TCR-α) subunit, the first heterologous TCR subunit chain is a heterologous TCR-beta (TCR-β) subunit chain and the second heterologous TCR subunit chain is a heterologous TCR-α subunit chain, and wherein if the endogenous TCR subunit of the cell is a TCR-subunit, the first heterologous TCR subunit chain is a heterologous TCR-α subunit chain and the second heterologous TCR subunit chain is a heterologous TCR-β subunit chain.

In some embodiments, the heterologous nucleic acid inserted into the human T cell encodes, in the following order, (i) a first self-cleaving peptide sequence: (ii) a heterologous polypeptide as described herein: (iii) a second self-cleaving peptide sequence: (iv) a first heterologous TCR subunit chain, wherein the TCR subunit chain comprises a variable region and a constant region of the TCR subunit; (v) a third self-cleaving peptide sequence: (vi) a variable region of a second heterologous TCR subunit chain; and (vii) a portion of the N-terminus of the endogenous TCR subunit, wherein, if the endogenous TCR subunit of the cell is a TCR-alpha (TCR-α) subunit, the first heterologous TCR subunit chain is a heterologous TCR-beta (TCR-β) subunit chain and the second heterologous TCR subunit chain is a heterologous TCR-α subunit chain, and wherein if the endogenous TCR subunit of the cell is a TCR-β subunit, the first heterologous TCR subunit chain is a heterologous TCR-α subunit chain and the second heterologous TCR subunit chain is a heterologous TCR-β subunit chain.

In some embodiments, the nucleic acid construct encodes, in the following order, (i) a first self-cleaving peptide sequence: (ii) a synthetic antigen receptor: (iii) a second self-cleaving peptide sequence: (iv) a heterologous polypeptide described herein; and (v) a third self-cleaving peptide sequence or a polyA sequence.

In some embodiments, the nucleic acid construct encodes, in the following order, (i) a first self-cleaving peptide sequence: (ii) a heterologous polypeptide: (iii) a second self-cleaving peptide sequence: (iv) a synthetic antigen receptor; and (v) a third self-cleaving peptide sequence or a polyA sequence.

In some embodiments, the nucleic acid construct comprises a nucleic acid sequence that is at least 95% identical to a nucleic acid sequence selected from the group consisting of SEQ ID NO: 1-SEQ ID NO: 32. SEQ ID NO: 98, SEQ ID NO: 100, SEQ ID NO: 102 and SEQ ID NO. 104.

Also provided is a method of modifying a human T cell comprising (a) introducing into the human T cell (i) a targeted nuclease that cleaves a target region in the TCR locus of a human T cell to create a target insertion site in the genome of the cell; and (ii) a nucleic acid construct encoding a polypeptide a polypeptide selected from the group consisting of, a polypeptide comprising a human Fas extracellular domain or portion thereof linked to a human OX40 intracellular domain (and optionally, 1-10 (e.g., 7) amino acids of the Fas intracellular domain) via a transmembrane domain: (Fas-OX40); a polypeptide comprising a human TNFRSF12 extracellular domain linked to a human OX40 intracellular domain (and optionally 1-10 (e.g., 7) amino acids of the TNFRSF12 intracellular domain) via a transmembrane domain: a polypeptide comprising a human LTBR extracellular domain linked to a human OX40 intracellular domain (and optionally 1-10 (e.g. 7) amino acids of the LTBR intracellular domain) via a transmembrane domain; a truncated human LTBR protein comprising the human LTBR extracellular domain, transmembrane domain and about 1-10 (c g. 7) amino acids of the intracellular domain; a truncated human TNFRSF12 protein comprising the human TNFRSF12 extracellular domain, transmembrane domain and about 1-10 (e.g. 7) amino acids of the intracellular domain; a truncated human BTLA protein comprising the human BTLA extracellular domain, transmembrane domain and about 1-10 (e.g. 7) amino acids of the intracellular domain; a polypeptide comprising a human LAG-3 extracellular domain linked to a human 4-1BB intracellular domain (and optionally 1-10 (e.g. 7) amino acids of the LAG3 intracellular domain) via a transmembrane domain: a polypeptide comprising a human DR5 extracellular domain linked to a human IL-4R intracellular domain (and optionally 1-10 (e.g. 7) amino acids of the DR5 intracellular domain) via a transmembrane domain: a polypeptide comprising a human DR4 extracellular domain linked to a human IL-4R intracellular domain (and optionally 1-10 (e.g. 7) amino acids of the DR4 intracellular domain) via a transmembrane domain: a polypeptide comprising a human TNFRSFIA extracellular domain linked to a human IL-4R intracellular domain (and optionally 1-10 (e.g. 7) amino acids of the TNFRSFIA intracellular domain) via a transmembrane domain; a polypeptide comprising a human LTBR extracellular domain linked to a human IL-4R intracellular domain (and optionally 1-10 (e.g. 7) amino acids of the LTBR intracellular domain) via a transmembrane domain: a polypeptide comprising a human IL-4RA extracellular domain linked to a human ICOS intracellular domain via a transmembrane domain: a polypeptide comprising a human LAG3 extracellular domain or a portion thereof (and optionally 1-20 amino acids of the ICOS extracellular domain) linked to a human ICOS intracellular domain via a transmembrane domain, a polypeptide comprising a human CTLA4 extracellular domain linked to a human CD28 intracellular domain (and optionally 1-10 (e.g. 7) amino acids of the CTLA-4 intracellular domain) via a transmembrane domain, a polypeptide comprising a buman CD200R extracellular domain linked to a human ICOS intracellular domain (and optionally 1-10 (e.g. 7) amino acids of the CD200R intracellular domain) via a transmembrane domain, a polypeptide comprising a human CD200R extracellular domain linked to a polypeptide encoding amino acids 129-199 of human ICOS: a polypeptide comprising a human DR5 extracellular domain linked to a human CD28 intracellular domain (and optionally 1-10 (e.g. 7) amino acids of the DR5 intracellular domain) via a transmembrane domain; and a polypeptide comprising an IL21R protein, a LAT1 protein, a BATF protein, a BATF3 protein, a BATF2 protein, an ID2 protein, and ID3 protein, an IRF8 protein, a MYC protein, a POU2F1 protein, a TFAP4 protein, a SMAD4 protein, a NFATCI protein, an EXH2 protein, an EOMES protein, a SOX5 protein, an IRF2BP2 protein, a SOX3 protein, a PRDMI protein, IL2RA or a RELB protein; and (b) allowing recombination to occur, thereby inserting the nucleic acid construct in the target insertion site to generate a modified human T cell.

In some methods, the polypeptide comprises an amino acid sequence at least 95% identical to a protein selected from the group consisting of SEQ ID NO: 33-SEQ ID NO: 64. SEQ ID NO: 99, SEQ ID NO: 101, SEQ ID NO: 103 and SEQ ID NO: 105.

In some methods, target insertion site is in exon 1 of a TCR-alpha subunit constant gene (TRAC) or in exon 1 of a TCR-beta subunit constant gene (TRBC).

In some methods, the nucleic acid construct is inserted by introducing a viral vector comprising the nucleic acid construct into the cell. In some embodiments, the targeted nuclease is selected from the group consisting of an RNA-guided nuclease domain, a transcription activator-like effector nuclease (TALEN), a zinc finger nuclease (ZFN) and a megaTAL

In some methods, the targeted nuclease, a guide RNA and the DNA template are introduced into the cell as a ribonucleoprotein complex (RNP)-DNA template complex, wherein the RNP-DNA template complex comprises: (i) the RNP, wherein the RNP comprises the targeted nuclease and the guide RNA; and (ii) the nucleic acid construct.

In some methods, the T cell expresses an antigen-specific T-cell receptor (TCR) or synthetic antigen receptor that recognizes a target antigen. In some embodiments, the T cell is a regulatory T cell, effector T cell, a memory T cell or naïve T cell. In some embodiments, the effector T cell is a CD8+ T cells or a CD4+ T cell. In some embodiments, the effector T cell is a CD8+CD4+ T cell. In some embodiments, the T cell is a primary cell.

Also provided are modified T cell produced by any of the methods described herein.

Further provided is a method of enhancing an immune response in a human subject comprising administering any of the T cells described herein. In some embodiments, the T cell expresses an antigen-specific TCR that recognizes a target antigen in the subject. In some embodiments, the human subject has cancer and the target antigen is a cancer-specific antigen. In some embodiments, the human subject has an autoimmune disorder or an allergic disorder and the antigen is an antigen associated with the autoimmune disorder or the allergic disorder. In some embodiments, the subject has an infection and the target antigen is an antigen associated with the infection. In some embodiments, the T-cell is autologous. In some embodiments, the T-cell is allogenic. In some embodiments, the T cell is an induced pluripotent stem cell (iPSC)-derived T cell.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 is a schematic illustration of the pooled knock-in platform and subsequent functional single stimulation screens. A switch receptor and a transcription factor library including an NY-ESO-1-specific TCR were non-virally integrated into the TRAC locus of primary human T cells by ribonucleoprotein (RNP) electroporation. The edited T cell pool was used in various single stimulation conditions and construct abundance was compared in input vs output T cell populations by amplicon sequencing.

FIGS. 2A-I show a Next Generation Sequencing (NGS) Pipeline and Quality Control Metrics of Pooled Knock-in Libraries. (A) Unique barcodes for every construct (“5′ BC” and “3′ BC”) are encoded in degenerate bases in linker sequences flanking the gene of interest (“Gene X”). 5′ and 3′ BCs allow for sequencing of genomic DNA (gDNA) or cDNA through distinct amplification strategies. DNA mismatches are introduced into one homology arm of the HDR template, allowing only on-target knock-ins to be amplified with primers bound to the endogenous homology arm sequence in the gDNA sequencing strategy. Extracted RNA is transcribed and the 3″ barcode is sequenced using primers specific for that inserted region. (B) Percent of amplicon sequencing reads with GFP or RFP barcodes in indicated sorted populations were obtained 7 days after knock-in. Duplexed knock-in libraries were pooled at indicated stages and the (3′) barcode was sequenced from cDNA. Improved construct design for Pooled Knock-in version 2 (PoKI v2) is compared to previous pooled knock-in strategies (PoKI v1. Roth et al. 2020) Percent reads with correctly assigned barcodes in sorted populations was notably improved over PoKI v1 when pooling at the assembly state. Amount of template switching was calculated for the n=2 member pilot library (lower left panel) and an n>200 member library (lower right panel) and again compared to the previous version of the pooled KI platform (Roth et al.). Bars represent mean. N=2 individual donors. (C) Percent of total reads of pooled knock-in libraries in 6 human donors was calculated. Transcription factor (TF) and switch receptor (SR) libraries were knocked in as one large library and computationally separated into individual libraries for analysis. All construct barcodes were consistently well-represented with even library distribution (TF and SF Gini coefficients=0.23 and 0.20, respectively). (D) A weak negative correlation between construct size and library representation was observed in the plasmid pool, HDR template pool, and of knock-in reads in 6 human donors (R2=0.26, 0.21, and 0.25, respectively). Even the largest library members (4.5 kb inserts) were well represented. Four constructs above 1.5% were omitted from the HDR template library plot to maintain axis consistency. (E) The reproducibility of pooled knock-in across technical and biological replicates was analyzed. Sequencing of the 3′ BC from mRNA was highly reproducible across technical and biological replicates (R2=0.99 and 0.96, respectively). Biological replicates via the 5′ gDNA sequencing strategy yielded a similarly strong correlation (R2=0.99). (F) The correlation between gDNA and mRNA BC sequencing strategies was analyzed 5′ BCs sequenced off gDNA and 3′ BC sequenced off mRNA from the same pooled knock-in experimental donor were well correlated (R2=0.78). (G) The correlation between biological replicates across coverage range was analyzed. Both mRNA and gDNA sequencing strategies were assessed at decreasing sequencing coverage. Correlations were also obtained from cell populations before (Input) and after (Stim) stimulation. Values were obtained as described in FIG. 2E. Even at low coverage (50×), donors were highly correlated across all strategies and experimental conditions. (H) Selective DNA sequencing of knock-in barcodes with UMI was performed. After transcription, the TCR+Gene X mRNA transcripts from the individual cell are reverse transcribed using a gene-specific primer along with a universal molecular identifier (UMI). Following reverse transcription, a primer binding immediately upstream of the 3′ BC produces an amplicon containing both the 3′ barcode and the UMI. Next-generation sequencing of this amplicon allows for correlation between UMIs and BC counts. (1) Next-generation sequencing of the 3′ BC+UMI amplicon reveal a high correlation between UMIs and BC counts (R2=1.00).

FIGS. 3A-B show the identification of top positive and negative hits after single stimulation abundance screen. (A) Primary human T cells were edited to express the switch receptor (left panel) or transcription factor (right panel) library plus NY-ESO TCR. Amplicon sequencing was performed before and after different stimulation conditions to determine log 2 fold change in construct abundance in output vs input population. Heatmaps identify top negative (blue, depleted) as well as top positive (red, enriched) hits throughout the different single stimulation conditions. N=6 individual donors. (B) Primary human T cells were edited as described in FIG. 3A and abundance of T cell constructs was evaluated prior to and after excessive CD3/CD28 stimulation (bead:cell ratio 5:1). Next generation sequencing across 6 individual donors identifies BATF (log 2 fold change 1.05, q value 0.000009), BATF3 (1.05, 0.000017), MYC (0.99, 0.000012), ID2 (0.72, 0.00008) and ID3 (0.89, 0.000001) as top positive hits in this stimulation condition. Average log 2 fold change over input population is shown. False discovery rate was calculated using the Benjamini-Krieger-Yekutieli method. N=6 individual donors.

FIGS. 4A-E provide the characteristics of multiple stimulation screen to identify exhaustion-resistant T cell constructs. (A) A schematic illustration of the multiple stimulation screen is shown T cells were edited as described in FIG. 1A, left panel and then stimulated with A375 cells every two days for a total of five stimulations. Amplicon sequencing and protein expression analysis (flow cytometry) were performed at every time-point to evaluate abundance of T cell constructs and expression of exhaustion markers. (B) Control T cells (NY-ESO TCR plus NGFRt) were subjected to the multiple stimulation screen described in FIG. 4A. Knock-in percentage (NGFR+) was determined by flow cytometry during the course of the assay and compared to unstimulated T cells. Multiple stimulations with target cells enriched for knock-in positive cells (13.8% prior to stimulation vs 83.7% after five stimulations) proofing that the assay is able to put selective pressure on the pooled knock-in cell population. N=4 individual donors, mean plus SEM is shown. (C) T cells differentiated throughout the assay measured by surface expression of CD45RA and CD62L before and after multiple stimulation assay (flow cytometry). The majority of edited T cells (54.5%) showed an effector memory phenotype (CD45RA-/CD62L) after five stimulations with target cells. N=4 individual donors, mean is shown. (D) Intracellular TOX expression of T cells was analyzed by flow cytometry and increased throughout the course of the assay hinting at exhaustion induction in the T cells. N=4 individual donors, mean plus SEM is shown. (E) Expression of surface exhaustion molecules LAG-3. PD-1, TIM-3 and CD39 was analyzed by flow cytometry through the course of the assay. Whereas PD-1 expression peaks earlier during the multiple stimulation assay, the other exhaustion markers stay highly expressed after five stimulations.

FIGS. 5A-C show the identification of top positive and negative hits after multiple stimulation abundance screen. (A-B) Primary human T cells were edited to express an NY-ESO TCR and the switch receptor (A) and transcription factor (B) library. Constructs were subjected to the multiple stimulation screen as described in FIG. 4A. Average log 2 fold change of construct abundance compared to input population at every time-point of the multiple stimulation assay is shown. Heatmaps identify top negative (blue, depleted) as well as top positive (red, enriched) hits throughout the different single stimulation conditions. N=4 individual donors. (C) Abundance of top positive and top negative hits as well as controls GFP and RFP was evaluated over time and showed increase in abundance for BATF and BATF3 while the top negative hits, Eomes and NFATCI, were decreased in abundance. N=4 individual donors, mean plus SEM shown.

FIGS. 6A-D show arrayed abundance assays for four exemplary constructs. A 50/50 co-culture was set up for a control knock-in construct (NY-ESO-specific TCR plus NGFR) and each one of the respective exemplary knock-ins (NY-ESO-specific TCR in combination with (A) IRF8. (B) BATF, (C) JUN or (D) Eomes). Changes in abundance were detected during the course of the multiple stimulation assay and normalized to input abundance. As predicted in the pooled knock-in screen, IRF8 and BATF increased in abundance over time whereas JUN stayed stable and Eomes decreased.

FIGS. 7A-D confirm improved in vitro killing of target cells by one of the top hits identified in the multiple stimulation screens (IRF8). A375 target cells were co-cultured with T cells engineered to express the NY-ESO-specific TCR in combination with either the control construct (NGFR) or the construct of interest (IRF8) at different E/T ratios. A375 cells without T cells served as control. (A) and (B) show the assay without pre-stimulation, (C) and (D) show the assay after the T cells were subject to the multiple stimulation assay.

FIGS. 8A-B show increased cytokine release of NY-ESO/RF8 cells compared to control cells. NY-ESO/IRF8 and NY-ESO/NGFR control T cells were stimulated once (CD3/CD28/CD2) (A) or re-stimulated (CD3/CD28/CD2) after they had gone through the multiple stim assay (B). Intracellular expression of effector cytokines IFN-g, IL-2 and TNF-α was analyzed by flow cytometry.

FIG. 9 shows the level of effector cytokines in the supernatant of NY-ESO/IRF8 vs NY-ESO/NGFR control T cells at the end of the multiple stimulation assay. Cytokine concentrations were analyzed using a flow-based assay and confirmed increased effector cytokine release in NY-ESO/IRF8 T cells.

FIGS. 10A-B describe the expression of activation markers (A) and exhaustion markers (B) on NY-ESO/IRF8 vs NY-ESO/NGFR control cells after going through the multiple stimulation assay and then being re-stimulated (CD3/CD28/CD2). Expression level was analyzed by flow cytometry and showed higher levels of activation marker CD69 and lower levels of exhaustion marker TIM-3 on NY-ESO/IRF8 cells.

FIGS. 11A-E shows the results of human T cell knock-in experiments. (A) Single knock-in of the tonic signaling GD2 CAR and TFAP4 or control (NGFR) into primary human T cells was done. TFAP4 and NGFR GD2 CAR T cells were co-cultured at a 50/50 ratio and abundance levels were evaluated over time. (B) TFAP4 or control T cells were co-cultured with GD2-expressing target cells. Number of GFP-positive target cells was analyzed using the Incucyte (E:T ratio of 1:4). TFAP4 overexpression increased killing capacity of GD2 CAR T cells. (C) Number of Annexin+ cells was analyzed in the assay described in (B) and showed increased levels of Annexin+ cells in TFAP4 conditions across different E:T ratios. (D) NSG mice were challenged with 0.5M GD2 expressing Nalm-6 cells IV and treated with 2M anti-GD2 CAR T cells with or without TFAP4 overexpression three days later. Anti-GD2 CAR T cells with TFAP4 knock-in showed improved leukemia control measured by luciferase assay in two individual donors (n=5 mice per donor per group). (E) TFAP4 overexpression increases CD25 levels on T cells as measured by flow cytometry.

FIGS. 12A-B show a schematic illustration of the pooled knock-in platform and subsequent functional single stimulation screens. A switch receptor and a transcription factor library including an NY-ESO-1-specific TCR were non-virally integrated into the TRAC locus of primary human T cells by ribonucleoprotein (RNP) electroporation. The edited T cell pool was used in various single stimulation conditions and construct abundance was compared in input vs output T cell populations by amplicon sequencing.

FIGS. 13A-B provide an overview of the different screens performed in the TCR/CAR settings (NY-ESO TCR vs CD19 CAR vs tonic signaling GD2 CAR) with no, single or multiple stimulations with target cells. TFAP4 was identified as the top hit in the tonic signaling GD2 CAR assay when comparing abundance levels on day 16 vs day 4 after electroporation. Log2 fold changes shown.

### DEFINITIONS

As used in this specification and the appended claims, the singular forms “a.” “an.” and “the” include plural reference unless the context clearly dictates otherwise.

The term “nucleic acid” or “nucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, guide RNA (e.g., a single guide RNA), or micro RNA.

As used herein, the term “endogenous” with reference to a nucleic acid, for example, a gene, or a protein in a cell is a nucleic acid or protein that occurs in that particular cell as it is found in nature, for example, at its natural genomic location or locus. Moreover, a cell “endogenously expressing” a nucleic acid or protein expresses that nucleic acid or protein as it is found in nature.

As used herein the phrase “heterologous” refers to what is not normally found in nature. The term “heterologous nucleotide sequence” refers to a nucleotide sequence not normally found in a given cell in nature. As such, a heterologous nucleotide sequence may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

A “promoter” is defined as one or more a nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

“Polypeptide.” “peptide.” and “protein” are used interchangeably herein to refer to a polymer of amino acid residues. As used herein, the terms encompass amino acid chains of any length, including full-length proteins, wherein the amino acid residues are linked by covalent peptide bonds.

As used herein, the term “complementary” or “complementarity” refers to specific base pairing between nucleotides or nucleic acids. Complementary nucleotides are, generally, A and T (or A and U), and G and C. The guide RNAs described herein can comprise sequences, for example, DNA targeting sequences that are perfectly complementary or substantially complementary (e.g., having 1-4 mismatches) to a genomic sequence.

The “CRISPR/Cas” system refers to a widespread class of bacterial systems for defense against foreign nucleic acid. CRISPR/Cas systems are found in a wide range of eubacterial and archacal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

Cas9 homologs are found in a wide variety of cubacteria, including, but not limited to bacteria of the following taxonomic groups: Actinobacteria, Aquificae, Bacteroidetes-Chlorobi, Chlamydiae-Verrucomicrobia, Chiroflexi, Cyanobacteria, Firmicutes, Proteobacteria, Spirochaetes, and Thermotogde. An exemplary Cas9 protein is the Streptococcus pyogenes Cas9 protein. Additional Cas9 proteins and homologs thereof are described in, e.g., Chylinksi, et al., RNA Biol. 2013 May 1; 10 (5): 726-737; Nat. Rev. Microbiol. 2011 June: 9 (6): 467-477; Hou, et al., Proc Natl Acad Sci USA. 2013 Sep. 24; 110 (39): 15644-9: Sampson et al., Nature. 2013 May 9; 497 (7448): 254-7; and Jinek, et al., Science. 2012 Aug. 17; 337 (6096): 816-21. Variants of any of the Cas9 nucleases provided herein can be optimized for efficient activity or enhanced stability in the host cell. Thus, engineered Cas9 nucleases are also contemplated. See, for example. “Slaymaker et al., “Rationally engineered Cas9 nucleases with improved specificity,” Science 351 (6268): 84-88 (2016)).

As used herein, the term “Cas9” refers to an RNA-mediated nuclease (e.g., of bacterial or archeal orgin, or derived therefrom). Exemplary RNA-mediated nucleases include the foregoing Cas9 proteins and homologs thereof. Other RNA-mediated nucleases include Cpf1 (See, e.g., Zetsche et al., Cell, Volume 163, Issue 3, p759-771, 22 Oct. 2015) and homologs thereof. As used herein, the term “ribonucleoprotein” complex and the like refers to a complex between a targeted nuclease, for example. Cas9, and a crRNA (e.g., guide RNA or single guide RNA), the Cas9 protein and a trans-activating crRNA (tracrRNA), the Cas9 protein and a guide RNA, or a combination thereof (e.g., a complex containing the Cas9 protein, a tracrRNA, and a crRNA guide RNA). It is understood that in any of the embodiments described herein, a Cas9 nuclease can be substituted with a Cpf1 nuclease or any other guided nuclease.

As used herein, the phrase “modifying” in the context of modifying a genome of a cell refers to inducing a structural change in the sequence of the genome at a target genomic region. For example, the modifying can take the form of inserting a nucleotide sequence into the genome of the cell. For example, a nucleotide sequence encoding a polypeptide can be inserted into the genomic sequence the TCR locus of a T cell. As used throughout a “TCR locus” is a location in the genome where the gene encoding a TCRα subunit, a TCRβ subunit, a TCRγ subunit, or a TCRδ subunit is located.

Such modifying can be performed, for example, by inducing a double stranded break within a target genomic region, or a pair of single stranded nicks on opposite strands and flanking the target genomic region. Methods for inducing single or double stranded breaks at or within a target genomic region include the use of a Cas9 nuclease domain, or a derivative thereof, and a guide RNA, or pair of guide RNAs, directed to the target genomic region.

As used herein, the phrase “introducing” in the context of introducing a nucleic acid or a complex comprising a nucleic acid, for example, an RNP-DNA template complex, refers to the translocation of the nucleic acid sequence or the RNP-DNA template complex from outside a cell to inside the cell. In some cases, introducing refers to translocation of the nucleic acid or the complex from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

As used herein, the term “selectable marker” refers to a gene which allows selection of a host cell, for example, a T cell, comprising a marker. The selectable markers may include, but are not limited to: fluorescent markers, luminescent markers and drug selectable markers, cell surface receptors, and the like. In some embodiments, the selection can be positive selection; that is, the cells expressing the marker are isolated from a population, e.g. to create an enriched population of cells expressing the selectable marker. Separation can be by any convenient separation technique appropriate for the selectable marker used. For example, if a fluorescent marker is used, cells can be separated by fluorescence activated cell sorting, whereas if a cell surface marker has been inserted, cells can be separated from the heterogeneous population by affinity separation techniques, e.g. magnetic separation, affinity chromatography, “panning” with an affinity reagent attached to a solid matrix, fluorescence activated cell sorting or other convenient technique.

As used herein, a “cell” can be a human T cell or a cell capable of differentiating into a T cell, for example, a T cell that expresses a TCR receptor molecule. These include hematopoietic stem cells and cells derived from hematopoietic stem cells.

As used herein, the phrase “hematopoictic stem cell” refers to a type of stem cell that can give rise to a blood cell. Hematopoietic stem cells can give rise to cells of the myeloid or lymphoid lineages, or a combination thereof. Hematopoietic stem cells are predominantly found in the bone marrow, although they can be isolated from peripheral blood, or a fraction thereof. Various cell surface markers can be used to identify, sort, or purify hematopoietic stem cells. In some cases, hematopoietic stem cells are identified as c-kit⁺ and lin⁻. In some cases, human hematopoietic stem cells are identified as CD34⁺, CD59⁺, Thy1/CD90⁺, CD38^{lo/-}, C-kit/CD117⁺, lin⁻. In some cases, human hematopoietic stem cells are identified as CD34⁻, CD59⁺, Thy1/CD90⁺, CD38^{lo/-}, C-kit/CD117⁺, lin⁻. In some cases, human hematopoietic stem cells are identified as CD133⁺, CD59⁺, Thy1/CD90⁺, CD38^{lo/-}, C-kit/CD117⁺, lin⁻. In some cases, mouse hematopoietic stem cells are identified as CD34^{lo/-}, SCA-1⁺, Thy1^{+/lo}, CD38⁺, C-kit⁺, lin⁻. In some cases, the hematopoietic stem cells are CD150+CD48-CD244⁻.

As used herein, the phrase “hematopoietic cell” refers to a cell derived from a hematopoietic stem cell. The hematopoietic cell may be obtained or provided by isolation from an organism, system, organ, or tissue (e.g., blood, or a fraction thereof). Alternatively, an hematopoietic stem cell can be isolated and the hematopoictic cell obtained or provided by differentiating the stem cell. Hematopoictic cells include cells with limited potential to differentiate into further cell types. Such hematopoietic cells include, but are not limited to, multipotent progenitor cells, lineage-restricted progenitor cells, common myeloid progenitor cells, granulocyte-macrophage progenitor cells, or megakaryocyte-erythroid progenitor cells. Hematopoietic cells include cells of the lymphoid and myeloid lineages, such as lymphocytes, erythrocytes, granulocytes, monocytes, and thrombocytes. In some embodiments, the hematopoietic cell is an immune cell, such as a T cell, B cell, macrophage, a natural killer (NK) cell or dendritic cell. In some embodiments the cell is an innate immune cell.

As used herein, the phrase “T cell” refers to a lymphoid cell that expresses a T cell receptor molecule. T cells include human alpha beta (αβ) T cells and human gamma delta (γδ) T cells. T cells include, but are not limited to, naïve T cells, stimulated T cells, primary T cells (e.g., uncultured), cultured T cells, immortalized T cells, helper T cells, cytotoxic T cells, memory T cells, regulatory T cells, natural killer T cells, combinations thereof, or sub-populations thereof. T cells can be CD4⁺, CD8⁺, or CD4⁺ and CD8⁺. T cells can also be CD4⁻, CD8⁻, or CD4⁻ and CD8⁻. T cells can be helper cells, for example helper cells of type TAI, T_{H}2, T_{H}3, T_{H}9, T_{H}17, or T_{FH}. T cells can be cytotoxic T cells. Regulatory T cells can be FOXP3⁺ or FOXP3⁻. T cells can be alpha/beta T cells or gamma/delta T cells. In some cases, the T cell is a CD4⁺CD25^{hi}CD127^{lo} regulatory T cell. In some cases, the T cell is a regulatory T cell selected from the group consisting of type 1 regulatory (Tr1), T_{H}3. CD8+CD28−, Treg17, and Qa-1 restricted T cells, or a combination or sub-population thereof. In some cases, the T cell is a FOXP3⁺ T cell. In some cases, the T cell is a CD4⁺CD25^{lo}CD127^{hi} effector T cell. In some cases, the T cell is a CD4⁺CD25^{lo}CD127^{hi}CD45RA^{hi}CD45RO⁻ naïve T cell. A T cell can be a recombinant T cell that has been genetically manipulated.

As used herein, the phrase “primary” in the context of a primary cell is a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. For example, primary T cells can be activated by contact with (e.g., culturing in the presence of) CD3, CD28 agonists, IL-2, IFN-γ, or a combination thereof.

“Treating” refers to any indicia of success in the treatment or amelioration or prevention of the disease, condition, or disorder, including any objective or subjective parameter such as abatement: remission; diminishing of symptoms or making the disease condition more tolerable to the patient; slowing in the rate of degeneration or decline: or making the final point of degeneration less debilitating.

As used herein, the term “homology directed repair” or HDR refers to a cellular process in which cut or nicked ends of a DNA strand are repaired by polymerization from a homologous template nucleic acid. Thus, the original sequence is replaced with the sequence of the template. In some cases, an exogenous template nucleic acid, for example, a DNA template, can be introduced to obtain a specific HDR-induced change of the sequence at a target site. In this way, specific mutations can be introduced at a cut site, for example, a cut site created by a targeted nuclease. A single-stranded DNA template or a double-stranded DNA template can be used by a cell as a template for editing or modifying the genome of a cell, for example, by HDR. Generally, the single-stranded DNA template or a double-stranded DNA template has at least one region of homology to a target site. In some cases, the single-stranded DNA template or double-stranded DNA template has two homologous regions, for example, a 5′ end and a 3′ end, flanking a region that contains the DNA template to be inserted at a target cut or insertion site.

The term “substantial identity” or “substantially identical.” as used in the context of polynucleotide or polypeptide sequences, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein: preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul. Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

### DETAILED DESCRIPTION OF THE INVENTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art: therefore, information well known to the skilled artisan is not necessarily included.

The present disclosure is directed to compositions and methods for modifying the genome of a T cell. The inventors have discovered that human T cells can be modified to alter T cell specificity and function.

### Compositions

Provided herein is a human T cell that heterologously expresses one or more polypeptides, wherein the one or more polypeptides are encoded by a nucleic acid construct inserted into the TCR locus of the cell. Any of the polypeptides described herein can be heterologously expressed in a human T cell. In some examples, two or more, three or more, four or more or five or more polypeptides described herein are heterologously expressed in a human T cell. In some examples the one or more polypeptides are encoded by one or more nucleic acid constructs.

Exemplary polypeptides include, but are not limited to, the amino acid sequences set forth as SEQ ID Nos: 33-64. A polypeptide comprising an amino acid sequence that is at least 80%, 85%, 90%, 99%, or 100% identical to any one of the amino acid sequences set forth as SEQ ID Nos: 33-64 can also be expressed in a human T cell. Other polypeptides that can be heterologously expressed include polypeptides comprising the amino acid sequences set forth as SEQ ID Nos: 65-97. A polypeptide comprising an amino acid sequence that is at least 80%, 85%, 90%, 99%, or 100% identical to any one of the amino acid sequences set forth as SEQ ID Nos: 65-97 can also be heterologously expressed in a human T cell.

In some embodiments, the polypeptide comprises a human Fas extracellular domain or portion thereof linked to a human OX40 intracellular domain (and optionally. 1-10 (e.g., 7) amino acids of the Fas intracellular domain) via a transmembrane domain. In some embodiments, the transmembrane domain is a human Fas transmembrane domain or a human OX40 transmembrane domain. In some embodiments, the polypeptide comprises or consists of SEQ ID NO: 33. In some embodiments, a relevant domain comprises an amino acid sequence at least 95% or 100% identical to the sequence set forth in Table 1.

In some embodiments, the polypeptide comprises a human TNFRSF12 extracellular domain linked to a human OX40 intracellular domain (and optionally 1-10 (e.g., 7) amino acids of the TNFRSF12 intracellular domain) via a transmembrane domain. In some embodiments, the transmembrane domain is a TNFRSF12 transmembrane domain or a human OX40 transmembrane domain. In some embodiments, the polypeptide comprises or consists of SEQ ID NO: 34. In some embodiments, a relevant domain comprises an amino acid sequence at least 95% or 100% identical to the sequence set forth in Table 1.

In some embodiments, the polypeptide comprises a human LTBR extracellular domain linked to a human OX40 intracellular domain (and optionally 1-10 (e.g. 7) amino acids of the LTBR intracellular domain) via a transmembrane domain. In some embodiments, the transmembrane domain is a LTBR transmembrane domain or a human OX40 transmembrane domain. In some embodiments, the polypeptide comprises or consists of SEQ ID NO: 35. In some embodiments, a relevant domain comprises an amino acid sequence at least 95% or 100% identical to the sequence set forth in Table 1.

In some embodiments, the polypeptide is a truncated human LTBR protein comprising the human LTBR extracellular domain, transmembrane domain and about 1-10 (e.g. 7) amino acids of the intracellular domain. In some embodiments, the polypeptide comprises or consists of SEQ ID NO: 36. In some embodiments, a relevant domain comprises an amino acid sequence at least 95% or 100% identical to the sequence set forth in Table 1.

In some embodiments, the polypeptide is a truncated human TNFRSF12 protein comprising the human TNFRSF12 extracellular domain, transmembrane domain and about 1-10 (e.g. 7) amino acids of the intracellular domain. In some embodiments, the polypeptide comprises or consists of SEQ ID NO: 37. In some embodiments, a relevant domain comprises an amino acid sequence at least 95% or 100% identical to the sequence set forth in Table 1.

In some embodiments, the polypeptide comprises a human LAG-3 extracellular domain linked to a human 4-1BB intracellular domain (and optionally 1-10 (e.g. 7) amino acids of the LAG3 intracellular domain) via a transmembrane domain. In some embodiments, the transmembrane domain is a LAG-3 transmembrane domain or a 4-1BB transmembrane domain. In some embodiments, the polypeptide comprises or consists of SEQ ID NO: 40. In some embodiments, a relevant domain comprises an amino acid sequence at least 95% or 100% identical to the sequence set forth in Table 1.

In some embodiments, a polypeptide comprises a human DR5 extracellular domain linked to a human IL-4R intracellular domain (and optionally 1-10 (e.g. 7) amino acids of the DR5 intracellular domain) via a transmembrane domain. In some embodiments, the transmembrane domain is a human IL-4R transmembrane domain or a human DR5 transmembrane domain. In some embodiments, the polypeptide comprises or consists of SEQ ID NO: 41. In some embodiments, a relevant domain comprises an amino acid sequence at least 95% or 100% identical to the sequence set forth in Table 1.

In some embodiments, the polypeptide comprises a human DR4 extracellular domain linked to a human IL-4R intracellular domain (and optionally 1-10 (e.g. 7) amino acids of the DR4 intracellular domain) via a transmembrane domain. In some embodiments, the transmembrane domain is a human IL-4R transmembrane domain or a human DR4 transmembrane domain. In some embodiments, the polypeptide comprises or consists of SEQ ID NO: 42. In some embodiments, a relevant domain comprises an amino acid sequence at least 95% or 100% identical to the sequence set forth in Table 1.

In some embodiments, the polypeptide comprises a human TNFRSFIA extracellular domain linked to a human IL-4R intracellular domain (and optionally 1-10 (e.g. 7) amino acids of the TNFRSFIA intracellular domain) via a transmembrane domain. In some embodiments, the transmembrane domain is a human TNFRSFIA or a human IL-4R transmembrane domain. In some embodiments, the polypeptide comprises or consists of SEQ ID NO: 43. In some embodiments, a relevant domain comprises an amino acid sequence at least 95% or 100% identical to the sequence set forth in Table 1.

In some embodiments the polypeptide comprises a human LTBR extracellular domain linked to a human IL-4R intracellular domain (and optionally 1-10 (e.g. 7) amino acids of the LTBR intracellular domain) via a transmembrane domain. In some embodiments, the transmembrane domain is a human LTBR or a human IL-4R transmembrane domain. In some embodiments, the polypeptide comprises or consists of SEQ ID NO: 44. In some embodiments, a relevant domain comprises an amino acid sequence at least 95% or 100% identical to the sequence set forth in Table 1.

In some embodiments, the polypeptide comprises a human IL-4RA extracellular domain linked to a human ICOS intracellular domain via a transmembrane domain. In some embodiments, the transmembrane domain is a human ICOS or a human IL-4R transmembrane domain. In some embodiments, the polypeptide comprises or consists of SEQ ID NO: 45. In some embodiments, a relevant domain comprises an amino acid sequence at least 95% or 100% identical to the sequence set forth in Table 1.

In some embodiments, the polypeptide comprises a human LAG3 extracellular domain or a portion thereof (and optionally 1-20 amino acids of the ICOS extracellular domain) linked to a human ICOS intracellular domain via a transmembrane domain. In some embodiments, the transmembrane domain is a human ICOS or a human LAG3 transmembrane domain. In some embodiments, the polypeptide comprises or consists of SEQ ID NO: 46. In some embodiments, a relevant domain comprises an amino acid sequence at least 95% or 100% identical to the sequence set forth in Table 1.

In some embodiments, the polypeptide comprises a human CTLA4 extracellular domain or a portion thereof (and optionally 1-10 (e.g. 7) amino acids of the CTLA4 intracellular domain) linked to a human CD28 intracellular domain via a transmembrane domain. In some embodiments, the transmembrane domain is a human CTLA4 or a human CD28 transmembrane domain. In some embodiments, the polypeptide comprises or consists of SEQ ID NO: 99. In some embodiments, a relevant domain comprises an amino acid sequence at least 95% or 100% identical to the sequence set forth in Table 1.

In some embodiments, the polypeptide comprises a human DR5 extracellular domain or a portion thereof (and optionally 1-10 (e g. 7) amino acids of the DR5 intracellular domain) linked to a human CD28 intracellular domain via a transmembrane domain. In some embodiments, the transmembrane domain is a human DR5 or a human CD28 transmembrane domain. In some embodiments, the polypeptide comprises or consists of SEQ ID NO: 103. In some embodiments, a relevant domain comprises an amino acid sequence at least 95% or 100% identical to the sequence set forth in Table 1.

In some embodiments, the polypeptide comprises a human CD200R extracellular domain or a portion thereof (and optionally, the ICOS extracellular domain or a portion thereof) linked to a human ICOS intracellular domain via a transmembrane domain. In some embodiments, the transmembrane domain is a human CD200R or a human ICOS transmembrane domain. In some embodiments, the polypeptide comprises or consists of SEQ ID NO: 101. In some embodiments, a relevant domain comprises an amino acid sequence at least 95% or 100% identical to the sequence set forth in Table 1.

In some embodiments, the polypeptide comprises a full-length IL21R protein, a LAT1 protein, a BATF protein, a BATF3 protein, a BATF2 protein, an ID2 protein, an ID3 protein, an IRF8 protein, a MYC protein, a POU2F1 protein, a TFAP4 protein, a SMAD4 protein, a NFATCI protein, an EZH2 protein, an EOMES protein, a SOX5 protein, an IRF2BP2 protein, a SOX3 protein, a PRDMI protein, or a RELB protein,

TABLE 1

| Human protein | Domain | SEQ ID NO: |
|---|---|---|
| Fas | Extracellular | 65 |
| Fas | Transmembrane | 66 |
| Fas | Intracellular | 67 |
| OX40 | Extracellular | 68 |
| OX40 | Transmembrane | 69 |
| OX40 | Intracellular | 70 |
| 4-1BB | Extracellular | 71 |
| 4-1BB | Transmembrane | 72 |
| 4-1BB | Intracellular | 73 |
| ICOS | Extracellular | 74 |
| ICOS | Transmembrane | 75 |
| ICOS | Transmembrane | 76 |
| TNFRSF12 | Extracellular | 77 |
| TNFRSF12 | Transmembrane | 78 |
| TNFRSF12 | Intracellular | 79 |
| LTBR | Extracellular | 80 |
| LTBR | Transmembrane | 81 |
| LTBR | Intracellular | 82 |
| LAG3 | Extracellular | 83 |
| LAG3 | Transmembrane | 84 |
| LAG3 | Intracellular | 85 |
| DR5 | Extracellular | 86 |
| DR5 | Transmembrane | 87 |
| DR5 | Intracellular | 88 |
| IL4-R | Extracellular | 89 |
| IL4-R | Transmembrane | 90 |
| IL4-R | Intracellular | 91 |
| DR4 | Extracellular | 92 |
| DR4 | Transmembrane | 93 |
| DR4 | Intracellular | 94 |
| IL-4RA | Extracellular | 95 |
| IL-4RA | Transmembrane | 96 |
| IL-4RA | Intracellular | 97 |
| CTLA4 | Extracellular | 106 |
| CTLA4 | Transmembrane | 107 |
| CTLA4 | Intracellular | 108 |
| CD28 | Extracellular | 109 |
| CD28 | Transmembrane | 110 |
| CD28 | Intracellular | 111 |
| CD200R | Extracellular | 112 |
| CD200R | Transmembrane | 113 |
| CD200R | Intracellular | 114 |

Nucleic acid sequences described herein, for example, SEQ ID Nos: 1-32, and nucleic acid sequences encoding any of the polypeptides described herein can be inserted into the TCR locus of a T cell. In some embodiments, a nucleic acid sequence encoding any one of SEQ ID Nos: 33-97 or 106-114 is inserted into the TCR locus of the T cell. In some embodiments, a nucleic acid sequence that is at least 80%, 85%, 90%, 99%, or 100% identical to any one of the nucleic acid sequences set forth as SEQ ID Nos: 1-32, any one of the nucleic acids set forth ast SEQ ID NOs: 98, 100, 102 or 104, or a nucleic acid sequence that encodes any one of SEQ ID Nos: 33-97 or 106-114, is inserted into the TCR locus of the T cell.

Any polypeptide sequence, nucleic acid sequence, T cell comprising a polypeptide or nucleic acid sequence, or a method that uses a T cell, polypeptide or nucleic acid sequence described herein can be claimed.

Insertion of a heterologous coding sequence into the TCR locus means that the expression of the heterologous protein will be controlled by the endogenous TCR promoter and in some embodiments will be expressed as part of a larger fusion protein with a TCR polypeptide that is subsequently cleaved to form separate TCR and heterologous polypeptides. The TCR polypeptide can be endogenous or also added to the TCR locus to provide a novel TCR affinity (for example, but not limited to, to a cancer antigen) to the T-cell. In some embodiments, the nucleic acid construct is inserted in a target insertion site in exon 1 of a TCR-alpha subunit constant gene (TRAC). In some embodiments, the nucleic acid construct is inserted in a target insertion site in exon 1 of a TCR-beta subunit constant gene (TRBC), for example, in exon 1 of a TRBC1 gene or exon1 of a TRBC2 gene. Upon insertion of the nucleic acid construct into the TCR locus of a cell, the construct is under the control of an endogenous TCR promoter, for example a TRACI promoter or a TRBC promoter. As set forth below, the nucleic acid constructs provided herein encode a TCR or synthetic antigen receptor that is co-expressed with the polypeptide. Once the construct is incorporated into the genome of the T cell by HDR, and under the control of the endogenous promoter, the T cells can be cultured under conditions that allow transcription of the inserted construct into a single mRNA sequence encoding a fusion polypeptide that is then processed into separate heterologous polypeptides (e.g., for example by cleavage of a peptide sequence linking the polypeptides). Insertion of any of the nucleic acid constructs described herein encoding the components of a heterologous T cell receptor and a heterologous polypeptide will produce a T cell with the specificity of the heterologous TCR receptor and the function of the heterologous polypeptide. In some embodiments, the T cell expresses an antigen-specific TCR that recognizes a target antigen. In some embodiments, the T cell expresses an antigen-specific TCR that binds to an antigen in an HLA-independent manner, i.e., a TCR that recognizes surface epitopes independently of the HLA profile of the tumor cell. (See, for example, International Patent Application Publication No. WO2019157454). Similarly, insertion of any of the nucleic acid constructs described herein encoding a synthetic antigen receptor and a heterologous polypeptide will produce a T cell with the specificity of the heterologous TCR receptor and the function of the heterologous polypeptide. In some embodiments, the T cell expresses a synthetic antigen receptor that recognizes a target antigen. In some embodiments, the synthetic antigen receptor is a CAR. In some embodiments, the synthetic antigen receptor is a SynNotch receptor. In some embodiments, the synthetic antigen receptor is a Synthetic Intramembrane Proteolysis Receptor (SNIPR). See, for example. Zhu et al., “Design and modular assembly of synthetic intramembrane proteolysis receptors for custom gene regulation in therapeutic cells.” bioRxiv 2021.05.21.445218; doi: https://doi.org/10.1101/2021.05.21.445218.

In some embodiments, the heterologous nucleic acid inserted into the human T cell encodes, in the following order. (i) a first self-cleaving peptide sequence. (ii) a first heterologous TCR subunit chain, wherein the TCR subunit chain comprises a variable region and a constant region of the TCR subunit: (iii) a second self-cleaving peptide sequence: (iv) a heterologous polypeptide as described herein: (v) a third self-cleaving peptide sequence: (vi) a variable region of a second heterologous TCR subunit chain; and (vii) a portion of the N-terminus of the endogenous TCR subunit, wherein, if the endogenous TCR subunit of the cell is a TCR-alpha (TCR-α) subunit, the first heterologous TCR subunit chain is a heterologous TCR-beta (TCR-β) subunit chain and the second heterologous TCR subunit chain is a heterologous TCR-α subunit chain, and wherein if the endogenous TCR subunit of the cell is a TCR-B subunit, the first heterologous TCR subunit chain is a heterologous TCR-α subunit chain and the second heterologous TCR subunit chain is a heterologous TCR-β subunit chain.

In some embodiments, the heterologous nucleic acid inserted into the human T cell encodes, in the following order, (i) a first self-cleaving peptide sequence; (ii) a heterologous polypeptide as described herein: (iii) a second self-cleaving peptide sequence; (iv) a first heterologous TCR subunit chain, wherein the TCR subunit chain comprises a variable region and a constant region of the TCR subunit: (v) a third self-cleaving peptide sequence: (vi) a variable region of a second heterologous TCR subunit chain; and (vii) a portion of the N-terminus of the endogenous TCR subunit, wherein, if the endogenous TCR subunit of the cell is a TCR-alpha (TCR-α) subunit, the first heterologous TCR subunit chain is a heterologous TCR-beta (TCR-β) subunit chain and the second heterologous TCR subunit chain is a heterologous TCR-α subunit chain, and wherein if the endogenous TCR subunit of the cell is a TCR-B subunit, the first heterologous TCR subunit chain is a heterologous TCR-α subunit chain and the second heterologous TCR subunit chain is a heterologous TCR-β subunit chain.

In the compositions and methods described herein, if the endogenous TCR subunit is a TCR-alpha (TCR-α) subunit, the first beterologous TCR subunit chain is a heterologous TCR-beta (TCR-β) subunit chain and the second heterologous TCR subunit chain is a heterologous TCR-α subunit chain. In some methods, if the endogenous TCR subunit is a TCR-β subunit, the first heterologous TCR subunit chain is a heterologous TCR-α subunit chain and the second heterologous TCR subunit chain is a heterologous TCR-β subunit chain.

As used throughout, the term “endogenous TCR subunit” is the TCR subunit, for example, TCR-α or TCR-B that is endogenously expressed by the cell that the nucleic acid construct is introduced into. As set forth above, the nucleic acid constructs described herein encode multiple amino acid sequences that are expressed as a multicistronic sequence that is processed, i.e., self-cleaved, to produce two or more amino acid sequences, for example, a TCR-α subunit, a TCR-B subunit and the polypeptide encoded by the construct, or a synthetic antigen receptor (e.g. a CAR (See, for example, Guedan et al. “Engineering and Design of Chimeric Antigen Receptors,” Mol. Ther Methods & Clinical Development 12:145-156 (2019)) or SynNotch receptor (See, for example, Cho et al. “Engineering Axl specific CAR and SynNotch receptor for cancer therapy.” Nature Scientific Reports 8, Article No: 3846 (2018)) and the polypeptide encoded by the construct.

In some nucleic acid constructs, the size of the nucleic acid encoding the N-terminal portion of the endogenous TCR subunit will depend on the number of nucleotides in the endogenous TRAC or TRBC nucleic acid sequence between the start of TRAC exon 1 or TRBC exon 1 and the targeted insertion site. For example, if the number of nucleotides between the start of TRAC exon 1 and the insertion site is less than or greater than 25 nucleotides, a nucleic acid of less than or greater than 25 nucleotides encoding the N-terminal portion of the endogenous TCR-α subunit can be in the construct.

In the examples above, translation of the mRNA sequence transcribed from the construct results in expression of one protein that self-cleaves into four, separate polypeptide sequences. i.e., an inactive, endogenous variable region peptide lacking a transmembrane domain, (which can be, e.g., degraded in the endoplasmic reticulum or secreted following translation), a full-length heterologous antigen-specific TCR-β chain or TCR-α chain, a polypeptide sequence as described herein, and a full length heterologous antigen-specific TCR-a chain or TCR-β chain. The full-length antigen specific TCR-B chain and the full length antigen-specific TCR-α chain form a TCR with desired antigen-specificity. In some embodiments, the polypeptide enhances or imparts a desired function(s) in the T cell. mRNA transcribed from any of the other nucleic acid constructs described herein are similarly processed in a T cell.

In some embodiments, the nucleic acid construct encodes, in the following order, (i) a first self-cleaving peptide sequence; (ii) a first heterologous TCR subunit chain, wherein the TCR subunit chain comprises the variable region and the constant region of the TCR subunit: (iii) a second self-cleaving peptide sequence: (iv) a second heterologous TCR subunit chain, wherein the TCR subunit chain comprises the variable region and the constant region of the TCR subunit; (v) a third self-cleaving peptide sequence; (vi) a heterologous polypeptide described herein; and (vii) a fourth self-cleaving peptide sequence or a poly A sequence, wherein if the endogenous TCR subunit is a TCR-alpha (TCR-α) subunit, the first heterologous TCR subunit chain is a heterologous TCR-beta (TCR-β) subunit chain and the second heterologous TCR subunit chain is a heterologous TCR-α subunit chain, and wherein if the endogenous TCR subunit is a TCR-B subunit, the first heterologous TCR subunit chain is a heterologous TCR-α subunit chain and the second heterologous TCR subunit chain is a heterologous TCR-β subunit chain.

In some embodiments, the nucleic acid construct encodes, in the following order, (i) a first self-cleaving peptide sequence: (ii) a synthetic antigen receptor: (iii) a second self-cleaving peptide sequence: (iv) a heterologous polypeptide described herein; and (v) a third self-cleaving peptide sequence or a polyA sequence.

In some embodiments, the nucleic acid construct encodes, in the following order, (i) a first self-cleaving peptide sequence; (ii) a heterologous polypeptide; (iii) a second self-cleaving peptide sequence; (iv) a synthetic antigen receptor; and (v) a third self-cleaving peptide sequence or a polyA sequence.

Examples of self-cleaving peptides include, but are not limited to, self-cleaving viral 2A peptides, for example, a porcine teschovirus-1 (P2A) peptide, a Thosea asigna virus (T2A) peptide, an equine rhinitis A virus (E2A) peptide, or a foot-and-mouth disease virus (F2A) peptide. Self-cleaving 2A peptides allow expression of multiple gene products from a single construct. (Sec, for example, Chng et al. “Cleavage efficient 2A peptides for high level monoclonal antibody expression in CHO cells,” MAbs 7 (2): 403-412 (2015)). In some embodiments, the nucleic acid construct comprises two or more self-cleaving peptides. In some embodiments, the two or more self-cleaving peptides are all the same. In other embodiments, at least one of the two or more self-cleaving peptides is different.

In some embodiments, one or more linker sequences separate the components of the nucleic acid construct. The linker sequence can be two, three, four, five, six, seven, eight, nine, ten amino acids or greater in length.

In some embodiments, the nucleic acid construct comprises flanking homology arm sequences having homology to a human TCR locus. In the compositions and methods described herein, the length of one or both homology arm sequences is at least about 50, 100, 150, 200, 250, 300, 350, 400 or 450 nucleotides. In some cases, a nucleotide sequence that is homologous to a genomic sequence is at least 80%, 90%, 95%, 99% or 100% complementary to the genomic sequence. In some embodiments, one or both homology arm sequences optionally comprises a mismatched nucleotide sequence compared to a homologous sequence in the genomic sequence in the TCR locus flanking the insertion site in the TCR locus.

In some embodiments, the nucleic acid construct optionally encodes a selectable marker that can be used to separate or isolate subpopulations of modified T cells. In some embodiments, the nucleic acid construct optionally comprises a barcode sequence that indicates the identity of the polypeptide.

Any of the polypeptides described herein can be encoded by any of the nucleic acid constructs described herein. In some embodiments, the polypeptide sequence encoded by the heterologous nucleic acid construct is at least 95% identical to an amino acid sequence selected from the group consisting of SEQ ID NO: 33-64.

Also provided are polypeptides that are at least 95% identical to SEQ ID NO 33, SEQ ID NO: 34, SEQ ID NO: 35, SEQ ID NO: 40, SEQ ID NO: 41, SEQ ID NO: 42. SEQ ID NO: 43, SEQ ID NO: 44, SEQ ID NO: 45 or SEQ ID NO: 46. Nucleic acids encoding these polypeptides are also provided herein.

Also provided is a human T cell comprising any of the nucleic acid sequences described herein. Populations (e.g., a plurality) of human T cells comprising any of the nucleic acid sequences described herein are also provided.

Any of the nucleic acid constructs encoding any of the polypeptides described herein can be used to make modified T cells. In some embodiments, the method comprises (a) introducing into the human T cell (i) a targeted nuclease that cleaves a target region in the TCR locus of a human T cell to create a target insertion site in the genome of the cell; and (ii) a nucleic acid construct encoding any of the polypeptides described herein, for example,
a polypeptide comprising a human Fas extracellular domain or portion thereof linked to a human OX40 intracellular domain (and optionally, 1-10 (e.g., 7) amino acids of the Fas intracellular domain) via a transmembrane domain: (Fas-OX40);
a polypeptide comprising a human TNFRSF12 extracellular domain linked to a human OX40 intracellular domain (and optionally 1-10 (e.g., 7) amino acids of the TNFRSF12 intracellular domain) via a transmembrane domain;
a polypeptide comprising a human LTBR extracellular domain linked to a human OX40 intracellular domain (and optionally 1-10 (e.g. 7) amino acids of the LTBR intracellular domain) via a transmembrane domain;
a truncated human LTBR protein comprising the human LTBR extracellular domain, transmembrane domain and about 1-10 (e.g. 7) amino acids of the intracellular domain.
a truncated human TNFRSF12 protein comprising the human TNFRSF12 extracellular domain, transmembrane domain and about 1-10 (e.g. 7) amino acids of the intracellular domain;
a polypeptide comprising a human LAG-3 extracellular domain linked to a buman 4-1BB intracellular domain (and optionally 1-10 (e.g. 7) amino acids of the LAG3 intracellular domain) via a transmembrane domain;
a polypeptide comprising a human DR5 extracellular domain linked to a human IL-4R intracellular domain (and optionally 1-10 (e.g. 7) amino acids of the DR5 intracellular domain) via a transmembrane domain;
a polypeptide comprising a human DR4 extracellular domain linked to a human IL-4R intracellular domain (and optionally 1-10 (e.g. 7) amino acids of the DR4 intracellular domain) via a transmembrane domain;
a polypeptide comprising a human TNFRSFIA extracellular domain linked to a human IL-4R intracellular domain (and optionally 1-10 (e.g. 7) amino acids of the TNFRSFIA intracellular domain) via a transmembrane domain;
a polypeptide comprising a human LTBR extracellular domain linked to a human IL-4R intracellular domain (and optionally 1-10 (e.g. 7) amino acids of the LTBR intracellular domain) via a transmembrane domain;
a polypeptide comprising a human IL-4RA extracellular domain linked to a human ICOS intracellular domain via a transmembrane domain;
a polypeptide comprising a human LAG3 extracellular domain or a portion thereof (and optionally 1-20 amino acids of the ICOS extracellular domain) linked to a human ICOS intracellular domain via a transmembrane domain;
a polypeptide comprising an IL21R protein, a LAT1 protein, a BATF protein, a BATF3 protein, a BATF2 protein, an ID2 protein, an ID3 protein, an IRF8 protein, a MYC protein, a POU2F1 protein, a TFAP4 protein, a SMAD4 protein, a NFATCI protein, an EZH2 protein, an EOMES protein, a SOX5 protein, an IRF2BP2 protein, a SOX3 protein, a PRDMI protein, or a RELB protein; and
(b) allowing recombination to occur, thereby inserting the nucleic acid construct in the target insertion site to generate a modified buman T cell.

In some embodiments, the nucleic acid is inserted into a T cell by introducing into the T cell, (a) a targeted nuclease that cleaves a target region in exon 1 of a TCR-α subunit constant gene (TRAC) to create an insertion site in the genome of the T cell; and (b) the nucleic acid construct, wherein the nucleic acid construct is incorporated into the insertion site by homology directed repair (HDR). In some embodiments, the nucleic acid construct is inserted into a T cell by introducing into the T cell, (a) a targeted nuclease that cleaves a target region in exon 1 of a TCR-β subunit constant gene (TRBC), for example, TRBC1 or TRBC 2, to create an insertion site in the genome of the T cell; and (b) the nucleic acid construct, wherein the nucleic acid sequence is incorporated into the insertion site by homology directed repair (HDR).

In some embodiments, the nucleic acid construct is inserted by introducing a viral vector comprising the nucleic acid construct into the cell. Examples of viral vectors include, but are not limited to, adeno-associated viral (AAV) vectors, retroviral vectors or lentiviral vectors. In some embodiments, the lentiviral vector is an integrase-deficient lentiviral vector.

In some embodiments, the nucleic acid construct is inserted by introducing a non-viral vector comprising the nucleic acid construct into the cell. In non-viral delivery methods, the nucleic acid can be naked DNA, or in a non-viral plasmid or vector. For non-viral delivery methods, the DNA template can be inserted using a non-viral genome targeting protocol based on a Cas9 shuttle system and an anionic polymer. Transposon-based gene transfer can also be used. See, for example, Tipance et al. “Preclinical and clinical advances m transposon-based gene therapy,” Biosci Rep. 37 (6): BSR20160614 (2017)

In some cases, the nucleic acid sequence is introduced into the cell as a linear DNA template. In some cases, the nucleic acid sequence is introduced into the cell as a double-stranded DNA template. In some cases, the DNA template is a single-stranded DNA template. In some cases, the single-stranded DNA template is a pure single-stranded DNA template. As used herein, by “pure single-stranded DNA” is meant single-stranded DNA that substantially lacks the other or opposite strand of DNA. By “substantially lacks” is meant that the pure single-stranded DNA lacks at least 100-fold more of one strand than another strand of DNA. In some cases, the DNA template is a double-stranded or single-stranded plasmid or mini-circle.

In some embodiments, the targeted nuclease is selected from the group consisting of an RNA-guided nuclease domain, a transcription activator-like effector nuclease (TALEN), a zinc finger nuclease (ZFN) and a megaTAL (See, for example, Merkert and Martin “Site-Specific Genome Engineering in Human Pluripotent Stem Cells,” Int. J. Mol. Sci. 18 (7): 1000 (2016)). In some embodiments, the RNA-guided nuclease is a Cas9 nuclease and the method further comprises introducing into the cell a guide RNA that specifically hybridizes to a target region in the genome of the cell, for example, a target region in exon 1 of the TRAC gene in a T cell. In other embodiments, the RNA-guided nuclease is a Cas9 nuclease and the method further comprises introducing into the cell a guide RNA that specifically hybridizes to a target region in exon 1 of the TRBC gene.

As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the DNA targeting sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence

Generally, the DNA targeting sequence is designed to complement (e.g., perfectly complement) or substantially complement the target DNA sequence. In some cases, the DNA targeting sequence can incorporate wobble or degenerate bases to bind multiple genetic elements. In some cases, the 19 nucleotides at the 3′ or 5′ end of the binding region are perfectly complementary to the target genetic element or elements. In some cases, the binding region can be altered to increase stability. For example, non-natural nucleotides, can be incorporated to increase RNA resistance to degradation. In some cases, the binding region can be altered or designed to avoid or reduce secondary structure formation in the binding region. In some cases, the binding region can be designed to optimize G-C content. In some cases, G-C content is preferably between about 40% and about 60% (e.g., 40%, 45%, 50%, 55%, 60%). In some embodiments, the Cas9 protein can be in an active endonuclease form, such that when bound to target nucleic acid as part of a complex with a guide RNA or part of a complex with a DNA template, a double strand break is introduced into the target nucleic acid. In the methods provided herein, a Cas9 polypeptide or a nucleic acid encoding a Cas9 polypeptide can be introduced into the cell. The double strand break can be repaired by HDR to insert the DNA template into the genome of the cell. Various Cas9 nucleases can be utilized in the methods described herein. For example, a Cas9 nuclease that requires an NGG protospacer adjacent motif (PAM) immediately 3′ of the region targeted by the guide RNA can be utilized. Such Cas9 nucleases can be targeted to, for example, a region in exon 1 of the TRAC or exon 1 of the TRAB that contains an NGG sequence. As another example. Cas9 proteins with orthogonal PAM motif requirements can be used to target sequences that do not have an adjacent NGG PAM sequence. Exemplary Cas9 proteins with orthogonal PAM sequence specificities include, but are not limited to those described in Esvelt et al., Nature Methods 10:1116-1121 (2013).

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region, for example exon 1 of a TRAC gene or exon 1 of a TRBC gene. Nickase pairs can provide enhanced specificity because off-target effects are likely to result in single nicks, which are generally repaired without lesion by base-excision repair mechanisms. Exemplary Cas9 nickases include Cas9 nucleases having a D10A or H840A mutation (See, for example. Ran et al. “Double nicking by RNA-guided CRISPR Cas9 for enhanced genome editing specificity.” Cell 154 (6): 1380-1389 (2013).

In some embodiments, the Cas9 nuclease, the guide RNA and the nucleic acid sequence are introduced into the cell as a ribonucleoprotein complex (RNP)-nucleic acid sequence (e.g. a DNA template) complex, wherein the RNP-nucleic acid sequence complex comprises: (i) the RNP, wherein the RNP comprises the Cas9 nuclease and the guide RNA; and (ii) the nucleic acid sequence or construct.

In some embodiments, the molar ratio of RNP to DNA template can be from about 3:1 to about 100:1. For example, the molar ratio can be from about 5:1 to 10:1, from about 5:1 to about 15.1, 5:1 to about 20:1; 5:1 to about 25:1; from about 8:1 to about 12:1, from about 8:1 to about 15:1, from about 8:1 to about 20:1, or from about 8:1 to about 25:1.

In some embodiments, the DNA template in the RNP-DNA template complex is at a concentration of about 2.5 pM to about 25 pM. In some embodiments, the amount of DNA template is about 1 μg to about 10 μg.

In some cases, the RNP-DNA template complex is formed by incubating the RNP with the DNA template for less than about one minute to about thirty minutes, at a temperature of about 20° C. to about 25° C. In some embodiments, the RNP-DNA template complex and the cell are mixed prior to introducing the RNP-DNA template complex into the cell.

In some embodiments the nucleic acid sequence or the RNP-DNA template complex is introduced into the cells by electroporation. Methods, compositions, and devices for electroporating cells to introduce a RNP-DNA template complex can include those described in the examples herein. Additional or alternative methods, compositions, and devices for electroporating cells to introduce a RNP-DNA template complex can include those described in WO/2006/001614 or Kim, J. A. et al. Biosens. Bioelectron. 23, 1353-1360 (2008). Additional or alternative methods, compositions, and devices for electroporating cells to introduce a RNP-DNA template complex can include those described in U.S. Patent Appl. Pub Nos. 2006/0094095; 2005/0064596; or 2006/0087522. Additional or alternative methods, compositions, and devices for electroporating cells to introduce a RNP-DNA template complex can include those described in Li, L. H. et al. Cancer Res. Treat. 1, 341-350 (2002): U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842. Additional or alternative methods, compositions, and devices for electroporating cells to introduce a RNP-DNA template complex can include those described in Geng, T. et al., J. Control Release 144, 91-100 (2010); and Wang, J., et al. Lab. Chip 10, 2057-2061 (2010).

In some embodiments, the RNP is delivered to the cells in the presence of an anionic polymer. In some embodiments, the anionic polymer is an anionic polypeptide or an anionic polysaccharide. In some embodiments, the anionic polymer is an anionic polypeptide (e.g., a polyglutamic acid (PGA), a polyaspartic acid, or polycarboxyglutamic acid). In some embodiments, the anionic polymer is an anionic polysaccharide (e.g., hyaluronic acid (HA), heparin, heparin sulfate, or glycosaminoglycan). In some embodiments, the anionic polymer is poly(acrylic acid) (PAA), poly(methacrylic acid) (PMAA), poly(styrene sulfonate), or polyphosphate. In some embodiments, the anionic polymer has a molecular weight of at least 15 kDa (e.g., between 15 kDa and 50 kDa). In some embodiments, the anionic polymer and the Cas protein are in a molar ratio of between 10:1 and 120.1, respectively (e.g., 10:1, 20:1, 30:1, 40:1, 50:1, 60:1, 70:1, 80:1, 90:1, 100:1, 110:1, or, 120:1). In some embodiments of this aspect, the molar ratio of sgRNA:Cas protein is between 0.25:1 and 4:1 (e.g., 0.25:1, 0.5:1, 1:1, 1.2:1, 1.4:1, 1.6:1, 1.8:1, 2:1, 2.2:1, 2.4:1, 2.6:1, 2.8:1, 3:1, 3.2:1, 3.4:1, 3.6:1, 3.8:1, or 4:1).

In some embodiments, the donor template comprises a homology directed repair (HDR) template and one or more DNA-binding protein target sequences. In some embodiments, the donor template has one DNA-binding protein target sequence and one or more protospacer adjacent motif (PAM). The complex containing the DNA-binding protein (e.g., a RNA-guided nuclease), the donor gRNA, and the donor template can shuttle the donor template, without cleavage of the DNA-binding protein target sequence, to the desired intracellular location (e.g., the nucleus) such that the HDR template can integrate into the cleaved target nucleic acid. In some embodiments, the DNA-binding protein target sequence and the PAM are located at the 5′ terminus of the HDR template. Particularly, in some embodiments, the PAM can be located at the 5′ terminus of the DNA-binding protein target sequence. In other embodiments, the PAM can be located at the 3′ terminus of the DNA-binding protein target sequence. In some embodiments, the DNA-binding protein target sequence and the PAM are located at the 3′ terminus of the HDR template. Particularly, in some embodiments, the PAM can be located at the 5′ terminus of the DNA-binding protein target sequence. In other embodiments, the PAM is located at the 3′ terminus of the DNA-binding protein target sequence. In some embodiments, the donor template has two DNA-binding protein target sequences and two PAMs. Particularly, in some embodiments, a first DNA-binding protein target sequence and a first PAM are located at the 5′ terminus of the HDR template and a second DNA-binding protein target sequence and a second PAM are located at the 3′ terminus of the HDR template. In some embodiments, the first PAM is located at the 5′ terminus of the first DNA-binding protein target sequence and the second PAM is located at the 5′ of the second DNA-binding protein target sequence. In other embodiments, the first PAM is located at the 5′ terminus of the first DNA-binding protein target sequence and the second PAM is located at the 3′ of the second DNA-binding protein target sequence. In yet other embodiments, the first PAM is located at the 3″ terminus of the first DNA-binding protein target sequence and the second PAM is located at the 5′ of the second DNA-binding protein target sequence. In yet other embodiments, the first PAM is located at the 3′ terminus of the first DNA-binding protein target sequence and the second PAM is located at the 3′ of the second DNA-binding protein target sequence.

In some embodiments, the nucleic acid sequence or RNP-DNA template complex are introduced into about 1×10⁵ to about 2×10⁶ cells T cells. For example, the nucleic acid sequence or RNP-DNA template complex can be introduced into about 1×10⁵ cells to about 5×10⁵ cells, about 1×10⁵ cells to about 1×10⁶ cells, 1×10⁵ cells to about 1.5×10⁶ cells, 1×10⁵ cells to about 2×10⁶ cells, about 1×10⁶ cells to about 1.5×10⁶ cells or about 1×10⁶ cells to about 2×10⁶ cells.

In the methods and compositions provided herein, the human T cells can be primary T cells. In some embodiments, the T cell is a regulatory T cell, an effector T cell, a memory T cell or a naïve T cell. In some embodiments, the effector T cell is a CD8⁺ T cell. In some embodiments, the T cell is an CD4+ cell. In some embodiments, the T cell is a CD4⁺CD8⁺ T cell. In some embodiments, the T cell is a CD4⁻CD8⁻T cell. In some embodiments, the T cell is a T cell that expresses a TCR receptor or differentiates into a T cell that expresses a TCR receptor.

### Methods of Treatment

Any of the methods and compositions described herein can be used to modify T cells obtained from a human subject. Any of the methods and compositions described herein can be used to modify T cells obtained from a human subject to enhance an immune response in the subject. Any of the methods and compositions described herein can be used to modify T cells obtained from a human subject to treat or prevent a disease (e.g., cancer, an infectious disease, an autoimmune disease, transplantation rejection, graft vs. host disease or other inflammatory disorder in a subject).

As used herein by subject is meant an individual. The subject can be an adult subject or a pediatric subject. Pediatric subjects include subjects ranging in age from birth to eighteen years of age.

Provided herein is a method of enhancing an immune response in a human subject comprising administering any of the modified T cells described herein, i.e., T cells that heterologously express a polypeptide described herein, for example,
a polypeptide comprising a human Fas extracellular domain or portion thereof linked to a human OX40 intracellular domain (and optionally, 1-10 (e.g., 7) amino acids of the Fas intracellular domain) via a transmembrane domain: (Fas-OX40);
a polypeptide comprising a human TNFRSF12 extracellular domain linked to a human OX40 intracellular domain (and optionally 1-10 (e.g., 7) amino acids of the TNFRSF12 intracellular domain) via a transmembrane domain;
a polypeptide comprising a human LTBR extracellular domain linked to a human OX40 intracellular domain (and optionally 1-10 (e.g. 7) amino acids of the LTBR intracellular domain) via a transmembrane domain;
a truncated human LTBR protein comprising the human LTBR extracellular domain, transmembrane domain and about 1-10 (e.g. 7) amino acids of the intracellular domain.
a truncated human TNFRSF12 protein comprising the human TNFRSF12 extracellular domain, transmembrane domain and about 1-10 (e.g. 7) amino acids of the intracellular domain;
a polypeptide comprising a human LAG-3 extracellular domain linked to a human 4-1BB intracellular domain (and optionally 1-10 (e.g. 7) amino acids of the LAG3 intracellular domain) via a transmembrane domain;
a polypeptide comprising a human DR5 extracellular domain linked to a human IL-4R intracellular domain (and optionally 1-10 (e.g. 7) amino acids of the DR5 intracellular domain) via a transmembrane domain;
a polypeptide comprising a human DR4 extracellular domain linked to a human IL-4R intracellular domain (and optionally 1-10 (e.g. 7) amino acids of the DR4 intracellular domain) via a transmembrane domain;
a polypeptide comprising a human TNFRSFIA extracellular domain linked to a human IL-4R intracellular domain (and optionally 1-10 (e.g. 7) amino acids of the TNFRSFIA intracellular domain) via a transmembrane domain;
a polypeptide comprising a human LTBR extracellular domain linked to a human IL-4R intracellular domain (and optionally 1-10 (e.g. 7) amino acids of the LTBR intracellular domain) via a transmembrane domain;
a polypeptide comprising a human IL-4RA extracellular domain linked to a human ICOS intracellular domain via a transmembrane domain;
a polypeptide comprising a human LAG3 extracellular domain or a portion thereof (and optionally 1-20 amino acids of the ICOS extracellular domain) linked to a human ICOS intracellular domain via a transmembrane domain: or
a polypeptide comprising an IL21R protein, a LAT1 protein, a BATF protein, a BATF3 protein, a BATF2 protein, an ID2 protein, an ID3 protein, an IRF8 protein, a MYC protein, a POU2F1 protein, a TFAP4 protein, a SMAD4 protein, a NFATCI protein, an EZH2 protein, an EOMES protein, a SOX5 protein, an IRF2BP2 protein a SOX3 protein, a PRDMI protein, or a RELB protein.

In some embodiments, T cells are obtained from the subject and modified using any of the methods provided herein to express an antigen-specific TCR or synthetic antigen receptor, prior to administering the modified T cells to the subject. In some embodiments, the subject has cancer and the target antigen is a cancer-specific antigen. In some embodiments, the subject has an autoimmune disorder and the antigen is an antigen associated with the autoimmune disorder. In some embodiments, the subject has an infection and target antigen is an antigen associated with the infection.

Also provided is a method for treating cancer in a human subject comprising: a) obtaining T cells from the subject; b) modifying the T cells using any of the methods provided herein to express an antigen-specific TCR or a synthetic antigen receptor that recognizes a target antigen in the subject; and c) administering the modified T cells to the subject, wherein the human subject has cancer and the target antigen is a cancer-specific antigen. As used throughout, the phrase “cancer-specific antigen” means an antigen that is unique to cancer cells or is expressed more abundantly in cancer cells than in in non-cancerous cells. In some embodiments, the cancer-specific antigen is a tumor-specific antigen.

As used herein, cancer is a disease characterized by the rapid and uncontrolled growth of aberrant cells. Cancer cells can spread locally or through the bloodstream and lymphatic system to other parts of the body. In some embodiments, the cancer is a solid tumor. In some embodiments, the cancer is a blood or hematological cancer. Exemplary cancers include, but are not limited to, breast cancer, prostate cancer, ovarian cancer, glioblastoma, cervical cancer, skin cancer, pancreatic cancer, colorectal cancer, bladder cancer, endometrial cancer, renal cancer, liver cancer, brain cancer, lymphoma, leukemia (for example, acute myeloid leukemia), myeloma, lung cancer, and the like. It is understood that the methods provided herein can also be used to target circulating cancer cells, for example, cells shed by a solid tumor into the bloodstream of a subject.

In some embodiments, the T cells for treating cancer express a polypeptide comprising an amino acid sequence that is at least 95% identical to LAG3/4-1BB (SEQ ID NO: 40), DR5-IL-4R (SEQ ID NO: 41), DR4-IL-4R (SEQ ID NO: 42), TNFRSFIA-IL-4R (SEQ ID NO: 43), LTBR-IL-4R (SEQ ID NO: 44), IL-4RA-ICOS (SEQ ID NO: 45), LAG-3 ICOS (SEQ ID NO: 46), NFATCI (SEQ ID NO: 57), EZH2 (SEQ ID NO: 58), EOMES (SEQ ID NO: 59), SOX5 (SEQ ID NO: 60), IRF2BP2 (SEQ ID NO: 61). SOX3 (SEQ ID NO: 62), PRDMI (SEQ ID NO: 63), or RELB (SEQ ID NO: 64). In some embodiments for treating cancer, the T cells express a polypeptide that is at least 95% identical to SEQ ID NO: 99, 101, 103 or 105.

In some embodiments, the T cells for treating cancer express a polypeptide comprising an amino acid sequence that is at least 95% identical to Fas-OX40 (SEQ ID NO: 33), TNFRSF12-OX40 (SEQ ID NO: 34), LTBR-OX40 (SEQ ID NO: 35). LTBRtrunc (SEQ ID NO: 36), TNFRSF12trune (SEQ ID NO: 37), IL-21R (SEQ ID NO: 38), LAT1 (SEQ ID NO: 39) BATF (SEQ ID NO: 47), BATF3 9 (SEQ ID NO: 48), BATF2 (SEQ ID NO: 49), ID2 (SEQ ID NO: 50), ID3 (SEQ ID NO: 51), IRF8 (SEQ ID NO: 52), MYC (SEQ ID NO: 53), POU2F1 (SEQ ID NO: 54), TFAP4 (SEQ ID NO: 55) or SMAD4 (SEQ ID NO: 56).

In some embodiments, tumor infiltrating lymphocytes, a heterogeneous and cancer-specific T-cell population, are obtained from a cancer subject and expanded ex vivo. The characteristics of the patient's cancer determine a set of tailored cellular modifications, and these modifications are applied to the tumor infiltrating lymphocytes using any of the methods described herein.

Also provided herein is a method of treating an autoimmune disease, an allergic disorder or transplant rejection in a human subject comprising: a) obtaining T cells from the subject: b) modifying the T cells using any of the methods provided herein to express an antigen-specific TCR or synthetic antigen receptor that recognizes a target antigen in the subject; and c) administering the modified T cells to the subject, wherein the human subject has an autoimmune disorder and the target antigen is antigen associated with the autoimmune disorder. In some embodiments, the T cells are regulatory T cells.

As used herein, an autoimmune disease is a disease where the immune system cannot differentiate between a subject's own cells and foreign cells, thus causing the immune system to mistakenly attack healthy cells in the body. Examples of autoimmune disorders include, but are not limited to, inflammatory bowel disease, multiple sclerosis, psoriasis, rheumatoid arthritis, systemic lupus erythematosus, Graves' disease, type 1 diabetes, Sjogren's syndrome, autoimmune thyroid disease, and celiac disease.

In some embodiments for treating an autoimmune disorder, an allergic disorder or transplant rejection, the T cells express a polypeptide that is at least 95% identical to LAG3/4-1BB (SEQ ID NO: 40), DR5-IL-4R (SEQ ID NO: 41), DR4-IL-4R (SEQ ID NO: 42), TNFRSFIA-IL-4R (SEQ ID NO. 43), LTBR-IL-4R (SEQ ID NO: 44), IL-4RA-ICOS (SEQ ID NO: 45), LAG-3 ICOS (SEQ ID NO: 46), NFATCI (SEQ ID NO: 57), EZH2 (SEQ ID NO: 58), EOMES (SEQ ID NO: 59), SOX5 (SEQ ID NO: 60). IRF2BP2 (SEQ ID NO: 61), SOX3 (SEQ ID NO: 62), PRDMI (SEQ ID NO: 63), or RELB (SEQ ID NO. 64). In some embodiments for treating an autoimmune disorder, an allergic disorder or transplant rejection, the T cells express a polypeptide that is at least 95% identical to SEQ ID NO: 99, 101, 103 or 105.

Also provided herein is a method of treating an infection in a human subject comprising: a) obtaining T cells from the subject: b) modifying the T cells using any of the methods provided herein to express an antigen-specific TCR or a synthetic antigen receptor that recognizes a target antigen in the subject; and c) administering the modified T cells to the subject, wherein the subject has an infection and the target antigen is an antigen associated with the infection in the subject.

In some embodiments for treating infection, the T cells express a polypeptide comprising an amino acid sequence that is at least 95% identical to Fas-OX40 (SEQ ID NO: 33), TNFRSF12-OX40 (SEQ ID NO: 34), LTBR-OX40 (SEQ ID NO: 35). LTBRtrunc (SEQ ID NO: 36), TNFRSF12trunc (SEQ ID NO: 37), IL-21R (SEQ ID NO: 38), LAT1 (SEQ ID NO: 39) BATF (SEQ ID NO: 47), BATF3 9 (SEQ ID NO: 48), BATF2 (SEQ ID NO: 49), ID2 (SEQ ID NO: 50), ID3 (SEQ ID NO: 51), IRF8 (SEQ ID NO: 52), MYC (SEQ ID NO: 53). POU2F1 (SEQ ID NO: 54), TFAP4 (SEQ ID NO: 55) or SMAD4 (SEQ ID NO: 56).

In some embodiments, the T cell is autologous (i.e., from the same subject who will receive the modified cells) or allogenic (i.e., from a subject other than the subject who will receive the modified cells). In some examples, the T cell is an iPSC-derived T cell. Sec, for example, Nagano et al. Mol. Therapy Methods & Clinical Development 16:126-135 (2020). Any of the methods of treatment provided herein can further comprise expanding the population of T cells before the T cells are modified. Any of the methods of treatment provided herein can further comprise expanding the population of T cells after the T cells are modified and prior to administration to the subject.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### Isolation and Culture of Primary Human T Cells

T cell isolation and cultures were conducted as previously described (Roth et al., Nature 559:405-409 (2018); and Roth et al., Cell 181:728-744 (2020)). Briefly, human T cells were isolated from either fresh whole blood, leukoreduction chamber residuals following Trima Apheresis (Vitalant, San Francisco, CA), or peripheral blood (PB) leukapheresis pack (STEMCELL) from healthy donors. Peripheral blood mononuclear cells (PBMCs) were isolated from whole blood samples by Lymphoprep centrifugation (STEMCELL) using SepMate tubes (STEMCELL). T cells were isolated from PBMCs from all cell sources by magnetic negative selection using an EasySep Human T Cell Isolation Kit (STEMCELL). Fresh blood was taken from healthy human donors under a protocol approved by the UCSF Committee on Human Research (CHR #13-11950).

Freshly isolated primary cells were cultured in XVivo15 medium (Lonza) supplemented with 5% fetal bovine serum (FBS), 50 μM 2mercaptoethanol, and 10 mM N-acetyl L-cystine. Prior to nucleofection, T cells were stimulated for 44 to 52 hours at a density of 1 million cells per mL of media with anti-human CD3/CD28 Dynabeads (ThermoFisher), at a bead to cell ratio of 1:1. Cells were also cultured in XVivo15 media containing IL-2 (500 U ml-1; UCSF Pharmacy), IL-7 (5 ng ml-1: ThermoFisher), and IL-15 (5 ng ml-1: Life Tech). After nucleofection, T cells were cultured in XVivo15 media containing IL-2 (500 U ml-1) and maintained at approximately 1 million cells per mL of media. Every 2-3 days, cells were topped up with additional media and fresh IL-2 (final concentration of 500 U ml-1).

### Generation of Plasmid Libraries for Pooled Knock-in

The 229 constructs included in the pooled knock-in library were designed using the Twist Bioscience codon optimization tool and were commercially synthesized and cloned (Twist Bioscience) into a custom pUC19 plasmid containing the NY-ESO-1 TCR replacement HDR sequence. Two barcodes unique for each library member were also introduced into degenerate bases immediately 5′ and 3′ of the region of the individual gene insert. Individual pooled plasmid libraries were created by pooling single construct plasmids into respective libraries (Transcription factors, 100 members; switch receptors, 129 members) or in one complete pool, along with knock-in controls.

The CAR plasmid pool was created in a pooled assembly fashion by amplifying constructs from TCR plasmid pool described above as a DNA template. PCR amplification (Kapa Hot Start polymerase) produced a pooled library of amplicons with small overhangs homologous to a pUC19 plasmid containing CD19/4-1BB or GD2/CD28 CAR HDR sequence. This amplicon pool treated with Dpn1 restriction enzyme (NEB) to remove residual circular TCR plasmids, SPRI purified (1.0×), and eluted into H20. Gibson Assemblies (NEB) were then used to construct a plasmid pool containing all 229 library members and knock-in controls, plus the new CAR sequence. The CAR plasmid pool was SPRI purified as before and transformed into Endura electrocompetent cells (Lucigen) and Maxiprepped (Zymo) for further use.

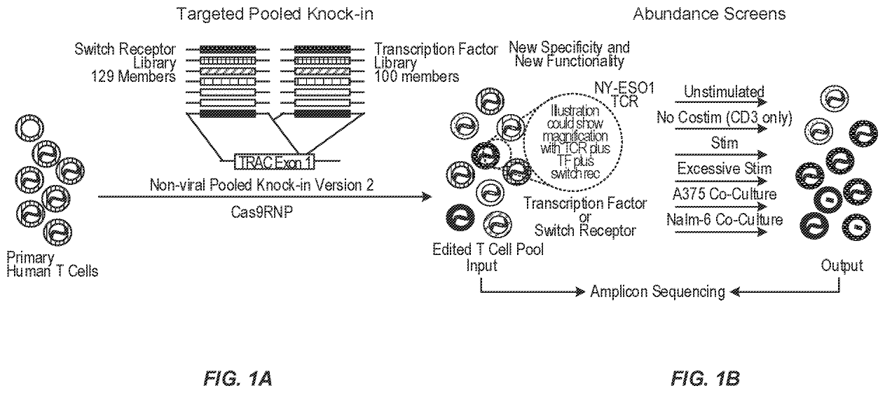

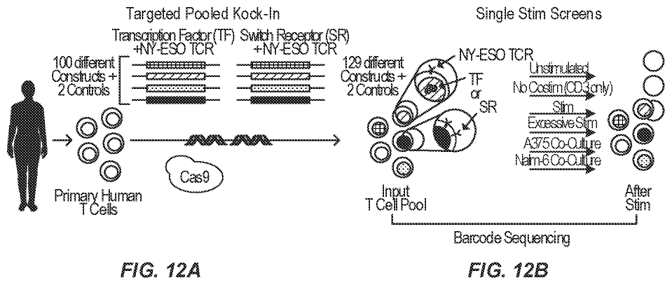

FIGS. 1 and 12 are illustrations of the pooled knock-in platform and subsequent functional single stimulation screens.

### HDR Template Generation

HDR templates were produced as previously described (Roth et al., 2018. Roth et al., 2020). In brief, TCR or CAR plasmid pools were used as templates for high-output PCR amplification (Kapa Hot Start polymerase). The resulting amplicons, deemed double-stranded homology directed repair DNA templates (HDRTs), contained a pool of 229 novel/synthetic DNA inserts plus knock-in controls flanked by ˜300 bp homology arms and shuttle sequences (Nguyen et al., 2019). HDRTs were SPRI purified (1.0×) and eluted into H2O. The concentrations of eluted HDRTs were normalized to 1 μg/μL. HDRT amplification was confirmed by gel electrophoresis in a 1.0% agarose gel. All DNA sequences used in the study are listed in Table S1.

### Cas9 RNP Electroporation

RNPs were produced by complexing a two-component gRNA to Cas9. The two-component gRNA consisted of a crRNA and a tracrRNA, both chemically synthesized (Dharmacon and IDT) and lyophilized. Upon arrival, lyophilized RNA was resuspended in a nuclease free buffer at a concentration of 160 μM and stored in aliquots at −80° C. Poly(L-glutamic acid) (PGA) MW 15-50 kDa (Sigma) was resuspended to 100 mg/mL in water, sterile filtered, and stored in aliquots at −80 C. Cas9-NLS (QB3 Macrolab) was recombinantly produced, purified, and stored at 40 μM in 20 mM HEPES-KOH, pH 7.5, 150 mM KCl, 10% glycerol, 1 mM DTT.

To produce RNPs, the crRNA and tracrRNA aliquots were thawed, mixed 1:1 by volume, and annealed by incubation at 37° C. for 30 min to form an 80 μM gRNA solution. Next. PGA mixed with freshly-prepared gRNA at 0.8:1 volume ratio prior to complexing with Cas9 protein for final volume ratio gRNA:PGA:Cas9 of 1:0.8:1. These were incubated at 37° C. for 15 min to form a 14.3 μM RNP solution.

RNPs and HDRTs were mixed with T cells before electroporation. Bulk T cells were spun down, resuspended in electroporation buffer P3 (LONZA), then each well was seeded at 750M cells/20 μl in a 96 well plate. The mixture was transferred to an electroporation plate (LONZA) and pulsed with the code EH115.

### Flow Cytometry and FACS

For flow cytometric analysis, T cells or cell lines were centrifuged at 300 g for 5 min and resuspended in flow buffer (PBS/2% FCS) containing the respective antibody mix. Cells were stained for 10 min at RT, washed once and analyzed on an Attune N×T Flow Cytometer (ThermoFisher, Waltham, Massachusetts, USA). For analysis of bone marrow ex vivo, material was strained (40 um, ThermoFisher, Waltham, Massachusetts, USA), centrifuged and incubated in ACK Lysing Buffer (ThermoFisher, Waltham, Massachusetts, USA) for 2 min at RT. Reaction was stopped by adding flow buffer containing 2 mM EDTA and cells were washed once. Pellets were resuspended in flow buffer/2 mM EDTA plus FcR Blocking Reagent, mouse (Miltenyi Biotec, Bergisch Gladbach, Germany). After incubation for 15 min at RT, antibodies were added. Cells were stained on ice for 45 min, washed once, resuspended in flow buffer/2 mM EDTA plus CountBright Absolute Counting Beads (ThermoFisher, Waltham, Massachusetts, USA) and analyzed on a BD LSRFortessa (BD Biosciences, San Jose, California, USA). Sorts were done on a BD FACSAria (BD Biosciences, San Jose, California, USA).

### Intracellular Cytokine Stains

T cells genetically engineered to express the NY-ESO-specific TCR and the construct of interest were re-stimulated with ImmunoCult Human CD3/CD28/CD2 T Cell Activator (25 uL/ml) at a T cell concentration of 1 M/ml for 4 hours. Re-stimulation was done cither prior to multiple stimulation assay or after the 5^{th} stimulation of the assay. Brefeldin A Solution 1,000× (BioLegend, San Diego, CA) was added to inhibit protein transport. Intracellular cytokines were analyzed by flow cytometry using the FIX & PERM Cell Fixation & Permeabilization Kit (ThermoFisher).

### In Vitro Single Stimulation Screens

One day prior to set-up of the screen, 2.5e6 A375s were plated per T75 flask in complete RPMI media (RPMI plus NEAA, Glutamine, Hepes, Pen/Strep, sodium pyruvate (all ThermoFisher, Waltham, Massachusetts, USA) and 10% FCS (Sigma-Aldrich, St. Louis. Missouri, USA)) assuming that they double within 24 hours. One day later (=seven days after electroporation), edited T cell pools were counted and washed once. 10e6 T cells were transferred to TRI Reagent (Sigma-Aldrich, St. Louis, Missouri, USA) representing the input population for amplicon sequencing. 10e6 T cells per screening condition were transferred to one T75 flask in 20 ml of X-VIVO 15 (Lonza, Basel, Switzerland) supplemented with 5% FCS, 2-Mercaptoethanol (ThermoFisher, Waltham, Massachusetts. USA) and 30 U/ml IL-2 (Proleukin). For A375 conditions, cRPMI was removed and flasks were filled up with 20 ml of X-VIVO 15 plus additives and 10e6 T cells. For Nalm-6 conditions, Nalm-6 cells were counted and Se6 Nalm-6 cells were added per T75 flask. In the stimulation conditions, T cells were stimulated with Dynabeads CD3/CD28 CTS (ThermoFisher, Waltham, Massachusetts, USA) at a 1:1 bead: cell ratio (“stim”) or a 5:1 ratio (“excessive stim”). For CD3 stimulation only (“without costim” condition), T cells were incubated with NY-ESO-1 specific dextramer (Immudex, Copenhagen, Denmark) for 12 min at RT (1:50 dilution), washed once and transferred to a T75 flasks. After two days, 10 ml of X-VIVO 15 were added to all conditions including supplements and 30 U/ml IL-2. Another two days later, cells were counted and 10e6 cells were transferred to TRI Reagent for RNA isolation and amplicon sequencing.

### In Vitro Multiple Stimulation Screens

One day prior to the start of the multiple stimulation screen, A375 cells were counted and transferred to 24-well plates (50.000 cells per well in 1 ml of complete RPMI media) assuming that they double within 24 hours. One day later, edited T cell pools were counted and 10e6 cells were frozen in TRI reagent for amplicon sequencing (input population). Media of the A375 cells was removed. 100.000 edited T cells (NY-ESO multimer positive, approximately 1:1 effector: target ratio) were transferred to each well of the 24-well plate and co-cultured with the A375 cells in 2 ml of X-VIVO 15 containing supplements plus 50 U/ml IL-2. 24 hours later, fresh A375 cells were plated as described above. One day later, media of the new A375 plate was removed and replaced by 1 ml of fresh X-VIVO 15 plus 1 ml of the T cell suspension from the first plate including 50 U/ml IL-2 calculated on the total volume per well. The rest of the T cells were counted and 10e6 cells were transferred to TRI Reagent for amplicon sequencing. The procedure was repeated every other day for a total number of five stimulations with target cells.

### In Vitro GD2 CAR Screens

Primary human T cells were electroporated with the GD2 CAR library as described above. As the GD2 CAR provides tonic signaling/chronic stimulation, T cells were cultured without addition of target cells. Cells were sorted on day 16 and day 4 after electroporation, amplicon sequencing was performed as described earlier and the log 2 fold change was calculated (day 16/day 4). Cells were cultured in X-Vivo 15 containing supplements plus 50U/ml IL-2.

### TOX Stain

Intracellular transcription factor stains were done using the eBioscience Foxp3/Transcription Factor Staining Buffer Set (ThermoFisher, Waltham. Massachusetts, USA) kit according to the supplier's information.

### In Vitro Proliferation Assay

For proliferation assays, T cells were stained using the CellTrace CFSE or CTV Cell Proliferation Kit (ThermoFisher, Waltham, Massachusetts, USA) according to the supplier's information. Briefly, up to 20e6 cells were resuspended at 1e6 cells per ml PBS and incubated with IX CTV or CFSE solution for 20 minutes at 37 C. Reaction was stopped by adding 30 ml of media. After an additional 5 min incubation at 37 C, cells were washed and used for validation assays.

### In Vitro Killing Assay

For flow-based killing assay, target cells were labelled with CellTrace CFSE or CTV Cell Proliferation Kit (ThermoFisher. Waltham, Massachusetts, USA) as described above. Assay was set up in round bottom 96-well plates using 20.000 target cells per well plus T cells in various effector: target ratios (X-VIVO 15 plus supplements and 30 U/ml IL-2). For read-out, 1× Propidium Iodide Solution (BioLegend, San Diego, California, USA) was added immediately before measurement. Number of target cells per well was calculated by excluding debris, gating on single cells, live cells (PI negative) and then on CFSE/CTV positive target cells. Percentage of killed targets was calculated by comparing the number of viable target cells in the experimental condition with the number of viable target cells in a target-only control.

For IncuCyte assays, RFP-transduced A375 cells were plated one day prior to start of the assay in optical 96-well flat bottom plates (1,500 A375 cells per well). One day later, T cells were added in various effector: target ratios (complete RPMI, 500 U/mL IL-2, 1× Glucose Solution (ThermoFisher, Waltham. Massachusetts. USA)). Cell counts (RFP+) were analyzed every six hours for a total 3-6 days using the IncuCyte Live Cell Analysis System (Essen BioScience, Ann Arbor, Michigan, USA).

For GD2 CAR IncuCyte assays, 96-well flat bottom plates were coated with 0.01% poly-L-omithine (PLO) solution (Sigma). After 1 hour at ambient temperature, PLO was removed and plates were dried. Sorted anti-GD2 CAR T cells were co-cultured with GFP-positive GD2-positive Nalm-6 cells. IncuCyte Annexin V Red Reagent (Essen Bioscience) was added according to the supplier's information.

### In Vitro Competition Assay

To evaluate abundance of single constructs over time, T cells genetically engineered to express the NY-ESO-specific TCR and the construct of interest were co-cultured with control T cells (NY-ESO-TCR plus NGFR) at a 1:1 ratio. Mixed T cell populations were co-cultured with A375 target cells during the multiple stimulation assay and abundance of different T cell constructs was analyzed by flow cytometry. Relative abundance was normalized to 50/50 input abundance prior to stimulation.

### LEGENDplex Analysis

At the end of multiple stimulation assay, supernatants of T cells co-cultured with A375s were harvested and cytokine concentration was analyzed using LEGENDplex Human CD8/NK Panel 13-plex according to the supplier's information (BioLegend).

### Xenograft Mouse Model

NSG mice were inoculated with 0.5M GFP/Luciferase-positive GD2-positive Nalm-6 cells via tail vein injection. Three days later, 2M anti-GD2 CAR-positive cells were injected IV (tail vein). Leukemia signal was analyzed 1-2×/week using in vivo imaging system (IVIS Lumina).

### Generation of Plasmid Libraries for Combinatorial Knock-In

GD2 CAR/pUC19 backbone was amplified by PCR. Inserts 1 and 2 were amplified from pooled libraries by PCR using two different primer pairs which removed constant sequences of the constructs and added a specific combo overhang as shown in FIG. 12A. PCR products were DpnI digested, gel and bead-purified (backbone) or only bead-purified (insert pool 1/2) before using NEBuilder HiFi DNA Assembly Master Mix (NEB) to create the combinatorial library. The Gibson product was bead-purified, transformed into Endura electrocompetent cells (Lucigen) and maxiprepped for further use. HDR template was generated as described above.

### Results

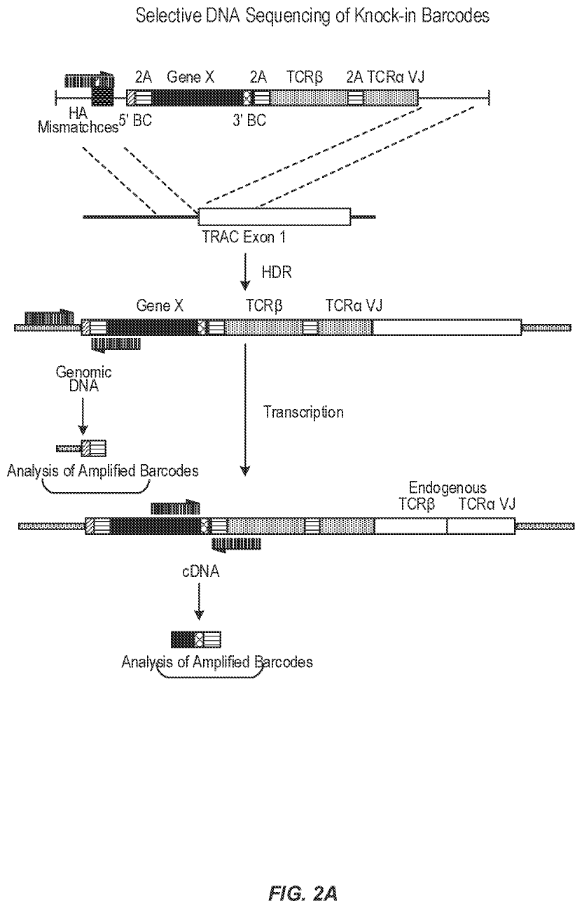

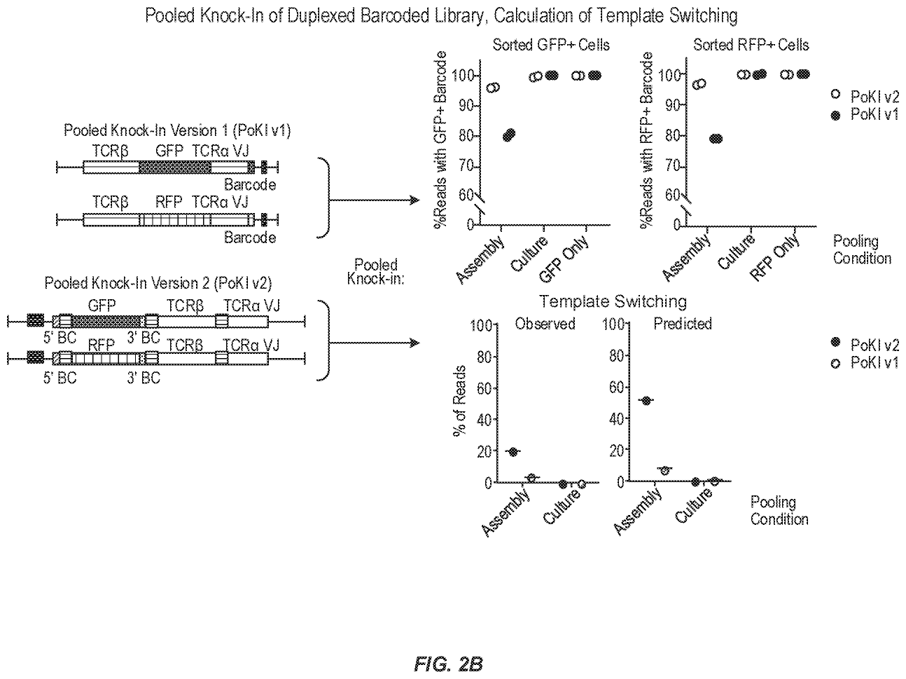

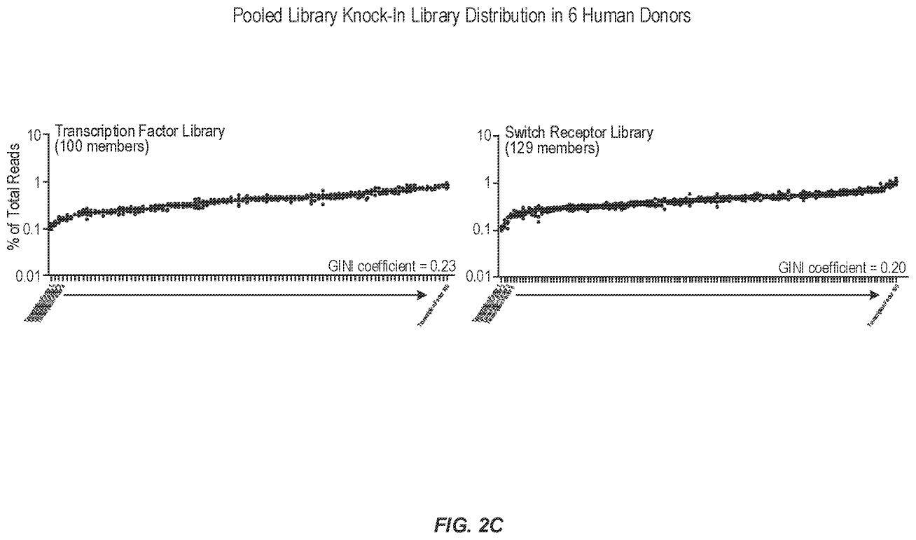

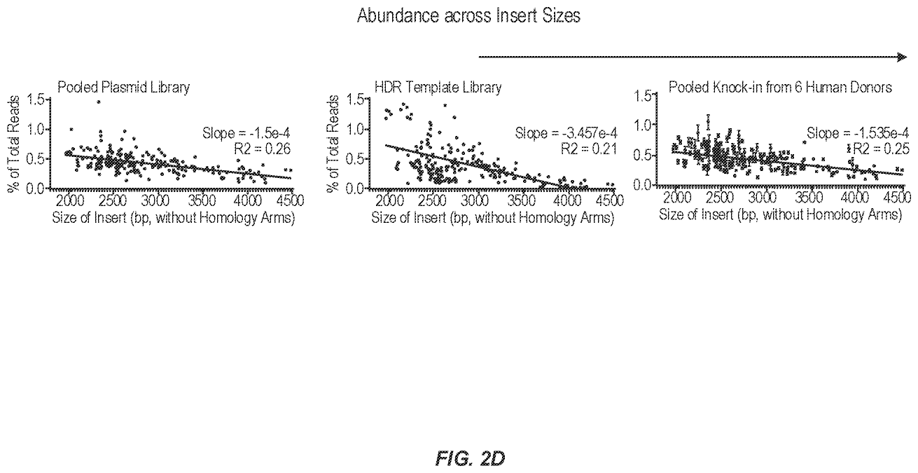

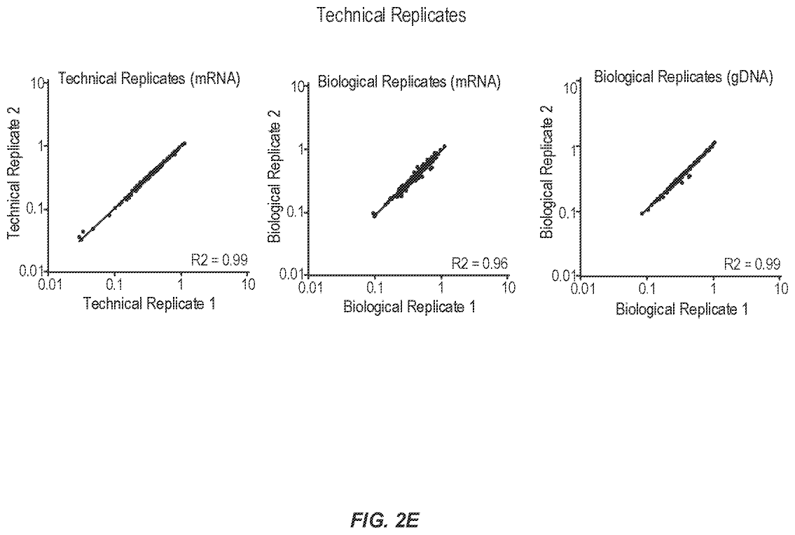

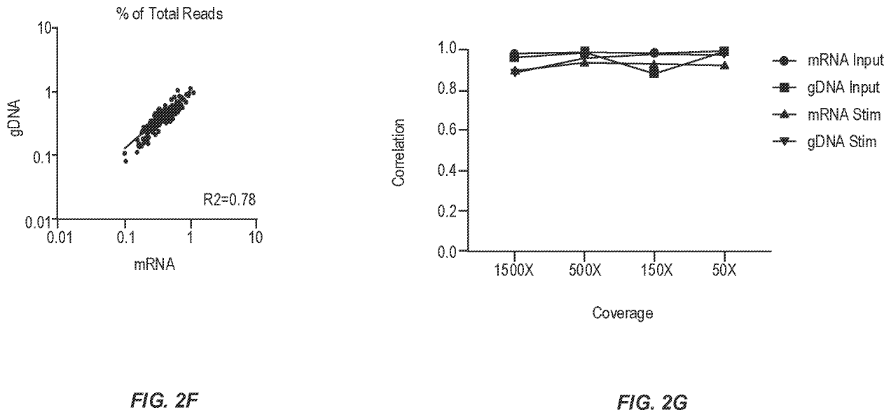

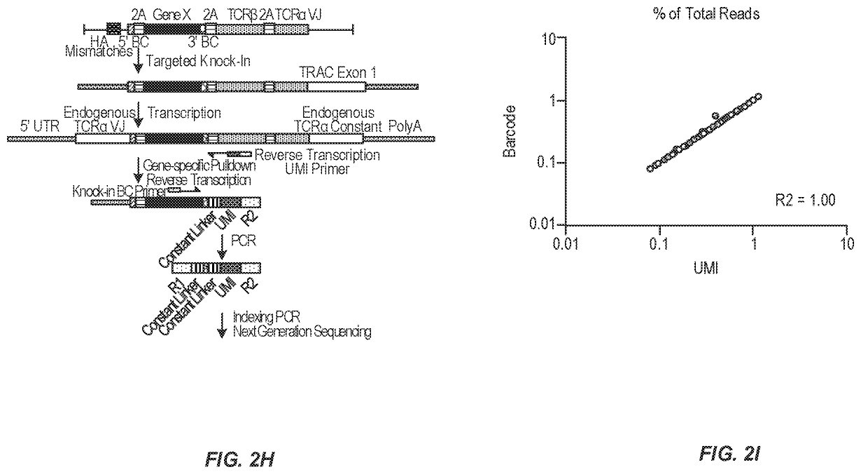

Using the methods described above, reproducible knock-in screens were performed. As shown in FIG. 2A, unique barcodes for every construct (“S′ BC” and “3” BC″) were encoded in degenerate bases in linker sequences flanking the gene of interest (“Gene X”). 5′ and 3′ BCs allowed for sequencing of genomic DNA (gDNA) or eDNA through distinct amplification strategies. DNA mismatches were introduced into one homology arm of the HDR template, allowing only on-target knock-ins to be amplified with primers bound to the endogenous homology arm sequence in the gDNA sequencing strategy. Extracted RNA was transcribed and the 3′ barcode is sequenced using primers specific for that inserted region.

FIG. 2B shows that duplexed knock-in libraries were pooled at indicated stages and the (3′) barcode was sequenced from cDNA. Improved construct design for Pooled Knock-in version 2 (PoKI v2) was compared to previous pooled knock-in strategies (PoKI v1, Roth et al. 2020). Percent reads with correctly assigned barcodes in sorted populations was notably improved over PoKI v1 when pooling at the assembly state.

As shown in FIG. 2C transcription factor (TF) and switch receptor (SR) libraries were knocked in as one large library and computationally separated into individual libraries for analysis. All construct barcodes were consistently well-represented with even library distribution (TF and SF Gini coefficients=0.23 and 0.20, respectively).

FIG. 2D shows that a negative correlation between construct size and library representation was observed in the plasmid pool. HDR template pool, and of knock-in reads in 6 human donors (R2=0.26, 0.21, and 0.25, respectively). Even the largest library members (4.5 kb inserts) were well represented. Four constructs above 1.5% were omitted from the HDR template library plot to maintain axis consistency.

FIG. 2E shows the reproducibility of pooled knock-in across technical and biological replicates. Sequencing of the 3′ BC from mRNA was highly reproducible across technical and biological replicates (R2=0.99 and 0.96, respectively). Biological replicates via the 5′ gDNA sequencing strategy yielded a similarly strong correlation (R2=0.99).

FIG. 2F shows the correlation between gDNA and mRNA BC sequencing strategies. 5′ BCs sequenced off gDNA and 3′ BC sequenced off mRNA from the same pooled knock-in experimental donor were well correlated (R2=0.78).

FIG. 2G shows the correlation between biological replicates across coverage range. Both mRNA and gDNA sequencing strategies were assessed at decreasing sequencing coverage. Correlations were also obtained from cell populations before (Input) and after (Stim) stimulation. Values were obtained as described in FIG. 2E. Even at low coverage (50×), donors were highly correlated across all strategies and experimental conditions.

FIG. 2H shows selective DNA sequencing of knock-in barcodes with UMI. After transcription, the TCR+Gene X mRNA transcripts from the individual cell are reverse transcribed using a gene-specific primer along with a universal molecular identifier (UMI). Following reverse transcription, a primer binding immediately upstream of the 3′ BC produces an amplicon containing both the 3′ barcode and the UMI. Next-generation sequencing of this amplicon allows for correlation between UMIs and BC counts.

FIG. 2I shows the results of next-generation sequencing of the 3′ BC+UMI amplicon reveals a high correlation between UMIs and BC counts (R2=1.00).

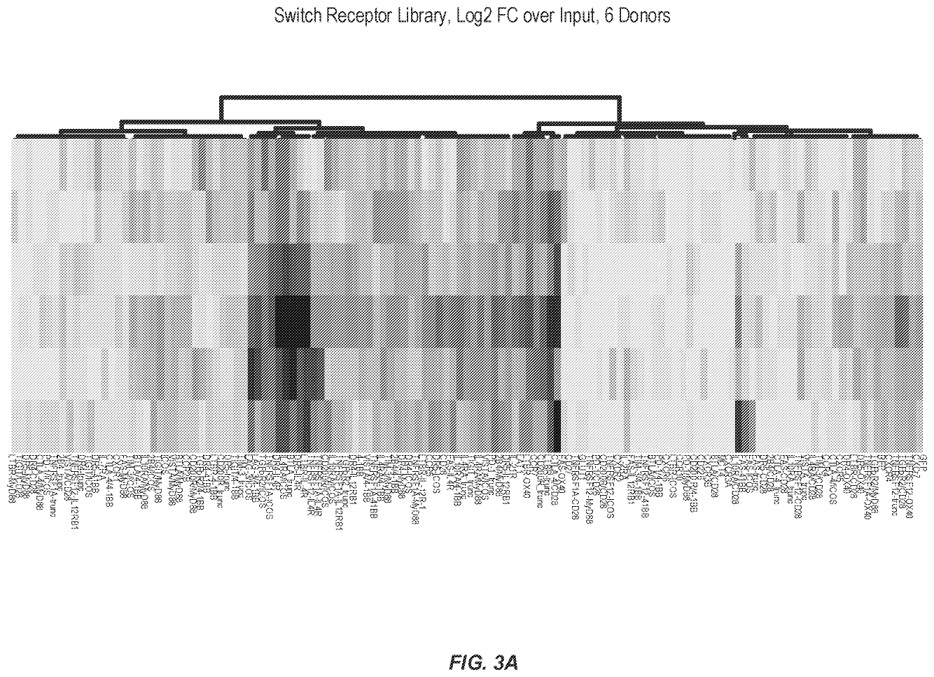

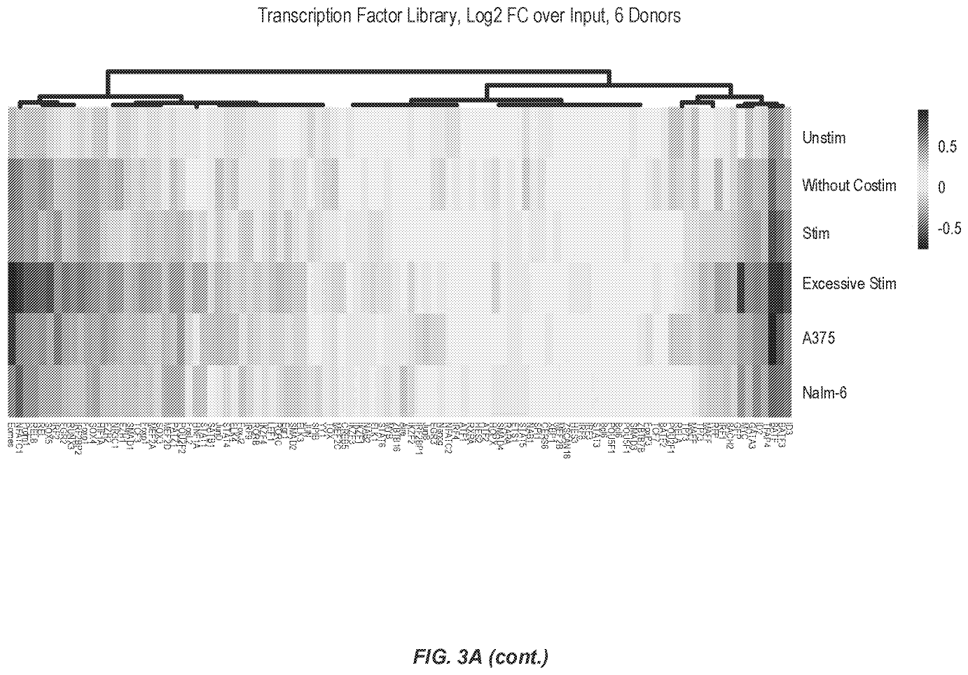

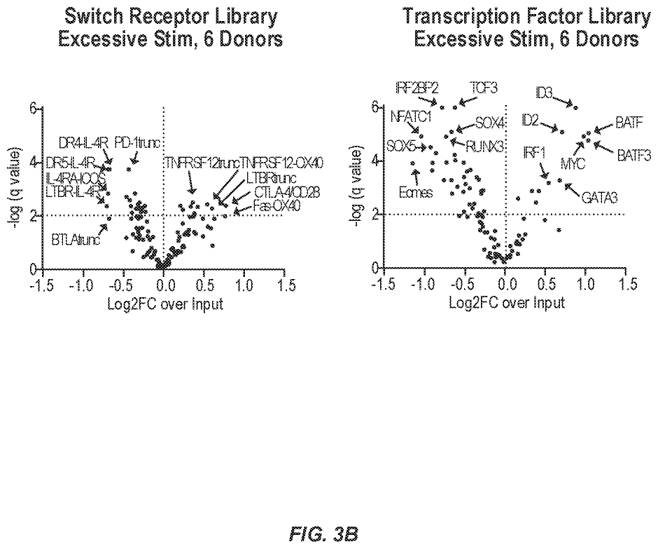

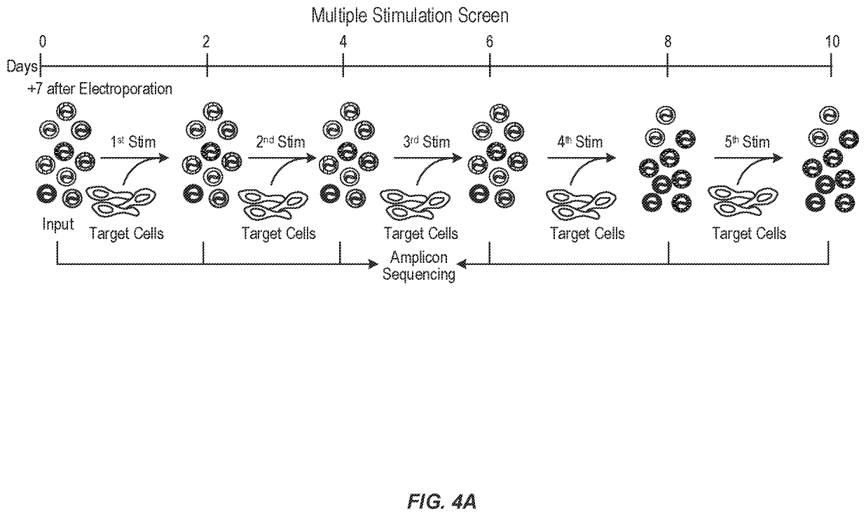

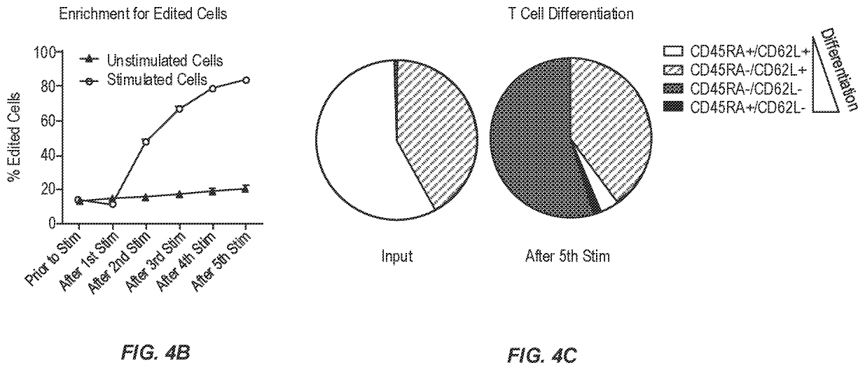

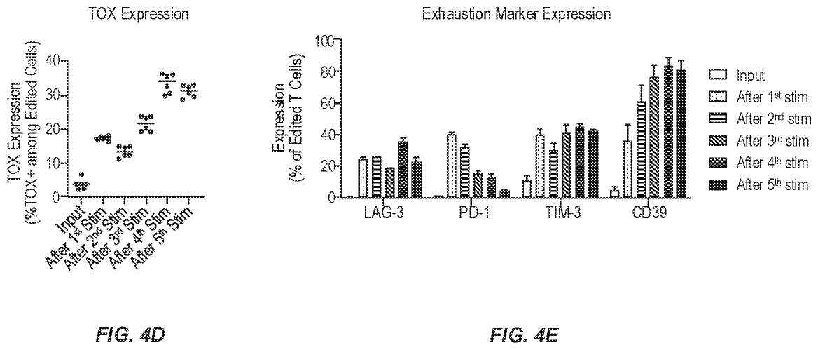

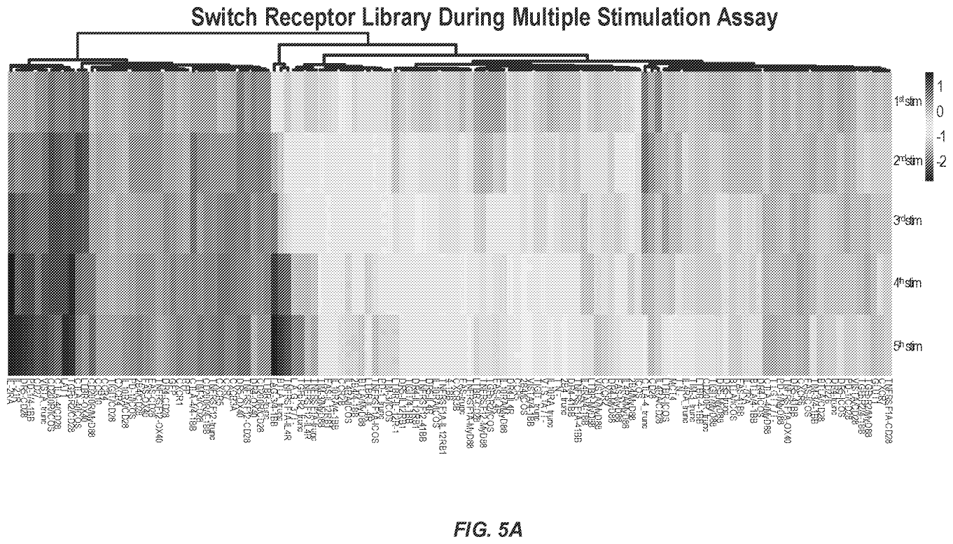

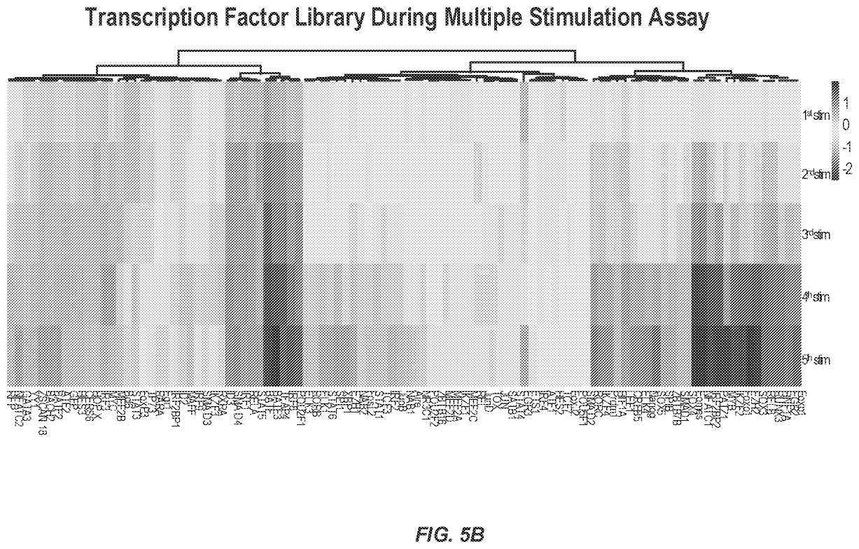

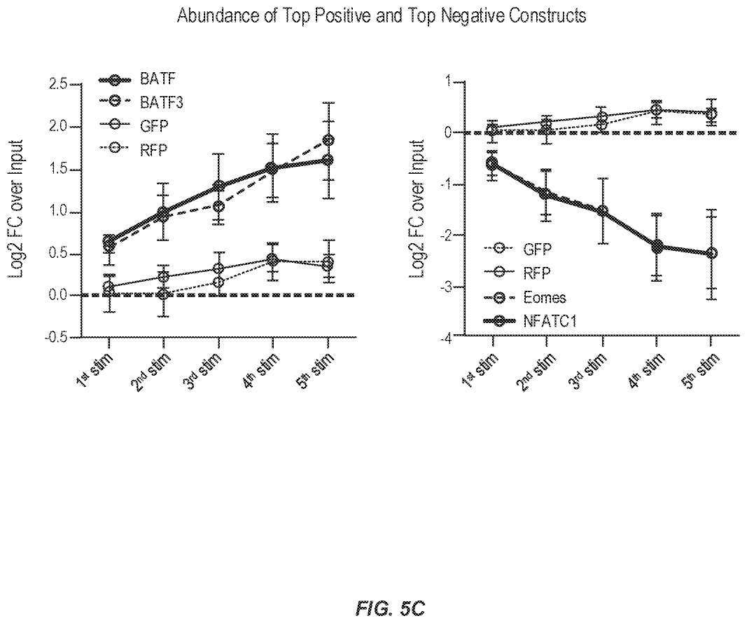

As shown in FIGS. 3A-B, a number of positive and negative hits were identified after the single stimulation abundance screen. Exhaustion-resistant T cell constructs were also identified using a multiple stimulation screen (FIGS. 4A-E). As shown in FIGS. 5A-C, a number of positive and negative hits were identified in the multiple stimulation abundance screen.

The nucleic acid and polypeptide sequences of the hits identified in the single and multiple stimulation screens are set forth in Table 2.

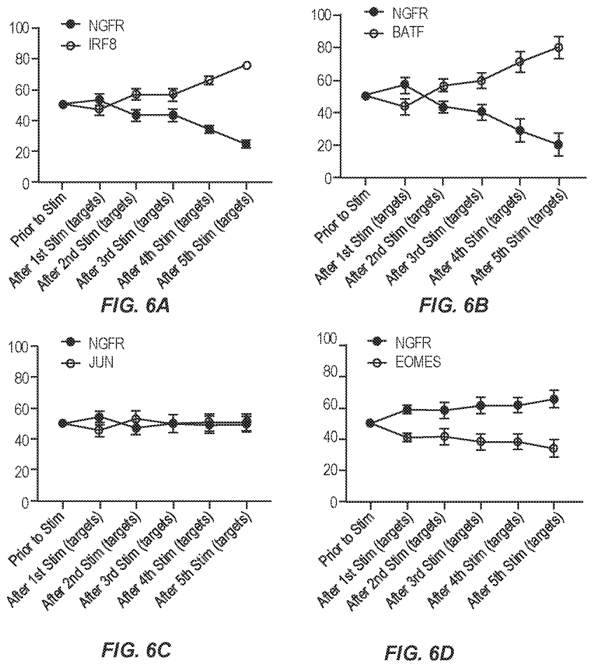

A number of positive and negative hits from single stimulation and multiple stimulation abundance screens were electroporated separately and analyzed further. As shown in FIGS. 6A-D top positive hits (ie IRF8 and BATF) as well as neutral constructs (ie JUN) and top negative hits (ie EOMES) perform as predicted by the screen in terms of relative abundance compared to a control construct (NGFR).

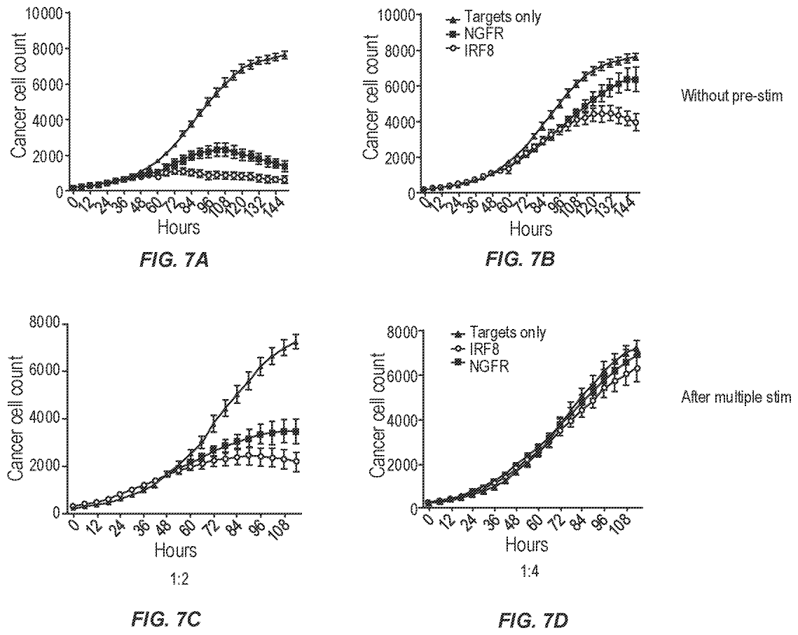

One of the top hits in the multiple stimulation abundance screen, IRF8, was electroporated separately and further evaluated in functionality assays. As shown in FIGS. 7A-D, killing assays confirm stronger cytotoxicity of NY-ESO/IRF8 cells compared to NY-ESO/NGFR cells against A375 target cells, either without pre-stimulation (A,B) or after going through the multiple stimulation assay (C,D).

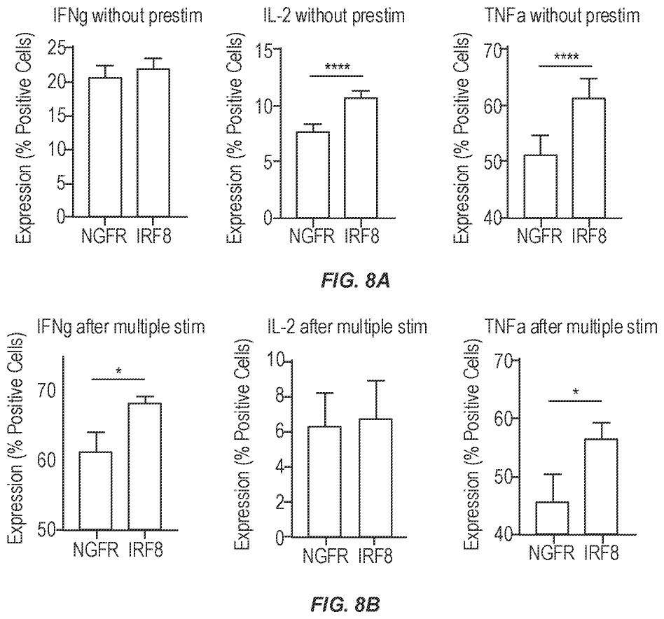

FIGS. 8A-B show increased cytokine release of NY-ESO/IRF8 T cells after stimulation with CD3/CD28/CD2, either without pre-stimulation (A) or after going through the multiple stimulation assay (five pre-stimulations, B).

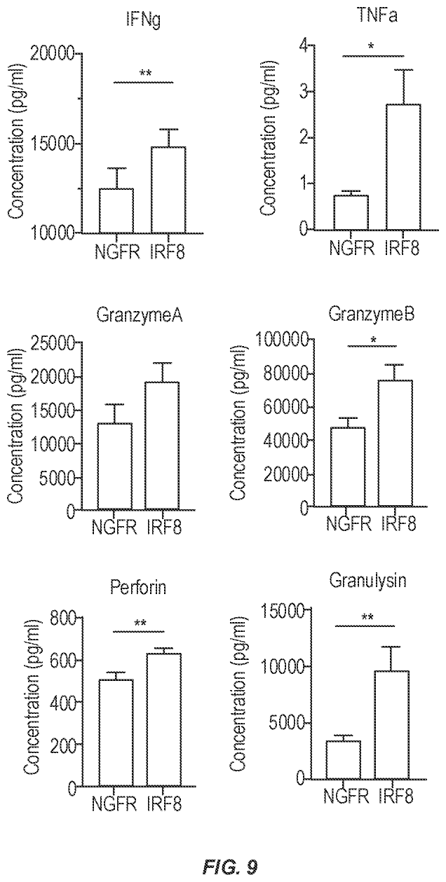

FIG. 9 shows increased levels of cytokines in the supernatant of NY-ESO/IRF8 T cells co-cultured with A375s at the end of the multiple stimulation assay.

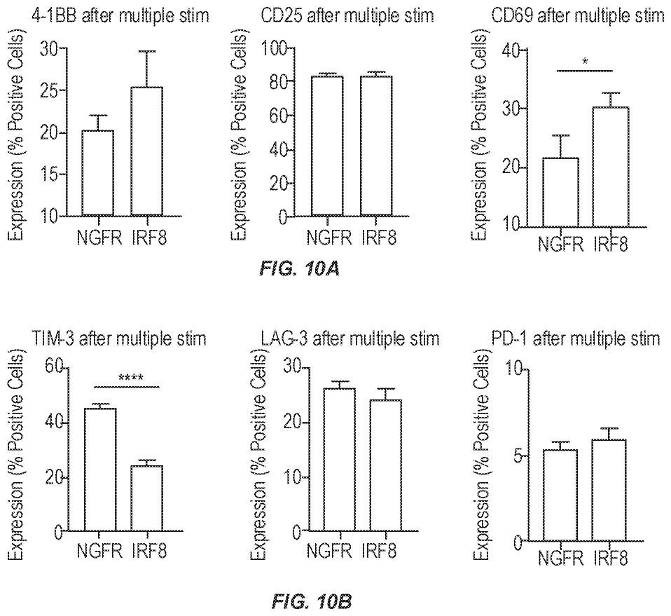

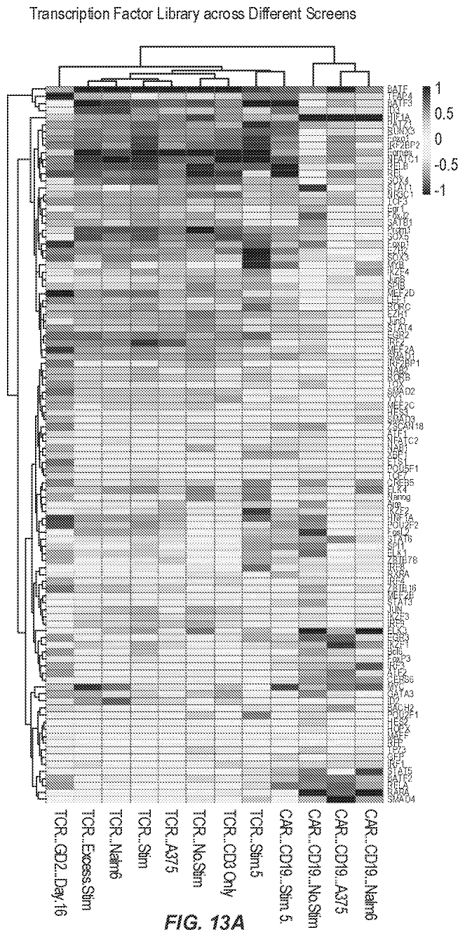

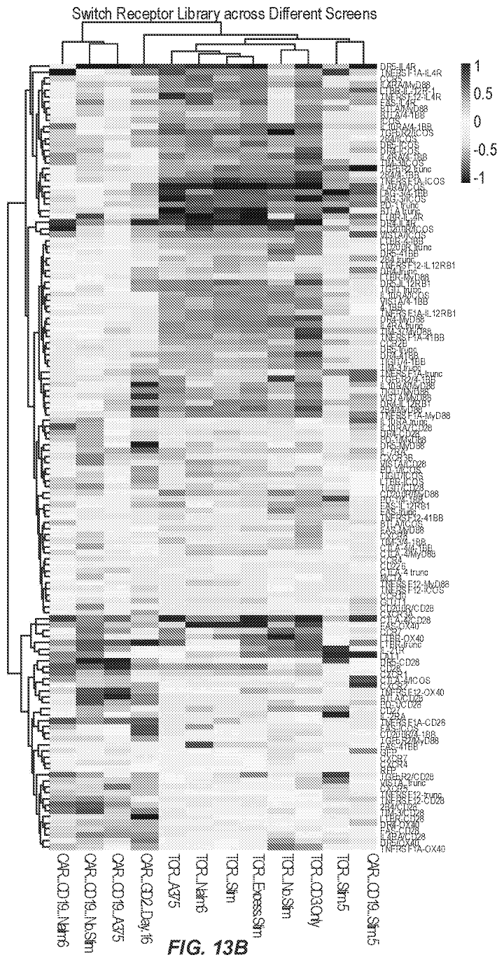

FIGS. 10A-B show increased expression of activation marker CD69 and decreased expression of exhaustion marker TIM-3 in NY-ESO/IRF8 T cells after being re-stimulated at the end of the multiple stimulation assay. FIGS. 13A-B show that, after performing several different screens in the TCR/CAR settings (NY-ESO TCR vs CD19 CAR vs tonic signaling GD2 CAR) with no, single or multiple stimulations with target cells, TFAP4 was identified as the top hit in the tonic signaling GD2 CAR assay when comparing abundance levels on day 16 vs day 4 after electroporation.

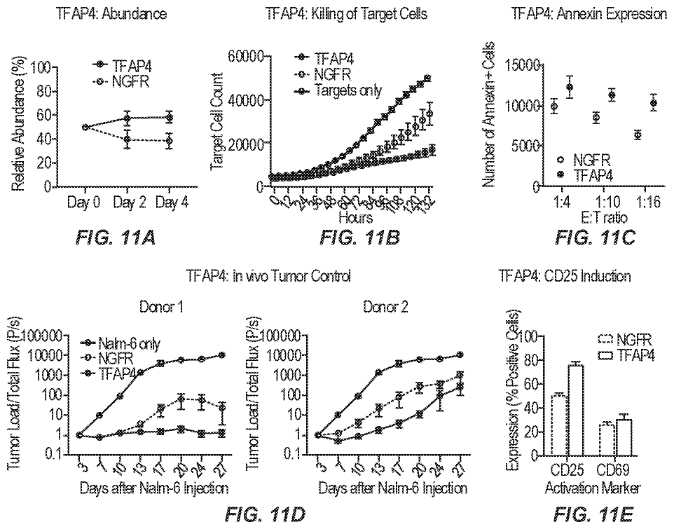

FIGS. 11A-11E show the results of single knock-in of the tonic signaling GD2 CAR and TFAP4 or control (NGFR) into primary human T cells. As shown in FIG. 11B, TFAP4 overexpression increased killing capacity of GD2 CAR T cells. FIG. 11C shows that Annexin+ cells, analyzed in the assay described in (B), showed increased levels of Annexin+ cells in TFAP4 conditions across different E:T ratios. FIG. 11D shows that after NSG mice were challenged with 0.5M GD2 expressing Nalm-6 cells IV, and treated with 2M anti-GD2 CAR T cells, with or without TFAP4 overexpression three days later, anti-GD2 CAR T cells with TFAP4 knock-in showed improved leukemia control measured by luciferase assay in two individual donors (n=5 mice per donor per group). FIG. 11E shows that TFAP4 overexpression increases CD25 levels on T cells as measured by flow cytometry.

TABLE 1
Domain sequences

SEQ ID NO: 65:
MLGIWTLLPLVLTSVARLSSKSVNAQVTDINSKGLELRKTVTTVETQNLEGLHHDGQ
FCHKPCPPGERKARDCTVNGDEPDCVPCQEGKEYTDKAHFSSKCRRCRLCDEGHGL
EVEINCTRTQNTKCRCKPNFFCNSTVCEHCDPCTKCEHGIIKECTLTSNTKCKEEGSRS
N
SEQ ID NO: 66:
LGWLCLLLLPIPLIVWV
SEQ ID NO: 67:
KRKEVQKTCRKHRKENQGSHESPTLNPETVAINLSDVDLSKYITTIAGVMTLSQVKG
FVRKNGVNEAKIDEIKNDNVQDTAEQKVQLLRNWHQLHGKKEAYDTLIKDLKKAN
LCTLAEKIQTIILKDITSDSENSNFRNEIQSLV
SEQ ID NO: 68:
MCVGARRLGRGPCAALLLLGLGLSTVTGLHCVGDTYPSNDRCCHECRPGNGMVSR
CSRSQNTVCRPCGPGFYNDVVSSKPCKPCTWCNLRSGSERKQLCTATQDTVCRCRA
GTQPLDSYKPGVDCAPCPPGHFSPGDNQACKPWTNCTLAGKHTLQPASNSSDAICED
RDPPATQPQETQGPPARPITVQPTEAWPRTSQGPSTRPVEVPGGRA
SEQ ID NO: 69:
VAAILGLGLVLGLLGPLAILL
SEQ ID NO: 70:
ALYLLRRDQRLPPDAHKPPGGGSFRTPIQEEQADAHSTLAKI
SEQ ID NO: 71:
MGNSCYNIVATLLLVLNFERTRSLQDPCSNCPAGTFCDNNRNQICSPCPPNSFSSAGG
QRTCDICRQCKGVFRTRKECSSTSNAECDCTPGFHCLGAGCSMCEQDCKQGQELTK
KGCKDCCFGTFNDQKRGICRPWTNCSLDGKSVLVNGTKERDVVCGPSPADLSPGAS
SVTPPAPAREPGHSPQ
SEQ ID NO: 72:
IISFFLALTSTALLFLLFFLTLRFSVV
SEQ ID NO: 73:
KRGRKKLLYIFKQPFMRPVQTTQEEDGCSCRFPEEEEGGCEL
SEQ ID NO: 74:
MKSGLWYFFLFCLRIKVLTGEINGSANYEMFIFHNGGVQILCKYPDIVQQFK
MQLLKGGQILCDLTKTKGSGNTVSIKSLKFCHSQLSNNSVSFFLYNLDHSHANYYFC
NLSIFDPPPFKVTLTGGYLHIYESQLCCQLK
SEQ ID NO: 75:
FWLPIGCAAFVVVCILGCILI
SEQ ID NO: 76:
CWLTKKKYSSSVHDPNGEYMFMRAVNTAKKSRLTDVTL
SEQ ID NO: 77:
MARGSLRRLLRLLVLGLWLALLRSVAGEQAPGTAPCSRGSSWSADLDKCMDCASC
RARPHSDFCLGCAAAPPAPFRLLWP
SEQ ID NO: 78:
ILGGALSLTFVLGLLSGFLVW
SEQ ID NO: 79:
RRCRRREKFTTPIEETGGEGCPAVALIQ
SEQ ID NO: 80:
MLLPWATSAPGLAWGPLVLGLFGLLAASQPQAVPPYASENQTCRDQEKEYYEPQHR
ICCSRCPPGTYVSAKCSRIRDTVCATCAENSYNEHWNYLTICQLCRPCDPVMGLEEIA
PCTSKRKTQCRCQPGMFCAAWALECTHCELLSDCPPGTEAELKDEVGKGNNHCVPC
KAGHFQNTSSPSARCQPHTRCENQGLVEAAPGTAQSDTTCKNPLEPLPPEMSGTML
M
SEQ ID NO: 81:
LAVLLPLAFFLLLATVFSCIW
SEQ ID NO: 82:
KSHPSLCRKLGSLLKRRPQGEGPNPVAGSWEPPKAHPYFPDLVQPLLPISGD
VSPVSTGLPAAPVLEAGVPQQQSPLDLTREPQLEPGEQSQVAHGTNGIHVTGGSMTIT
GNIYIYNGPVLGGPPGPGDLPATPEPPYPIPEEGDPGPPGLSTPHQEDGKAWHLAETE
HCGATPSNRGPRNQFITHD
SEQ ID NO: 83:
MWEAQFLGLLFLQPLWVAPVKPLQPGAEVPVVWAQEGAPAQLPCSPTIPLQDLSLL
RRAGVTWQHQPDSGPPAAAPGHPLAPGPHPAAPSSWGPRPRRYTVLSVGPGGLRSG
RLPLQPRVQLDERGRQRGDFSLWLRPARRADAGEYRAAVHLRDRALSCRLRLRLGQ
ASMTASPPGSLRASDWVILNCSFSRPDRPASVHWFRNRGQGRVPVRESPHHHLAESF
LFLPQVSPMDSGPWGCILTYRDGFNVSIMYNLTVLGLEPPTPLTVYAGAGSRVGLPC
RLPAGVGTRSFLTAKWTPPGGGPDLLVTGDNGDFTLRLEDVSQAQAGTYTCHIHLQ
EQQLNATVTLAIITVTPKSFGSPGSLGKLLCEVTPVSGQERFVWSSLDTPSQRSFSGPW
LEAQEAQLLSQPWQCQLYQGERLLGAAVYFTELSSPGAQRSGRAPGALPAGHL
SEQ ID NO: 84:
LLFLILGVLSLLLLVTGAFGF
SEQ ID NO: 85:
HLWRRQWRPRRFSALEQGIHPPQAQSKIEELEQEPEPEPEPEPEPEPEPEPEQL
SEQ ID NO: 86:
MEQRGQNAPAASGARKRHGPGPREARGARPGPRVPKTLVLVVAAVLLLVSAESALI
TQQDLAPQQRAAPQQKRSSPSEGLCPPGHHISEDGRDCISCKYGQDYSTHWNDLLFC
LRCTRCDSGEVELSPCTTTRNTVCQCEEGTFREEDSPEMCRKCRTGCPRGMVKVGD
CTPWSDIECVHKESGTKHSGEVPAVEETVTSSPGTPASPCS
SEQ ID NO: 87:
LSGIGVTVAAVVLIVAVFV
SEQ ID NO: 88:
CKSLLWKKVLPYLKGICSGGGGDPERVDRSSQRPGAEDNVLNEIVSILQPTQVPEQE
MEVQEPAEPTGVNMLSPGESEHLLEPAEAERSQRRRLLVPANEGDPTETLRQCFDDF
ADLVPFDSWEPLMRKLGLMDNEIKVAKAEAAGHRDTLYTMLIKWVNKTGRDASVH
TLLDALETLGERLAKQKIEDHLLSSGKFMYLEGNADSAMS
SEQ ID NO: 89:
MGWLCSGLLFPVSCLVLLQVASSGNMKVLQEPTCVSDYMSISTCEWKMNGPTNCST
ELRLLYQLVFLLSEAHTCIPENNGGAGCVCHLLMDDVVSADNYTLDLWAGQQLLW
KGSFKPSEHVKPRAPGNLTVHTNVSDTLLLTWSNPYPPDNYLYNHLTYAVNIWSEN
DPADFRIYNVTYLEPSLRIAASTLKSGISYRARVRAWAQCYNTTWSEWSPSTKWHNS
YREPFEQH
SEQ ID NO: 90:
LLLGVSVSCIVILAVCLLCYVSIT
SEQ ID NO: 91:
KIKKEWWDQIPNPARSRLVAIIIQDAQGSQWEKRSRGQEPAKCPHWKNCLTKLLPCF
LEHNMKRDEDPHKAAKEMPFQGSGKSAWCPVEISKTVLWPESISVVRCVELFEAPVE
CEEEEEVEEEKGSFCASPESSRDDFQEGREGIVARLTESLFLDLLGEENGGFCQQDMG
ESCLLPPSGSTSAHMPWDEFPSAGPKEAPPWGKEQPLHLEPSPPASPTQSPDNLTCTE
TPLVIAGNPAYRSFSNSLSQSPCPRELGPDPLLARHLEEVEPEMPCVPQLSEPTTVPQP
EPETWEQILRRNVLQHGAAAAPVSAPTSGYQEFVHAVEQGGTQASAVVGLGPPGEA
GYKAFSSLLASSAVSPEKCGFGASSGEEGYKPFQDLIPGCPGDPAPVPVPLFTFGLDRE
PPRSPQSSHLPSSSPEHLGLEPGEKVEDMPKPPLPQEQATDPLVDSLGSGIVYSALTCH
LCGHLKQCHGQEDGGQTPVMASPCCGCCCGDRSSPPTTPLRAPDPSPGGVPLEASLC
PASLAPSGISEKSKSSSSFHPAPGNAQSSSQTPKIVNFVSVGPTYMRVS
SEQ ID NO: 92:
MAPPPARVHLGAFLAVTPNPGSAASGTEAAAATPSKVWGSSAGRIEPRGGGRGALPT
SMGQHGPSARARAGRAPGPRPAREASPRLRVHKTFKFVVVGVLLQVVPSSAATIKLH
DQSIGTQQWEHSPLGELCPPGSHRSEHPGACNRCTEGVGYTNASNNLFACLPCTACK
SDEEERSPCTTTRNTACQCKPGTFRNDNSAEMCRKCSRGCPRGMVKVKDCTPWSDI
ECVHKESGNGHN
SEQ ID NO: 93:
IWVILVVTLVVPLLLVAVLIVCC
SEQ ID NO: 94:
CIGSGCGGDPKCMDRVCFWRLGLLRGPGAEDNAHNEILSNADSLSTFVSEQQMESQ
EPADLTGVTVQSPGEAQCLLGPAEAEGSQRRRLLVPANGADPTETLMLFFDKFANIV
PFDSWDQLMRQLDLTKNEIDVVRAGTAGPGDALYAMLMKWVNKTGRNASIHTLLD
ALERMEERHAREKIQDLLVDSGKFIYLEDGTGSAVSLE
SEQ ID NO: 95:
MGWLCSGLLFPVSCLVLLQVASSGNMKVLQEPTCVSDYMSISTCEWKMNGPTNCST
ELRLLYQLVFLLSEAHTCIPENNGGAGCVCHLLMDDVVSADNYTLDLWAGQQLLW
KGSFKPSEHVKPRAPGNLTVHTNVSDTLLLTWSNPYPPDNYLYNHLTYAVNIWSEN
DPADFRIYNVTYLEPSLRIAASTLKSGISYRARVRAWAQCYNTTWSEWSPSTKWHNS
YREPFEQH
SEQ ID NO: 96:
LLLGVSVSCIVILAVCLLCYVSIT
SEQ ID NO: 97:
KIKKEWWDQIPNPARSRLVAIIIQDAQGSQWEKRSRGQEPAKCPHWKNCLTKLLPCF
LEHNMKRDEDPHKAAKEMPFQGSGKSAWCPVEISKTVLWPESISVVRCVELFEAPVE
CEEEEEVEEEKGSFCASPESSRDDFQEGREGIVARLTESLFLDLLGEENGGFCQQDMG
ESCLLPPSGSTSAHMPWDEFPSAGPKEAPPWGKEQPLHLEPSPPASPTQSPDNLTCTE
TPLVIAGNPAYRSFSNSLSQSPCPRELGPDPLLARHLEEVEPEMPCVPQLSEPTTVPQP
EPETWEQILRRNVLQHGAAAAPVSAPTSGYQEFVHAVEQGGTQASAVVGLGPPGEA
GYKAFSSLLASSAVSPEKCGFGASSGEEGYKPFQDLIPGCPGDPAPVPVPLFTFGLDRE
PPRSPQSSHLPSSSPEHLGLEPGEKVEDMPKPPLPQEQATDPLVDSLGSGIVYSALTCH
LCGHLKQCHGQEDGGQTPVMASPCCGCCCGDRSSPPTTPLRAPDPSPGGVPLEASLC
PASLAPSGISEKSKSSSSFHPAPGNAQSSSQTPKIVNFVSVGPTYMRVS
SEQ ID NO: 106:
MACLGFQRHKAQLNLATRTWPCTLLFFLLFIPVFCKAMHVAQPAVVLASSRGIASFV
CEYASPGKATEVRVTVLRQADSQVTEVCAATYMMGNELTFLDDSICTGTSSGNQVN
LTIQGLRAMDTGLYICKVELMYPPPYYLGIGNGTQIYVIDPEPCPDSD
SEQ ID NO: 107:
FLLWILAAVSSGLFFYSFLLT
SEQ ID NO: 108:
AVSLSKMLKKRSPLTTGVYVKMPPTEPECEKQFQPYFIPIN
SEQ ID NO: 109:
MLRLLLALNLFPSIQVTGNKILVKQSPMLVAYDNAVNLSCKYSYNLFSREFRASLHK
GLDSAVEVCVVYGNYSQQLQVYSKTGFNCDGKLGNESVTFYLQNLYVNQTDIYFCK
IEVMYPPPYLDNEKSNGTIIHVKGKHLCPSPLFPGPSKP
SEQ ID NO: 110:
FWVLVVVGGVLACYSLLVTVAFIIFWV
SEQ ID NO: 111:
RSKRSRLLHSDYMNMTPRRPGPTRKHYQPYAPPRDFAAYRS
SEQ ID NO: 112:
MLCPWRTANLGLLLILTIFLVAASSSLCMDEKQITQNYSKVLAEVNTSWPVKMATN
AVLCCPPIALRNLIIITWEIILRGQPSCTKAYRKETNETKETNCTDERITWVSRPDQNSD
LQIRPVAITHDGYYRCIMVTPDGNFHRGYHLQVLVTPEVTLFQNRNRTAVCKAVAG
KPAAQISWIPEGDCATKQEYWSNGTVTVKSTCHWEVHNVSTVTCHVSHLTGNKSLY
IELLPVPGAKKSAKL
SEQ ID NO: 113:
YIPYIILTIIILTIVGFIWLL
SEQ ID NO: 114:
KVNGCRKYKLNKTESTPVVEEDEMQPYASYTEKNNPLYDTTNKVKASEALQSEVDT
DLHTL

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety.

TABLE 2

| | | nucleic acid | amino acid |
|---|---|---|---|
| Domain | Domain | sequence encoding polypeptide | sequence of polypeptide |
| | | ATGCTGGGCATCTGGACCCTCCTACCTCTGGTT | MLGIWILLPLVLTSVARLSSKSVNAQVT |
| | | CTTACGTCTGTTGCTAGATTATCGTCCAAAAGT | DINSKGLELRKTVTTVETQNLEGLHHDG |
| | | GTTAATGCCCAAGTGACTGACATCAACTCCAAG | QFCHKPCPPGERKARDCTVNGDEPDCVP |
| | | GGATTGGAATTGAGGAAGACTGTTACTACAGTT | CQEGKEYTDKAHFSSKCRRCRLCDEGH |
| Fas | OX40 | GAGACTCAGAACTTGGAAGGCCTGCATCATGAT | GLEVEINCTRTQNTKCRCKPNFFCNSTV |
| | | GGCCAATTCTGCCATAAGCCCTGTCCTCCAGGT | CEHCDPCTKCEHGIKECTLTSNTKCKEE |
| | | GAAAGGAAAGCTAGGGACTGCACAGTCAATGG | GSRSNLGWLCLLLLPIPLIVWVKRKEVQ |
| | | GGATGAACCAGACTGCGTGCCCTGCCAAGAAG | KALYLLRRDQRLPPDAHKPPGGGSFRTP |
| | | GGAAGGAGTACACAGACAAAGCCCATTTTTCTT | IQEEQADAHSTLAKI (SED ID NO: 33) |
| | | CCAAATGCAGAAGATGTAGATTGTGTGATGAA | |
| | | GGACATGGCTTAGAAGTGGAAATAAACTGCAC | |
| | | CCGGACCCAGAATACCAAGTGCAGATGTAAAC | |
| | | CAAACTTTTTTTGTAACTCTACTGTATGTGAACA | |
| | | CTGTGACCCTTGCACCAAATGTGAACATGGAAT | |
| | | CATCAAGGAATGCACACTCACCAGCAACACCA | |
| | | AGTGCAAAGAGGAAGGATCCAGATCTAACTTG | |
| | | GGGTGGCTTTGTCTTCTTCTTTTGCCAATTCCAC | |
| | | TAATTGTTTGGGTGAAGAGAAAGGAAGTACAG | |
| | | AAAgccctgtACCTGCTCCGGAGGGACCAGAGGCT | |
| | | GCCCCCCGATGCCCACAAGCCCCCTGGGGGAG | |
| | | GCAGTTTCCGGACCCCCATCCAAGAGGAGCAG | |
| | | GCCGACGCCCACTCCACCCTGGCCAAGATC | |
| | | (SEQ ID NO: 1) | |
| TNFRSF12 | OX40 | ATGGCCCGCGGAAGTCTTCGCCGTTTGCTCCGT | MARGSLRRLLRLLVLGLWLALLRSVAG |
| | | CTTCTTGTTCTCGGCCTGTGGCTCGCTCTGCTCC | EQAPGTAPCSRGSSWSADLDKCMDCAS |
| | | GGAGTGTAGCGGGCGAACAAGCACCTGGGACT | CRARPHSDFCLGCAAAPPAPFRLLWPIL |
| | | GCACCGTGTTCACGGGGCTCCTCATGGTCCGCC | GGALSLTFVLGLLSGFLVWRRCRRREAL |
| | | GATCTTGATAAATGTATGGATTGTGCCAGCTGT | YLLRRDQRLPPDAHKPPGGGSFRTPIQEE |
| | | AGAGCCAGGCCCCATTCTGATTTCTGTCTTGGG | QADAHSTLAKI (SEQ ID NO: 34) |
| | | TGTGCCGCTGCCCCACCGGCACCTTTTAGACTT | |
| | | CTGTGGCCTATTCTGGGCGGAGCCCTCTCATTG | |
| | | ACATTTGTCCTTGGACTTCTCTCCGGGTTCCTTG | |
| | | TATGGCGGCGGTGTCGGCGCCGCGAAgccctgtAC | |
| | | CTGCTCCGGAGGGACCAGAGGCTGCCCCCCGAT | |
| | | GCCCACAAGCCCCCTGGGGGAGGCAGTTTCCG | |
| | | GACCCCCATCCAAGAGGAGCAGGCCGACGCCC | |
| | | ACTCCACCCTGGCCAAGATC (SEQ ID NO: 2) | |
| LTBR | OX40 | ATGCTCCTGCCTTGGGCCACCTCTGCCCCCGGC | MLLPWATSAPGLAWGPLVLGLFGLLAA |
| | | CTGGCCTGGGGGCCTCTGGTGCTGGGCCTCTTC | SQPQAVPPYASENQTCRDQEKEYYEPQH |
| | | GGGCTCCTGGCAGCATCGCAGCCCCAGGCGGT | RICCSRCPPGTYVSAKCSRIRDTVCATCA |
| | | GCCTCCATATGCGTCGGAGAACCAGACCTGCAG | ENSYNEHWNYLTICQLCRPCDPVMGLE |
| | | GGACCAGGAAAAGGAATACTATGAGCCCCAGC | EIAPCTSKRKTQCRCQPGMFCAAWALE |
| | | ACCGCATCTGCTGCTCCCGCTGCCCGCCAGGCA | CTHCELLSDCPPGTEAELKDEVGKGNNH |
| | | CCTATGTCTCAGCTAAATGTAGCCGCATCCGGG | CVPCKAGHFQNTSSPSARCQPHTRCENQ |
| | | ACACAGTTTGTGCCACATGTGCCGAGAATTCCT | GLVEAAPGTAQSDTTCKNPLEPLPPEMS |
| | | ACAACGAGCACTGGAACTACCTGACCATCTGCC | GTMLMLAVLLPLAFFLLLATVFSCIWKS |
| | | AGCTGTGCCGCCCCTGTGACCCAGTGATGGGCC | HPSLCALYLLRRDQRLPPDAHKPPGGGS |
| | | TCGAGGAGATTGCCCCCTGCACAAGCAAACGG | FRTPIQEEQADAHSTLAKI (SEQ DI |
| | | AAGACCCAGTGCCGCTGCCAGCCGGGAATGTTC | NO: 35) |
| | | TGTGCTGCCTGGGCCCTCGAGTGTACACACTGC | |
| | | GAGCTACTTTCTGACTGCCCGCCTGGCACTGAA | |
| | | GCCGAGCTCAAAGATGAAGTTGGGAAGGGTAA | |
| | | CAACCACTGCGTCCCCTGCAAGGCCGGGCACTT | |
| | | CCAGAATACCTCCTCCCCCAGCGCCCGCTGCCA | |
| | | GCCCCACACCAGGTGTGAGAACCAAGGTCTGG | |
| | | TGGAGGCAGCTCCAGGCACTGCCCAGTCCGAC | |
| | | ACAACCTGCAAAAATCCATTAGAGCCACTGCCC | |
| | | CCAGAGATGTCAGGAACCATGCTGATGCTGGCC | |
| | | GTTCTGCTGCCACTGGCCTTCTTTCTGCTCCTTG | |
| | | CCACCGTCTTCTCCTGCATCTGGAAGAGCCACC | |
| | | CTTCTCTCTGCgccctgtACCTGCTCCGGAGGGACC | |
| | | AGAGGCTGCCCCCCGATGCCCACAAGCCCCCTG | |
| | | GGGGAGGCAGTTTCCGGACCCCCATCCAAGAG | |
| | | GAGCAGGCCGACGCCCACTCCACCCTGGCCAA | |
| | | GATC (SEQ ID NO: 3) | |
| LTBR | truncated | ATGCTCCTGCCTTGGGCCACCTCTGCCCCCGGC | MLLPWATSAPGLAWGPLVLGLEGLLAA |
| | | CTGGCCTGGGGGCCTCTGGTGCTGGGCCTCTTC | SQPQAVPPYASENQTCRDQEKEYYEPQH |
| | | GGGCTCCTGGCAGCATCGCAGCCCCAGGCGGT | RICCSRCPPGTYVSAKCSRIRDTVCATCA |
| | | GCCTCCATATGCGTCGGAGAACCAGACCTGCAG | ENSYNEHWNYLTICQLCRPCDPVMGLE |
| | | GGACCAGGAAAAGGAATACTATGAGCCCCAGC | EIAPCTSKRKTQCRCQPGMFCAAWALE |
| | | ACCGCATCTGCTGCTCCCGCTGCCCGCCAGGCA | CTHCELLSDCPPGTEAELKDEVGKGNNH |
| | | CCTATGTCTCAGCTAAATGTAGCCGCATCCGGG | CVPCKAGHFQNTSSPSARCQPHTRCENQ |
| | | ACACAGTTTGTGCCACATGTGCCGAGAATTCCT | GLVEAAPGTAQSDTTCKNPLEPLPPEMS |
| | | ACAACGAGCACTGGAACTACCTGACCATCTGCC | GTMLMLAVLLPLAFFLLLATVFSCIWKS |
| | | AGCTGTGCCGCCCCTGTGACCCAGTGATGGGCC | HPSLCALYLLRRDQRLPPDAHKPPGGGS |
| | | TCGAGGAGATTGCCCCCTGCACAAGCAAACGG | FRTPIQEEQADAHSTLAKI (SEQ ID |
| | | AAGACCCAGTGCCGCTGCCAGCCGGGAATGTTC | NO: 36) |
| | | TGTGCTGCCTGGGCCCTCGAGTGTACACACTGC | |
| | | GAGCTACTTTCTGACTGCCCGCCTGGCACTGAA | |
| | | GCCGAGCTCAAAGATGAAGTTGGGAAGGGTAA | |
| | | CAACCACTGCGTCCCCTGCAAGGCCGGGCACTT | |
| | | CCAGAATACCTCCTCCCCCAGCGCCCGCTGCCA | |
| | | GCCCCACACCAGGTGTGAGAACCAAGGTCTGG | |
| | | TGGAGGCAGCTCCAGGCACTGCCCAGTCCGAC | |
| | | ACAACCTGCAAAAATCCATTAGAGCCACTGCCC | |
| | | CCA (SEQ ID NO: 4) | |
| TNFRSF | truncated | ATGGCCCGCGGAAGTCTTCGCCGTTTGCTCCGT | MARGSLRRLLRLLVLGLWLALLRSVAG |
| | | CTTCTTGTTCTCGGCCTGTGGCTCGCTCTGCTCC | EQAPGTAPCSRGSSWSADLDKCMDCAS |
| | | GGAGTGTAGCGGGCGAACAAGCACCTGGGACT | CRARPHSDFCLGCAAAPPAPFRLLWPIL |
| | | GCACCGTGTTCACGGGGCTCCTCATGGTCCGCC | GGALSLTFVLGLLSGFLVWRRCRRRE |
| | | GATCTTGATAAATGTATGGATTGTGCCAGCTGT | (SEQ ID NO: 37) |
| | | AGAGCCAGGCCCCATTCTGATTTCTGTCTTGGG | |
| | | TGTGCCGCTGCCCCACCGGCACCTTTTAGACTT | |
| | | CTGTGGCCTATTCTGGGCGGAGCCCTCTCATTG | |
| | | ACATTTGTCCTTGGACTTCTCTCCGGGTTCCTTG | |
| | | TATGGCGGCGGTGTCGGCGCCGCGAA (SEQ ID | |
| | | NO: 5) | |
| IL-21R | | ATGGCCCCGCGGCGGGCGCGCGGCTGCCGGAC | MPRGWAAPLLLLLLQGGWGCPDLVCYT |
| | | CCTCGGTCTCCCGGCGCTGCTACTGCTGCTGCT | DYLQTVICILEMWNLHPSTLTLTWQDQY |
| | | GCTCCGGCCGCCGGCGACGCGGGGCATCACGT | EELKDEATSCSLHRSAHNATHATYTCH |
| | | GCCCTCCCCCCATGTCCGTGGAACACGCAGACA | MDVFHFMADDIFSVNITDQSGNYSQECG |
| | | TCTGGGTCAAGAGCTACAGCTTGTACTCCAGGG | SFLLAESIKPAPPFNVTVTFSGQYNISWR |
| | | AGCGGTACATTTGTAACTCTGGTTTCAAGCGTA | SDYEDPAFYMLKGKLQYELQYRNRGDP |
| | | AAGCCGGCACGTCCAGCCTGACGGAGTGCGTG | WAVSPRRKLISVDSRSVSLLPLEFRKDSS |
| | | TTGAACAAGGCCACGAATGTCGCCCACTGGAC | YELQVRAGPMPGSSYQGTWSEWSDPVI |
| | | AACCCCCAGTCTCAAATGCATTAGAGACCCTGC | FQTQSEELKEGWNPHLLLLLLLVIVFIPA |
| | | CCTGGTTCACCAAAGGCCAGCGCCACCCTCCAC | FWSLKTHPLWRLWKKIWAVPSPERFFM |
| | | AGTAACGACGGCAGGGGTGACCCCACAGCCAG | PLYKGCSGDFKKWVGAPFTGSSLELGP |
| | | AGAGCCTCTCCCCTTCTGGAAAAGAGCCCGCAG | WSPEVPSTLEVYSCHPPRSPAKRLQLTEL |
| | | CTTCATCTCCCAGCTCAAACAACACAGCGGCCA | QEPAELVESDGVPKPSFWPTAQNSGGSA |
| | | CAACAGCAGCTATTGTCCCGGGCTCCCAGCTGA | YSEERDRPYGLVSIDTVTVLDAEGPCTW |
| | | TGCCTTCAAAATCACCTTCCACAGGAACCACAG | PCSCEDDGYPALDLDAGLEPSPGLEDPL |
| | | AGATAAGCAGTCATGAGTCCTCCCACGGCACCC | LDAGTTVLSCGCVSAGSPGLGGPLGSLL |
| | | CCTCTCAGACAACAGCCAAGAACTGGGAACTC | DRLKPPLADGEDWAGGLPWGGRSPGGV |
| | | ACAGCATCCGCCTCCCACCAGCCGCCAGGTGTG | SESEAGSPLAGLDMDTFDSGFVGSDCSS |
| | | TATCCACAGGGCCACAGCGACACCACTGTGGCT | PVECDFTSPGDEGPPRSYLRQWVVIPPPL |
| | | ATCTCCACGTCCACTGTCCTGCTGTGTGGGCTG | SSPGPQAS (SEQ ID NO: 38) |
| | | AGCGCTGTGTCTCTCCTGGCATGCTACCTCAAG | |
| | | TCAAGGCAAACTCCCCCGCTGGCCAGCGTTGAA | |
| | | ATGGAAGCCATGGAGGCTCTGCCGGTGACTTGG | |
| | | GGGACCAGCAGCAGAGATGAAGACTTGGAAAA | |
| | | CTGCTCTCACCACCTA | |
| | | (SEQ ID NO: 6) | |
| LAT1 | | ATGGGGGTGCGGGCCCGAAGCGGCGCGCGCT | MAGAGPKRRALAAPAAEEKEEAREKML |
| | | AGCGGCGCCGGCGGCCGAGGAGAAGGAAGAG | AAKSADGSAPAGEGEGVTLQRNITLLNG |
| | | GCGCGGGAGAAGATGCTGGCCGCCAAGAGCGC | VAIIVGTIIGSGIFVTPTGVLKEAGSPGLA |
| | | GGACGGCTCGGCGCCGGCAGGCGAGGGCGAGG | LVVWAACGVFSIVGALCYAELGTTISKS |
| | | GCGTGACCCTGCAGCGGAACATCACGCTGCTCA | GGDYAYMLEVYGSLPAFLKLWIELLIIRP |
| | | ACGGCGTGGCCATCATCGTGGGGACCATTATCG | SSQYIVALVFATYLLKPLFPTCPVPEEAA |
| | | GCTCGGGCATCTTCGTGACGCCCACGGGCGTGC | KLVACLCVLLLTAVNCYSVKAATRVQD |
| | | TCAAGGAGGCAGGCTCGCCGGGGCTGGCGCTG | AFAAAKLLALALIILLGFVQIGKGDVSNL |
| | | GTGGTGTGGGCCGCGTGCGGCGTCTTCTCCATC | DPNFSFEGTKLDVGNIVLALYSGLFAYG |
| | | GTGGGCGCGCTCTGCTACGCGGAGCTCGGCACC | GWNYLNFVTEEMINPYRNLPLAIISLPIV |
| | | ACCATCTCCAAATCGGGCGGCGACTACGCCTAC | TLVYVLTNLAYFTTLSTEQMLSSEAVAV |
| | | ATGCTGGAGGTCTACGGCTCGCTGCCCGCCTTC | DFGNYHLGVMSWIIPVFVGLSCFGSVNG |
| | | CTCAAGCTCTGGATCGAGCTGCTCATCATCCGG | SLFTSSRLFFVGSREGHLPSILSMIHPQLL |
| | | CCTTCATCGCAGTACATCGTGGCCCTGGTCTTC | TPVPSLVFTCVMTLLYAFSKDIFSVINFFS |
| | | GCCACCTACCTGCTCAAGCCGCTCTTCCCCACC | FFNWLCVALAIGMIWLRHRKPELERPIK |
| | | TGCCCGGTGCCCGAGGAGGCAGCCAAGCTCGT | VNLALPVFFILACLFLIAVSFWKTPVECG |
| | | GGCCTGCCTCTGCGTGCTGCTGCTCACGGCCGT | IGFTIILSGLPVYFFGVWWKNKPKWLLQ |
| | | GAACTGCTACAGCGTGAAGGCCGCCACCCGGG | GIFSTTVLCQKLMQVVPQET (SEQ ID |
| | | TCCAGGATGCCTTTGCCGCCGCCAAGCTCCTGG | NO: 39) |
| | | CCCTGGCCCTGATCATCCTGCTGGGCTTCGTCC | |
| | | AGATCGGGAAGGGTGATGTGTCCAATCTAGATC | |
| | | CCAACTTCTCATTTGAAGGCACCAAACTGGATG | |
| | | TGGGGAACATTGTGCTGGCATTATACAGCGGCC | |
| | | TCTTTGCCTATGGAGGATGGAATTACTTGAATT | |
| | | TCGTCACAGAGGAAATGATCAACCCCTACAGA | |
| | | AACCTGCCCCTGGCCATCATCATCTCCCTGCCC | |
| | | ATCGTGACGCTGGTGTACGTGCTGACCAACCTG | |
| | | GCCTACTTCACCACCCTGTCCACCGAGCAGATG | |
| | | CTGTCGTCCGAGGCCGTGGCCGTGGACTTCGGG | |
| | | AACTATCACCTGGGCGTCATGTCCTGGATCATC | |
| | | CCCGTCTTCGTGGGCCTGTCCTGCTTCGGCTCCG | |
| | | TCAATGGGTCCCTGTTCACATCCTCCAGGCTCTT | |
| | | CTTCGTGGGGTCCCGGGAAGGCCACCTGCCCTC | |
| | | CATCCTCTCCATGATCCACCCACAGCTCCTCAC | |
| | | CCCCGTGCCGTCCCTCGTGTTCACGTGTGTGAT | |
| | | GACGCTGCTCTACGCCTTCTCCAAGGACATCTT | |
| | | CTCCGTCATCAACTTCTTCAGCTTCTTCAACTGG | |
| | | CTCTGCGTGGCCCTGGCCATCATCGGCATGATC | |
| | | TGGCTGCGCCACAGAAAGCCTGAGCTTGAGCG | |
| | | GCCCATCAAGGTGAACCTGGCCCTGCCTGTGTT | |
| | | CTTCATCCTGGCCTGCCTCTTCCTGATCGCCGTC | |
| | | TCCTTCTGGAAGACACCCGTGGAGTGTGGCATC | |
| | | GGCTTCACCATCATCCTCAGCGGGCTGCCCGTC | |
| | | TACTTCTTCGGGGTCTGGTGGAAAAACAAGCCC | |
| | | AAGTGGCTCCTCCAGGGCATCTTCTCCACGACC | |
| | | GTCCTGTGTCAGAAGCTCATGCAGGTGGTCCCC | |
| | | CAGGAGACA (SEQ ID NO: 7) | |
| LAG3 | 4-1BB | ATGTGGGAAGCCCAATTTCTCGGCCTGCTTTTC | MWEAQFLGLLFLQPLWVAPVKPLQPGA |
| | | TTGCAACCCCTGTGGGTTGCGCCTGTCAAACCC | EVPVVWAQEGAPAQLPCSPTIPLQDLSL |
| | | CTGCAACCTGGTGCCGAAGTGCCCGTCGTTTGG | LRRAGVTWQHQPDSGPPAAAPGHPLAP |
| | | GCACAAGAAGGAGCACCAGCTCAACTTCCATG | GPHPAAPSSWGPRPRRYTVLSVGPGGLR |
| | | TTCCCCTACCATTCCTCTTCAAGACCTTAGTCTG | SGRLPLQPRVQLDERGRQRGDFSLWLRP |
| | | TTGCGCCGGGCCGGAGTTACGTGGCAACACCA | ARRADAGEYRAAVHLRDRALSCRLRLR |
| | | ACCCGATTCCGGACCACCAGCGGCTGCACCTGG | LGQASMTASPPGSLRASDWVILNCSFSR |
| | | ACACCCATTGGCGCCTGGGCCACATCCAGCCGC | PDRPASVHWFRNRGQGRVPVRESPHHH |
| | | CCCGTCTAGCTGGGGACCTAGACCTAGACGGTA | LAESFLFLPQVSPMDSGPWGCILTYRDG |
| | | TACAGTATTGTCCGTCGGCCCTGGTGGACTCCG | FNVSIMYNLTVLGLEPPTPLTVYAGAGS |
| | | GTCTGGTCGACTCCCGCTTCAACCAAGAGTGCA | RVGLPCRLPAGVGTRSFLTAKWTPPGGG |
| | | ACTCGACGAACGCGGGAGACAACGTGGAGATT | PDLLVTGDNGDFTLRLEDVSQAQAGTY |
| | | TTAGCCTGTGGTTGCGTCCTGCGAGACGTGCCG | TCHIHLQEQQLNATVTLAIITVTPKSFGS |
| | | ATGCTGGGGAATATCGTGCAGCCGTCCATTTGC | PGSLGKLLCEVTPVSGQERFVWSSLDTP |
| | | GAGATAGAGCTCTTTCATGTCGGCTGCGCCTCA | SQRSFSGPWLEAQEAQLLSQPWQCQLY |
| | | GGCTCGGTCAAGCAAGCATGACCGCGTCCCCGC | QGERLLGAAVYFTELSSPGAQRSGRAPA |
| | | CGGGTTCACTGCGCGCTTCAGATTGGGTGATCC | PAREPGHSPQIISFFLALTSTALLFLLFFL |
| | | TCAATTGTAGCTTTTCCAGACCAGATAGACCCG | TLRFSVVKRGRKKLLYIFKQPFMRPVQTT |
| | | CTTCAGTACACTGGTTTAGGAATCGTGGTCAAG | QEEDGCCRFPEEEEGGCEL |
| | | GGCGCGTTCCGGTACGCGAATCTCCTCACCATC | (SEQ ID NO: 40) |
| | | ATCTGGCTGAGTCCTTTCTGTTTCTTCCACAGGT | |
| | | GTCCCCTATGGATTCCGGACCTTGGGGTTGTAT | |
| | | TCTTACATATCGGGACGGTTTTAATGTGTCAAT | |
| | | TATGTACAATCTGACCGTCTTGGGGCTCGAACC | |
| | | ACCCACGCCGCTGACTGTATATGCCGGCGCCGG | |
| | | ATCACGAGTTGGCCTTCCATGTAGATTGCCCGC | |
| | | CGGAGTCGGCACGAGGTCATTTCTGACCGCTAA | |
| | | ATGGACCCCACCCGGTGGTGGACCAGATTTGTT | |
| | | GGTAACCGGCGATAACGGAGATTTCACACTCA | |
| | | GACTTGAAGACGTATCTCAAGCTCAAGCCGGA | |
| | | ACATATACTTGTCACATACACTTGCAAGAGCAA | |
| | | CAATTGAACGCGACCGTTACGCTGGCTATTATT | |
| | | ACTGTAACGCCTAAGTCATTCGGTTCTCCCGGG | |
| | | TCATTGGGCAAACTTCTCTGCGAAGTTACGCCT | |
| | | GTCAGCGGCCAGGAGCGGTTCGTTTGGTCCAGC | |
| | | TTGGATACTCCTAGCCAACGAAGCTTTTCTGGT | |
| | | CCCTGGCTTGAAGCCCAAGAAGCACAACTGCTG | |
| | | TCACAACCCTGGCAGTGTCAACTTTATCAAGGC | |
| | | GAACGCCTGCTGGGTGCCGCGGTATATTTTACG | |
| | | GAACTTTCCTCACCCGGCGCACAGCGTTCAGGA | |
| | | CGGGCACCAGCGCCCGCTCGCGAACCCGGCCA | |
| | | TTCTCCTCAAATTATATCATTCTTCCTCGCACTT | |
| | | ACCAGTACGGCCCTTCTCTTTCTTTTGTTCTTTC | |
| | | TGACTCTTCGCTTTTCAGTGGTGAAGCGAGGTC | |
| | | GCAAGAAGCTGCTCTACATCTTTAAGCAGCCTT | |
| | | TCATGCGGCCCGTGCAGACGACCCAGGAAGAG | |
| | | GACGGTTGCTCATGTAGATTCCCTGAGGAAGAA | |
| | | GAGGGCGGCTGCGAGTTG | |
| | | (SEQ ID NO: 8) | |
| DR5 | IL-4R | atggaacaacggggacagaacgccccggccgcttcgg | MEQRGQNAPAASGARKRHGPGPREARG |
| | | gggcccggaaaaggcacggcccaggacccagggaggc | ARPGPRVPKTLVLVVAAVLLLVSAESAL |
| | | gcggggagccaggcctgggccccgggtccccaagacc | ITQQDLAPQQRAAPQQKRSSPSEGLCPP |
| | | cttgtgctcgttgtcgccgcggtcctgctgttggtct | GHHISEDGRDCISCKYGQDYSTHWNDLL |
| | | cagctgagtctgctctgatcacccaacaagacctagc | FCLRCTRCDSGEVELSPCTTTRNTVCQC |
| | | tccccagcagagagcggccccacaacaaaagaggtcc | EEGTFREEDSPEMCRKCRTGCPRGMVK |
| | | agcccctcagagggattgtgtccacctggacaccata | VGDCTPWSDIECVHKESGTKHSGEVPAV |
| | | tctcagaagacggtagagattgcatctcctgcaaata | EETVTSSPGTPASPCSLSGIIIGVTVAAVV |
| | | tggacaggactatagcactcactggaatgacctcctt | LIVAVFVCKSLLWKKIKKEWWDQIPNPA |
| | | ttctgcttgcgctgcaccaggtgtgattcaggtgaag | RSRLVAIIIQDAQGSQWEKRSRGQEPAK |
| | | tggagctaagtccctgcaccacgaccagaaacacagt | CPHWKNCLTKLLPCFLEHNMKRDEDPH |
| | | gtgtcagtgcgaagaaggcaccttccgggaagaagat | KAAKEMPFQGSGKSAWCPVEISKTVLW |
| | | tctcctgagatgtgccggaagtgccgcacagggtgtc | PESISVVRCVELFEAPVECEEEEEVEEEK |
| | | ccagagggatggtcaaggtcggtgattgtacaccctg | GSFCASPESSRDDFQEGREGIVARLTESL |
| | | gagtgacatcgaatgtgtccacaaagaatcaggtaca | FLDLLGEENGGFCQQDMGESCLLPPSGS |
| | | aagcacagtggggaagtcccagctgtggaggagacgg | TSAHMPWDEFPSAGPKEAPPWGKEQPL |
| | | tgacctccagcccagggactcctgcctctccctgttc | HLEPSPPASPTQSPDNLTCTETPLVIAGNP |
| | | tctctcaggcatcatcataggagtcacagttgcagcc | AYRSFSNSLSQSPCPRELGPDPLLARHLE |
| | | gtagtcttgattgtggctgtgtttgtttgcaagtctt | EVEPEMPCVPQLSEPTTVPQPEPETWEQI |
| | | tactgtggaagAAGATTAAGAAAGAATGGTGGGATCA | LRRNVLQHGAAAAPVSAPTSGYQEFVH |
| | | GATTCCCAACCCAGCCCGCAGCCGCCTCGTGGCTATA | AVEQGGTQASAVVGLGPPGEAGYKAFS |
| | | ATAATCCAGGATGCTCAGGGGTCACAGTGGGAGAAGC | SLLASSAVSPEKCGFGASSGEEGYKPFQ |
| | | GGTCCCGAGGCCAGGAACCAGCCAAGTGCCCACACTG | DLIPGCPGDPAPVPVPLFTFGLDREPPRSP |
| | | GAAGAATTGTCTTACCAAGCTCTTGCCCTGTTTTCTG | QSSHLPSSSPEHLGLEPGEKVEDMPKPPL |
| | | GAGCACAACATGAAAAGGGATGAAGATCCTCACAAGG | PQEQATDPLVDSLGSGIVYSALTCHLCG |
| | | CTGCCAAAGAGATGCCTTTCCAGGGCTCTGGAAAATC | HLKQCHGQEDGGQTPVMASPCCGCCCG |
| | | AGCATGGTGCCCAGTGGAGATCAGCAAGACAGTCCTC | DRSSPPTTPLRAPDPSPGGVPLEASLCPA |
| | | TGGCCAGAGAGCATCAGCGTGGTGCGATGTGTGGAGT | SLAPSGISEKSKSSSSFHPAPGNAQSSSQT |
| | | TGTTTGAGGCCCCGGTGGAGTGTGAGGAGGAGGAGGA | PKIVNFVSVGPTYMRVS (SEQ ID NO: |
| | | GGTAGAGGAAGAAAAAGGGAGCTTCTGTGCATCGCCT | 41) |
| | | GAGAGCAGCAGGGATGACTTCCAGGAGGGAAGGGAGG | |
| | | GCATTGTGGCCCGGCTAACAGAGAGCCTGTTCCT | |
| | | GGACCTGCTCGGAGAGGAGAATGGGGGCTTTT | |
| | | GCCAGCAGGACATGGGGGAGTCATGCCTTCTTC | |
| | | CACCTTCGGGAAGTACGAGTGCTCACATGCCCT | |
| | | GGGATGAGTTCCCAAGTGCAGGGCCCAAGGAG | |
| | | GCACCTCCCTGGGGCAAGGAGCAGCCTCTCCAC | |
| | | CTGGAGCCAAGTCCTCCTGCCAGCCCGACCCAG | |
| | | AGTCCAGACAACCTGACTTGCACAGAGACGCC | |
| | | CCTCGTCATCGCAGGCAACCCTGCTTACCGCAG | |
| | | CTTCAGCAACTCCCTGAGCCAGTCACCGTGTCC | |
| | | CAGAGAGCTGGGTCCAGACCCACTGCTGGCCA | |
| | | GACACCTGGAGGAAGTAGAACCCGAGATGCCC | |
| | | TGTGTCCCCCAGCTCTCTGAGCCAACCACTGTG | |
| | | CCCCAACCTGAGCCAGAAACCTGGGAGCAGAT | |
| | | CCTCCGCCGAAATGTCCTCCAGCATGGGGCAGC | |
| | | TGCAGCCCCCGTCTCGGCCCCCACCAGTGGCTA | |
| | | TCAGGAGTTTGTACATGCGGTGGAGCAGGGTG | |
| | | GCACCCAGGCCAGTGCGGTGGTGGGCTTGGGTC | |
| | | CCCCAGGAGAGGCTGGTTACAAGGCCTTCTCAA | |
| | | GCCTGCTTGCCAGCAGTGCTGTGTCCCCAGAGA | |
| | | AATGTGGGTTTGGGGCTAGCAGTGGGGAAGAG | |
| | | GGGTATAAGCCTTTCCAAGACCTCATTCCTGGC | |
| | | TGCCCTGGGGACCCTGCCCCAGTCCCTGTCCCC | |
| | | TTGTTCACCTTTGGACTGGACAGGGAGCCACCT | |
| | | CGCAGTCCGCAGAGCTCACATCTCCCAAGCAGC | |
| | | TCCCCAGAGCACCTGGGTCTGGAGCCGGGGGA | |
| | | AAAGGTAGAGGACATGCCAAAGCCCCCACTTC | |
| | | CCCAGGAGCAGGCCACAGACCCCCTTGTGGAC | |
| | | AGCCTGGGCAGTGGCATTGTCTACTCAGCCCTT | |
| | | ACCTGCCACCTGTGCGGCCACCTGAAACAGTGT | |
| | | CATGGCCAGGAGGATGGTGGCCAGACCCCTGT | |
| | | CATGGCCAGTCCTTGCTGTGGCTGCTGCTGTGG | |
| | | AGACAGGTCCTCGCCCCCTACAACCCCCCTGAG | |
| | | GGCCCCAGACCCCTCTCCAGGTGGGGTTCCACT | |
| | | GGAGGCCAGTCTGTGTCCGGCCTCCCTGGCACC | |
| | | CTCGGGCATCTCAGAGAAGAGTAAATCCTCATC | |
| | | ATCCTTCCATCCTGCCCCTGGCAATGCTCAGAG | |
| | | CTCAAGCCAGACCCCCAAAATCGTGAACTTTGT | |
| | | CTCCGTGGGACCCACATACATGAGGGTCTCT | |
| | | (SEQ ID NO: 9) | |
| DR4 | IL-4R | ATGGCTCCACCCCCGGCTAGAGTTCACCTCGGC | MAPPPARVHLGAFLAVTPNPGSAASGTE |
| | | GCTTTTCTTGCTGTCACACCTAACCCAGGTTCA | AAAATPSKVWGSSAGRIEPRGGGRGALP |
| | | GCCGCAAGCGGAACTGAAGCTGCGGCAGCTAC | TSMGQHGPSARARAGRAPGPRPAREASP |
| | | TCCTTCTAAGGTTTGGGGAAGCAGCGCTGGTCG | RLRVHKTFKFVVVGVLLQVVPSSAATIK |
| | | CATCGAACCCCGGGGTGGTGGTAGGGGTGCTCT | LHDQSIGTQQWEHSPLGELCPPGSHRSE |
| | | TCCGACATCTATGGGTCAACATGGTCCTTCAGC | HPGACNRCTEGVGYTNASNNLFACLPC |
| | | TCGAGCAAGGGCCGGAAGAGCACCGGGGCCAC | TACKSDEEERSPCTTTRNTACQCKPGTF |
| | | GGCCTGCCCGTGAGGCTAGTCCCCGCCTGCGAG | RNDNSAEMCRKCSRGCPRGMVKVKDC |
| | | TACATAAAACATTTAAATTCGTGGTAGTGGGAG | TPWSDIECVHKESGNGHNIWVILVVTLV |
| | | TTCTTCTTCAAGTTGTGCCAAGTAGTGCCGCTA | VPLLLVAVLIVCCCIGSGCGKIKKEWWD |
| | | CTATTAAGCTCCACGACCAGAGCATTGGGACCC | QIPNPARSRLVAIIIQDAQGSQWEKRSRG |
| | | AACAGTGGGAGCACAGTCCACTTGGCGAACTG | QEPAKCPHWKNCLTKLLPCFLEHNMKR |
| | | TGCCCACCCGGCAGTCACCGCTCTGAGCACCCC | DEDPHKAAKEMPFQGSGKSAWCPVEIS |
| | | GGGGCGTGCAATCGATGTACTGAAGGCGTAGG | KTVLWPESISVVRCVELFEAPVECEEEEE |
| | | CTATACGAACGCATCAAATAACCTGTTCGCCTG | VEEEKGSFCASPESSRDDFQEGREGIVAR |
| | | TCTTCCCTGCACCGCCTGCAAGTCCGACGAGGA | LTESLFLDLLGEENGGFCQQDMGESCLL |
| | | AGAAAGGTCACCATGTACTACAACACGCAATA | PPSGSTSAHMPWDEFPSAGPKEAPPWGK |
| | | CCGCCTGCCAATGTAAGCCCGGGACATTTCGCA | EQPLHLEPSPPASPTQSPDNLTCTETPLVI |
| | | ACGATAACTCAGCCGAAATGTGTCGTAAATGTT | AGNPAYRSFSNSLSQSPCPRELGPDPLLA |
| | | CTAGGGGATGTCCAAGGGGCATGGTAAAAGTG | RHLEEVEPEMPCVPQLSEPTTVPQPEPET |
| | | AAAGACTGCACACCTTGGAGCGATATAGAATG | WEQILRRNVLQHGAAAAPVSAPTSGYQ |
| | | CGTTCACAAGGAGTCCGGAAACGGTCACAACA | EFVHAVEQGGTQASAVVGLGPPGEAGY |
| | | TTTGGGTCATCCTTGTCGTCACCCTCGTGGTACC | KAFSSLLASSAVSPEKCGFGASSGEEGY |
| | | TCTGCTTCTGGTCGCAGTCCTCATCGTTTGCTGC | KPFQDLIPGCPGDPAPVPVPLFTFGLDRE |
| | | TGTATTGGATCCGGATGCGGCAAGATTAAGAA | PPRSPQSSHLPSSSPEHLGLEPGEKVEDM |
| | | AGAATGGTGGGATCAGATTCCCAACCCAGCCC | PKPPLPQEQATDPLVDSLGSGIVYSALTC |
| | | GCAGCCGCCTCGTGGCTATAATAATCCAGGATG | HLCGHLKQCHGQEDGGQTPVMASPCCG |
| | | CTCAGGGGTCACAGTGGGAGAAGCGGTCCCGA | CCCGDRSSPPTTPLRAPDPSPGGVPLEAS |
| | | GGCCAGGAACCAGCCAAGTGCCCACACTGGAA | LCPASLAPSGISEKSKSSSSFHPAPGNAQS |
| | | GAATTGTCTTACCAAGCTCTTGCCCTGTTTTCTG | SSQTPKIVNFVSVGPTYMRVS (SEQ ID |
| | | GAGCACAACATGAAAAGGGATGAAGATCCTCA | NO: 42) |
| | | CAAGGCTGCCAAAGAGATGCCTTTCCAGGGCTC | |
| | | TGGAAAATCAGCATGGTGCCCAGTGGAGATCA | |
| | | GCAAGACAGTCCTCTGGCCAGAGAGCATCAGC | |
| | | GTGGTGCGATGTGTGGAGTTGTTTGAGGCCCCG | |
| | | GTGGAGTGTGAGGAGGAGGAGGAGGTAGAGGA | |
| | | AGAAAAAGGGAGCTTCTGTGCATCGCCTGAGA | |
| | | GCAGCAGGGATGACTTCCAGGAGGGAAGGGAG | |
| | | GGCATTGTGGCCCGGCTAACAGAGAGCCTGTTC | |
| | | CTGGACCTGCTCGGAGAGGAGAATGGGGGCTT | |
| | | TTGCCAGCAGGACATGGGGGAGTCATGCCTTCT | |
| | | TCCACCTTCGGGAAGTACGAGTGCTCACATGCC | |
| | | CTGGGATGAGTTCCCAAGTGCAGGGCCCAAGG | |
| | | AGGCACCTCCCTGGGGCAAGGAGCAGCCTCTCC | |
| | | ACCTGGAGCCAAGTCCTCCTGCCAGCCCGACCC | |
| | | AGAGTCCAGACAACCTGACTTGCACAGAGACG | |
| | | CCCCTCGTCATCGCAGGCAACCCTGCTTACCGC | |
| | | AGCTTCAGCAACTCCCTGAGCCAGTCACCGTGT | |
| | | CCCAGAGAGCTGGGTCCAGACCCACTGCTGGCC | |
| | | AGACACCTGGAGGAAGTAGAACCCGAGATGCC | |
| | | CTGTGTCCCCCAGCTCTCTGAGCCAACCACTGT | |
| | | GCCCCAACCTGAGCCAGAAACCTGGGAGCAGA | |
| | | TCCTCCGCCGAAATGTCCTCCAGCATGGGGCAG | |
| | | CTGCAGCCCCCGTCTCGGCCCCCACCAGTGGCT | |
| | | ATCAGGAGTTTGTACATGCGGTGGAGCAGGGT | |
| | | GGCACCCAGGCCAGTGCGGTGGTGGGCTTGGG | |
| | | TCCCCCAGGAGAGGCTGGTTACAAGGCCTTCTC | |
| | | AAGCCTGCTTGCCAGCAGTGCTGTGTCCCCAGA | |
| | | GAAATGTGGGTTTGGGGCTAGCAGTGGGGAAG | |
| | | AGGGGTATAAGCCTTTCCAAGACCTCATTCCTG | |
| | | GCTGCCCTGGGGACCCTGCCCCAGTCCCTGTCC | |
| | | CCTTGTTCACCTTTGGACTGGACAGGGAGCCAC | |
| | | CTCGCAGTCCGCAGAGCTCACATCTCCCAAGCA | |
| | | GCTCCCCAGAGCACCTGGGTCTGGAGCCGGGG | |
| | | GAAAAGGTAGAGGACATGCCAAAGCCCCCACT | |
| | | TCCCCAGGAGCAGGCCACAGACCCCCTTGTGGA | |
| | | CAGCCTGGGCAGTGGCATTGTCTACTCAGCCCT | |
| | | TACCTGCCACCTGTGCGGCCACCTGAAACAGTG | |
| | | TCATGGCCAGGAGGATGGTGGCCAGACCCCTGT | |
| | | CATGGCCAGTCCTTGCTGTGGCTGCTGCTGTGG | |
| | | AGACAGGTCCTCGCCCCCTACAACCCCCCTGAG | |
| | | GGCCCCAGACCCCTCTCCAGGTGGGGTTCCACT | |
| | | GGAGGCCAGTCTGTGTCCGGCCTCCCTGGCACC | |
| | | CTCGGGCATCTCAGAGAAGAGTAAATCCTCATC | |
| | | ATCCTTCCATCCTGCCCCTGGCAATGCTCAGAG | |
| | | CTCAAGCCAGACCCCCAAAATCGTGAACTTTGT | |
| | | CTCCGTGGGACCCACATACATGAGGGTCTCT | |
| | | (SEQ ID NO: 10) | |
| TNFRSF1A | IL-4R | ATGGGCCTCTCCACCGTGCCTGACCTGCTGCTG | MGLSTVPDLLLPLVLLELLVGIYPSGVIG |
| | | CCACTGGTGCTCCTGGAGCTGTTGGTGGGAATA | LVPHLGDREKRDSVCPQGKYIHPQNNSI |
| | | TACCCCTCAGGGGTTATTGGACTGGTCCCTCAC | CCTKCHKGTYLYNDCPGPGQDTDCREC |
| | | CTAGGGGACAGGGAGAAGAGAGATAGTGTGTG | ESGSFTASENHLRHCLSCSKCRKEMGQV |
| | | TCCCCAAGGAAAATATATCCACCCTCAAAATAA | EISSCTVDRDTVCGCRKNQYRHYWSEN |
| | | TTCGATTTGCTGTACCAAGTGCCACAAAGGAAC | LFQCFNCSLCLNGTVHLSCQEKQNTVCT |
| | | CTACTTGTACAATGACTGTCCAGGCCCGGGGCA | CHAGFFLRENECVSCSNCKKSLECTKLC |
| | | GGATACGGACTGCAGGGAGTGTGAGAGCGGCT | LPQIENVKGTEDSGTTVLLPLVIFFGLCL |
| | | CCTTCACCGCTTCAGAAAACCACCTCAGACACT | LSLLFIGLMYRYQRWKIKKEWWDQIPNP |
| | | GCCTCAGCTGCTCCAAATGCCGAAAGGAAATG | ARSRLVAIIIQDAQGSQWEKRSRGQEPA |
| | | GGTCAGGTGGAGATCTCTTCTTGCACAGTGGAC | KCPHWKNCLTKLLPCFLEHNMKRDEDP |
| | | CGGGACACCGTGTGTGGCTGCAGGAAGAACCA | HKAAKEMPFQGSGKSAWCPVEISKTVL |
| | | GTACCGGCATTATTGGAGTGAAAACCTTTTCCA | WPESISVVRCVELFEAPVECEEEEEVEEE |
| | | GTGCTTCAATTGCAGCCTCTGCCTCAATGGGAC | KGSFCASPESSRDDFQEGREGIVARLTES |
| | | CGTGCACCTCTCCTGCCAGGAGAAACAGAACA | LFLDLLGEENGGFCQQDMGESCLLPPSG |
| | | CCGTGTGCACCTGCCATGCAGGTTTCTTTCTAA | STSAHMPWDEFPSAGPKEAPPWGKEQP |
| | | GAGAAAACGAGTGTGTCTCCTGTAGTAACTGTA | LHLEPSPPASPTQSPDNLTCTETPLVIAG |
| | | AGAAAAGCCTGGAGTGCACGAAGTTGTGCCTA | NPAYRSFSNSLSQSPCPRELGPDPLLARH |
| | | CCCCAGATTGAGAATGTTAAGGGCACTGAGGA | LEEVEPEMPCVPQLSEPTTVPQPEPETWE |
| | | CTCAGGCACCACAGTGCTGTTGCCCCTGGTCAT | QILRRNVLQHGAAAAPVSAPTSGYQEFV |
| | | TTTCTTTGGTCTTTGCCTTTTATCCCTCCTCTTCA | HAVEQGGTQASAVVGLGPPGEAGYKAF |
| | | TTGGTTTAATGTATCGCTACCAACGGTGGAAGA | SSLLASSAVSPEKCGFGASSGEEGYKPFQ |
| | | TTAAGAAAGAATGGTGGGATCAGATTCCCAAC | DLIPGCPGDPAPVPVPLFTFGLDREPPRSP |
| | | CCAGCCCGCAGCCGCCTCGTGGCTATAATAATC | QSSHLPSSSPEHLGLEPGEKVEDMPKPPL |
| | | CAGGATGCTCAGGGGTCACAGTGGGAGAAGCG | PQEQATDPLVDSLGSGIVYSALTCHLCG |
| | | GTCCCGAGGCCAGGAACCAGCCAAGTGCCCAC | HLKQCHGQEDGGQTPVMASPCCGCCCG |
| | | ACTGGAAGAATTGTCTTACCAAGCTCTTGCCCT | DRSSPPTTPLRAPDPSPGGVPLEASLCPA |
| | | GTTTTCTGGAGCACAACATGAAAAGGGATGAA | SLAPSGISEKSKSSSSFHPAPGNAQSSSQT |
| | | GATCCTCACAAGGCTGCCAAAGAGATGCCTTTC | PKIVNFVSVGPTYMRVS (SEQ ID NO: |
| | | CAGGGCTCTGGAAAATCAGCATGGTGCCCAGT | 43) |
| | | GGAGATCAGCAAGACAGTCCTCTGGCCAGAGA | |
| | | GCATCAGCGTGGTGCGATGTGTGGAGTTGTTTG | |
| | | AGGCCCCGGTGGAGTGTGAGGAGGAGGAGGAG | |
| | | GTAGAGGAAGAAAAAGGGAGCTTCTGTGCATC | |
| | | GCCTGAGAGCAGCAGGGATGACTTCCAGGAGG | |
| | | GAAGGGAGGGCATTGTGGCCCGGCTAACAGAG | |
| | | AGCCTGTTCCTGGACCTGCTCGGAGAGGAGAAT | |
| | | GGGGGCTTTTGCCAGCAGGACATGGGGGAGTC | |
| | | ATGCCTTCTTCCACCTTCGGGAAGTACGAGTGC | |
| | | TCACATGCCCTGGGATGAGTTCCCAAGTGCAGG | |
| | | GCCCAAGGAGGCACCTCCCTGGGGCAAGGAGC | |
| | | AGCCTCTCCACCTGGAGCCAAGTCCTCCTGCCA | |
| | | GCCCGACCCAGAGTCCAGACAACCTGACTTGCA | |
| | | CAGAGACGCCCCTCGTCATCGCAGGCAACCCTG | |
| | | CTTACCGCAGCTTCAGCAACTCCCTGAGCCAGT | |
| | | CACCGTGTCCCAGAGAGCTGGGTCCAGACCCAC | |
| | | TGCTGGCCAGACACCTGGAGGAAGTAGAACCC | |
| | | GAGATGCCCTGTGTCCCCCAGCTCTCTGAGCCA | |
| | | ACCACTGTGCCCCAACCTGAGCCAGAAACCTGG | |
| | | GAGCAGATCCTCCGCCGAAATGTCCTCCAGCAT | |
| | | GGGGCAGCTGCAGCCCCCGTCTCGGCCCCCACC | |
| | | AGTGGCTATCAGGAGTTTGTACATGCGGTGGAG | |
| | | CAGGGTGGCACCCAGGCCAGTGCGGTGGTGGG | |
| | | CTTGGGTCCCCCAGGAGAGGCTGGTTACAAGGC | |
| | | CTTCTCAAGCCTGCTTGCCAGCAGTGCTGTGTC | |
| | | CCCAGAGAAATGTGGGTTTGGGGCTAGCAGTG | |
| | | GGGAAGAGGGGTATAAGCCTTTCCAAGACCTC | |
| | | ATTCCTGGCTGCCCTGGGGACCCTGCCCCAGTC | |
| | | CCTGTCCCCTTGTTCACCTTTGGACTGGACAGG | |
| | | GAGCCACCTCGCAGTCCGCAGAGCTCACATCTC | |
| | | CCAAGCAGCTCCCCAGAGCACCTGGGTCTGGA | |
| | | GCCGGGGGAAAAGGTAGAGGACATGCCAAAGC | |
| | | CCCCACTTCCCCAGGAGCAGGCCACAGACCCCC | |
| | | TTGTGGACAGCCTGGGCAGTGGCATTGTCTACT | |
| | | CAGCCCTTACCTGCCACCTGTGCGGCCACCTGA | |
| | | AACAGTGTCATGGCCAGGAGGATGGTGGCCAG | |
| | | ACCCCTGTCATGGCCAGTCCTTGCTGTGGCTGC | |
| | | TGCTGTGGAGACAGGTCCTCGCCCCCTACAACC | |
| | | CCCCTGAGGGCCCCAGACCCCTCTCCAGGTGGG | |
| | | GTTCCACTGGAGGCCAGTCTGTGTCCGGCCTCC | |
| | | CTGGCACCCTCGGGCATCTCAGAGAAGAGTAA | |
| | | ATCCTCATCATCCTTCCATCCTGCCCCTGGCAAT | |
| | | GCTCAGAGCTCAAGCCAGACCCCCAAAATCGT | |
| | | GAACTTTGTCTCCGTGGGACCCACATACATGAG | |
| | | GGTCTCT (SEQ ID NO: 11) | |
| LTBR | IL-4R | ATGCTCCTGCCTTGGGCCACCTCTGCCCCCGGC | MLLPWATSAPGLAWGPLVLGLFGLLAA |
| | | CTGGCCTGGGGGCCTCTGGTGCTGGGCCTCTTC | SQPQAVPPYASENQTCRDQEKEYYEPQH |
| | | GGGCTCCTGGCAGCATCGCAGCCCCAGGCGGT | RICCSRCPPGTYVSAKCSRIRDTVCATCA |
| | | GCCTCCATATGCGTCGGAGAACCAGACCTGCAG | ENSYNEHWNYLTICQLCRPCDPVMGLE |
| | | GGACCAGGAAAAGGAATACTATGAGCCCCAGC | EIAPCTSKRKTQCRCQPGMFCAAWALE |
| | | ACCGCATCTGCTGCTCCCGCTGCCCGCCAGGCA | CTHCELLSDCPPGTEAELKDEVGKGNNH |
| | | CCTATGTCTCAGCTAAATGTAGCCGCATCCGGG | CVPCKAGHFQNTSSPSARCQPHTRCENQ |
| | | ACACAGTTTGTGCCACATGTGCCGAGAATTCCT | GLVEAAPGTAQSDTTCKNPLEPLPPEMS |
| | | ACAACGAGCACTGGAACTACCTGACCATCTGCC | GTMLMLAVLLPLAFFLLLATVFSCIWKS |
| | | AGCTGTGCCGCCCCTGTGACCCAGTGATGGGCC | HPSLCKIKKEWWDQIPNPARSRLVAIIIQ |
| | | TCGAGGAGATTGCCCCCTGCACAAGCAAACGG | DAQGSQWEKRSRGQEPAKCPHWKNCL |
| | | AAGACCCAGTGCCGCTGCCAGCCGGGAATGTTC | TKLLPCFLEHNMKRDEDPHKAAKEMPF |
| | | TGTGCTGCCTGGGCCCTCGAGTGTACACACTGC | QGSGKSAWCPVEISKTVLWPESISVVRC |
| | | GAGCTACTTTCTGACTGCCCGCCTGGCACTGAA | VELFEAPVECEEEEEVEEEKGSFCASPES |
| | | GCCGAGCTCAAAGATGAAGTTGGGAAGGGTAA | SRDDFQEGREGIVARLTESLFLDLLGEEN |
| | | CAACCACTGCGTCCCCTGCAAGGCCGGGCACTT | GGFCQQDMGESCLLPPSGSTSAHMPWD |
| | | CCAGAATACCTCCTCCCCCAGCGCCCGCTGCCA | EFPSAGPKEAPPWGKEQPLHLEPSPPASP |
| | | GCCCCACACCAGGTGTGAGAACCAAGGTCTGG | TQSPDNLTCTETPLVIAGNPAYRSFSNSL |
| | | TGGAGGCAGCTCCAGGCACTGCCCAGTCCGAC | SQSPCPRELGPDPLLARHLEEVEPEMPCV |
| | | ACAACCTGCAAAAATCCATTAGAGCCACTGCCC | PQLSEPTTVPQPEPETWEQILRRNVLQHG |
| | | CCAGAGATGTCAGGAACCATGCTGATGCTGGCC | AAAAPVSAPTSGYQEFVHAVEQGGTQA |
| | | GTTCTGCTGCCACTGGCCTTCTTTCTGCTCCTTG | SAVVGLGPPGEAGYKAFSSLLASSAVSP |
| | | CCACCGTCTTCTCCTGCATCTGGAAGAGCCACC | EKCGFGASSGEEGYKPFQDLIPGCPGDP |
| | | CTTCTCTCTGCAAGATTAAGAAAGAATGGTGGG | APVPVPLFTFGLDREPPRSPQSSHLPSSSP |
| | | ATCAGATTCCCAACCCAGCCCGCAGCCGCCTCG | EHLGLEPGEKVEDMPKPPLPQEQATDPL |
| | | TGGCTATAATAATCCAGGATGCTCAGGGGTCAC | VDSLGSGIVYSALTCHLCGHLKQCHGQE |
| | | AGTGGGAGAAGCGGTCCCGAGGCCAGGAACCA | DGGQTPVMASPCCGCCCGDRSSPPTTPL |
| | | GCCAAGTGCCCACACTGGAAGAATTGTCTTACC | RAPDPSPGGVPLEASLCPASLAPSGISEK |
| | | AAGCTCTTGCCCTGTTTTCTGGAGCACAACATG | SKSSSSFHPAPGNAQSSSQTPKIVNFVSV |
| | | AAAAGGGATGAAGATCCTCACAAGGCTGCCAA | GPTYMRVS (SEQ ID NO: 44) |
| | | AGAGATGCCTTTCCAGGGCTCTGGAAAATCAGC | |
| | | ATGGTGCCCAGTGGAGATCAGCAAGACAGTCC | |
| | | TCTGGCCAGAGAGCATCAGCGTGGTGCGATGTG | |
| | | TGGAGTTGTTTGAGGCCCCGGTGGAGTGTGAGG | |
| | | AGGAGGAGGAGGTAGAGGAAGAAAAAGGGAG | |
| | | CTTCTGTGCATCGCCTGAGAGCAGCAGGGATGA | |
| | | CTTCCAGGAGGGAAGGGAGGGCATTGTGGCCC | |
| | | GGCTAACAGAGAGCCTGTTCCTGGACCTGCTCG | |
| | | GAGAGGAGAATGGGGGCTTTTGCCAGCAGGAC | |
| | | ATGGGGGAGTCATGCCTTCTTCCACCTTCGGGA | |
| | | AGTACGAGTGCTCACATGCCCTGGGATGAGTTC | |
| | | CCAAGTGCAGGGCCCAAGGAGGCACCTCCCTG | |
| | | GGGCAAGGAGCAGCCTCTCCACCTGGAGCCAA | |
| | | GTCCTCCTGCCAGCCCGACCCAGAGTCCAGACA | |
| | | ACCTGACTTGCACAGAGACGCCCCTCGTCATCG | |
| | | CAGGCAACCCTGCTTACCGCAGCTTCAGCAACT | |
| | | CCCTGAGCCAGTCACCGTGTCCCAGAGAGCTGG | |
| | | GTCCAGACCCACTGCTGGCCAGACACCTGGAG | |
| | | GAAGTAGAACCCGAGATGCCCTGTGTCCCCCAG | |
| | | CTCTCTGAGCCAACCACTGTGCCCCAACCTGAG | |
| | | CCAGAAACCTGGGAGCAGATCCTCCGCCGAAA | |
| | | TGTCCTCCAGCATGGGGCAGCTGCAGCCCCCGT | |
| | | CTCGGCCCCCACCAGTGGCTATCAGGAGTTTGT | |
| | | ACATGCGGTGGAGCAGGGTGGCACCCAGGCCA | |
| | | GTGCGGTGGTGGGCTTGGGTCCCCCAGGAGAG | |
| | | GCTGGTTACAAGGCCTTCTCAAGCCTGCTTGCC | |
| | | AGCAGTGCTGTGTCCCCAGAGAAATGTGGGTTT | |
| | | GGGGCTAGCAGTGGGGAAGAGGGGTATAAGCC | |
| | | TTTCCAAGACCTCATTCCTGGCTGCCCTGGGGA | |
| | | CCCTGCCCCAGTCCCTGTCCCCTTGTTCACCTTT | |
| | | GGACTGGACAGGGAGCCACCTCGCAGTCCGCA | |
| | | GAGCTCACATCTCCCAAGCAGCTCCCCAGAGCA | |
| | | CCTGGGTCTGGAGCCGGGGGAAAAGGTAGAGG | |
| | | ACATGCCAAAGCCCCCACTTCCCCAGGAGCAG | |
| | | GCCACAGACCCCCTTGTGGACAGCCTGGGCAGT | |
| | | GGCATTGTCTACTCAGCCCTTACCTGCCACCTG | |
| | | TGCGGCCACCTGAAACAGTGTCATGGCCAGGA | |
| | | GGATGGTGGCCAGACCCCTGTCATGGCCAGTCC | |
| | | TTGCTGTGGCTGCTGCTGTGGAGACAGGTCCTC | |
| | | GCCCCCTACAACCCCCCTGAGGGCCCCAGACCC | |
| | | CTCTCCAGGTGGGGTTCCACTGGAGGCCAGTCT | |
| | | GTGTCCGGCCTCCCTGGCACCCTCGGGCATCTC | |
| | | AGAGAAGAGTAAATCCTCATCATCCTTCCATCC | |
| | | TGCCCCTGGCAATGCTCAGAGCTCAAGCCAGAC | |
| | | CCCCAAAATCGTGAACTTTGTCTCCGTGGGACC | |
| | | CACATACATGAGGGTCTCT (SEQ ID NO: 12) | |
| IL-4RA | ICOS | ATGGGGTGGCTTTGCTCTGGGCTCCTGTTCCCT | MGWLCSGLLFPVSCLVLLQVASSGNMK |
| | | GTGAGCTGCCTGGTCCTGCTGCAGGTGGCAAGC | VLQEPTCVSDYMSISTCEWKMNGPTNCS |
| | | TCTGGGAACATGAAGGTCTTGCAGGAGCCCACC | TELRLLYQLVFLLSEAHTCIPENNGGAG |
| | | TGCGTCTCCGACTACATGAGCATCTCTACTTGC | CVCHLLMDDVVSADNYTLDLWAGQQL |
| | | GAGTGGAAGATGAATGGTCCCACCAATTGCAG | LWKGSFKPSEHVKPRAPGNLTVHTNVS |
| | | CACCGAGCTCCGCCTGTTGTACCAGCTGGTTTT | DTLLLTWSNPYPPDNYLYNHLTYAVNI |
| | | TCTGCTCTCCGAAGCCCACACGTGTATCCCTGA | WSENDPADFRIYNVTYLEPSLRIAASTLK |
| | | GAACAACGGAGGCGCGGGGTGCGTGTGCCACC | SGISYRARVRAWAQCYNTTWSEWSPST |
| | | TGCTCATGGATGACGTGGTCAGTGCGGATAACT | KWHNSYREPFEQHLFWLPIGCAAFVVV |
| | | ATACACTGGACCTGTGGGCTGGGCAGCAGCTGC | CILGCILICWLTKKKYSSSVHDPNGEYM |
| | | TGTGGAAGGGCTCCTTCAAGCCCAGCGAGCATG | FMRAVNTAKKSRLTDVTL (SEQ ID NO: |
| | | TGAAACCCAGGGCCCCAGGAAACCTGACAGTT | 45) |
| | | CACACCAATGTCTCCGACACTCTGCTGCTGACC | |
| | | TGGAGCAACCCGTATCCCCCTGACAATTACCTG | |
| | | TATAATCATCTCACCTATGCAGTCAACATTTGG | |
| | | AGTGAAAACGACCCGGCAGATTTCAGAATCTAT | |
| | | AACGTGACCTACCTAGAACCCTCCCTCCGCATC | |
| | | GCAGCCAGCACCCTGAAGTCTGGGATTTCCTAC | |
| | | AGGGCACGGGTGAGGGCCTGGGCTCAGTGCTA | |
| | | TAACACCACCTGGAGTGAGTGGAGCCCCAGCA | |
| | | CCAAGTGGCACAACTCCTACAGGGAGCCCTTCG | |
| | | AGCAGCACCTCTTCTGGTTACCCATAGGATGTG | |
| | | CAGCCTTTGTTGTAGTCTGCATTTTGGGATGCAT | |
| | | ACTTATTTGTTGGCTTACAAAAAAGAAGTATTC | |
| | | ATCCAGTGTGCACGACCCTAACGGTGAATACAT | |
| | | GTTCATGAGAGCAGTGAACACAGCCAAAAAAT | |
| | | CTAGACTCACAGATGTGACCCTA (SEQ ID NO: | |
| | | 13) | |
| LAG-3 | ICOS | ATGTGGGAAGCACAATTTCTCGGACTCCTCTTC | MWEAQFLGLLFLQPLWVAPVKPLQPGA |
| | | CTTCAACCTCTGTGGGTCGCACCCGTTAAACCC | EVPVVWAQEGAPAQLPCSPTIPLQDLSL |
| | | CTGCAACCCGGCGCCGAAGTACCTGTCGTATGG | LRRAGVTWQHQPDSGPPAAAPGHPLAP |
| | | GCTCAAGAAGGAGCACCGGCGCAACTTCCGTG | GPHPAAPSSWGPRPRRYTVLSVGPGGLR |
| | | TTCACCAACTATTCCTCTGCAAGACTTGTCTCTC | SGRLPLQPRVQLDERGRQRGDFSLWLRP |
| | | TTGAGGCGGGCAGGAGTGACCTGGCAACACCA | ARRADAGEYRAAVHLRDRALSCRLRLR |
| | | ACCCGATTCCGGACCCCCTGCAGCAGCTCCAGG | LGQASMTASPPGSLRASDWVILNCSFSR |
| | | ACACCCACTCGCGCCTGGGCCCCATCCTGCTGC | PDRPASVHWFRNRGQGRVPVRESPHHH |
| | | CCCGTCTTCTTGGGGACCTCGCCCTAGGAGATA | LAESFLFLPQVSPMDSGPWGCILTYRDG |
| | | TACCGTCCTTAGTGTAGGCCCAGGCGGATTGAG | FNVSIMYNLTVLGLEPPTPLTVYAGAGS |
| | | ATCTGGTCGACTTCCGCTCCAACCTCGAGTTCA | RVGLPCRLPAGVGTRSFLTAKWTPPGGG |
| | | ATTGGACGAACGGGGACGCCAAAGGGGTGACT | PDLLVTGDNGDFTLRLEDVSQAQAGTY |
| | | TTTCACTCTGGCTCAGACCTGCACGCCGGGCTG | TCHIHLQEQQLNATVTLAIITVTPKSFGS |
| | | ATGCTGGAGAATATCGTGCTGCCGTTCATCTTC | PGSLGKLLCEVTPVSGQERFVWSSLDTP |
| | | GGGATCGTGCGTTGTCATGTCGTCTGCGGCTCC | SQRSFSGPWLEAQEAQLLSQPWQCQLY |
| | | GTCTCGGACAAGCTTCTATGACAGCTTCTCCGC | QGERLLGAAVYFTELSSPGAQRSGRAHI |
| | | CCGGCAGCCTGCGGGCTTCTGATTGGGTGATCC | YESQLCCQLKFWLPIGCAAFVVVCILGCI |
| | | TCAATTGTTCTTTTAGTCGACCCGATAGACCCG | LICWLIKKKYSSSVHDPNGEYMFMRAV |
| | | CTTCAGTTCACTGGTTTCGCAATAGGGGACAAG | NTAKKSRLTDVTL (SEQ ID NO: 46) |
| | | GACGTGTGCCCGTGAGGGAAAGTCCTCACCATC | |
| | | ATCTTGCTGAGTCTTTTCTGTTTCTGCCGCAGGT | |
| | | GTCTCCAATGGATAGTGGCCCATGGGGTTGTAT | |
| | | TTTGACGTATAGGGACGGGTTTAATGTAAGTAT | |
| | | AATGTACAATTTGACAGTCTTGGGGCTTGAACC | |
| | | ACCGACCCCTCTGACCGTTTATGCAGGCGCGGG | |
| | | GTCTCGCGTCGGACTCCCTTGTCGACTTCCAGC | |
| | | AGGCGTCGGCACAAGATCCTTTCTGACAGCAAA | |
| | | ATGGACGCCACCAGGTGGCGGTCCAGATTTGCT | |
| | | CGTCACAGGCGATAACGGAGATTTCACACTCAG | |
| | | ACTCGAAGACGTAAGTCAAGCACAAGCAGGCA | |
| | | CATATACGTGTCACATTCACTTGCAAGAGCAAC | |
| | | AACTGAACGCTACCGTAACCCTGGCCATTATTA | |
| | | CTGTTACCCCTAAGAGTTTCGGTAGCCCAGGCA | |
| | | GCCTTGGCAAACTCCTCTGCGAAGTCACGCCCG | |
| | | TGTCAGGCCAGGAGCGGTTCGTTTGGTCCAGTC | |
| | | TTGATACACCGTCTCAAAGATCTTTTAGTGGTC | |
| | | CATGGCTCGAAGCCCAAGAAGCTCAACTTTTGT | |
| | | CACAACCATGGCAGTGTCAACTTTATCAAGGAG | |
| | | AACGCCTGTTGGGCGCCGCTGTCTATTTTACCG | |
| | | AACTTAGTTCTCCCGGGGCACAGCGAAGCGGA | |
| | | AGGGCTCACATCTACGAGTCCCAGCTCTGCTGT | |
| | | CAACTCAAATTTTGGCTGCCAATTGGCTGCGCG | |
| | | GCTTTCGTCGTCGTGTGTATCCTGGGCTGTATCC | |
| | | TGATCTGCTGGCTGACGAAGAAGAAATACTCCT | |
| | | CAAGCGTCCATGATCCAAATGGAGAGTATATGT | |
| | | TTATGCGAGCTGTCAATACGGCGAAGAAGTCAC | |
| | | GACTGACCGACGTTACATTG (SEQ ID NO: 14) | |
| BATF | | ATGCCTCACAGCTCCGACAGCAGTGACTCCAGC | MPHSSDSSDSSFSRSPPPGKQDSSDDVRR |
| | | TTCAGCCGCTCTCCTCCCCCTGGCAAACAGGAC | VQRREKNRIAAQKSRQRQTQKADTLHL |
| | | TCATCTGATGATGTGAGAAGAGTTCAGAGGAG | ESEDLEKQNAALRKEIKQLTEELKYFTS |
| | | GGAGAAAAATCGTATTGCCGCCCAGAAGAGCC | VLNSHEPLCSVLAASTPSPPEVVYSAHAF |
| | | GACAGAGGCAGACACAGAAGGCCGACACCCTG | HQPHVSSPRFQP |
| | | CACCTGGAGAGCGAAGACCTGGAGAAACAGAA | (SEQ ID NO: 47) |
| | | CGCGGCTCTACGCAAGGAGATCAAGCAGCTCA | |
| | | CAGAGGAACTGAAGTACTTCACGTCGGTGCTGA | |
| | | ACAGCCACGAGCCCCTGTGCTCGGTGCTGGCCG | |
| | | CCAGCACGCCCTCGCCCCCCGAGGTGGTGTACA | |
| | | GCGCCCACGCATTCCACCAACCTCATGTCAGCT | |
| | | CCCCGCGCTTCCAGCCC (SEQ ID NO: 15) | |
| BATF3 | | ATGTCGCAAGGGCTCCCGGCCGCCGGCAGCGTC | MSQGLPAAGSVLQRSVAAPGNQPQPQP |
| | | CTGCAGAGGAGCGTCGCGGCGCCCGGGAACcag | QQQSPEDDDRKVRRREKNRVAAQRSRK |
| | | ccgcagccgcagccgcagcagcagAGCCCTGAGGATG | KQTQKADKLHEEYESLEQENTMLRREIG |
| | | ATGACAGGAAGGTCCGAAGGAGAGAAAAAAACCG | KLTEELKHLTEALKEHEKMCPLLLCPMN |
| | | AGTTGCTGCTCAGAGAAGTCGGAAGAAGCAGA | FVPVPPRPDPVAGCLPR (SEQ ID NO: |
| | | CCCAGAAGGCTGACAAGCTCCATGAGGAATAT | 48) |
| | | GAGAGCCTGGAGCAAGAAAACACCATGCTGCG | |
| | | GAGAGAGATCGGGAAGCTGACAGAGGAGCTGA | |
| | | AGCACCTGACAGAGGCACTGAAGGAGCACGAG | |
| | | AAGATGTGCCCGCTGCTGCTCTGCCCTATGAAC | |
| | | TTTGTGCCAGTGCCTCCCCGGCCGGACCCTGTG | |
| | | GCCGGCTGCTTGCCCCGA | |
| | | (SEQ ID NO: 16) | |
| BATF2 | | ATGTCGCAAGGGCTCCCGGCCGCCGGCAGCGTC | MSQGLPAAGSVLQRSVAAPGNQPQPQP |
| | | CTGCAGAGGAGCGTCGCGGCGCCCGGGAACcag | QQQSPEDDDRKVRRREKNRVAAQRSRK |
| | | ccgcagccgcagccgcagcagcagAGCCCTGAGGATG | KQTQKADKLHEEYESLEQENTMLRREIG |
| | | ATGACAGGAAGGTCCGAAGGAGAGAAAAAAACCG | KLTEELKHLTEALKEHEKMCPLLLCPMN |
| | | AGTTGCTGCTCAGAGAAGTCGGAAGAAGCAGA | FVPVPPRPDPVAGCLPR |
| | | CCCAGAAGGCTGACAAGCTCCATGAGGAATAT | (SEQ ID NO: 49) |
| | | GAGAGCCTGGAGCAAGAAAACACCATGCTGCG | |
| | | GAGAGAGATCGGGAAGCTGACAGAGGAGCTGA | |
| | | AGCACCTGACAGAGGCACTGAAGGAGCACGAG | |
| | | AAGATGTGCCCGCTGCTGCTCTGCCCTATGAAC | |
| | | TTTGTGCCAGTGCCTCCCCGGCCGGACCCTGTG | |
| | | GCCGGCTGCTTGCCCCGA (SEQ ID NO: 17) | |
| ID2 | | ATGAAAGCCTTCAGTCCCGTGAGGTCCGTTAGG | MKAFSPVRSVRKNSLSDHSLGISRSKTPV |
| | | AAAAACAGCCTGTCGGACCACAGCCTGGGCAT | DDPMSLLYNMNDCYSKLKELVPSIPQNK |
| | | CTCCCGGAGCAAAACCCCTGTGGACGACCCGAT | KVSKMEILQHVIDYILDLQIALDSHPTIVS |
| | | GAGCCTGCTATACAACATGAACGACTGCTACTC | LHHQRPGQNQASRTPLTTLNTDISILSLQ |
| | | CAAGCTCAAGGAGCTGGTGCCCAGCATCCCCCA | ASEFPSELMSNDSKALCG |
| | | GAACAAGAAGGTGAGCAAGATGGAAATCCTGC | (SEQ ID NO: 50) |
| | | AGCACGTCATCGACTACATCTTGGACCTGCAGA | |
| | | TCGCCCTGGACTCGCATCCCACTATTGTCAGCC | |
| | | TGCATCACCAGAGACCCGGGCAGAACCAGGCG | |
| | | TCCAGGACGCCGCTGACCACCCTCAACACGGAT | |
| | | ATCAGCATCCTGTCCTTGCAGGCTTCTGAATTC | |
| | | CCTTCTGAGTTAATGTCAAATGACAGCAAAGCA | |
| | | CTGTGTGGC (SEQ ID NO: 18) | |
| ID3 | | ATGAAGGCGCTGAGCCCGGTGCGCGGCTGCTA | MKALSPVRGCYEAVCCLSERSLAIARGR |
| | | CGAGGCGGTGTGCTGCCTGTCGGAACGCAGTCT | GKGPAAEEPLSLLDDMNHCYSRLRELVP |
| | | GGCCATCGCCCGGGGCCGAGGGAAGGGCCCGG | GVPRGTQLSQVEILQRVIDYILDLQVVLA |
| | | CAGCTGAGGAGCCGCTGAGCTTGCTGGACGAC | EPAPGPPDGPHLPIQTAELTPELVISNDK |
| | | ATGAACCACTGCTACTCCCGCCTGCGGGAACTG | RSFCH |
| | | GTACCCGGAGTCCCGAGAGGCACTCAGCTTAGC | (SEQ ID NO: 51) |
| | | CAGGTGGAAATCCTACAGCGCGTCATCGACTAC | |
| | | ATTCTCGACCTGCAGGTAGTCCTGGCCGAGCCA | |
| | | GCCCCTGGACCCCCTGATGGCCCCCACCTTCCC | |
| | | ATCCAGACAGCCGAGCTCACTCCGGAACTTGTC | |
| | | ATCTCCAACGACAAAAGGAGCTTTTGCCAC | |
| | | (SEQ ID NO: 19) | |
| IRF8 | | ATGTGTGACCGGAATGGTGGTCGGCGGCTTCGA | MCDRNGGRRLRQWLIEQIDSSMYPGLI |
| | | CAGTGGCTGATCGAGCAGATTGACAGTAGCAT | WENEEKSMFRIPWKHAGKQDYNQEVD |
| | | GTATCCAGGACTGATTTGGGAGAATGAGGAGA | ASIFKAWAVFKGKFKEGDKAEPATWKT |
| | | AGAGCATGTTCCGGATCCCTTGGAAACACGCTG | RLRCALNKSPDFEEVTDRSQLDISEPYKV |
| | | GCAAGCAAGATTATAATCAGGAAGTGGATGCC | YRIVPEEEQKCKLGVATAGCVNEVTEM |
| | | TCCATTTTTAAGGCCTGGGCAGTTTTTAAAGGG | ECGRSEIDELIKEPSVDDYMGMIKRSPSP |
| | | AAGTTTAAAGAAGGGGACAAAGCTGAACCAGC | PEACRSQLLPDWWAQQPSTGVPLVTGY |
| | | CACTTGGAAGACGAGGTTACGCTGTGCTTTGAA | TTYDAHHSAFSQMVISFYYGGKLVGQA |
| | | TAAGAGCCCAGATTTTGAGGAAGTGACGGACC | TTTCPEGCRLSLSQPGLPGTKLYGPEGLE |
| | | GGTCCCAACTGGACATTTCCGAGCCATACAAAG | LVRFPPADAIPSERQRQVTRKLFGHLER |
| | | TTTACCGAATTGTTCCTGAGGAAGAGCAAAAAT | GVLLHSSRQGVFVKRLCQGRVFCSGNA |
| | | GCAAACTAGGCGTGGCAACTGCTGGCTGCGTG | VVCKGRPNKLERDEVVQVFDTSQFFREL |
| | | AATGAAGTTACAGAGATGGAGTGCGGTCGCTCT | QQFYNSQGRLPDGRVVLCFGEEFPDMA |
| | | GAAATCGACGAGCTGATCAAGGAGCCTTCTGTG | PLRSKLILVQIEQLYVRQLAEEAGKSCG |
| | | GACGATTACATGGGGATGATCAAAAGGAGCCC | AGSVMQAPEEPPPDQVFRMFPDICASHQ |
| | | TTCCCCGCCGGAGGCCTGTCGGAGTCAGCTCCT | RSFFRENQQITV |
| | | TCCAGACTGGTGGGCGCAGCAGCCCAGCACAG | (SEQ ID NO: 52) |
| | | GCGTGCCGCTGGTGACGGGGTACACCACCTACG | |
| | | ACGCGCACCATTCAGCATTCTCCCAGATGGTGA | |
| | | TCAGCTTCTACTATGGGGGCAAGCTGGTGGGCC | |
| | | AGGCCACCACCACCTGCCCCGAGGGCTGCCGCC | |
| | | TGTCCCTGAGCCAGCCTGGGCTGCCCGGCACCA | |
| | | AGCTGTATGGGCCCGAGGGCCTGGAGCTGGTG | |
| | | CGCTTCCCGCCGGCCGACGCCATCCCCAGCGAG | |
| | | CGACAGAGGCAGGTGACGCGGAAGCTGTTCGG | |
| | | GCACCTGGAGCGCGGGGTGCTGCTGCACAGCA | |
| | | GCCGGCAGGGCGTGTTCGTCAAGCGGCTGTGCC | |
| | | AGGGCCGCGTGTTCTGCAGCGGCAACGCCGTG | |
| | | GTGTGCAAAGGCAGGCCCAACAAGCTGGAGCG | |
| | | TGATGAGGTGGTCCAGGTCTTCGACACCAGCCA | |
| | | GTTCTTCCGAGAGCTGCAGCAGTTCTATAACAG | |
| | | CCAGGGCCGGCTTCCTGACGGCAGGGTGGTGCT | |
| | | GTGCTTTGGGGAAGAGTTTCCGGATATGGCCCC | |
| | | CTTGCGCTCCAAACTCATTCTCGTGCAGATTGA | |
| | | GCAGCTGTATGTCCGGCAACTGGCAGAAGAGG | |
| | | CTGGGAAGAGCTGTGGAGCCGGCTCTGTGATGC | |
| | | AGGCCCCCGAGGAGCCGCCGCCAGACCAGGTC | |
| | | TTCCGGATGTTTCCAGATATTTGTGCCTCACACC | |
| | | AGAGATCATTTTTCAGAGAAAACCAACAGATC | |
| | | ACCGTC (SEQ ID NO: 20) | |
| MYC | | ATGCCCCTCAACGTTAGCTTCACCAACAGGAAC | MPLNVSFTNRNYDLDYDSVQPYFYCDE |
| | | TATGACCTCGACTACGACTCGGTGCAGCCGTAT | EENFYQQQQQSELQPPAPSEDIWKKFEL |
| | | TTCTACTGCGACGAGGAGGAGAACTTCTACCAG | LPTPPLSPSRRSGLCSPSYVAVTPFSLRG |
| | | CAGCAGCAGCAGAGCGAGCTGCAGCCCCCGGC | DNDGGGGSFSTADQLEMVTELLGGDMV |
| | | GCCCAGCGAGGATATCTGGAAGAAATTCGAGC | NQSFICDPDDETFIKNIIIQDCMWSGFSA |
| | | TGCTGCCCACCCCGCCCCTGTCCCCTAGCCGCC | AAKLVSEKLASYQAARKDSGSPNPARG |
| | | GCTCCGGGCTCTGCTCGCCCTCCTACGTTGCGG | HSVCSTSSLYLQDLSAAASECIDPSVVFP |
| | | TCACACCCTTCTCCCTTCGGGGAGACAACGACG | YPLNDSSSPKSCASQDSSAFSPSSDSLLSS |
| | | GCGGTGGCGGGAGCTTCTCCACGGCCGACCAG | TESSPQGSPEPLVLHEETPPTTSSDSEEEQ |
| | | CTGGAGATGGTGACCGAGCTGCTGGGAGGAGA | EDEEEIDVVSVEKRQAPGKRSESGSPSA |
| | | CATGGTGAACCAGAGTTTCATCTGCGACCCGGA | GGHSKPPHSPLVLKRCHVSTHQHNYAA |
| | | CGACGAGACCTTCATCAAAAACATCATCATCCA | PPSTRKDYPAAKRVKLDSVRVLRQISNN |
| | | GGACTGTATGTGGAGCGGCTTCTCGGCCGCCGC | RKCTSPRSSDTEENVKRRTHNVLERQRR |
| | | CAAGCTCGTCTCAGAGAAGCTGGCCTCCTACCA | NELKRSFFALRDQIPELENNEKAPKVVIL |
| | | GGCTGCGCGCAAAGACAGCGGCAGCCCGAACC | KKATAYILSVQAEEQKLISEEDLLRKRRE |
| | | CCGCCCGCGGCCACAGCGTCTGCTCCACCTCCA | QLKHKLEQLRNSCA |
| | | GCTTGTACCTGCAGGATCTGAGCGCCGCCGCCT | (SEQ ID NO: 53) |
| | | CAGAGTGCATCGACCCCTCGGTGGTCTTCCCCT | |
| | | ACCCTCTCAACGACAGCAGCTCGCCCAAGTCCT | |
| | | GCGCCTCGCAAGACTCCAGCGCCTTCTCTCCGT | |
| | | CCTCGGATTCTCTGCTCTCCTCGACGGAGTCCTC | |
| | | CCCGCAGGGCAGCCCCGAGCCCCTGGTGCTCCA | |
| | | TGAGGAGACACCGCCCACCACCAGCAGCGACT | |
| | | CTGAGGAGGAACAAGAAGATGAGGAAGAAATC | |
| | | GATGTTGTTTCTGTGGAAAAGAGGCAGGCTCCT | |
| | | GGCAAAAGGTCAGAGTCTGGATCACCTTCTGCT | |
| | | GGAGGCCACAGCAAACCTCCTCACAGCCCACT | |
| | | GGTCCTCAAGAGGTGCCACGTCTCCACACATCA | |
| | | GCACAACTACGCAGCGCCTCCCTCCACTCGGAA | |
| | | GGACTATCCTGCTGCCAAGAGGGTCAAGTTGGA | |
| | | CAGTGTCAGAGTCCTGAGACAGATCAGCAACA | |
| | | ACCGAAAATGCACCAGCCCCAGGTCCTCGGAC | |
| | | ACCGAGGAGAATGTCAAGAGGCGAACACACAA | |
| | | CGTCTTGGAGCGCCAGAGGAGGAACGAGCTAA | |
| | | AACGGAGCTTTTTTGCCCTGCGTGACCAGATCC | |
| | | CGGAGTTGGAAAACAATGAAAAGGCCCCCAAG | |
| | | GTAGTTATCCTTAAAAAAGCCACAGCATACATC | |
| | | CTGTCCGTCCAAGCAGAGGAGCAAAAGCTCATT | |
| | | TCTGAAGAGGACTTGTTGCGGAAACGACGAGA | |
| | | ACAGTTGAAACACAAACTTGAACAGCTACGGA | |
| | | ACTCTTGTGCG (SEQ ID NO: 21) | |
| POU2F1 | | ATGGCGGACGGAGGAGCAGCGAGTCAAGATGA | MADGGAASQDESSAAAAAAADSRMNN |
| | | GAGTTCAGCCGCGGCGGCAGCAGCAGCAGACT | PSETSKPSMESGDGNTGTQTNGLDFQKQ |
| | | CAAGAATGAACAATCCGTCAGAAACCAGTAAA | PVPVGGAISTAQAQAFLGHLHQVQLAG |
| | | CCATCTATGGAGAGTGGAGATGGCAACACAGgc | TSLQAAAQSLNVQSKSNEESGDSQQPSQ |
| | | acacaaaccaatggtctggactttcagaagcagcctg | PSQQPSVQAAIPQTQLMLAGGQITGLTL |
| | | TGCCTGTAGGAGGAGCAATCTCAACAGCCCAGGCGCA | TPAQQQLLLQQAQAQAQLLAAAVQQHS |
| | | GGCTTTCCTTGGACATCTCCATCAGGTCCAACTCGCT | ASQQHSAAGATISASAATPMTQIPLSQPI |
| | | GGAACAAGTTTACAGGCTGCTGCTCAGTCTTTAA | QIAQDLQQLQQLQQQNLNLQQFVLVHP |
| | | ATGTACAGTCTAAATCTAATGAAGAATCGGGG | TTNLQPAQFIISQTPQGQQGLLQAQNLLT |
| | | GATTCGCAGCAGCCAAGCCAGCCTTCCCAGCAG | QLPQQSQANLLQSQPSITLTSQPATPTRTI |
| | | CCTTCAGTGCAGGCAGCCATTCCCCAGACCCAG | AATPIQTLPQSQSTPKRIDTPSLEEPSDLE |
| | | CTTATGCTAGCTGGAGGACAGATAACTGGGCTT | ELEQFAKTFKQRRIKLGFTQGDVGLAM |
| | | ACTTTGACGCCTGCCCAGCAACAGTTACTACTC | GKLYGNDFSQTTISRFEALNLSFKNMCK |
| | | CAGcaggcacaggcacaggcacagCTGCTGGCTGCTG | LKPLLEKWLNDAENLSSDSSLSSPSALNS |
| | | CAGTGCAGCAGCACTCCGCCAGCCAGCAGCACAGT | PGIEGLSRRRKKRTSIETNIRVALEKSFLE |
| | | GCTGCTGGAGCCACCATCTCCGCCTCTGCTGCC | NQKPTSEEITMIADQLNMEKEVIRVWFC |
| | | ACGCCCATGACGCAGATCCCCCTGTCTCAGCCC | NRRQKEKRINPPSSGGTSSSPIKAIFPSPT |
| | | ATACAGATCGCACAGGATcttcaacaactgcaacagc | SLVATTPSLVTSSAATTLTVSPVLPLTSAA |
| | | ttcaacagcAGAATCTCAACCTGCAACAGTTTGTGTT | VTNLSVTGTSDTTSNNTATVISTAPPASS |
| | | GGTGCATCCAACCACCAATTTGCAGCCAGCGCAGT | AVTSPSLSPSPSASASTSEASSASETSTTQ |
| | | TTATCATCTCACAGACGCCCCAGGGCCAGCAGG | TTSTPLSSPLGTSQVMVTASGLQTAAAA |
| | | GTCTCCTGCAAGCGCAAAATCTTCTAACGCAAC | ALQGAAQLPANASLAAMAAAAGLNPSL |
| | | TACCTCAGCAAAGCCAAGCCAACCTCCTACAGT | MAPSQFAAGGALLSLNPGTLSGALSPAL |
| | | CGCAGCCAAGCATCACCCTCACCTCCCAGCCAG | MSNSTLATIQALASGGSLPITSLDATGNL |
| | | CAACCCCAACACGCACAATAGCAGCAACCCCA | VFANAGGAPNIVTAPLFLNPQNLSLLTS |
| | | ATTCAGACACTTCCACAGAGCCAGTCAACACCA | NPVSLVSAAAASAGNSAPVASLHATSTS |
| | | AAGCGAATTGATACTCCCAGCTTGGAGGAGCCC | AESIQNSLFTVASASGAASTTTTASKAQ |
| | | AGTGACCTTGAGGAGCTTGAGCAGTTTGCCAAG | (SEQ ID NO: 54) |
| | | ACCTTCAAACAAAGACGAATCAAACTTGGATTC | |
| | | ACTCAGGGTGATGTTGGGCTCGCTATGGGGAAA | |
| | | CTATATGGAAATGACTTCAGCCAAACTACCATC | |
| | | TCTCGATTTGAAGCCTTGAACCTCAGCTTTAAG | |
| | | AACATGTGCAAGTTGAAGCCACTTTTAGAGAAG | |
| | | TGGCTAAATGATGCAGAGAACCTCTCATCTGAT | |
| | | TCGTCCCTCTCCAGCCCAAGTGCCCTGAATTCT | |
| | | CCAGGAATTGAGGGCTTGAGCCGTAGGAGGAA | |
| | | GAAACGCACCAGCATAGAGACCAACATCCGTG | |
| | | TGGCCTTAGAGAAGAGTTTCTTGGAGAATCAAA | |
| | | AGCCTACCTCGGAAGAGATCACTATGATTGCTG | |
| | | ATCAGCTCAATATGGAAAAAGAGGTGATTCGT | |
| | | GTTTGGTTCTGTAACCGCCGCCAGAAAGAAAAA | |
| | | AGAATCAACCCACCAAGCAGTGGTGGGACCAG | |
| | | CAGCTCACCTATTAAAGCAATTTTCCCCAGCCC | |
| | | AACTTCACTGGTGGCGACCACACCAAGCCTTGT | |
| | | GACTAGCAGTGCAGCAACTACCCTCACAGTCAG | |
| | | CCCTGTCCTCCCTCTGACCAGTGCTGCTGTGAC | |
| | | GAATCTTTCAGTTACAGGCACTTCAGACACCAC | |
| | | CTCCAACAACACAGCAACCGTGATTTCCACAGC | |
| | | GCCTCCAGCTTCCTCAGCAGTCACGTCCCCCTC | |
| | | TCTGAGTCCCTCCCCTTCTGCCTCAGCCTCCACC | |
| | | TCCGAGGCATCCAGTGCCAGTGAGACCAGCAC | |
| | | AACACAGACCACCTCCACTCCTTTGTCCTCCCC | |
| | | TCTTGGGACCAGCCAGGTGATGGTGACAGCATC | |
| | | AGGTTTGCAAACAGCAGCAGCTGCTGCCCTTCA | |
| | | AGGAGCTGCACAGTTGCCAGCAAATGCCAGTCT | |
| | | TGCTGCCATGGCAGCTGCTGCAGGACTAAACCC | |
| | | AAGCCTGATGGCACCCTCACAGTTTGCGGCTGG | |
| | | AGGTGCCTTACTCAGTCTGAATCCAGGGACCCT | |
| | | GAGCGGTGCTCTCAGCCCAGCTCTAATGAGCAA | |
| | | CAGTACACTGGCAACTATTCAAGCTCTTGCTTC | |
| | | TGGTGGCTCTCTTCCAATAACATCACTTGATGC | |
| | | AACTGGGAACCTGGTATTTGCCAATGCGGGAG | |
| | | GAGCCCCCAACATCGTGACTGCCCCTCTGTTCC | |
| | | TGAACCCTCAGAACCTCTCTCTGCTCACCAGCA | |
| | | ACCCTGTTAGCTTGGTCTCTGCCGCCGCAGCAT | |
| | | CTGCAGGGAACTCTGCACCTGTAGCCAGCCTTC | |
| | | ACGCCACCTCCACCTCTGCTGAGTCCATCCAGA | |
| | | ACTCTCTCTTCACAGTGGCCTCTGCCAGCGGGG | |
| | | CTGCGTCCACCACCACCACCGCCTCCAAGGCAC | |
| | | AG (SEQ ID NO: 22) | |
| TFAP4 | | ATGGAGTATTTCATGGTGCCCACTCAGAAGGTG | MEYFMVPTQKVPSLQHFRKTEKEVIGGL |
| | | CCCTCTTTGCAACATTTCAGGAAAACAGAGAAA | CSLANIPLTPETQRDQERRIRREIANSNER |
| | | GAAGTGATAGGAGGGCTCTGTAGCCTTGCCAAC | RRMQSINAGFQSLKTLIPHTDGEKLSKA |
| | | ATTCCACTAACCCCCGAGACTCAGCGGGACCAG | AILQQTAEYIFSLEQEKTRLLQQNTQLKR |
| | | GAGCGGCGGATTCGGCGGGAGATCGCCAACAG | FIQELSGSSPKRRRAEDKDEGIGSPDIWE |
| | | CAACGAGCGGAGACGCATGCAGAGCATCAACG | DEKAEDLRREMIELRQQLDKERSVRMM |
| | | CGGGATTCCAGTCCCTCAAGACCCTCATCCCCC | LEEQVRSLEAHMYPEKLKVIAQQVQLQ |
| | | ACACAGACGGAGAGAAGCTCAGCAAGGCAGCC | QQQEQVRLLHQEKLEREQQQLRTQLLPP |
| | | ATTCTCCAGCAGACAGCCGAGTACATCTTCTCC | PAPTHHPTVIVPAPPPPPSHHINVVTMGP |
| | | CTGGAGCAGGAGAAGACCAGGCTCTTGCAGCA | SSVINSVSTSRQNLDTIVQAIQHIEGTQEK |
| | | GAACACACAGCTCAAGCGCTTCATCCAGGAGCT | QELEEEQRRAVIVKPVRSCPEAPTSDTAS |
| | | GAGCGGCTCGTCCCCCAAGCGACGGGGGGCAG | DSEASDSDAMDQSREEPSGDGELP |
| | | AGGACAAGGACGAAGGCATAGGCTCCCCGGAC | (SEQ ID NO: 55) |
| | | ATCTGGGAGGACGAGAAGGCGGAGGACCTGCG | |
| | | GCGGGAGATGATTGAGCTGCGGCAGCAGCTGG | |
| | | ACAAGGAGCGCTCGGTGCGCATGATGCTGGAG | |
| | | GAGCAGGTGCGCTCGCTGGAGGCCCACATGTA | |
| | | CCCGGAAAAGCTCAAGGTGATTGCGCAGCAGG | |
| | | TGCAGCTGCAGCAGCAGCAGGAACAGGTGAGG | |
| | | CTGCTGCACCAGGAGAAGCTGGAGCGGGAACA | |
| | | GCAGCAGCTGCGGACCCAGCTTCTGCCCCCTCC | |
| | | GGCCCCCACCCACCACCCCACGGTGATCGTGCC | |
| | | AGCACCGCCTCCTCCTCCCTCCCACCACATCAA | |
| | | TGTCGTCACCATGGGCCCCTCCTCGGTCATCAA | |
| | | CTCTGTTTCCACATCCCGGCAAAATCTGGACAC | |
| | | CATCGTGCAGGCAATCCAGCACATCGAGGGCA | |
| | | CCCAGGAAAAGCAGGAGCTGGAGGAGGAGCAG | |
| | | CGGCGAGCTGTCATCGTGAAGCCTGTCCGCAGC | |
| | | TGCCCGGAGGCCCCCACCTCTGACACCGCCTCC | |
| | | GACTCCGAGGCCTCAGACAGTGACGCCATGGA | |
| | | CCAGAGCCGGGAGGAGCCGTCGGGGGACGGGG | |
| | | AGCTTCCC (SEQ ID NO: 23) | |
| SMAD4 | | ATGGACAATATGTCTATTACGAATACACCAACA | MDNMSITNTPTSNDACLSIVHSLMCHRQ |
| | | AGTAATGATGCCTGTCTGAGCATTGTGCATAGT | GGESETFAKRAIESLVKKLKEKKDELDS |
| | | TTGATGTGCCATAGACAAGGTGGAGAGAGTGA | LITAITTNGAHPSKCVTIQRTLDGRLQVA |
| | | AACATTTGCAAAAAGAGCAATTGAAAGTTTGGT | GRKGFPHVIYARLWRWPDLHKNELKHV |
| | | AAAGAAGCTGAAGGAGAAAAAAGATGAATTGG | KYCQYAFDLKCDSVCVNPYHYERVVSP |
| | | ATTCTTTAATAACAGCTATAACTACAAATGGAG | GIDLSGLTLQSNAPSSMMVKDEYVHDFE |
| | | CTCATCCTAGTAAATGTGTTACCATACAGAGAA | GQPSLSTEGHSIQTIQHPPSNRASTETYST |
| | | CATTGGATGGGAGGCTTCAGGTGGCTGGTCGGA | PALLAPSESNATSTANFPNIPVASTSQPA |
| | | AAGGATTTCCTCATGTGATCTATGCCCGTCTCT | SILGGSHSEGLLQIASGPQPGQQQNGFTG |
| | | GGAGGTGGCCTGATCTTCACAAAAATGAACTA | QPATYHHNSTTTWTGSRTAPYTPNLPHH |
| | | AAACATGTTAAATATTGTCAGTATGCGTTTGAC | QNGHLQHHPPMPPHPGHYWPVHNELAF |
| | | TTAAAATGTGATAGTGTCTGTGTGAATCCATAT | QPPISNHPAPEYWCSIAYFEMDVQVGET |
| | | CACTACGAACGAGTTGTATCACCTGGAATTGAT | FKVPSSCPIVTVDGYVDPSGGDRFCLGQ |
| | | CTCTCAGGATTAACACTGCAGAGTAATGCTCCA | LSNVHRTEAIERARLHIGKGVQLECKGE |
| | | TCAAGTATGATGGTGAAGGATGAATATGTGCAT | GDVWVRCLSDHAVFVQSYYLDREAGR |
| | | GACTTTGAGGGACAGCCATCGTTGTCCACTGAA | APGDAVHKIYPSAYIKVFDLRQCHRQM |
| | | GGACATTCAATTCAAACCATCCAGCATCCACCA | QQQAATAQAAAAAQAAAVAGNIPGPGS |
| | | AGTAATCGTGCATCGACAGAGACATACAGCAC | VGGIAPAISLSAAAGIGVDDLRRLCILRM |
| | | CCCAGCTCTGTTAGCCCCATCTGAGTCTAATGC | SFVKGWGPDYPRQSIKETPCWIEIHLHR |
| | | TACCAGCACTGCCAACTTTCCCAACATTCCTGT | ALQLLDEVLHTMPIADPQPLD |
| | | GGCTTCCACAAGTCAGCCTGCCAGTATACTGGG | (SEQ ID NO: 56) |
| | | GGGCAGCCATAGTGAAGGACTGTTGCAGATAG | |
| | | CATCAGGGCCTCAGCCAGGACAGCAGCAGAAT | |
| | | GGATTTACTGGTCAGCCAGCTACTTACCATCAT | |
| | | AACAGCACTACCACCTGGACTGGAAGTAGGAC | |
| | | TGCACCATACACACCTAATTTGCCTCACCACCA | |
| | | AAACGGCCATCTTCAGCACCACCCGCCTATGCC | |
| | | GCCCCATCCCGGACATTACTGGCCTGTTCACAA | |
| | | TGAGCTTGCATTCCAGCCTCCCATTTCCAATCAT | |
| | | CCTGCTCCTGAGTATTGGTGTTCCATTGCTTACT | |
| | | TTGAAATGGATGTTCAGGTAGGAGAGACATTTA | |
| | | AGGTTCCTTCAAGCTGCCCTATTGTTACTGTTGA | |
| | | TGGATACGTGGACCCTTCTGGAGGAGATCGCTT | |
| | | TTGTTTGGGTCAACTCTCCAATGTCCACAGGAC | |
| | | AGAAGCCATTGAGAGAGCAAGGTTGCACATAG | |
| | | GCAAAGGTGTGCAGTTGGAATGTAAAGGTGAA | |
| | | GGTGATGTTTGGGTCAGGTGCCTTAGTGACCAC | |
| | | GCGGTCTTTGTACAGAGTTACTACTTAGACAGA | |
| | | GAAGCTGGGCGTGCACCTGGAGATGCTGTTCAT | |
| | | AAGATCTACCCAAGTGCATATATAAAGGTCTTT | |
| | | GATTTGCGTCAGTGTCATCGACAGATGCAGCAG | |
| | | CAGGCGGCTACTGCACAAGCTGCAGCAGCTGC | |
| | | CCAGGCAGCAGCCGTGGCAGGAAACATCCCTG | |
| | | GCCCAGGATCAGTAGGTGGAATAGCTCCAGCT | |
| | | ATCAGTCTGTCAGCTGCTGCTGGAATTGGTGTT | |
| | | GATGACCTTCGTCGCTTATGCATACTCAGGATG | |
| | | AGTTTTGTGAAAGGCTGGGGACCGGATTACCCA | |
| | | AGACAGAGCATCAAAGAAACACCTTGCTGGAT | |
| | | TGAAATTCACTTACACCGGGCCCTCCAGCTCCT | |
| | | AGACGAAGTACTTCATACCATGCCGATTGCAGA | |
| | | CCCACAACCTTTAGAC (SEQ ID NO: 24) | |
| NFATC1 | | ATGCCCAGCACTTCATTCCCCGTGCCCTCTAAA | MPSTSFPVPSKFPLGPAAAVFGRGETLGP |
| | | TTCCCCCTGGGTCCCGCAGCCGCCGTATTTGGT | APRAGGTMKSAEEEHYGYASSNVSPAL |
| | | CGCGGTGAGACCCTGGGCCCAGCACCAAGAGC | PLPTAHSTLPAPCHNLQTSTPGIIPPADHP |
| | | AGGTGGTACTATGAAAAGTGCAGAAGAGGAGC | SGYGAALDGGPAGYFLSSGHTRPDGAP |
| | | ATTACGGATACGCCAGTAGCAATGTGTCACCAG | ALESPRIEITSCLGLYHNNNQFFHDVEVE |
| | | CTCTCCCACTGCCTACTGCCCATAGCACGCTCC | DVLPSSKRSPSTATLSLPSLEAYRDPSCL |
| | | CTGCGCCTTGTCATAATCTGCAAACATCTACGC | SPASSLSSRSCNSEASSYESNYSYPYASP |
| | | CTGGAATTATACCCCCAGCCGACCATCCATCTG | QTSPWQSPCVSPKTTDPEEGFPRGLGAC |
| | | GCTATGGCGCCGCACTGGATGGTGGCCCAGCCG | TLLGSPRHSPSTSPRASVTEESWLGARSS |
| | | GGTATTTTCTGTCATCAGGGCATACTCGTCCGG | RPASPCNKRKYSLNGRQPPYSPHHSPTPS |
| | | ACGGAGCACCAGCACTCGAATCCCCGCGGATT | PHGSPRVSVTDDSWLGNTTQYTSSAIVA |
| | | GAAATCACTAGCTGTCTGGGACTCTATCATAAT | AINALTTDSSLDLGDGVPVKSRKTTLEQ |
| | | AACAATCAATTCTTTCATGACGTAGAAGTCGAG | PPSVALKVEPVGEDLGSPPPPADFAPEDY |
| | | GATGTACTGCCCTCTAGCAAGAGGTCACCAAGC | SSFQHIRKGGFCDQYLAVPQHPYQWAK |
| | | ACCGCTACTCTTTCTCTCCCATCCTTGGAAGCAT | PKPLSPTSYMSPTLPALDWQLPSHSGPYE |
| | | ATAGGGATCCAAGTTGTCTCTCTCCCGCTTCCTC | LRIEVQPKSHHRAHYETEGSRGAVKASA |
| | | ACTTAGCAGTAGAAGTTGTAATAGCGAAGCAA | GGHPIVQLHGYLENEPLMLQLFIGTADD |
| | | GCAGCTATGAATCAAATTATAGCTATCCCTATG | RLLRPHAFYQVHRITGKTVSTTSHEAILS |
| | | CATCACCACAAACAAGTCCCTGGCAATCCCCAT | NTKVLEIPLLPENSMRAVIDCAGILKLRN |
| | | GTGTTTCCCCTAAAACGACTGATCCAGAAGAAG | SDIELRKGETDIGRKNTRVRLVFRVHVP |
| | | GATTCCCAAGGGGACTTGGAGCTTGTACGCTCC | QPSGRTLSLQVASNPIECSQRSAQELPLV |
| | | TTGGATCACCCCGCCATAGTCCTAGTACTTCAC | EKQSTDSYPVVGGKKMVLSGHNFLQDS |
| | | CACGAGCATCCGTAACAGAAGAATCCTGGCTC | KVIFVEKAPDGHHVWEMEAKTDRDLCK |
| | | GGCGCGAGAAGCAGTCGGCCGGCCTCACCATG | PNSLVVEIPPFRNQRITSPVHVSFYVQNG |
| | | TAATAAACGGAAATATTCTCTTAATGGTAGGCA | KRKRSQYQRFTYLPANVPIIKTEPTDDYE |
| | | ACCACCATATAGTCCTCATCATTCCCCTACCCCT | PAPTCGPVSQGLSPLPRPYYSQQLAMPP |
| | | AGCCCCCATGGATCTCCCAGAGTGTCAGTCACT | DPSSCLVAGFPPCPQRSTLMPAAPGVSP |
| | | GATGATTCTTGGCTCGGGAATACAACGCAATAT | KLHDLSPAAYTKGVASPGHCHLGLPQP |
| | | ACATCCTCAGCAATTGTCGCGGCTATTAATGCT | AGEAPAVQDVPRPVATHPGSPGQPPPAL |
| | | CTCACGACAGATTCCAGTCTCGATCTCGGGGAC | LPQQVSAPPSSSCPPGLEHSLCPSSPSPPL |
| | | GGAGTGCCCGTGAAAAGCCGGAAAACAACACT | PPATQEPTCLQPCSPACPPATGRPQHLPS |
| | | CGAACAACCCCCATCTGTCGCACTTAAAGTCGA | TVRRDESPTAGPRLLPEVHEDGSPNLAPI |
| | | ACCTGTAGGAGAAGATCTCGGAAGTCCACCAC | PVTVKREPEELDQLYLDDVNEIIRNDLSS |
| | | CGCCTGCTGATTTTGCCCCTGAGGATTATTCTA | TSTHS (SEQ ID NO: 57) |
| | | GTTTTCAACATATTCGCAAAGGTGGGTTTTGTG | |
| | | ATCAATATTTGGCCGTCCCTCAACATCCTTATC | |
| | | AATGGGCCAAACCTAAACCGCTCAGCCCCACC | |
| | | AGCTATATGTCTCCCACGTTGCCAGCACTTGAT | |
| | | TGGCAACTCCCAAGCCATTCCGGGCCATACGAA | |
| | | CTCCGAATCGAAGTCCAACCGAAATCACATCAT | |
| | | CGCGCACATTATGAAACTGAAGGGTCACGTGG | |
| | | CGCTGTAAAAGCGTCCGCTGGCGGGCATCCAAT | |
| | | TGTCCAACTCCACGGGTATCTGGAAAACGAACC | |
| | | TTTGATGCTTCAACTCTTTATCGGAACCGCAGA | |
| | | TGATCGACTTCTCCGGCCACATGCATTTTATCA | |
| | | AGTTCATCGGATTACCGGAAAGACAGTAAGTA | |
| | | CGACTTCTCATGAAGCAATACTGAGTAATACTA | |
| | | AGGTGCTCGAAATTCCCCTTCTCCCAGAAAATA | |
| | | GTATGAGAGCTGTGATCGATTGCGCAGGTATTC | |
| | | TCAAGTTGAGGAATTCTGATATCGAGCTCAGGA | |
| | | AGGGCGAAACAGATATTGGACGTAAGAATACG | |
| | | CGCGTGCGACTCGTCTTTCGGGTGCATGTACCT | |
| | | CAGCCTAGTGGGCGGACTCTCAGCCTTCAAGTT | |
| | | GCAAGTAATCCGATTGAGTGTAGCCAAAGAAG | |
| | | TGCCCAAGAATTGCCGTTGGTCGAAAAGCAATC | |
| | | TACTGATTCCTACCCTGTAGTTGGTGGCAAGAA | |
| | | GATGGTACTCTCAGGACATAATTTTCTCCAAGA | |
| | | TTCTAAAGTGATCTTTGTCGAAAAGGCGCCCGA | |
| | | CGGTCATCACGTATGGGAAATGGAAGCTAAGA | |
| | | CCGATAGGGATCTCTGTAAACCAAACAGCCTTG | |
| | | TCGTCGAAATTCCGCCCTTCAGAAACCAACGTA | |
| | | TCACTTCTCCGGTGCATGTGTCATTTTATGTGTG | |
| | | TAATGGCAAACGCAAACGTTCCCAATATCAACG | |
| | | CTTTACATATTTGCCTGCGAATGTACCTATCATT | |
| | | AAGACCGAGCCAACCGACGACTACGAACCAGC | |
| | | CCCCACGTGCGGCCCTGTTTCCCAAGGCCTCTC | |
| | | ACCCCTGCCCCGCCCCTATTATAGTCAACAACT | |
| | | GGCAATGCCCCCTGATCCTTCTTCTTGTCTGGTC | |
| | | GCGGGATTTCCACCATGCCCCCAACGTTCTACT | |
| | | CTCATGCCCGCCGCTCCAGGTGTTAGTCCGAAA | |
| | | CTGCATGATCTGAGCCCTGCCGCATATACTAAA | |
| | | GGTGTGGCATCACCTGGTCATTGCCATCTGGGG | |
| | | CTGCCCCAACCCGCAGGCGAAGCTCCTGCTGTG | |
| | | CAAGATGTCCCTCGCCCTGTTGCTACACATCCA | |
| | | GGAAGTCCAGGCCAACCACCACCTGCGCTCTTG | |
| | | CCGCAACAAGTCTCAGCCCCACCGTCCTCTTCA | |
| | | TGTCCGCCCGGCCTGGAGCATAGTCTTTGTCCT | |
| | | TCTTCACCATCACCCCCGCTGCCACCAGCGACT | |
| | | CAGGAACCAACATGTCTCCAACCGTGTTCTCCC | |
| | | GCCTGTCCACCAGCAACCGGTAGGCCACAACAT | |
| | | CTCCCTAGCACCGTTAGGCGCGATGAATCCCCT | |
| | | ACAGCGGGCCCTAGGTTGCTCCCGGAAGTTCAC | |
| | | GAAGATGGGTCTCCCAACCTTGCTCCCATACCA | |
| | | GTGACCGTGAAAAGAGAACCAGAGGAACTGGA | |
| | | TCAACTGTATCTTGACGATGTTAACGAGATCAT | |
| | | CAGGAACGATCTGAGCTCTACATCAACACATTC | |
| | | T (SEQ ID NO: 25) | |
| EZH2 | | ATGGGCCAGACTGGGAAGAAATCTGAGAAGGG | MGQTGKKSEKGPVCWRKRVKSEYMRL |
| | | ACCAGTTTGTTGGCGGAAGCGTGTAAAATCAGA | RQLKRFRRADEVKSMFSSNRQKILERTEI |
| | | GTACATGCGACTGAGACAGCTCAAGAGGTTCA | LNQEWKQRRIQPVHILTSVSSLRGTRECS |
| | | GACGAGCTGATGAAGTAAAGAGTATGTTTAGTT | VTSDLDFPTQVIPLKTLNAVASVPIMYS |
| | | CCAATCGTCAGAAAATTTTGGAAAGAACGGAA | WSPLQQNFMVEDETVLHNIPYMGDEVL |
| | | ATCTTAAACCAAGAATGGAAACAGCGAAGGAT | DQDGTFIEELIKNYDGKVHGDRECGFIN |
| | | ACAGCCTGTGCACATCCTGACTTCTGTGAGCTC | DEIFVELVNALGQYNDDDDDDDGDDPE |
| | | ATTGCGCGGGACTAGGGAGTGTTCGGTGACCA | EREEKQKDLEDHRDDKESRPPRKFPSDK |
| | | GTGACTTGGATTTTCCAACACAAGTCATCCCAT | IFEAISSMFPDKGTAEELKEKYKELTEQQ |
| | | TAAAGACTCTGAATGCAGTTGCTTCAGTACCCA | LPGALPPECTPNIDGPNAKSVQREQSLHS |
| | | TAATGTATTCTTGGTCTCCCCTACAGCAGAATTT | FHTLFCRRCFKYDCFLHRKCNYSFHATP |
| | | TATGGTGGAAGATGAAACTGTTTTACATAACAT | NTYKRKNTETALDNKPCGPQCYQHLEG |
| | | TCCTTATATGGGAGATGAAGTTTTAGATCAGGA | AKEFAAALTAERIKTPPKRPGGRRRGRL |
| | | TGGTACTTTCATTGAAGAACTAATAAAAAATTA | PNNSSRPSTPTINVLESKDTDSDREAGTE |
| | | TGATGGGAAAGTACACGGGGATAGAGAATGTG | TGGENNDKEEEEKKDETSSSSEANSRCQ |
| | | GGTTTATAAATGATGAAATTTTTGTGGAGTTGG | TPIKMKPNIEPPENVEWSGAEASMFRVLI |
| | | TGAATGCCCTTGGTCAATATAatgatgatgacgatga | GTYYDNFCAIARLIGTKTCRQVYEFRVK |
| | | tgatgatgGAGACGATCCTGAAGAAAGAGAAGAAAAG | ESSIIAPAPAEDVDTPPRKKKRKHRLWA |
| | | CAGAAAGATCTGGAGGATCACCGAGATGATAA | AHCRKIQLKKDGSSNHVYNYQPCDHPR |
| | | AGAAAGCCGCCCACCTCGGAAATTTCCTTCTGA | QPCDSSCPCVIAQNFCEKFCQCSSECQNR |
| | | TAAAATTTTTGAAGCCATTTCCTCAATGTTTCCA | FPGCRCKAQCNTKQCPCYLAVRECDPD |
| | | GATAAGGGCACAGCAGAAGAACTAAAGGAAAA | LCLTCGAADHWDSKNVSCKNCSIQRGS |
| | | ATATAAAGAACTCACCGAACAGCAGCTCCCAG | KKHLLLAPSDVAGWGIFIKDPVQKNEFIS |
| | | GCGCACTTCCTCCTGAATGTACCCCCAACATAG | EYCGEIISQDEADRRGKVYDKYMCSFLF |
| | | ATGGACCAAATGCTAAATCTGTTCAGAGAGAG | NLNNDFVVDATRKGNKIRFANHSVNPN |
| | | CAAAGCTTACACTCCTTTCATACGCTTTTCTGTA | CYAKVMMVNGDHRIGIFAKRAIQTGEE |
| | | GGCGATGTTTTAAATATGACTGCTTCCTACATC | LFFDYRYSQADALKYVGIEREMEIP |
| | | GTAAGTGCAATTATTCTTTTCATGCAACACCCA | (SEQ ID NO: 58) |
| | | ACACTTATAAGCGGAAGAACACAGAAACAGCT | |
| | | CTAGACAACAAACCTTGTGGACCACAGTGTTAC | |
| | | CAGCATTTGGAGGGAGCAAAGGAGTTTGCTGCT | |
| | | GCTCTCACCGCTGAGCGGATAAAGACCCCACCA | |
| | | AAACGTCCAGGAGGCCGCAGAAGAGGACGGCT | |
| | | TCCCAATAACAGTAGCAGGCCCAGCACCCCCAC | |
| | | CATTAATGTGCTGGAATCAAAGGATACAGACA | |
| | | GTGATAGGGAAGCAGGGACTGAAACGGGGGGA | |
| | | GAGAACAATGATAaagaagaagaagagaagaaagaTG | |
| | | AAACTTCGAGCTCCTCTGAAGCAAATTCTCGGTGT | |
| | | CAAACACCAATAAAGATGAAGCCAAATATTGA | |
| | | ACCTCCTGAGAATGTGGAGTGGAGTGGTGCTGA | |
| | | AGCCTCAATGTTTAGAGTCCTCATTGGCACTTA | |
| | | CTATGACAATTTCTGTGCCATTGCTAGGTTAATT | |
| | | GGGACCAAAACATGTAGACAGGTGTATGAGTT | |
| | | TAGAGTCAAAGAATCTAGCATCATAGCTCCAGC | |
| | | TCCCGCTGAGGATGTGGATACTCCTCCAAGGAA | |
| | | AAAGAAGAGGAAACACCGGTTGTGGGCTGCAC | |
| | | ACTGCAGAAAGATACAGCTGAAAAAGGACGGC | |
| | | TCCTCTAACCATGTTTACAACTATCAACCCTGT | |
| | | GATCATCCACGGCAGCCTTGTGACAGTTCGTGC | |
| | | CCTTGTGTGATAGCACAAAATTTTTGTGAAAAG | |
| | | TTTTGTCAATGTAGTTCAGAGTGTCAAAACCGC | |
| | | TTTCCGGGATGCCGCTGCAAAGCACAGTGCAAC | |
| | | ACCAAGCAGTGCCCGTGCTACCTGGCTGTCCGA | |
| | | GAGTGTGACCCTGACCTCTGTCTTACTTGTGGA | |
| | | GCCGCTGACCATTGGGACAGTAAAAATGTGTCC | |
| | | TGCAAGAACTGCAGTATTCAGCGGGGCTCCAA | |
| | | AAAGCATCTATTGCTGGCACCATCTGACGTGGC | |
| | | AGGCTGGGGGATTTTTATCAAAGATCCTGTGCA | |
| | | GAAAAATGAATTCATCTCAGAATACTGTGGAG | |
| | | AGATTATTTCTCAAGATGAAGCTGACAGAAGA | |
| | | GGGAAAGTGTATGATAAATACATGTGCAGCTTT | |
| | | CTGTTCAACTTGAACAATGATTTTGTGGTGGAT | |
| | | GCAACCCGCAAGGGTAACAAAATTCGTTTTGCA | |
| | | AATCATTCGGTAAATCCAAACTGCTATGCAAAA | |
| | | GTTATGATGGTTAACGGTGATCACAGGATAGGT | |
| | | ATTTTTGCCAAGAGAGCCATCCAGACTGGCGAA | |
| | | GAGCTGTTTTTTGATTACAGATACAGCCAGGCT | |
| | | GATGCCCTGAAGTATGTCGGCATCGAAAGAGA | |
| | | AATGGAAATCCCT (SEQ ID NO: 26) | |
| EOMES | | ATGCAACTCGGAGAACAACTGCTCGTTAGTTCT | MQLGEQLLVSSVNLPGAHFYPLESARGG |
| | | GTCAATCTTCCCGGGGCACATTTCTATCCCCTC | SGGSAGHLPSAAPSPQKLDLDKASKKFS |
| | | GAATCAGCAAGGGGCGGGTCAGGTGGATCCGC | GSLSCEAVSGEPAAASAGAPAAMLSDT |
| | | CGGTCATCTGCCTTCTGCTGCTCCTTCCCCTCAA | DAGDAFASAAAVAKPGPPDGRKGSPCG |
| | | AAGCTGGATCTCGATAAGGCTAGCAAGAAATT | EEELPSAAAAAAAAAAAAAATARYSMD |
| | | CAGCGGATCCCTGTCATGTGAAGCAGTATCTGG | SLSSERYYLQSPGPQGSELAAPCSLFPYQ |
| | | TGAACCAGCTGCGGCGTCTGCTGGTGCTCCAGC | AAAGAPHGPVYPAPNGARYPYGSMLPP |
| | | CGCAATGTTGAGCGATACTGATGCAGGAGATG | GGFPAAVCPPGRAQFGPGAGAGSGAGG |
| | | CCTTCGCAAGTGCAGCAGCTGTCGCTAAACCAG | SSGGGGGPGTYQYSQGAPLYGPYPGAA |
| | | GACCACCCGATGGGAGAAAAGGGAGCCCGTGT | AAGSCGGLGGLGVPGSGFRAHVYLCNR |
| | | GGCGAAGAAGAATTGCCGTCTGCTGCCGCCGC | PLWLKFHRHQTEMIITKQGRRMFPFLSF |
| | | AGCGGCTGCTGCTGCTGCAGCCGCCGCCGCTAC | NINGLNPTAHYNVFVEVVLADPNHWRF |
| | | CGCCCGTTATTCTATGGATTCCTTGAGTAGCGA | QGGKWVTCGKADNNMQGNKMYVHPE |
| | | AAGGTATTATCTTCAAAGTCCTGGCCCGCAAGG | SPNTGSHWMRQEISFGKLKLTNNKGAN |
| | | TTCTGAATTGGCCGCCCCATGTAGCCTGTTTCCT | NNNTQMIVLQSLHKYQPRLHIVEVTEDG |
| | | TATCAAGCCGCTGCCGGCGCTCCTCATGGTCCC | VEDLNEPSKTQTFTFSETQFIAVTAYQNT |
| | | GTATATCCCGCCCCAAATGGCGCCAGATATCCA | DITQLKIDHNPFAKGFRDNYDSSHQIVPG |
| | | TATGGGTCAATGCTTCCCCCTGGTGGATTTCCT | GRYGVQSFFPEPFVNTLPQARYYNGERT |
| | | GCTGCTGTATGTCCCCCAGGACGGGCCCAATTT | VPQTNGLLSPQQSEEVANPPQRWLVTPV |
| | | GGGCCTGGGGCAGGGGCTGGTTCAGGGGCAGG | QQPGTNKLDISSYESEYTSSTLLPYGIKSL |
| | | TGGCTCTTCTGGTGGCGGCGGTGGGCCAGGTAC | PLQTSHALGYYPDPTFPAMAGWGGRGS |
| | | ATACCAATATTCACAAGGCGCCCCACTGTATGG | YQRKMAAGLPWTSRTSPTVFSEDQLSKE |
| | | TCCATATCCGGGCGCTGCTGCCGCTGGGAGCTG | KVKEEIGSSWIETPPSIKSLDSNDSGVYTS |
| | | TGGCGGCCTCGGCGGGCTTGGCGTGCCTGGAAG | ACKRRRLSPSNSSNENSPSIKCEDINAEE |
| | | CGGTTTTAGGGCACATGTGTATTTGTGTAATCG | YSKDTSKGMGGYYAFYTTP |
| | | ACCACTTTGGCTGAAGTTTCATAGGCATCAGAC | (SEQ ID NO: 59) |
| | | GGAAATGATAATCACTAAGCAAGGGCGAAGGA | |
| | | TGTTCCCATTTCTGTCCTTTAATATTAATGGTCT | |
| | | GAACCCAACCGCACATTATAACGTCTTTGTGGA | |
| | | AGTCGTCCTTGCAGATCCTAATCATTGGCGGTT | |
| | | TCAAGGCGGAAAGTGGGTTACGTGCGGAAAGG | |
| | | CGGATAACAATATGCAAGGGAATAAGATGTAC | |
| | | GTCCATCCTGAATCACCGAACACAGGGAGTCAT | |
| | | TGGATGAGGCAAGAAATAAGCTTTGGAAAGCT | |
| | | GAAGCTGACGAACAATAAGGGAGCCAACAATA | |
| | | ATAATACTCAAATGATCGTGCTTCAGTCACTTC | |
| | | ATAAGTATCAGCCAAGGCTTCACATAGTAGAG | |
| | | GTCACGGAAGACGGGGTCGAAGATCTGAACGA | |
| | | ACCATCCAAAACACAAACCTTCACATTTTCCGA | |
| | | GACCCAGTTTATCGCCGTCACAGCGTATCAGAA | |
| | | TACAGACATAACCCAGCTCAAAATAGACCACA | |
| | | ATCCTTTCGCCAAGGGATTTCGCGATAATTACG | |
| | | ACTCCTCACACCAAATAGTGCCCGGCGGCAGGT | |
| | | ATGGTGTGCAGAGTTTCTTTCCAGAACCGTTCG | |
| | | TGAATACATTGCCCCAGGCACGGTACTACAACG | |
| | | GGGAACGAACAGTCCCCCAAACTAATGGTTTGC | |
| | | TCAGCCCACAGCAATCCGAGGAAGTTGCAAAT | |
| | | CCGCCACAAAGATGGCTCGTAACTCCCGTGCAA | |
| | | CAGCCCGGCACGAATAAGCTGGATATATCTAGC | |
| | | TACGAGTCCGAGTACACAAGTTCCACCCTTCTT | |
| | | CCGTACGGGATCAAGAGCCTGCCACTGCAAAC | |
| | | CTCACACGCATTGGGCTACTATCCCGATCCCAC | |
| | | ATTCCCCGCCATGGCCGGCTGGGGCGGCAGAG | |
| | | GCTCATATCAACGCAAAATGGCCGCGGGTTTGC | |
| | | CCTGGACAAGCCGCACCAGTCCGACAGTGTTTT | |
| | | CAGAGGACCAACTGAGTAAAGAAAAGGTAAAG | |
| | | GAAGAGATCGGTTCAAGTTGGATCGAAACCCC | |
| | | ACCATCAATTAAGAGCCTCGACAGTAACGACA | |
| | | GCGGCGTGTATACTTCCGCCTGCAAAAGGAGAC | |
| | | GTCTCAGCCCCTCTAATTCTTCCAACGAGAACT | |
| | | CCCCGAGTATTAAATGCGAAGATATCAACGCA | |
| | | GAGGAATACAGCAAGGATACATCTAAGGGGAT | |
| | | GGGTGGCTACTACGCCTTCTATACTACACCT | |
| | | (SEQ ID NO: 27) | |
| SOX5 | | ATGCTTACTGACCCTGATTTACCTCAGGAGTTT | MLTDPDLPQEFERMSSKRPASPYGEADG |
| | | GAAAGGATGTCTTCCAAGCGACCAGCCTCTCCG | EVAMVTSRQKVEEEESDGLPAFHLPLHV |
| | | TATGGGGAAGCAGATGGAGAGGTAGCCATGGT | SFPNKPHSEEFQPVSLLTQETCGHRTPTS |
| | | GACAAGCAGACAGAAAGTGGAAGAAGAGGAG | QHNTMEVDGNKVMSSFAPHNSSTSPQK |
| | | AGTGACGGGCTCCCAGCCTTTCACCTTCCCTTG | ABEGGRQSGESLSSTALGTPERRKGSLA |
| | | CATGTGAGTTTTCCCAACAAGCCTCACTCTGAG | DVVDTLKQRKMEELIKNEPEETPSIEKLL |
| | | GAATTTCAGCCAGTTTCTCTGCTGACGCAAGAG | SKDWKDKLLAMGSGNFGEIKGTPESLA |
| | | ACTTGTGGCCATAGGACTCCCACTTCTCAGCAC | EKERQLMGMINQLTSLREQLLAAHDEQ |
| | | AATACAATGgAAGTTGATGGCAATAAAGTTATG | KKLAASQIEKQRQQMELAKQQQEQIAR |
| | | TCTTCATTTGCCCCACACAACTCATCTACCTCAC | QQQQLLQQQHKINLLQQQIQVQGQLPPL |
| | | CTCAGAAGGCAGAAGAAGGTGGGCGACAGAGT | MIPVFPPDQRTLAAAAQQGFLLPPGFSY |
| | | GGCGAGTCCTTGTCTAGTACAGCCCTGGGAACT | KAGCSDPYPVQLIPTTMAAAAAATPGLG |
| | | CCTGAACGGCGCAAGGGCAGTTTAGCTGATGTT | PLQLQQLYAAQLAAMQVSPGGKLPGIP |
| | | GTTGACACCTTGAAGCAGAGGAAAATGGAAGA | QGNLGAAVSPTSIHTDKSTNSPPPKSKDE |
| | | GCTCATCAAAAACGAGCCGGAAGAAACCCCCA | VAQPLNLSAKPKTSDGKSPTSPTSPHMP |
| | | GTATTGAAAAACTACTCTCAAAGGACTGGAAA | ALRINSGAGPLKASVPAALASPSARVSTI |
| | | GACAAGCTTCTTGCAATGGGATCGGGGAACTTT | GYLNDHDAVTKAIQEARQMKEQLRREQ |
| | | GGCGAAATAAAAGGGACTCCCGAGAGCTTAGC | QVLDGKVAVVNSLGLNNCRTEKEKTTL |
| | | TGAGAAAGAAAGGCAACTCATGGGTATGATCA | ESLTQQLAVKQNEEGKFSHAMMDENLS |
| | | ACCAGCTGACCAGCCTCCGAGAGCAGCTGTTGG | GDSDGSAGVSESRIYRESRGRGSNEPHIK |
| | | CTGCCCACGATGAGCAGAAGAAACTAGCTGCC | RPMNAFMVWAKDERRKILQAFPDMHN |
| | | TCTCAGATTGAGAAACAGCGTCAGCAAATGGA | SNISKILGSRWKAMTNLEKQPYYEEQAR |
| | | GCTGGCCAAGCAGCAACAAGAACAAATTGCAA | LSKQHLEKYPDYKYKPRPKRTCLVDGK |
| | | GACAGCAGCAGCAGCTTCTACAGCAACAACAC | KLRIGEYKAIMRNRRQEMRQYFNVGQQ |
| | | AAAATCAATTTGCTCCAGCAACAGATCCAGGTT | AQIPIATAGVVYPGAIAMAGMPSPHLPS |
| | | CAAGGTCAGCTGCCGCCATTAATGATTCCCGTA | EHSSVSSSPEPGMPVIQSTYGVKGEEPHI |
| | | TTCCCTCCTGATCAACGGACACTGGCTGCAGCT | KEEIQAEDINGEIYDEYDEEEDDPDVDY |
| | | GCCCAGCAAGGATTCCTCCTCCCTCCAGGCTTC | GSDSENHIAGQAN |
| | | AGCTATAAGGCTGGATGTAGTGACCCTTACCCT | (SEQ ID NO: 60) |
| | | GTTCAGCTGATCCCAACTACCATGGCAGCTGCT | |
| | | GCCGCAGCAACACCAGGCTTAGGCCCACTCCA | |
| | | ACTGCAGCAGTTATATGCTGCCCAGCTAGCTGC | |
| | | AATGCAGGTATCTCCAGGAGGGAAGCTGCCAG | |
| | | GCATACCCCAAGGCAACCTTGGTGCTGCTGTAT | |
| | | CTCCTACCAGCATTCACACAGACAAGAGCACA | |
| | | AACAGCCCACCACCCAAAAGCAAGGATGAAGT | |
| | | GGCACAGCCACTGAACCTATCAGCTAAACCCA | |
| | | AGACCTCTGATGGCAAATCACCCACATCACCCA | |
| | | CCTCTCCCCATATGCCAGCTCTGAGAATAAACA | |
| | | GTGGGGCAGGCCCCCTCAAAGCCTCTGTCCCAG | |
| | | CAGCGTTAGCTAGTCCTTCAGCCAGAGTTAGCA | |
| | | CAATAGGTTACTTAAATGACCATGATGCTGTCA | |
| | | CCAAGGCAATCCAAGAAGCTCGGCAAATGAAG | |
| | | GAGCAACTCCGACGGGAACAACAGGTGCTTGA | |
| | | TGGGAAGGTGGCTGTTGTGAATAGTCTGGGTCT | |
| | | CAATAACTGCCGAACAGAAAAGGAAAAAACAA | |
| | | CACTGGAGAGTCTGACTCAGCAACTGGCAGTTA | |
| | | AACAGAATGAAGAAGGAAAATTTAGCCATGCA | |
| | | ATGATGGATTTCAATCTGAGTGGAGATTCTGAT | |
| | | GGAAGTGCTGGAGTCTCAGAGTCAAGAATTTAT | |
| | | AGGGAATCCCGAGGGCGTGGTAGCAATGAACC | |
| | | CCACATAAAGCGTCCAATGAATGCCTTCATGGT | |
| | | GTGGGCTAAAGATGAACGGAGAAAGATCCTTC | |
| | | AAGCCTTTCCTGACATGCACAACTCCAACATCA | |
| | | GCAAGATATTGGGATCTCGCTGGAAAGCTATGA | |
| | | CAAACCTAGAGAAACAGCCATATTATGAGGAG | |
| | | CAAGCCCGTCTCAGCAAGCAGCACCTGGAGAA | |
| | | GTACCCTGACTATAAGTACAAGCCCAGGCCAA | |
| | | AGCGCACCTGCCTGGTGGATGGCAAAAAGCTG | |
| | | CGCATTGGTGAATACAAGGCAATCATGCGCAA | |
| | | CAGGCGGCAGGAAATGCGGCAGTACTTCAATG | |
| | | TTGGGCAACAAGCACAGATCCCCATTGCCACTG | |
| | | CTGGTGTTGTGTACCCTGGAGCCATCGCCATGG | |
| | | CTGGGATGCCCTCCCCTCACCTGCCCTCGGAGC | |
| | | ACTCAAGCGTGTCTAGCAGCCCAGAGCCTGGG | |
| | | ATGCCTGTTATCCAGAGCACTTACGGTGTGAAA | |
| | | GGAGAGGAGCCACATATCAAAGAAGAGATACA | |
| | | GGCCGAGGACATCAATGGAGAAATTTATGATG | |
| | | AGTACGACGAGGAAGAGGATGATCCAGATGTA | |
| | | GATTATGGGAGTGACAGTGAAAACCATATTGC | |
| | | AGGACAAGCCAAC (SEQ ID NO: 28) | |
| IRF2BP2 | | ATGGCTGCTGCTGTAGCCGTCGCTGCTGCTAGT | MAAAVAVAAASRRQSCYLCDLPRMPW |
| | | CGCCGCCAATCCTGTTATTTGTGCGATCTTCCG | AMIWDFTEPVCRGCVNYEGADRVEFVIE |
| | | AGGATGCCTTGGGCAATGATTTGGGATTTTACT | TARQLKRAHGCFPEGRSPPGAAASAAA |
| | | GAGCCTGTGTGTCGGGGTTGTGTGAATTATGAA | KPPPLSAKDILLQQQQQLGHGGPEAAPR |
| | | GGGGCAGATAGGGTGGAATTTGTGATTGAAAC | APQALERYPLAAAAERPPRLGSDFGSSR |
| | | TGCTAGGCAATTGAAAAGAGCCCATGGGTGTTT | PAASLAQPPTPQPPPVNGILVPNGFSKLE |
| | | TCCAGAAGGCAGGAGCCCGCCAGGTGCGGCTG | EPPELNRQSPNPRRGHAVPPTLVPLMNG |
| | | CAAGCGCTGCAGCAAAACCTCCTCCATTGTCAG | SATPLPTALGLGGRAAASLAAVSGTAAA |
| | | CGAAAGATATTCTGCTGCAACAACAACAACAA | SLGSAQPTDLGAHKRPASVSSSAAVEHE |
| | | CTCGGACATGGTGGACCAGAAGCCGCACCTCG | QREAAAKEKQPPPPAHRGPADSLSTAAG |
| | | GGCACCCCAAGCACTGGAAAGGTATCCTCTGGC | AAELSAEGAGKSRGSGEQDWVNRPKTV |
| | | AGCAGCTGCAGAACGGCCGCCAAGGCTTGGTT | RDTLLALHQHGHSGPFESKFKKEPALTA |
| | | CAGATTTTGGGTCTTCCCGACCTGCCGCCAGTC | GRLLGFEANGANGSKAVARTARKRKPS |
| | | TTGCTCAACCGCCTACCCCTCAACCTCCTCCTGT | PEPEGEVGPPKINGEAQPWLSTSTEGLKI |
| | | CAATGGTATTCTCGTACCTAATGGGTTTTCAAA | PMTPTSSFVSPPPPTASPHSNRTTPPEAA |
| | | ACTCGAAGAACCCCCAGAACTCAACAGGCAAT | QNGQSPMAALILVADNAGGSHASKDAN |
| | | CCCCAAATCCTAGAAGGGGACATGCTGTACCCC | QVHSTTRRNSNSPPSPSSMNQRRLGPRE |
| | | CTACTTTGGTTCCTTTGATGAATGGATCAGCTA | VGGQGAGNTGGLEPVHPASLPDSSLATS |
| | | CACCTTTGCCTACGGCCCTTGGACTGGGCGGTC | APLCCTLCHERLEDTHFVQCPSVPSHKF |
| | | GGGCGGCTGCTAGCCTCGCTGCTGTTAGCGGCA | CFPCSRQSIKQQGASGEVYCPSGEKCPL |
| | | CTGCAGCAGCATCTCTCGGTAGTGCTCAACCAA | VGSNVPWAFMQGEIATILAGDVKVKKE |
| | | CTGACCTCGGTGCACATAAACGCCCCGCCTCTG | RDS (SEQ ID NO: 61) |
| | | TCAGCAGTTCAGCCGCTGTTGAACATGAACAAA | |
| | | GGGAAGCAGCCGCGAAAGAAAAGCAGCCACCC | |
| | | CCACCAGCTCATAGGGGACCAGCAGATTCCCTT | |
| | | TCAACTGCCGCTGGTGCAGCAGAACTTTCCGCC | |
| | | GAGGGCGCCGGTAAATCCAGAGGCAGCGGGGA | |
| | | ACAAGATTGGGTTAATCGCCCCAAAACAGTTAG | |
| | | AGATACATTGCTTGCGCTCCATCAACATGGACA | |
| | | TTCCGGCCCATTTGAATCTAAATTCAAGAAAGA | |
| | | ACCTGCACTCACCGCTGGTAGACTCCTGGGCTT | |
| | | TGAAGCAAATGGCGCAAATGGATCCAAGGCTG | |
| | | TGGCCCGCACCGCTCGGAAGAGAAAACCGTCC | |
| | | CCCGAGCCCGAGGGAGAGGTTGGTCCACCCAA | |
| | | AATTAATGGCGAAGCGCAACCTTGGTTGAGTAC | |
| | | GTCTACCGAAGGTCTTAAAATACCTATGACACC | |
| | | CACCTCTAGTTTCGTCAGCCCGCCCCCACCAAC | |
| | | AGCGAGCCCCCACAGCAATCGCACGACTCCAC | |
| | | CCGAGGCCGCTCAAAACGGTCAATCACCTATGG | |
| | | CCGCACTCATACTTGTGGCTGATAACGCGGGTG | |
| | | GAAGCCACGCTAGTAAGGACGCAAATCAAGTG | |
| | | CATTCAACAACACGTCGGAACTCCAATTCCCCA | |
| | | CCATCCCCCAGCTCAATGAATCAGCGCCGACTT | |
| | | GGTCCAAGGGAAGTCGGCGGTCAAGGGGCCGG | |
| | | TAATACCGGCGGCTTGGAACCCGTTCATCCGGC | |
| | | GTCCCTTCCCGATAGTAGCCTCGCTACTTCTGC | |
| | | ACCACTCTGTTGTACGCTTTGTCATGAAAGATT | |
| | | GGAAGATACTCACTTCGTTCAATGTCCTAGTGT | |
| | | GCCATCCCATAAATTTTGTTTTCCCTGTAGTAGG | |
| | | CAGAGTATAAAGCAACAAGGCGCATCCGGGGA | |
| | | AGTGTACTGCCCGTCTGGCGAGAAGTGTCCGCT | |
| | | GGTCGGATCTAACGTTCCTTGGGCTTTCATGCA | |
| | | GGGTGAGATCGCTACAATTCTGGCCGGGGACGT | |
| | | TAAGGTTAAGAAGGAAAGGGATAGC (SEQ ID | |
| | | NO: 29) | |
| SOX3 | | ATGAGACCCGTCAGGGAAAATAGCTCTGGGGC | MRPVRENSSGARSPRVPADLARSILISLP |
| | | TCGCTCACCTCGCGTGCCCGCGGATCTTGCCCG | FPPDSLAHRPPSSAPTESQGLFTVAAPAP |
| | | AAGTATCCTGATCTCCCTGCCATTTCCACCCGA | GAPSPPATLAHLLPAPAMYSLLETELKN |
| | | TAGCCTCGCGCATCGGCCACCATCTAGCGCACC | PVGTPTQAAGTGGPAAPGGAGKSSANA |
| | | TACTGAATCTCAAGGGCTCTTTACAGTCGCTGC | AGGANSGGGSSGGASGGGGGTDQDRV |
| | | CCCCGCTCCCGGGGCCCCCTCACCCCCTGCTAC | KRPMNAFMVWSRGQRRKMALENPKMH |
| | | ATTGGCCCATCTGCTCCCTGCACCAGCTATGTA | NSEISKRLGADWKLLTDAEKRPFIDEAK |
| | | TAGTCTGCTCGAAACAGAGCTTAAGAATCCTGT | RLRAVHMKEYPDYKYRPRRKTKTLLKK |
| | | TGGCACTCCGACTCAGGCCGCTGGAACAGGTG | DKYSLPSGLLPPGAAAAAAAAAAAAAA |
| | | GACCAGCCGCTCCCGGCGGGGCCGGTAAATCCT | ASSPVGVGQRLDTYTHVNGWANGAYSL |
| | | CAGCAAATGCAGCTGGCGGGGCAAATAGCGGA | VQEQLGYAQPPSMSSPPPPPALPPMHRY |
| | | GGAGGATCCTCAGGCGGAGCCTCAGGTGGCGG | DMAGLQYSPMMPPGAQSYMNVAAAAA |
| | | TGGTGGAACCGATCAAGATAGAGTCAAGCGCC | AASGYGGMAPSATAAAAAAYGQQPAT |
| | | CTATGAATGCATTTATGGTCTGGAGTCGGGGTC | AAAAAAAAAAMSLGPMGSVVKSEPSSP |
| | | AAAGACGGAAGATGGCTCTCGAAAATCCAAAG | PPAIASHSQRACLGDLRDMISMYLPPGG |
| | | ATGCATAACTCAGAAATTTCTAAAAGACTGGGT | DAADAASPLPGGRLHGVHQHYQGAGT |
| | | GCGGATTGGAAGCTTTTGACGGATGCAGAGAA | AVNGTVPLTHI |
| | | AAGGCCCTTTATTGATGAAGCTAAAAGACTGAG | (SEQ ID NO: 62) |
| | | GGCTGTCCATATGAAAGAATACCCCGATTATAA | |
| | | ATATCGCCCTAGACGGAAAACCAAAACCCTCTT | |
| | | GAAGAAGGACAAATATAGCCTTCCTTCCGGGCT | |
| | | GCTCCCGCCAGGAGCAGCTGCGGCTGCGGCTGC | |
| | | AGCGGCCGCTGCTGCTGCCGCTGCGTCTTCCCC | |
| | | CGTTGGTGTTGGGCAACGGTTGGATACATATAC | |
| | | ACATGTAAATGGGTGGGCAAATGGAGCATATA | |
| | | GTCTCGTTCAAGAACAACTCGGGTATGCTCAAC | |
| | | CCCCTTCTATGAGTTCCCCACCCCCTCCTCCTGC | |
| | | ACTTCCACCAATGCATCGTTATGATATGGCTGG | |
| | | GCTTCAATATAGTCCCATGATGCCACCAGGTGC | |
| | | GCAATCTTATATGAATGTAGCCGCAGCCGCTGC | |
| | | AGCAGCATCCGGATATGGCGGAATGGCACCGT | |
| | | CTGCTACCGCCGCAGCAGCTGCTGCATATGGCC | |
| | | AACAACCAGCAACGGCGGCAGCAGCCGCCGCC | |
| | | GCTGCGGCTGCAATGAGTCTTGGGCCAATGGGA | |
| | | AGCGTGGTTAAAAGTGAACCATCATCACCGCCC | |
| | | CCTGCTATTGCATCCCATAGTCAACGTGCCTGT | |
| | | CTGGGAGATCTCCGGGATATGATATCTATGTAT | |
| | | CTGCCCCCGGGTGGCGATGCCGCTGATGCTGCT | |
| | | TCCCCCTTGCCGGGCGGACGGTTGCATGGTGTC | |
| | | CATCAACATTATCAAGGGGCAGGTACAGCCGTT | |
| | | AATGGGACAGTTCCCCTCACACATATT (SEQ ID | |
| | | NO: 30 | |
| PRDM1 | | ATGTTGGATATTTGCTTGGAAAAACGTGTGGGT | MLDICLEKRVGTTLAAPKCNSSTVRFQG |
| | | ACGACCTTGGCTGCCCCCAAGTGTAACTCCAGC | LAEGTKGTMKMDMEDADMTLWTEAEF |
| | | ACTGTGAGGTTTCAGGGATTGGCAGAGGGGAC | EEKCTYIVNDHPWDSGADGGTSVQAEA |
| | | CAAGGGGACCATGAAAATGGACATGGAGGATG | SLPRNLLFKYATNSEEVIGVMSKEYIPKG |
| | | CGGATATGACTCTGTGGACAGAGGCTGAGTTTG | TRFGPLIGEIYTNDTVPKNANRKYFWRI |
| | | AAGAGAAGTGTACATACATTGTGAACGACCAC | YSRGELHHFIDGFNEEKSNWMRYVNPA |
| | | CCCTGGGATTCTGGTGCTGATGGCGGTACTTCG | HSPREQNLAACQNGMNIYFYTIKPIPAN |
| | | GTTCAGGCGGAGGCATCCTTACCAAGGAATCTG | QELLVWYCRDFAERLHYPYPGELTMMN |
| | | CTTTTCAAGTATGCCACCAACAGTGAAGAGGTT | LTQTQSSLKQPSTEKNELCPKNVPKREY |
| | | ATTGGAGTGATGAGTAAAGAATACATACCAAA | SVKEILKLDSNPSKGKDLYRSNISPLTSE |
| | | GGGCACACGTTTTGGACCCCTAATAGGTGAAAT | KDLDDFRRRGSPEMPFYPRVVYPIRAPL |
| | | CTACACCAATGACACAGTTCCTAAGAACGCCAA | PEDFLKASLAYGIERPTYITRSPIPSSTTP |
| | | CAGGAAATATTTTTGGAGGATCTATTCCAGAGG | SPSARSSPDQSLKSSSPHSSPGNTVSPVGP |
| | | GGAGCTTCACCACTTCATTGACGGCTTTAATGA | GSQEHRDSYAYLNASYGTEGLGSYPGY |
| | | AGAGAAAAGCAACTGGATGCGCTATGTGAATC | APLPHLPPAFIPSYNAHYPKFLLPPYGMN |
| | | CAGCACACTCTCCCCGGGAGCAAAACCTGGCTG | CNGLSAVSSMNGINNFGLFPRLCPVYSN |
| | | CGTGTCAGAACGGGATGAACATCTACTTCTACA | LLGGGSLPHPMLNPTSLPSSLPSDGARRL |
| | | CCATTAAGCCCATCCCTGCCAACCAGGAACTTC | LQPEHPREVLVPAPHSAFSFTGAAASMK |
| | | TTGTGTGGTATTGTCGGGACTTTGCAGAAAGGC | DKACSPTSGSPTAGTAATAEHVVQPKAT |
| | | TTCACTACCCTTATCCCGGAGAGCTGACAATGA | SAAMAAPSSDEAMNLIKNKRNMTGYKT |
| | | TGAATCTCACACAAACACAGAGCAGTCTAAAG | LPYPLKKQNGKIKYECNVCAKTFGQLSN |
| | | CAACCGAGCACTGAGAAAAATGAACTCTGCCC | LKVHLRVHSGERPFKCQTCNKGFTQLA |
| | | AAAGAATGTCCCAAAGAGAGAGTACAGCGTGA | HLQKHYLVHTGEKPHECQVCHKRFSSTS |
| | | AAGAAATCCTAAAATTGGACTCCAACCCCTCCA | NLKTHLRLHSGEKPYQCKVCPAKFTQFV |
| | | AAGGAAAGGACCTCTACCGTTCTAACATTTCAC | HLKLHKRLHTRERPHKCSQCHKNYIHLC |
| | | CCCTCACATCAGAAAAGGACCTCGATGACTTTA | SLKVHLKGNCAAAPAPGLPLEDLTRINE |
| | | GAAGACGTGGGAGCCCCGAAATGCCCTTCTACC | EIEKFDISDNADRLEDVEDDISVISVVEK |
| | | CTCGGGTCGTTTACCCCATCCGGGCCCCTCTGC | EILAVVRKEKEETGLKVSLQRNMGNGL |
| | | CAGAAGACTTTTTGAAAGCTTCCCTGGCCTACG | LSSGCSLYESSDLPLMKLPPSNPLPLVPV |
| | | GGATCGAGAGACCCACGTACATCACTCGCTCCC | KVKQETVEPMDP |
| | | CCATTCCATCCTCCACCACTCCAAGCCCCTCTG | (SEQ ID NO: 63) |
| | | CAAGAAGCAGCCCCGACCAAAGCCTCAAGAGC | |
| | | TCCAGCCCTCACAGCAGCCCTGGGAATACGGTG | |
| | | TCCCCTGTGGGCCCCGGCTCTCAAGAGCACCGG | |
| | | GACTCCTACGCTTACTTGAACGCGTCCTACGGC | |
| | | ACGGAAGGTTTGGGCTCCTACCCTGGCTACGCA | |
| | | CCCCTGCCCCACCTCCCGCCAGCTTTCATCCCCT | |
| | | CGTACAACGCTCACTACCCCAAGTTCCTCTTGC | |
| | | CCCCCTACGGCATGAATTGTAATGGCCTGAGCG | |
| | | CTGTGAGCAGCATGAATGGCATCAACAACTTTG | |
| | | GCCTCTTCCCGAGGCTGTGCCCTGTCTACAGCA | |
| | | ATCTCCTCGGTGGGGGCAGCCTGCCCCACCCCA | |
| | | TGCTCAACCCCACTTCTCTCCCGAGCTCGCTGC | |
| | | CCTCAGATGGAGCCCGGAGGTTGCTCCAGCCGG | |
| | | AGCATCCCAGGGAGGTGCTTGTCCCGGCGCCCC | |
| | | ACAGTGCCTTCTCCTTTACCGGGGCCGCCGCCA | |
| | | GCATGAAGGACAAGGCCTGTAGCCCCACAAGC | |
| | | GGGTCTCCCACGGCGGGAACAGCCGCCACGGC | |
| | | AGAACATGTGGTGCAGCCCAAAGCTACCTCAG | |
| | | CAGCGATGGCAGCCCCCAGCAGCGACGAAGCC | |
| | | ATGAATCTCATTAAAAACAAAAGAAACATGAC | |
| | | CGGCTACAAGACCCTTCCCTACCCGCTGAAGAA | |
| | | GCAGAACGGCAAGATCAAGTACGAATGCAACG | |
| | | TTTGCGCCAAGACTTTCGGCCAGCTCTCCAATC | |
| | | TGAAGGTCCACCTGAGAGTGCACAGTGGAGAA | |
| | | CGGCCTTTCAAATGTCAGACTTGCAACAAGGGC | |
| | | TTTACTCAGCTCGCCCACCTGCAGAAACACTAC | |
| | | CTGGTACACACGGGAGAAAAGCCACATGAATG | |
| | | CCAGGTCTGCCACAAGAGATTTAGCAGCACCA | |
| | | GCAATCTCAAGACCCACCTGCGACTCCATTCTG | |
| | | GAGAGAAACCATACCAATGCAAGGTGTGCCCT | |
| | | GCCAAGTTCACCCAGTTTGTGCACCTGAAACTG | |
| | | CACAAGCGTCTGCACACCCGGGAGCGGCCCCA | |
| | | CAAGTGCTCCCAGTGCCACAAGAACTACATCCA | |
| | | TCTCTGTAGCCTCAAGGTTCACCTGAAAGGGAA | |
| | | CTGCGCTGCGGCCCCGGCGCCTGGGCTGCCCTT | |
| | | GGAAGATCTGACCCGAATCAATGAAGAAATCG | |
| | | AGAAGTTTGACATCAGTGACAATGCTGACCGGC | |
| | | TCGAGGACGTGGAGGATGACATCAGTGTGATCT | |
| | | CTGTAGTGGAGAAGGAAATTCTGGCCGTGGTCA | |
| | | GAAAAGAGAAAGAAGAAACTGGCCTGAAAGTG | |
| | | TCTTTGCAAAGAAACATGGGGAATGGACTCCTC | |
| | | TCCTCAGGGTGCAGCCTTTATGAGTCATCAGAT | |
| | | CTACCCCTCATGAAGTTGCCTCCCAGCAACCCA | |
| | | CTACCTCTGGTACCTGTAAAGGTCAAACAAGAA | |
| | | ACAGTTGAACCAATGGATCCT (SEQ ID NO: 31) | |
| RELB | | ATGCTCAGGTCAGGTCCCGCGTCAGGTCCAAGC | MLRSGPASGPSVPTGRAMPSRRVARPPA |
| | | GTTCCAACAGGGCGAGCGATGCCAAGCCGACG | APELGALGSPDLSSLSLAVSRSTDELEIID |
| | | GGTGGCTCGCCCACCCGCCGCACCCGAACTCGG | EYIKENGFGLDGGQPGPGEGLPRLVSRG |
| | | CGCTCTGGGATCTCCTGATCTGTCAAGTCTGTC | AASLSTVTLGPVAPPATPPPWGCPLGRL |
| | | ATTGGCTGTCAGTCGTAGTACTGACGAGCTTGA | VSPAPGPGPQPHLVITEQPKQRGMRFRY |
| | | AATTATTGATGAATATATTAAAGAAAATGGGTT | ECEGRSAGSILGESSTEASKTLPAIELRDC |
| | | TGGGTTGGATGGCGGCCAACCTGGTCCAGGAG | GGLREVEVTACLVWKDWPHRVHPHSL |
| | | AAGGACTCCCTAGGTTGGTCTCCCGGGGAGCCG | VGKDCTDGICRVRLRPHVSPRHSFNNLG |
| | | CCAGCTTGAGTACAGTGACACTCGGGCCAGTTG | IQCVRKKEIEAAIERKIQLGIDPYNAGSL |
| | | CCCCACCGGCTACTCCTCCTCCGTGGGGATGTC | KNHQEVDMNVVRICFQASYRDQQGQM |
| | | CACTTGGAAGACTGGTTAGCCCGGCTCCCGGAC | RRMDPVLSEPVYDKKSTNTSELRICRINK |
| | | CAGGACCCCAACCCCATCTTGTTATAACAGAAC | ESGPCTGGEELYLLCDKVQKEDISVVFS |
| | | AACCAAAACAAAGGGGAATGCGGTTTAGGTAT | RASWEGRADFSQADVHRQIAIVFKTPPY |
| | | GAATGTGAAGGGCGGTCTGCAGGGTCCATTCTG | EDLEIVEPVTVNVFLQRLTDGVCSEPLPF |
| | | GGTGAATCATCAACGGAAGCGTCAAAGACACT | TYLPRDHDSYGVDKKRKRGMPDVLGEL |
| | | CCCAGCAATTGAATTGAGGGACTGCGGCGGCCT | NSSDPHGIESKRRKKKPAILDHFLPNHGS |
| | | CAGAGAAGTCGAAGTAACCGCTTGTTTGGTCTG | GPFLPPSALLPDPDFFSGTVSLPGLEPPGG |
| | | GAAAGATTGGCCCCATAGGGTTCATCCGCATTC | PDLLDDGFAYDPTAPTLFTMLDLLPPAP |
| | | TCTGGTCGGAAAGGATTGTACAGATGGTATATG | PHASAVVCSGGAGAVVGETPGPEPLTLD |
| | | TCGGGTCAGACTGAGACCCCATGTGTCCCCTCG | SYQAPGPGDGGTASLVGSNMFPNHYRE |
| | | ACATTCATTCAATAATTTGGGTATTCAATGCGT | AAFGGGLLSPGPEAT |
| | | CCGTAAGAAAGAAATCGAAGCAGCGATCGAAA | (SEQ ID NO: 64) |
| | | GAAAGATACAGTTGGGGATAGATCCTTATAATG | |
| | | CAGGTAGCCTTAAGAATCACCAAGAGGTCGAT | |
| | | ATGAACGTCGTCCGCATATGTTTTCAAGCAAGC | |
| | | TACCGAGATCAACAAGGGCAAATGCGGCGAAT | |
| | | GGACCCGGTTCTCTCAGAACCTGTGTACGATAA | |
| | | GAAGAGCACTAATACTAGCGAACTTCGTATCTG | |
| | | TCGCATCAATAAAGAGTCAGGCCCATGTACAG | |
| | | GCGGGGAAGAATTGTATCTTCTGTGTGATAAAG | |
| | | TACAAAAGGAAGATATCTCCGTTGTTTTCTCCA | |
| | | GAGCTTCTTGGGAAGGCCGAGCCGATTTTAGTC | |
| | | AAGCTGATGTCCATAGGCAAATCGCTATCGTCT | |
| | | TTAAAACGCCCCCTTATGAAGATCTTGAAATCG | |
| | | TGGAACCGGTCACGGTAAATGTTTTCCTTCAAA | |
| | | GACTGACAGACGGCGTTTGTAGTGAACCCCTTC | |
| | | CCTTTACATATCTTCCCCGGGATCACGATTCCTA | |
| | | TGGGGTTGATAAGAAAAGAAAGAGAGGTATGC | |
| | | CTGATGTGCTGGGCGAACTCAATTCATCCGATC | |
| | | CTCACGGTATTGAATCCAAGAGGAGAAAGAAG | |
| | | AAACCAGCGATTTTGGATCATTTTCTCCCAAAT | |
| | | CATGGATCCGGGCCCTTTCTGCCCCCAAGTGCA | |
| | | CTCTTGCCGGATCCCGATTTCTTTAGCGGTACA | |
| | | GTCTCACTCCCTGGGTTGGAACCACCCGGTGGA | |
| | | CCCGATCTTCTCGATGACGGTTTCGCATATGAT | |
| | | CCCACTGCACCGACCCTGTTTACTATGCTTGAT | |
| | | CTCTTGCCACCCGCTCCACCTCATGCGAGTGCC | |
| | | GTGGTTTGTTCAGGTGGCGCGGGCGCTGTTGTG | |
| | | GGTGAAACACCGGGGCCCGAGCCTCTCACCTTG | |
| | | GATTCATATCAAGCACCCGGACCTGGTGACGGC | |
| | | GGTACGGCTTCCCTGGTCGGGTCTAATATGTTT | |
| | | CCTAACCACTATAGAGAAGCTGCATTCGGTGGT | |
| | | GGTCTGCTGAGTCCTGGTCCCGAGGCTACC | |
| | | (SEQ ID NO: 32) | |
| CTLA-4 | CD28 | ATGGCTTGCCTTGGATTTCAGGGGCACAAGGCTCAGC | MACLGFQRHKAQLNLATRTWPCTLLFF |
| | | TGAACCTGGCTACCAGGACCTGGCCCTGCACTCTCCT | LLFIPVFCKAMHVAQPAVVLASSRGIASF |
| | | GTTTTTTCTTCTCTTCATCCCTGTCTTCTGCAAAGCA | VCEYASPGKATEVRVTVLRQADSQVTE |
| | | ATGCACGTGGCCCAGCCTGCTGTGGTACTGGCCAGCA | VCAATYMMGNELTFLDDSICTGTSSGN |
| | | GCCGAGGCATCGCCAGCTTTGTGTGTGAGTATGCATC | QVNLTIQGLRAMDTGLYICKVELMYPPP |
| | | TCCAGGCAAAGCCACTGAGGTCCGGGTGACAGTGCTT | YYLGIGNGTQIYVIDPEPCPDSDFLLWIL |
| | | CGGCAGGCTGACAGCCAGGTGACTGAAGTCTGTGCGG | AAVSSGLFFYSFLLTAVSLSKMRSKRSR |
| | | CAACCTACATGATGGGGAATGAGTTGACCTTCCTAGA | LLHSDYMNMTPRRPGPTRKHYQPYAPP |
| | | TGATTCCATCTGCACGGGCACCTCCAGTGGAAATCAA | RDFAAYRS (SEQ ID NO: 99) |
| | | GTGAACCTCACTATCCAAGGACTGAGGGCCATGGACA | |
| | | CGGGACTCTACATCTGCAAGGTGGAGCTCATGTACCC | |
| | | ACCGCCATACTACCTGGGCATAGGCAACGGAACCCAG | |
| | | ATTTATGTAATTGATCCAGAACCGTGCCCAGATTCTG | |
| | | ACTTCCTCCTCTGGATCCTTGCAGCAGTTAGTTCGGG | |
| | | GTTGTTTTTTTATAGCTTTCTCCTCACAGCTGTTTCT | |
| | | TTGAGCAAAATGAGGAGTAAGAGGAGCAGGCTCCTGC | |
| | | ACAGTGACTACATGAACATGACTCCCCGCCGCCCCGG | |
| | | GCCCACCCGCAAGCATTACCAGCCCTATGCCCCACCA | |
| | | CGCGACTTCGCAGCCTATCGCTCC | |
| | | (SEQ ID NO: 98) | |
| CD200R | ICOS | ATGCTCTGCCCTTGGAGAACTGCTAACCTAGGGCTACT | MLCPWRTANLGLLLILTIFLVAASSSLC |
| | | GTTGATTTTGACTATCTTCTTAGTGGCCGCTTCAAGCA | MDEKQITQNYSKVLAEVNTSWPVKMAT |
| | | GTTTATGTATGGATGAAAAACAGATTACACAGAACTAC | NAVLCCPPIALRNLIIITWEILRGQPSCTK |
| | | TCGAAAGTACTCGCAGAAGTTAACACTTCATGGCCTGT | AYKKETNETKETNCTDERITWVSRPDQN |
| | | AAAGATGGCTACAAATGCTGTGCTTTGTTGCCCTCCTA | SDLQIRTVAITHDGYYRCIMVTPDGNFH |
| | | TCGCATTAAGAAATTTGATCATAATAACATGGGAAATA | RGYHLQVLVTPEVTLFQNRNRTAVCKA |
| | | ATCCTGAGAGGCCAGCCTTCCTGCACAAAAGCCTACAA | VAGKPAAHISWIPEGDCATKQEYWSNG |
| | | GAAAGAAACAAATGAGACCAAGGAAACCAACTGTACTG | TVTVKSTCHWEVHNVSTVTCHVSHLTG |
| | | ATGAGAGAATAACCTGGGTCTCCAGACCTGATCAGAAT | NKSLYIELLPVHIYESQLCCQLKFWLPIG |
| | | TCGGACCTTCAGATTCGTACCGTGGCCATCACTCATGA | CAAFVVVCILGCILICWLTKKKYSSSVH |
| | | CGGGTATTACAGATGCATAATGGTAACACCTGATGGGA | DPNGEYMFMRAVNTAKKSRLTDVTL |
| | | ATTTCCATCGTGGATATCACCTCCAAGTGTTAGTTACA | (SEQ ID NO: 101) |
| | | CCTGAAGTGACCCTGTTTCAAAACAGGAATAGAACTGC | |
| | | AGTATGCAAGGCAGTTGCAGGGAAGCCAGCTGCGCATA | |
| | | TCTCCTGGATCCCAGAGGGCGATTGTGCCACTAAGCAA | |
| | | GAATACTGGAGCAATGGCACAGTGACTGTTAAGAGTAC | |
| | | ATGCCACTGGGAGGTCCACAATGTGTCTACCGTGACCT | |
| | | GCCACGTCTCCCATTTGACTGGCAACAAGAGTCTGTAC | |
| | | ATAGAGCTACTTCCTGTTCATATTTATGAATCACAACT | |
| | | TTGTTGCCAGCTGAAGTTCTGGTTACCCATAGGATGTG | |
| | | CAGCCTTTGTTGTAGTCTGCATTTTGGGATGCATACTT | |
| | | ATTTGTTGGCTTACAAAAAAGAAGTATTCATCCAGTGT | |
| | | GCACGACCCTAACGGTGAATACATGTTCATGAGAGCAG | |
| | | TGAACACAGCCAAAAAATCTAGACTCACAGATGTGACC | |
| | | CTA (SEQ ID NO: 100) | |
| DR5 | CD28 | Atggaacaacggggacagaacgccccggccgcttcgg | MEQRGQNAPAASGARKRHGPGPREARG |
| | | gggcccggaaaaggcacggcccaggacccagggaggc | ARPGPRVPKTLVLVVAAVLLLVSAESAL |
| | | gcggggagccaggcctgggccccggtccccaagaccc | ITQQDLAPQQRAAPQQKRSSPSEGLCPP |
| | | ttgtgctcgttgtcgccgcggtcctgctgttggtctc | GHHISEDGRDCISCKYGQDYSTHWNDLL |
| | | agctgagtctgctctgatcacccaacaagacctagct | FCLRCTRCDSGEVELSPCTTTRNTVCQC |
| | | ccccagcagagagcggccccacaacaaaagaggtcca | EEGTFREEDSPEMCRKCRTGCPRGMVK |
| | | gcccctcagagggattgtgtccacctggacaccatat | VGDCTPWSDIECVHKESGTKHSGEVPAV |
| | | ctcagaagacggtagagattgcatctcctgcaaatat | EETVTSSPGTPASPCSLSGIIIGVTVAAVV |
| | | ggacaggactatagcactcactggaatgacctccttt | LIVAVFVCKSLLWKRSKRSRLLHSDYM |
| | | tctgcttgcgctgcaccaggtgtgattcaggtgaagt | NMTPRRPGPTRKHYQPYAPPRDFAAYRS |
| | | ggagctaagtccctgcaccacgaccagaaacacagtg | (SEQ ID NO: 103) |
| | | tgtcagtgcgaagaaggcaccttccgggaagaagatt | |
| | | ctcctgagatgtgccggaagtgccgcacagggtgtcc | |
| | | cagagggatggtcaaggtcggtgattgtacaccctgg | |
| | | agtgacatcgaatgtgtccacaaagaatcaggtacaa | |
| | | agcacagtggggaagtcccagctgtggaggagacggt | |
| | | gacctccagcccagggactcctgcctctccctgttct | |
| | | ctctcaggcatcatcataggagtcacagttgcagccg | |
| | | tagtcttgattgtggctgtgtttgtttgcaagtcttt | |
| | | actgtggaagAGGAGTAAGAGGAGCAGGCTCCTGCAC | |
| | | AGTGACTACATGAACATGACTCCCCGCCGCCCCGGG | |
| | | CCCACCCGCAAGCATTACCAGCCCTATGCCCCACCAC | |
| | | GCGACTTCGCAGCCTATCGCTCC | |
| | | (SEQ ID NO: 102) | |
| IL2RA | | ATGGATTCATACCTGCTGATGTGGGGACTGCTC | MDSYLLMWGLLTFIMVPGCQAELCDDD |
| | | ACGTTCATCATGGTGCCTGGCTGCCAGGCAGAG | PPEIPHATFKAMAYKEGTMLNCECKRGF |
| | | CTCTGTGACGATGACCCGCCAGAGATCCCACAC | RRIKSGSLYMLCTGNSSHSSWDNQCQCT |
| | | GCCACATTCAAAGCCATGGCCTACAAGGAAGG | SSATRNTTKQVTPQPEEQKERKTTEMQS |
| | | AACCATGTTGAACTGTGAATGCAAGAGAGGTTT | PMQPVDQASLPGHCREPPPWENEATERI |
| | | CCGCAGAATAAAAAGCGGGTCACTCTATATGCT | YHFVVGQMVYYQCVQGYRALHRGPAE |
| | | CTGTACAGGAAACTCTAGCCACTCGTCCTGGGA | SVCKMTHGKTRWTQPQLICTGEMETSQ |
| | | CAACCAATGTCAATGCACAAGCTCTGCCACTCG | FPGEEKPQASPEGRPESETSCLVTTTDFQI |
| | | GAACACAACGAAACAAGTGACACCTCAACCTG | QTEMAATMETSIFTTEYQVAVAGCVFLL |
| | | AAGAACAGAAAGAAAGGAAAACCACAGAAAT | ISVLLLSGLTWQRRQRKSRRTI |
| | | GCAAAGTCCAATGCAGCCAGTGGACCAAGCGA | (SEQ ID NO: 105) |
| | | GCCTTCCAGGTCACTGCAGGGAACCTCCACCAT | |
| | | GGGAAAATGAAGCCACAGAGAGAATTTATCAT | |
| | | TTCGTGGTGGGGCAGATGGTTTATTATCAGTGC | |
| | | GTCCAGGGATACAGGGCTCTACACAGAGGTCCT | |
| | | GCTGAGAGCGTCTGCAAAATGACCCACGGGAA | |
| | | GACAAGGTGGACCCAGCCCCAGCTCATATGCA | |
| | | CAGGTGAAATGGAGACCAGTCAGTTTCCAGGT | |
| | | GAAGAGAAGCCTCAGGCAAGCCCCGAAGGCCG | |
| | | TCCTGAGAGTGAGACTTCCTGCCTCGTCACAAC | |
| | | AACAGATTTTCAAATACAGACAGAAATGGCTG | |
| | | CAACCATGGAGACGTCCATATTTACAACAGAGT | |
| | | ACCAGGTAGCAGTGGCCGGCTGTGTTTTCCTGC | |
| | | TGATCAGCGTCCTCCTCCTGAGTGGGCTCACCT | |
| | | GGCAGCGGAGACAGAGGAAGAGTAGAAGAAC | |
| | | AATC (SEQ ID NO: 104) | |

1. A human T cell that heterologously expresses one or more polypeptides selected from the group consisting of:
a polypeptide comprising a human Fas extracellular domain or portion thereof linked to a human OX40 intracellular domain (and optionally, 1-10 (e.g., 7) amino acids of the Fas intracellular domain) via a transmembrane domain;
a polypeptide comprising a human TNFRSF12 extracellular domain linked to a human OX40 intracellular domain (and optionally 1-10 (e.g., 7) amino acids of the TNFRSF12 intracellular domain) via a transmembrane domain;
a polypeptide comprising a human LTBR extracellular domain linked to a human OX40 intracellular domain (and optionally 1-10 (e.g. 7) amino acids of the LTBR intracellular domain) via a transmembrane domain;
a truncated human LTBR protein comprising the human LTBR extracellular domain, transmembrane domain and about 1-10 (e.g. 7) amino acids of the intracellular domain,
a truncated human TNFRSF12 protein comprising the human TNFRSF12 extracellular domain, transmembrane domain and about 1-10 (e.g. 7) amino acids of the intracellular domain;
a polypeptide comprising a human LAG-3 extracellular domain linked to a human 4-1BB intracellular domain (and optionally 1-10 (e.g. 7) amino acids of the LAG3 intracellular domain) via a transmembrane domain;
a polypeptide comprising a human DR5 extracellular domain linked to a human IL-4R intracellular domain (and optionally 1-10 (e.g. 7) amino acids of the DR5 intracellular domain) via a transmembrane domain;
a polypeptide comprising a human DR4 extracellular domain linked to a human IL-4R intracellular domain (and optionally 1-10 (e.g. 7) amino acids of the DR4 intracellular domain) via a transmembrane domain;
a polypeptide comprising a human TNFRSF1A extracellular domain linked to a human IL-4R intracellular domain (and optionally 1-10 (e.g. 7) amino acids of the TNFRSF1A intracellular domain) via a transmembrane domain;
a polypeptide comprising a human LTBR extracellular domain linked to a human IL-4R intracellular domain (and optionally 1-10 (e.g. 7) amino acids of the LTBR intracellular domain) via a transmembrane domain;
a polypeptide comprising a human IL-4RA extracellular domain linked to a human ICOS intracellular domain via a transmembrane domain;
a polypeptide comprising a human LAG3 extracellular domain or a portion thereof (and optionally 1-20 amino acids of the ICOS extracellular domain) linked to a human ICOS intracellular domain via a transmembrane domain;
a polypeptide comprising a human CTLA4 extracellular domain or a portion thereof (and optionally 1-10 (e.g. 7) amino acids of the CTLA4 intracellular domain) linked to a human CD28 intracellular domain via a transmembrane domain;
a polypeptide comprising a human CD200R extracellular domain or a portion thereof (and optionally, the ICOS extracellular domain or a portion thereof) linked to a human ICOS intracellular domain via a transmembrane domain;
a polypeptide comprising a human DR5 extracellular domain or a portion thereof (and optionally 1-10 (e.g. 7) amino acids of the DR5 intracellular domain) linked to a human CD28 intracellular domain via a transmembrane domain;
a polypeptide comprising an IL21R protein, a LAT1 protein, a BATF protein, a BATF3 protein, a BATF2 protein, an ID2 protein, an ID3 protein, an IRF8 protein, a MYC protein, a POU2F1 protein, a TFAP4 protein, a SMAD4 protein, a NFATC1 protein, an EZH2 protein, an EOMES protein, a SOX5 protein, an IRF2BP2 protein, a SOX3 protein, a PRDM1 protein, or a RELB protein,
wherein the one or more polypeptides are encoded by a heterologous nucleic acid construct inserted into a target genomic locus of the cell, optionally wherein the target genomic locus is the T-cell receptor (TCR) locus of the cell, optionally wherein the heterologous nucleic acid construct is non-virally inserted.

2. The human T cell of claim 1, wherein the T cell heterologously expresses a polypeptide comprising an amino acid sequence that is at least 95% identical to an amino acid sequence selected from the group consisting of SEQ ID NO: 33-SEQ ID NO: 64, SEQ ID NO: 99, SEQ ID NO: 101, SEQ ID NO: 103 and SEQ ID NO: 105.

3. The human T cell of claim 1, wherein the target insertion site is in exon 1 of a TCR-alpha subunit constant gene (TRAC) or in exon 1 of a TCR-beta subunit constant gene (TRBC).

4. (canceled)

5. (canceled)

6. The human T cell of any one of claim 1, wherein the heterologous nucleic acid construct comprises a nucleic acid sequence that is at least 95% identical to a nucleic acid sequence selected from the consisting of SEQ ID NO: 1-32, 98, 100, 102 and 104.

7. The human T cell of claim 1, wherein the T cell expresses an antigen-specific T-cell receptor (TCR) or synthetic antigen receptor that recognizes a target antigen.

8. (canceled)

9. The human T cell of claim 1, wherein the T cell is a regulatory T cell, effector T cell, a memory T cell or naïve T cell.

10. (canceled)

11. (canceled)

12. The human T cell of claim 1, wherein the T cell is a primary cell.

13. The human T cell of claim 1, wherein the nucleic acid construct encodes:
(i) a first self-cleaving peptide sequence;
(ii) a first heterologous TCR subunit chain, wherein the TCR subunit chain comprises a variable region and a constant region of the TCR subunit;
(iii) a second self-cleaving peptide sequence;
(iv) a polypeptide sequence that is at least 95% identical to an amino acid sequence selected from the group consisting of SEQ ID NO: 33-SEQ ID NO: 64, SEQ ID NO: 99, SEQ ID NO: 101, SEQ ID NO: 103 and SEQ ID NO: 105;
(v) a third self-cleaving peptide sequence;
(vi) a variable region of a second heterologous TCR subunit chain; and
(vii) a portion of the N-terminus of the endogenous TCR subunit, wherein, if the endogenous TCR subunit of the cell is a TCR-alpha (TCR-α) subunit, the first heterologous TCR subunit chain is a heterologous TCR-beta (TCR-β) subunit chain and the second heterologous TCR subunit chain is a heterologous TCR-α subunit chain, and wherein if the endogenous TCR subunit of the cell is a TCR-β subunit, the first heterologous TCR subunit chain is a heterologous TCR-α subunit chain and the second heterologous TCR subunit chain is a heterologous TCR-β subunit chain.

14. The human T cell of claim 1, wherein the heterologous nucleic acid construct encodes
(i) a first self-cleaving peptide sequence;
(ii) a polypeptide sequence that is at least 95% identical to an amino acid sequence selected from the group consisting of SEQ ID NO: 33-SEQ ID NO: 64, SEQ ID NO: 99, SEQ ID NO: 101, SEQ ID NO: 103 and SEQ ID NO: 105;
(iii) a second self-cleaving peptide sequence;
(iv) a first heterologous TCR subunit chain, wherein the TCR subunit chain comprises a variable region and a constant region of the TCR subunit
(v) a third self-cleaving peptide sequence;
(vi) a variable region of a second heterologous TCR subunit chain; and
(vii) a portion of the N-terminus of the endogenous TCR subunit, wherein, if the endogenous TCR subunit of the cell is a TCR-alpha (TCR-α) subunit, the first heterologous TCR subunit chain is a heterologous TCR-beta (TCR-β) subunit chain and the second heterologous TCR subunit chain is a heterologous TCR-α subunit chain, and wherein if the endogenous TCR subunit of the cell is a TCR-β subunit, the first heterologous TCR subunit chain is a heterologous TCR-α subunit chain and the second heterologous TCR subunit chain is a heterologous TCR-β subunit chain.

15. The human T cell of claim 1, wherein the nucleic acid construct encodes, in the following order,
(i) a first self-cleaving peptide sequence;
(ii) a polypeptide sequence that is at least 95% identical to an amino acid sequence selected from the group consisting of SEQ ID NO: 33-SEQ ID NO: 64, SEQ ID NO: 99, SEQ ID NO: 101, SEQ ID NO: 103 and SEQ ID NO: 105;
(iii) a second self-cleaving peptide sequence;
(iv) a synthetic antigen receptor; and
(v) a third self-cleaving peptide sequence or a polyA sequence.

16. The human T cell of claim 1, wherein the nucleic acid construct encodes, in the following order,
(i) a first self-cleaving peptide sequence;
(ii) a synthetic antigen receptor;
(iii) a second self-cleaving peptide sequence;
(iv) a polypeptide sequence that is at least 95% identical to an amino acid sequence selected from the group consisting of SEQ ID NO: 33-SEQ ID NO: 64, SEQ ID NO: 99, SEQ ID NO: 101, SEQ ID NO: 103 and SEQ ID NO: 105; and
(v) a third self-cleaving peptide sequence or a polyA sequence.

17. (canceled)

18. A nucleic acid comprising a nucleic acid sequence encoding a polypeptide comprising an amino acid sequence at least 95% identical to a protein selected from the group consisting of: SEQ ID NO: 33, SEQ ID NO: 34, SEQ ID NO: 35, SEQ ID NO: 40, SEQ ID NO: 41, SEQ ID NO: 42, SEQ ID NO: 43, SEQ ID NO: 44, SEQ ID NO: 45 and SEQ ID NO: 46.

19. (canceled)

20. A human T cell comprising the nucleic acid of claim 18.

21. A nucleic acid construct that encodes in the following order,
(i) a first self-cleaving peptide sequence;
(ii) a first heterologous TCR subunit chain, wherein the TCR subunit chain comprises a variable region and a constant region of the TCR subunit;
(iii) a second self-cleaving peptide sequence;
(iv) a polypeptide sequence that is at least 95% identical to an amino acid sequence selected from the group consisting of SEQ ID NO: 33-SEQ ID NO: 64, SEQ ID NO: 99, SEQ ID NO: 101, SEQ ID NO: 103 and SEQ ID NO: 105;
(v) a third self-cleaving peptide sequence;
(vi) a variable region of a second heterologous TCR subunit chain; and
(vii) a portion of the N-terminus of an endogenous T-cell TCR subunit, wherein, if the endogenous TCR subunit is a TCR-alpha (TCR-α) subunit, the first heterologous TCR subunit chain is a heterologous TCR-beta (TCR-β) subunit chain and the second heterologous TCR subunit chain is a heterologous TCR-α subunit chain, and wherein if the endogenous TCR subunit is a TCR-β subunit, the first heterologous TCR subunit chain is a heterologous TCR-α subunit chain and the second heterologous TCR subunit chain is a heterologous TCR-β subunit chain.

22. The nucleic acid construct of claim 21, where the nucleic acid construct comprises a nucleic acid sequence that is at least 95% identical to a nucleic acid sequence selected from the group consisting of SEQ ID NO: 1-SEQ ID NO: 32, 98, 100, 102 and 104.

23. A method of modifying a human T cell comprising
(a) introducing into the human T cell
(i) a targeted nuclease that cleaves a target region in the TCR locus of a human T cell to create a target insertion site in the genome of the cell; and
(ii) a nucleic acid construct encoding one or more polypeptides selected from the group consisting of:
a polypeptide comprising a human Fas extracellular domain or portion thereof linked to a human OX40 intracellular domain (and optionally, 1-10 (e.g., 7) amino acids of the Fas intracellular domain) via a transmembrane domain;
a polypeptide comprising a human TNFRSF12 extracellular domain linked to a human OX40 intracellular domain (and optionally 1-10 (e.g., 7) amino acids of the TNFRSF12 intracellular domain) via a transmembrane domain;
a polypeptide comprising a human LTBR extracellular domain linked to a human OX44 intracellular domain (and optionally 1-10 (e.g. 7) amino acids of the LTBR intracellular domain) via a transmembrane domain;
a truncated human LTBR protein comprising the human LTBR extracellular domain, transmembrane domain and about 1-10 (e.g. 7) amino acids of the intracellular domain,
a truncated human TNFRSF12 protein comprising the human TNFRSF12 extracellular domain, transmembrane domain and about 1-10 (e.g. 7) amino acids of the intracellular domain;
a truncated human BTLA protein comprising the human BTLA extracellular domain, transmembrane domain and about 1-10 (e.g. 7) amino acids of the intracellular domain;
a polypeptide comprising a human LAG-3 extracellular domain linked to a human 4-1BB intracellular domain (and optionally 1-10 (e.g. 7) amino acids of the LAG3 intracellular domain) via a transmembrane domain;
a polypeptide comprising a human DR5 extracellular domain linked to a human IL-4R intracellular domain (and optionally 1-10 (e.g. 7) amino acids of the DR5 intracellular domain) via a transmembrane domain;
a polypeptide comprising a human DR4 extracellular domain linked to a human IL-4R intracellular domain (and optionally 1-10 (e.g. 7) amino acids of the DR4 intracellular domain) via a transmembrane domain;
a polypeptide comprising a human TNFRSF1A extracellular domain linked to a human IL-4R intracellular domain (and optionally 1-10 (e.g. 7) amino acids of the TNFRSF1A intracellular domain) via a transmembrane domain;
a polypeptide comprising a human LTBR extracellular domain linked to a human IL-4R intracellular domain (and optionally 1-10 (e.g. 7) amino acids of the LTBR intracellular domain) via a transmembrane domain;
a polypeptide comprising a human IL-4RA extracellular domain linked to a human ICOS intracellular domain via a transmembrane domain;
a polypeptide comprising a human LAG3 extracellular domain or a portion thereof (and optionally 1-20 amino acids of the ICOS extracellular domain) linked to a human ICOS intracellular domain via a transmembrane domain;
a polypeptide comprising a human CTLA4 extracellular domain or a portion thereof (and optionally 1-10 (e.g. 7) amino acids of the CTLA4 intracellular domain) linked to a human CD28 intracellular domain via a transmembrane domain,
a polypeptide comprising a human CD200R extracellular domain or a portion thereof (and optionally, the ICOS extracellular domain or a portion thereof) linked to a human ICOS intracellular domain via a transmembrane domain,
a polypeptide comprising a human DR5 extracellular domain or a portion thereof (and optionally 1-10 (e.g. 7) amino acids of the DR5 intracellular domain) linked to a human CD28 intracellular domain via a transmembrane domain;
a polypeptide comprising an IL21R protein, a LAT1 protein, a BATF protein, a BATF3 protein, a BATF2 protein, an ID2 protein, and ID3 protein, an IRF8 protein, a MYC protein, a POU2F1 protein, a TFAP4 protein, a SMAD4 protein, a NFATC1 protein, an EXH2 protein, an EOMES protein, a SOX5 protein, an IRF2BP2 protein, a SOX3 protein, a PRDM1 protein, IL2RA, or a RELB protein;
(b) allowing recombination to occur, thereby inserting the nucleic acid construct in the target insertion site to generate a modified human T cell.

24. The method of claim 23, wherein the polypeptide comprises an amino acid sequence at least 95% identical to a protein selected from the group consisting of SEQ ID NO: 33-SEQ ID NO: 64, SEQ ID NO: 99, SEQ ID NO: 101, SEQ ID NO: 103 and SEQ ID NO: 105.

25. (canceled)

26. The method of claim 23, wherein the target insertion site is in exon 1 of a TCR-alpha subunit constant gene (TRAC) or in exon 1 of a TCR-beta subunit constant gene (TRBC).

27. The method of claim 23, wherein the nucleic acid construct is inserted by introducing a viral vector comprising the nucleic acid construct into the cell.

28. The method of claim 23, wherein the targeted nuclease is selected from the group consisting of an RNA-guided nuclease domain, a transcription activator-like effector nuclease (TALEN), a zinc finger nuclease (ZFN) and a megaTAL.

29. The method of claim 28, wherein the targeted nuclease, a guide RNA and the DNA template are introduced into the cell as a ribonucleoprotein complex (RNP)-DNA template complex, wherein the RNP-DNA template complex comprises:
(i) the RNP, wherein the RNP comprises the targeted nuclease and the guide RNA; and
(ii) the nucleic acid construct.

30. The method of claim 22, wherein the T cell is a regulatory T cell, effector T cell, a memory T cell or naïve T cell.

31. (canceled)

32. (canceled)

33. The method of claim 22, wherein the cell is a primary cell.

34. A modified T cell produced by the method of claim 22.

35. A method of enhancing an immune response in a human subject comprising administering the T cell of claim 1 to the subject.

36. The method of claim 35, wherein the T cell expresses an antigen-specific TCR or synthetic antigen receptor that recognizes a target antigen in the subject.

37. The method of claim 35, wherein the human subject has cancer, an infection or an autoimmune disorder.

38. (canceled)

39. The method of claim 37, wherein the subject has cancer and the T cell expresses a polypeptide comprising an amino acid sequence that is at least 95% identical to Fas-OX40 (SEQ ID NO: 33), TNFRSF12-OX40 (SEQ ID NO: 34), LTBR-OX40 (SEQ ID NO: 35), LTBRtrunc (SEQ ID NO: 36), TNFRSF12trunc (SEQ ID NO: 37), IL-21R (SEQ ID NO: 38), LAT1 (SEQ ID NO: 39) BATF (SEQ ID NO: 47), BATF3 9 (SEQ ID NO: 48), BATF2 (SEQ ID NO: 49), ID2 (SEQ ID NO: 50), ID3 (SEQ ID NO: 51, IRF8 (SEQ ID NO: 52), MYC (SEQ ID NO: 53), POU2F1 (SEQ ID NO: 54), TFAP4 (SEQ ID NO: 55), or SMAD4 (SEQ ID NO: 56).

40. The method of claim 37, wherein the subject has cancer and wherein the T cell expresses a polypeptide comprising an amino acid sequence that is at least 95% identical to LAG3/4-1BB (SEQ ID NO: 40), DR5-IL-4R (SEQ ID NO: 41), DR4-IL-4R (SEQ ID NO: 42), TNFRSFIA-IL-4R (SEQ ID NO: 43), LTBR-IL-4R (SEQ ID NO: 44), IL-4RA-ICOS (SEQ ID NO: 45), LAG-3 ICOS (SEQ ID NO: 46), NFATC1 (SEQ ID NO: 57), EZH2 (SEQ ID NO: 58), EOMES (SEQ ID NO: 59), SOX5 (SEQ ID NO: 60), IRF2BP2 (SEQ ID NO: 61), SOX3 (SEQ ID NO: 62), PRDMI (SEQ ID NO: 63), or RELB (SEQ ID NO: 64).

41. (canceled)

42. The method of claim 35, wherein the subject has an infection and wherein the T cell expresses a polypeptide comprising an amino acid sequence that is at least 95% identical to Fas-OX40 (SEQ ID NO: 33), TNFRSF12-OX40 (SEQ ID NO: 34), LTBR-OX40 (SEQ ID NO: 35), LTBRtrunc (SEQ ID NO: 36), TNFRSF12trunc (SEQ ID NO: 37), IL-21R (SEQ ID NO: 38), LAT1 (SEQ ID NO: 39) BATF (SEQ ID NO: 47), BATF3 9 (SEQ ID NO: 48), BATF2 (SEQ ID NO: 49), ID2 (SEQ ID NO: 50), ID3 (SEQ ID NO: 51), IRF8 (SEQ ID NO: 52), MYC (SEQ ID NO: 53), POU2F1 (SEQ ID NO: 54), TFAP4 (SEQ ID NO: 55) or SMAD4 (SEQ ID NO: 56).

43. (canceled)

44. The method of claim 35, wherein the subject has an autoimmune disorder and wherein the T cell expresses a polypeptide comprising an amino acid sequence that is at least 95% identical to LAG3/4-1BB (SEQ ID NO: 40), DR5-IL-4R (SEQ ID NO: 41), DR4-IL-4R (SEQ ID NO: 42), TNFRSF1A-IL-4R (SEQ ID NO: 43), LTBR-IL-4R (SEQ ID NO: 44), IL-4RA-ICOS (SEQ ID NO: 45), LAG-3 ICOS (SEQ ID NO: 46), NFATC1 (SEQ ID NO: 57), EZH2 (SEQ ID NO: 58), EOMES (SEQ ID NO: 59), SOX5 (SEQ ID NO: 60), IRF2BP2 (SEQ ID NO: 61), SOX3 (SEQ ID NO: 62), PRDM1 (SEQ ID NO: 63), or RELB (SEQ ID NO: 64).

45. The method of claim 35, wherein the T-cell is autologous or allogenic.

46. (canceled)

47. The method of claim 35, wherein the T cell is an iPSC-derived T cell.
